SEQUENCE LISTING (3 sequences)

SEQ ID NO: 1 - RNA, 20 nt, synthetic construct
  source 1..20: mol_type = other RNA
  modified_base 3: mod_base = OTHER; note = thymine
  modified_base 5..6: mod_base = OTHER; note = thymine
  modified_base 16..17: mod_base = OTHER; note = thymine
  modified_base 19: mod_base = OTHER; note = thymine
ggtcttaggagagacttatg

SEQ ID NO: 2 - RNA, 20 nt, synthetic construct
  source 1..20: mol_type = other RNA
  modified_base 9: mod_base = OTHER; note = thymine
  modified_base 12: mod_base = OTHER; note = thymine
  modified_base 14: mod_base = OTHER; note = thymine
  modified_base 16: mod_base = OTHER; note = thymine
gaccgcaatggtatctgcaa

SEQ ID NO: 3 - RNA, 20 nt, synthetic construct
  source 1..20: mol_type = other RNA
  modified_base 2..3: mod_base = OTHER; note = thymine
  modified_base 6..8: mod_base = OTHER; note = thymine
  modified_base 12: mod_base = OTHER; note = thymine
attcatttccgtcggagaac

(19) United States
(12) Patent Application Publication
(10) Pub. No.: US 2025/0152611 A1
(43) Pub. Date: May 15, 2025

(54) IMMUNOMODULATORY LIPIDS AND USES THEREOF

(71) Applicants: President and Fellows of Harvard College, Cambridge, MA (US); The General Hospital Corporation, Boston, MA (US)

(72) Inventors: Jon Clardy, Cambridge, MA (US); Chelsi Danielle Cassilly, Cambridge, MA (US); Munhyung Bae, Cambridge, MA (US); Ramnik Xavier, Brookline, MA (US)

(73) Assignees: President and Fellows of Harvard College, Cambridge, MA (US); The General Hospital Corporation, Boston, MA (US)

(21) Appl. No.: 18/730,214

(22) PCT Filed: Jan. 18, 2023
(86) PCT No.: PCT/US2023/060846

§ 371 Date: Jul. 18, 2024

Related U.S. Application Data
(60) Provisional application No. 63/367,544, filed on Jul. 1, 2022.
(60) Provisional application No. 63/300,792, filed on Jan. 19, 2022.

(51) Int. Cl. A61K 31/685; A61K 39/39; A61K 45/06; A61P 37/04; C07F 9/10

(52) U.S. Cl. CPC A61K 31/685; A61K 39/39; A61K 45/06; A61P 37/04; C07F 9/106

(57) ABSTRACT

The development of new immunoregulatory small molecules represents a significant advance in immunotherapy. Provided herein are compounds, such as compounds of Formulae (I) and (II), and pharmaceutically acceptable salts, hydrates, solvates, polymorphs, co-crystals, tautomers, stereoisomers, isotopically labeled derivatives, and prodrugs thereof, and compositions, methods, uses, and kits that may be used in immunotherapy. The compounds provided herein are responsible for immunomodulatory signaling through the TLR2 receptor and are therefore useful for the treatment and/or prevention of various diseases (e.g., metabolic diseases, inflammatory diseases, immune disorders, or proliferative diseases).

### RELATED APPLICATIONS

This application claims priority under 35 U.S.C. § 119(e) to each of U.S. Provisional Application No. 63/300,792, filed Jan. 19, 2022, and U.S. Provisional Application No. 63/367,544, filed Jul. 1, 2022, the entire contents of each of which are incorporated herein by reference.

### GOVERNMENT SUPPORT

This invention was made with Government support under AT009708 awarded by the National Institutes of Health. The Government has certain rights in the invention.

### REFERENCE TO AN ELECTRONIC SEQUENCE LISTING

The contents of the electronic sequence listing (H082470402W000-SEQ-CLL.xml; Size: 9,535 bytes; and Date of Creation: Jan. 17, 2023) is herein incorporated by reference in its entirety.

### BACKGROUND

There are numerous correlations between gut microbes and host responses, but the responsible molecular mechanisms remain largely unknown. Akkermansia muciniphila, which was discovered in the human gut microbiota in 2004, appears prominently in these correlations (1). This Gram-negative obligate anaerobe comprises ˜3% of healthy human gut populations, and it is primarily known as a phylogenetic outlier for its ability to degrade mucin, the intestinal mucus layer separating the epithelial cells forming the intestinal wall from the intestine's contents (2). Its abundance is inversely correlated with diseases including inflammatory bowel disease and type 2 diabetes but positively correlated with, e.g., responses to PD-1/PD-L1 checkpoint inhibitors in cancer immunotherapy (3-5). The significance of these correlations gained additional salience when a report identified A. muciniphila's unusual ability to induce intestinal adaptive immune responses during homeostasis in a subset of T cells (6).

Bacteria make branched-chain fatty acids (BCFAs) to increase membrane fluidity, the same function unsaturated fatty acids (FAs) have in animals (9). Anteiso FAs increase fluidity more than iso FAs, and iso FAs increase fluidity over normal FAs (10). BCFAs are common in bacteria, including many pathogens, but they are rarely produced by humans or are produced at low levels (11). Interestingly, BCFAs in human serum, independent of a connection with A. muciniphila or any other bacteria, have been strongly associated with human health, for example, an anticorrelation with developing type 2 diabetes (12).

### SUMMARY OF THE INVENTION

Naturally occurring fatty acids play an important role in regulating human immune responses, with implications in, e.g., metabolic syndrome, type 2 diabetes, inflammatory bowel disease, cancer immunotherapy, and homeostatic immunity (3-6, 16). The present disclosure relates in part to compounds (e.g., phospholipids) that can regulate human immune responses. In certain embodiments, the compounds are not natural products. In certain embodiments, compounds of the present disclosure are based on fatty acids made by gut bacteria that are immunomodulatory.

In one aspect, provided herein are compounds (e.g., compounds of Formulae (I) and (II)), and pharmaceutically acceptable salts, solvates, hydrates, polymorphs, co-crystals, tautomers, stereoisomers, isotopically labeled derivatives, and prodrugs thereof, and pharmaceutical compositions thereof. In certain embodiments, the compounds provided herein are responsible for immunomodulatory signaling and can therefore be used for the treatment and/or prevention of diseases (e.g., metabolic diseases, inflammatory diseases, immune disorders, proliferative diseases).

The present disclosure also provides methods of using the compounds and compositions provided herein, e.g., for treating and/or preventing a metabolic disease, inflammatory disease, immune disorder, or proliferative disease in a subject. In certain embodiments, the present disclosure provides methods of using compounds and compositions provided herein for modulating an immune response in a subject or biological sample. In some embodiments, the present disclosure provides methods of using compounds and compositions provided herein for inducing a cytokine release in a cell of a subject or biological sample. In certain embodiments, the present disclosure provides methods of using compounds and compositions provided herein for activating TLR2 receptors in a subject or biological sample. In another aspect, the present disclosure also provides uses of the compounds and compositions provided herein for the preparation of medicaments. In another aspect, the present invention provides kits comprising a compound or composition provided herein.

In one aspect, the disclosure provides compounds of Formula (I):

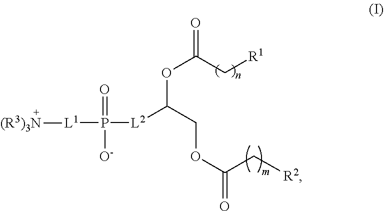

and pharmaceutically acceptable salts, solvates, hydrates, polymorphs, co-crystals, tautomers, stereoisomers, isotopically labeled derivatives, and prodrugs thereof, wherein L¹, L², R¹, R², R³ n, and m are as defined herein.

In certain embodiments, the compound of Formula (I) is not of one of the following formulae:

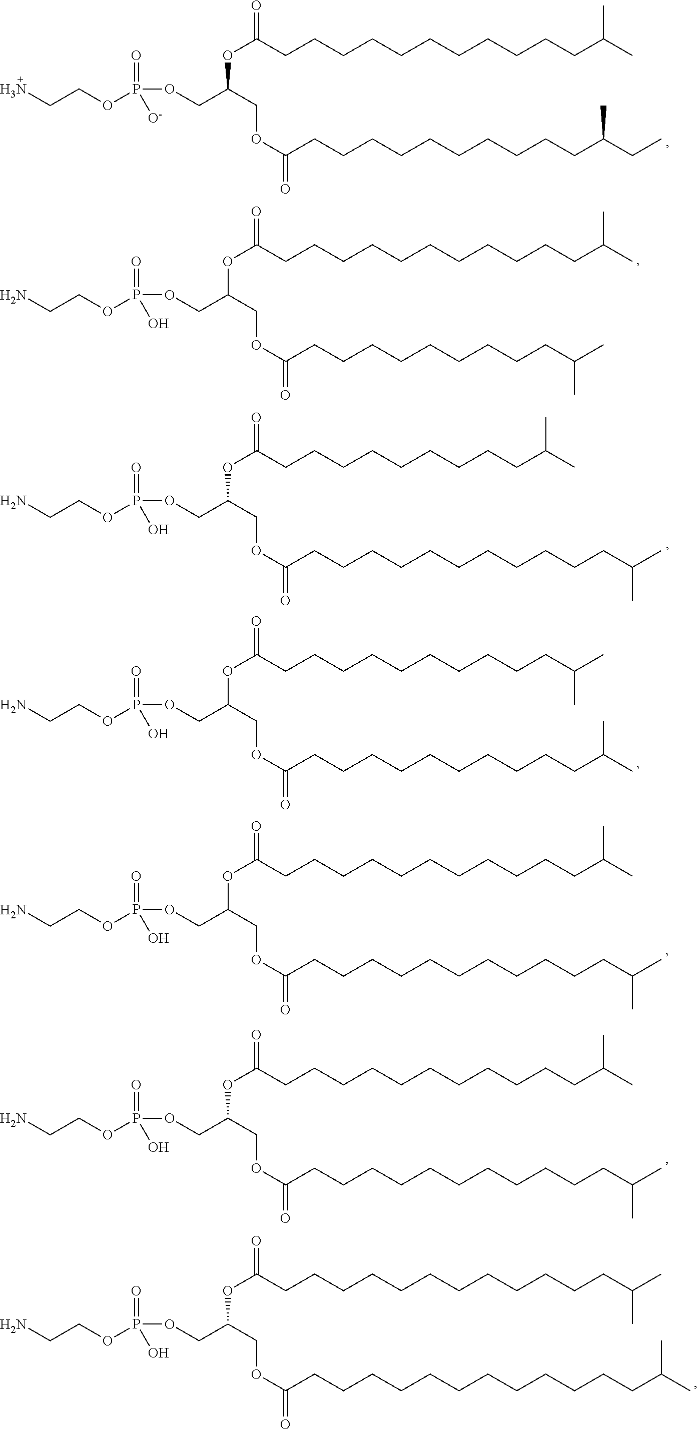

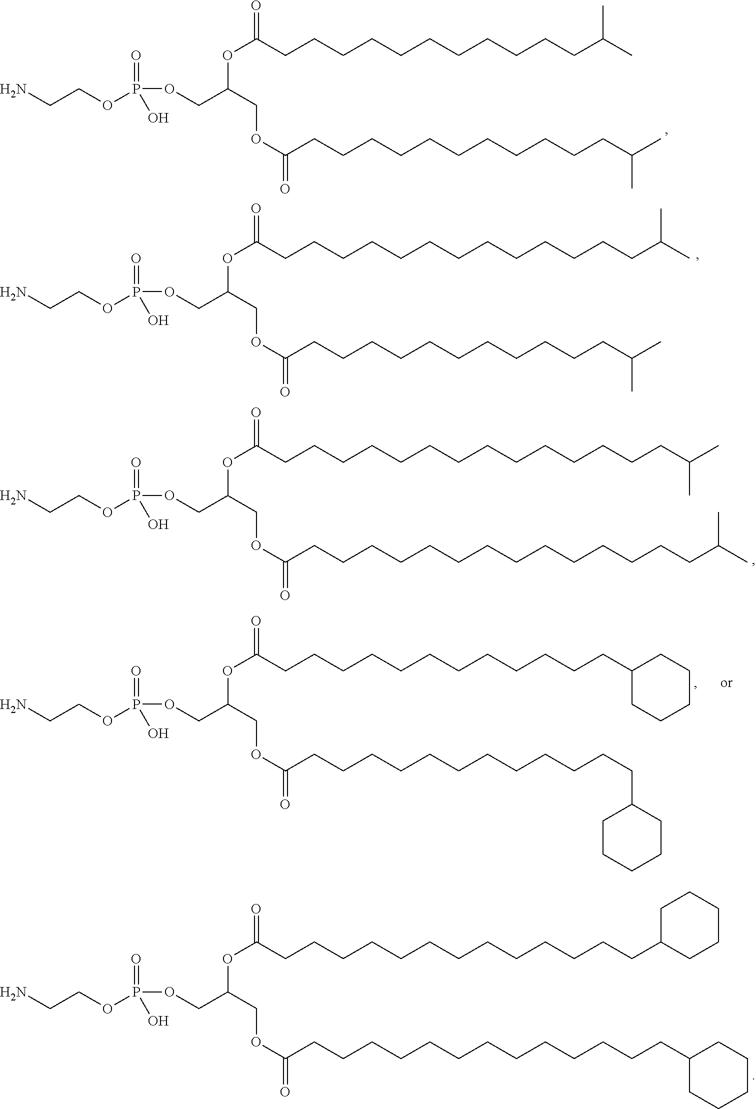

In certain embodiments, the compound of Formula (I) is of one of the foregoing formulae.

In certain embodiments, a compound of Formula (I) is of Formula (Ia):

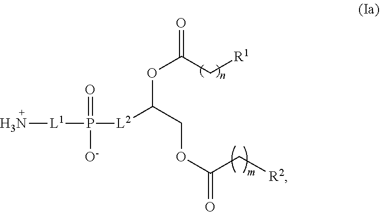

or a pharmaceutically acceptable salt, solvate, hydrate, polymorph, co-crystal, tautomer, stereoisomer, isotopically labeled derivative, or prodrug thereof, wherein L¹, L², R¹, R², n, and m are as defined herein.

In certain embodiments, a compound of Formula (I) is of Formula (Ib):

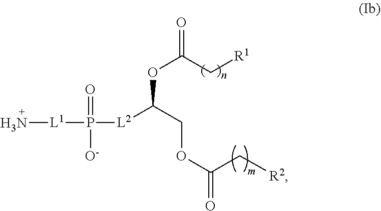

or a pharmaceutically acceptable salt, solvate, hydrate, polymorph, co-crystal, tautomer, stereoisomer, isotopically labeled derivative, or prodrug thereof, wherein L¹, L², R¹, R², n, and m are as defined herein.

In some embodiments, a compound of Formula (I) is of Formula (Ic):

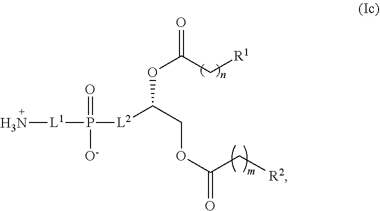

or a pharmaceutically acceptable salt, solvate, hydrate, polymorph, co-crystal, tautomer, stereoisomer, isotopically labeled derivative, or prodrug thereof, wherein L¹, L², R¹, R², n, and m are as defined herein.

In certain embodiments, a compound of Formula (I) is of Formula (Id):

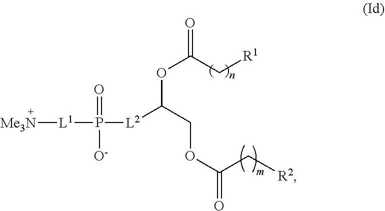

or a pharmaceutically acceptable salt, solvate, hydrate, polymorph, co-crystal, tautomer, stereoisomer, isotopically labeled derivative, or prodrug thereof, wherein L¹, L², R¹, R², n, and m are as defined herein.

In certain embodiments, a compound of Formula (I) is of Formula (Ie):

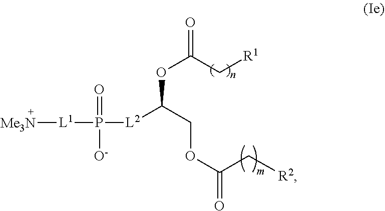

or a pharmaceutically acceptable salt, solvate, hydrate, polymorph, co-crystal, tautomer, stereoisomer, isotopically labeled derivative, or prodrug thereof, wherein L¹, L², R¹, R², n, and m are as defined herein.

In some embodiments, a compound of Formula (I) is of Formula (If):

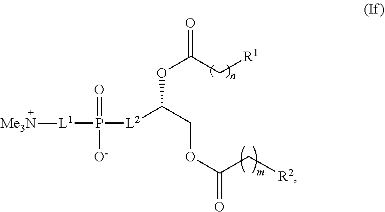

or a pharmaceutically acceptable salt, solvate, hydrate, polymorph, co-crystal, tautomer, stereoisomer, isotopically labeled derivative, or prodrug thereof, wherein L¹, L², R¹, R², n, and m are as defined herein.

In certain embodiments, a compound of Formula (I) is of one of the following formulae:

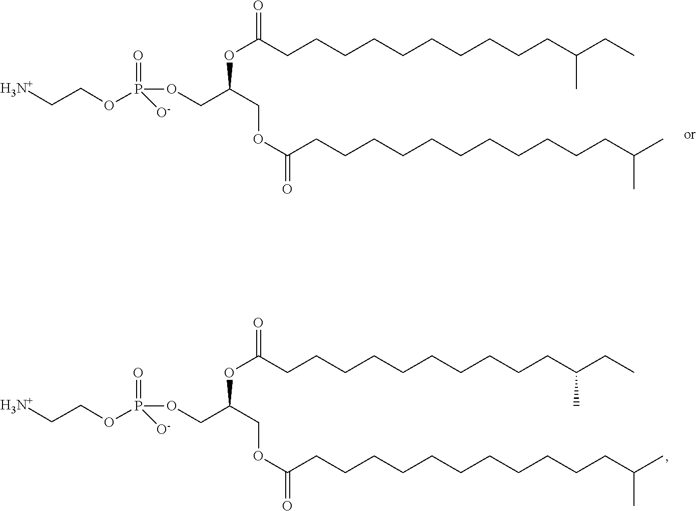 [SMILES: CCC(C)CCCCCCCCCCC(=O)O[C@@H](COC(=O)CCCCCCCCCCCC(C)C)COP(=O)([O-])OCC[NH3+] ; CC[C@H](C)CCCCCCCCCCC(=O)O[C@@H](COC(=O)CCCCCCCCCCCC(C)C)COP(=O)([O-])OCC[NH3+]]

or a pharmaceutically acceptable salt, solvate, hydrate, polymorph, co-crystal, tautomer, stereoisomer, isotopically labeled derivative, or prodrug thereof.

In another aspect, the disclosure provides compounds of Formula (II):

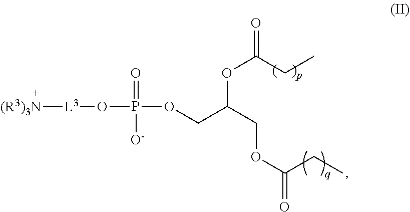

and pharmaceutically acceptable salts, solvates, hydrates, polymorphs, co-crystals, tautomers, stereoisomers, isotopically labeled derivatives, and prodrugs thereof, wherein R³, L³, p, and q are as defined herein.

In certain embodiments, the compound of Formula (II) is not of one of the following formulae:

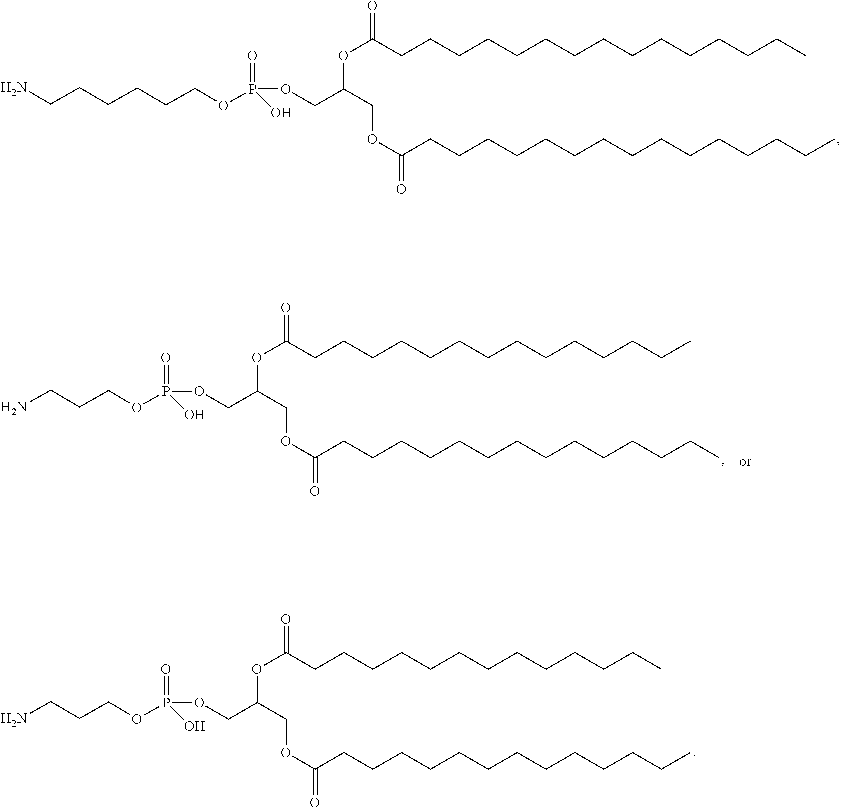 [SMILES: CCCCCCCCCCCCCCCC(=O)OCC(COP(=O)(O)OCCCCCCN)OC(=O)CCCCCCCCCCCCCCC ; CCCCCCCCCCCCCCC(=O)OCC(COP(=O)(O)OCCCN)OC(=O)CCCCCCCCCCCCCC ; CCCCCCCCCCCCCC(=O)OCC(COP(=O)(O)OCCCN)OC(=O)CCCCCCCCCCCCC]

In certain embodiments, the compound of Formula (II) is of one of the foregoing formulae.

In certain embodiments, a compound of Formula (II) is of Formula (IIa):

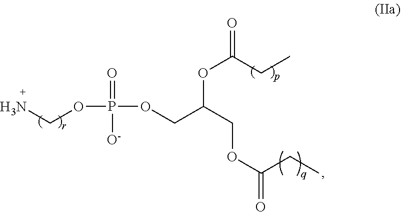

or a pharmaceutically acceptable salt, solvate, hydrate, polymorph, co-crystal, tautomer, stereoisomer, isotopically labeled derivative, or prodrug thereof, wherein r, p, and q are as defined herein.

In certain embodiments, a compound of Formula (II) is of Formula (IIb):

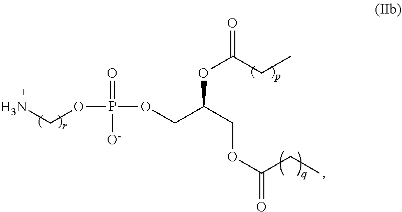

or a pharmaceutically acceptable salt, solvate, hydrate, polymorph, co-crystal, tautomer, stereoisomer, isotopically labeled derivative, or prodrug thereof, wherein r, p, and q are as defined herein.

In some aspects, a compound of Formula (II) is of Formula (IIc):

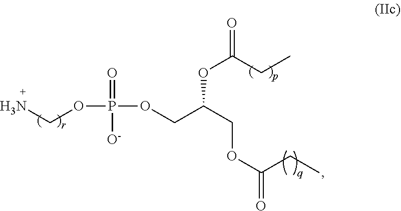

or a pharmaceutically acceptable salt, solvate, hydrate, polymorph, co-crystal, tautomer, stereoisomer, isotopically labeled derivative, or prodrug thereof, wherein r, p, and q are as defined herein.

In certain embodiments, a compound of Formula (II) is of Formula (IId):

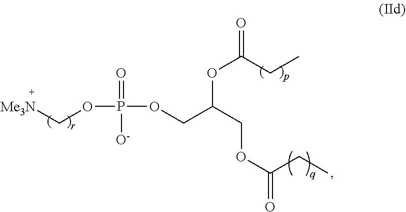

or a pharmaceutically acceptable salt, solvate, hydrate, polymorph, co-crystal, tautomer, stereoisomer, isotopically labeled derivative, or prodrug thereof, wherein r, p, and q are as defined herein.

In certain embodiments, a compound of Formula (II) is of Formula (IIe):

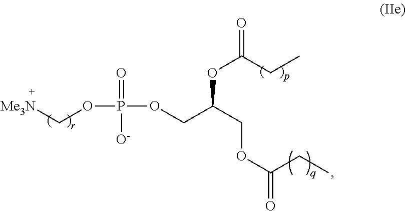

or a pharmaceutically acceptable salt, solvate, hydrate, polymorph, co-crystal, tautomer, stereoisomer, isotopically labeled derivative, or prodrug thereof, wherein r, p, and q are as defined herein.

In some aspects, a compound of Formula (II) is of Formula (IIf):

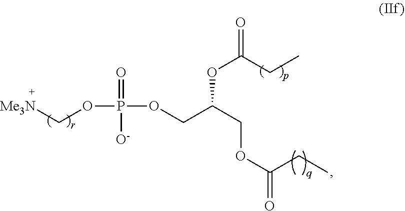

or a pharmaceutically acceptable salt, solvate, hydrate, polymorph, co-crystal, tautomer, stereoisomer, isotopically labeled derivative, or prodrug thereof, wherein r, p, and q are as defined herein.

In some embodiments, the present disclosure provides pharmaceutical compositions comprising a compound provided herein (e.g., a compound of Formula (I) or Formula (II)), or a pharmaceutically acceptable salt, solvate, hydrate, polymorph, co-crystal, tautomer, stereoisomer, isotopically labeled derivative, or prodrug thereof; and a pharmaceutically acceptable excipient. In certain embodiments, the pharmaceutical composition further comprises an additional pharmaceutical agent.

In another aspect, the present disclosure provides methods and uses for treating and/or preventing a disease (e.g., a metabolic disease, inflammatory disease, immune disorder, or proliferative disease) in a subject comprising administering to the subject a therapeutically effective amount of a compound provided herein (e.g., a compound of Formula (I) or Formula (II)), or a pharmaceutically acceptable salt, solvate, hydrate, polymorph, co-crystal, tautomer, stereoisomer, isotopically labelled derivative, or prodrug thereof, or a pharmaceutical composition thereof. In certain embodiments, the disease is a metabolic disease. In certain embodiments, the metabolic disease is type 2 diabetes. In some embodiments, the metabolic disease is metabolic syndrome. In certain embodiments, the disease is an inflammatory disease. In some embodiments, the disease is an immune disorder. In certain embodiments, the inflammatory disease is inflammatory bowel disease. In certain embodiments, the disease is a proliferative disease. In some embodiments, the proliferative disease is cancer.

In yet another aspect, the present disclosure provides methods and uses comprising contacting a cell, tissue, or biological sample with an effective amount of a compound provided herein (e.g., a compound of Formula (I) or Formula (II)), or a pharmaceutically acceptable salt, solvate, hydrate, polymorph, co-crystal, tautomer, stereoisomer, isotopically labeled derivative, or prodrug thereof, or a pharmaceutical composition thereof.

In further embodiments, the present disclosure provides kits comprising a compound provided herein (e.g., a compound of Formula (I) or Formula (II)), or a pharmaceutically acceptable salt, solvate, hydrate, polymorph, co-crystal, tautomer, stereoisomer, isotopically labeled derivative, or prodrug thereof; or a pharmaceutical composition provided herein; and instructions for using the compound, pharmaceutically acceptable salt, solvate, hydrate, polymorph, co-crystal, tautomer, stereoisomer, isotopically labeled derivative, prodrug, or pharmaceutical composition (e.g., for treating and/or preventing a disease in a subject, e.g., a metabolic disorder, inflammatory disease, immune disorder or proliferative disease).

The details of certain embodiments of the invention are set forth in the Detailed Description of Certain Embodiments, as described below. Other features, objects, and advantages of the invention will be apparent from the Definitions, Examples, Figures, and Claims. It should be understood that the aspects described herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

The accompanying drawings, which constitute a part of this specification, illustrate several embodiments of the invention and together with the description provide non-limiting examples of the invention.

FIGS. 1A-1G. Structural and Functional Analysis of A. muciniphila phosphoethanolamine (PE). FIG. 1A is a flow diagram for fractionation of A. muciniphila PE. Amounts in active fractions shown in red. Fatty acid composition of PE fraction also shown. FIG. 1B shows TNFα production by mouse bone marrow-derived dendritic cells (mBMDCs) treated with A. muciniphila lipid extract fractions as measured by enzyme-linked immunosorbent assay (ELISA). Fraction indicated in red was used for structural characterization. Pam3CysSerLys4 (Pam3CSK4) was used as a control agonist. Error bars=SD of technical replicates (n=4). FIG. 1C shows the structure of a15:0-i15:0 PE. FIG. 1D shows the relative abundance of fatty acids (FA) in A. muciniphila PE. FIG. 1E shows dose response of TNFα production by mBMDCs treated with a15:0-i15:0 PE as measured by ELISA. Error bars=SD of technical replicates (n=4). FIG. 1F shows a15:0-i15:0 PE and complete PE (AmPE) activate TNFα, IL-6, MCP-1, and IL-10 from mBMDCs as measured by flow cytometry. Pam3CSK4 and LPS were used as controls. Error bars=SD of technical replicates (n=4). FIG. 1G shows that TNFα release is lost in TLR2 knockout mBMDCs but not in TLR4 knockout mBMDCs as measured by ELISA. Pam3CSK4 was used as a TLR2 control agonist, and LPS was used as a TLR4 control agonist. Error bars=SD of technical replicates (n=4).

FIG. 2. Characterization of a15:0-i15:0 PE. Key COSY, HSQC, and HMBC correlations of a15:0-i15:0 PE.

FIGS. 3A-3E. Biosynthesis and laboratory synthesis of A. muciniphila PE. FIG. 3A shows key genes involved in putative biosynthetic pathway for A. muciniphila BAA-835 PE. Abbreviations: BCKDH, branched-chain alpha-keto acid dehydrogenase complex; GPAT, glycerol-3-phosphate acyl transferase; and AGPAT, acyl-glycerol-3-phosphate acyl transferase. FIG. 3B shows that leucine/isoleucine feeding increases TNFα induction by A. muciniphila in a TLR2-dependent fashion as measured by enzyme-linked immunosorbent assay (ELISA). Pam3CSK4 and LPS were used as controls. Error bars=SD of technical replicates (n=4). FIG. 3C shows an outline of synthetic scheme for a15:0-i15:0 PE and analogs thereof. FIG. 3D shows an overlay of mass spectrometric data from the natural and synthetic a15:0-i15:0. FIG. 3E shows TNFα induction by natural and synthetic a15:0-i15:0 PE. a15:0-i15:0 PE induces production in mBMDCs while n14:0-n14:0, n15:0-n15:0, n16:0-n16:0, a15:0-a15:0, and 15:0-415:0 PE have no detectable TNFα induction as measured by ELISA. However, i15:0-a15:0, the positional isomer, shows partial induction. Pam3CSK4 and LPS were used as controls. Error bars=SD of technical replicates (n=4).

FIGS. 4A-4C. Biosynthetic pathways in A. muciniphila and the TNFα activation of straight chain lipids. FIG. 4A shows proposed biosynthesis of a15:0-i15:0 PE in A. muciniphila BAA-835. FIG. 4B shows de novo biosynthesis of 1-leucine, 1-isoleucine, and 1-valine in A. muciniphila BAA-835. FIG. 4C shows TNFα induction in mBMDCs by single chain fatty acids from 13:0 to 17:0 as measured by ELISA assay. Pam3CSK4 and LPS were used as controls. Error bars=SD of technical replicates (n=4).

FIGS. 5A-5B. TLR2 Binding Model and T cell activation by a15:0-i15:0 PE. FIG. 5A shows IFNγ, IL-17A, IL-2, and IL-4 production from mBMDCs co-cultured with OVA-specific CD4+ T cells and OT-II peptides and treated with a15:0-i15:0 PE as measured by flow cytometry. LPS was used as a control. Error bars=SD of technical replicates (n=3). FIG. 5B shows three views of a15:0-i15:0 PE bound to TLR2 (left to right). View of TLR2 from (PDB 3a79) with modeled ligand, expanded view of ligand, further expanded view of the ligand tails showing the branches with C13 colored green and C12 purple.

FIG. 6. Human cell activation by PE of A. muciniphila. a15:0-i15:0 PE elicit TNFα, IL-6, IL-10, and MCP-1 from human myeloid cells as measured by flow cytometry.

FIGS. 7A-7G. TLR2-TLR1 binding model and T cell activation by a15:0-i15:0 PE. FIG. 7A shows a view of TLR2-TLR1 complex from Protein Data Bank (PDB ID: 2z7x) with modelled a15:0-i15:0 PE ligand in “bridging” conformation. FIG. 7B shows an overview of the modelled TLR2-TLR1-a15:0-i15:0 PE complex in surface representation. Dashed circle indicates buried lipid head group. FIG. 7C shows that TLR1 and TLR2 are required for natural and synthetic A. muciniphila lipid to induce TNFα production in human monocyte-derived dendritic cells (MDDCs). The production of TNFα was measured by ELISA 18 h after addition of natural or synthetic A. muciniphila lipids, Pam3CSK4, FSL-1, or LPS to cell culture media of human MDDCs following nucleofection. FIG. 7D shows IL-23A and IL-12B induction activity by natural and synthetic a15:0-i15:0 PE lipids. FIGS. 7E-7G show effects of treatment of human MDDCs with a15:0-i15:0 PE in combination with Pam3CSK4 or LPS. FIG. 7E shows a comparison of pre-18 hours and post-3 hours. FIG. 7F shows a comparison pre-3 hours and post-3 hours. FIG. 7G shows co-stimulation at 21 hours. With relatively long (18 h) delay times, low doses of a15:0-i15:0 PE suppress immune responses to Pam3CSK4 and moderate immune responses to LPS. Both effects disappear with shorter delay times (3 h in FIG. 7F or none in FIG. 7G). LPS and Pam3CSK4 were used at final concentrations of 100 ng/mL. Data in FIG. 7A (n=3), FIG. 7D (n=6), FIGS. 7E-G (n=4) are representative of two independent experiments and show mean values±SD. P-values in FIG. 7A were calculated by two-way analysis of variance (ANOVA) *P<0.05, **P<0.001, ****P<0.0001, ns, not significant.

FIG. 8. Heat-map of human derived monocyte activation by natural and synthetic A. muciniphila lipids. Monocytes were purified from peripheral blood by negative selection with magnetic beads. Cells were immediately placed in culture and stimulated with the indicated stimuli for 6 hours. After stimulation, cells were lysed for mRNA extraction and library preparation by SmartSeq2 for RNA sequencing. Monocytes were processed from two independent donors in technical triplicate. The final concentration of bacterial lipids was 50 g/ml. Pam3CSK4, FSL-1, and LPS at a final concentration of 100 ng/ml were used as controls.

FIGS. 9A-9F. Effects of treatment with synthetic A. muciniphila lipids in combination with Pam3CSK4 or LPS on human monocyte-derived dendritic cells. FIG. 9A shows a comparison of TNFα (pg/mL) at pre-3 hours and post-3 hours treatment with a15:0-i15:0 PE. FIG. 9B shows a comparison of TNFα (pg/mL) at pre-18 hours and post-3 hours treatment with a15:0-i15:0 PE. FIG. 9C shows TNFα (pg/mL) at 21 hours of co-stimulation with a15:0-i15:0 PE. FIG. 9D shows a comparison of TNFα (pg/mL) at pre-3 hours and post-3 hours treatment with i15:0-a15:0 PE. FIG. 9E shows a comparison of TNFα (pg/mL) at pre-18 hours and post-3 hours treatment with i15:0-a15:0 PE. FIG. 9F shows TNFα (pg/mL) at 21 hours of co-stimulation with i15:0-a15:0 PE. With long (18 h) delay times, low doses of synthetic a15:0-i15:0 PE (FIGS. 9A-9C) or i15:0-a15:0 PE (FIGS. 9D-9F) suppress immune responses to Pam3CSK4 and moderate immune responses to LPS. Both effects disappear with shorter delay times (3 h or none). LPS and Pam3CSK4 were used at final concentrations of 100 ng/mL. Data are presented as mean values±SD of technical replicates (n=4). Experiments were repeated independently at least twice with similar results.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂-6, C₂-5, C₂ 4, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (Cs) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

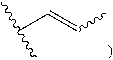

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has Ito 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₈ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₀ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

The term “branched” refers to an aliphatic group comprising one or more aliphatic substituents in a non-linear configuration. The term “unbranched” refers to a straight chain (i.e., linear) aliphatic group.

The term “fatty acid” refers to any organic moiety comprising carbon, hydrogen, and oxygen that is capable of forming an ester bond with an alcohol residue. Fatty acids may be branched or unbranched, or saturated or unsaturated. The terms “branched fatty acid” and “branched chain fatty acid” are used interchangeably.

The term “anteiso” is used to refer to aliphatic chain which has a branch at the antepenultimate carbon atom of the aliphatic chain. Anteiso fatty acids are a subclass of branched fatty acids.

The term “phospholipid” refers to a class of lipids having one or more hydrophobic tails and a phosphate head group. In some embodiments, phospholipids described herein are abbreviated, wherein “a” indicates an anteiso group, “i” indicates an isopropyl group, the number before each colon represents the number of carbon atoms in one of the hydrophobic tails, and the number after each colon represents the number of double bonds in the corresponding hydrophobic tail. For example, 12-methyltetradecanoyl-13-methyltetradecanoyl-sn-glycero-3-phosphoethanolamine is abbreviated a15:0-i15:0 PE.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, =NNR^{bb}C(═O)R^{aa}, ═NNR^{bb}C(═O)OR^{aa}, ═NNR^{bb}S(═O)₂R^{aa}, ═NR^{bb}, or ═NOR^{cc};

wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂Rc^{c}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{f})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff} C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff} SO₂R^{ee}, —SO₂N(R^{ff})₂, —SO₂R^{ee}, —SO₂OR^{cc}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or =S; wherein X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S=SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “oxo” refers to the group=0, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂— or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The disclosure is not intended to be limited in any manner by the above exemplary listing of substituents. Additional terms may be defined in other sections of this disclosure.

### Other Definitions

The following definitions are more general terms used throughout the present application.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The term “adjuvant” refers to a substance which, when added to an immunogenic agent (e.g., an antigen), non-specifically enhances or modulates an immune response to the agent in a subject upon administration. In certain embodiments, the immunogenic agent is an antigen. In some embodiments, the adjuvant enhances the immune response to the agent such that less of the agent may be used. In certain embodiments, the adjuvant modulates an immune response in immune cells. In some embodiments, the adjuvant supports the production of cytokines. In certain embodiments, the adjuvant is a vaccine adjuvant.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severeity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for modulating an immune response in a subject. In certain embodiments, a therapeutically effective amount is an amount sufficient for treating a disease (e.g., a metabolic disorder, an inflammatory disease, or a proliferative disease) in a subject. In certain embodiments, a therapeutically effective amount is an amount sufficient for modulating an immune response and treating a disease (e.g., a metabolic disorder, an inflammatory disease, or a proliferative disease) in a subject.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for modulating an immune response in a subject. In certain embodiments, a prophylactically effective amount is an amount sufficient for preventing a disease (e.g., a metabolic disorder, an inflammatory disease, or a proliferative disease) in a subject. In certain embodiments, a prophylactically effective amount is an amount sufficient for modulating an immune response and preventing a disease (e.g., a metabolic disorder, an inflammatory disease, or a proliferative disease) in a subject.

Anti-cancer agents encompass biotherapeutic anti-cancer agents as well as chemotherapeutic agents.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g., HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), platinum containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer.

Immune disorders, such as auto-immune disorders, include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

In certain embodiments, the inflammatory disorder and/or the immune disorder is a gastrointestinal disorder. In some embodiments, the gastrointestinal disorder is selected from gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)). In certain embodiments, the gastrointestinal disorder is inflammatory bowel disease (IBD).

In certain embodiments, the inflammatory condition and/or immune disorder is a skin condition. In some embodiments, the skin condition is pruritus (itch), psoriasis, eczema, burns or dermatitis. In certain embodiments, the skin condition is psoriasis. In certain embodiments, the skin condition is pruritis.

The terms “metabolic disorder” and “metabolic disease” are used interchangeably. The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### DETAILED DESCRIPTION OF CERTAIN EMBODIMENTS

Provided herein are compounds (e.g., compounds of Formulae (I) and (II)), and pharmaceutically acceptable salts, solvates, hydrates, polymorphs, co-crystals, tautomers, stereoisomers, isotopically labeled derivatives, or prodrugs thereof, and pharmaceutical compositions and kits thereof. Also provided herein are methods of treating and/or preventing a disease (e.g., a metabolic disease, inflammatory disease, immune disorder, or proliferative disease) in a subject comprising administering a therapeutically effective amount of a compound or composition provided herein to the subject. The compound or composition may be administered as a monotherapy or in combination with another therapy, as described herein. Other uses of the compounds and pharmaceutical compoisitions provided herein include methods of modulating an immune response or cytokine release (e.g., in a subject or biological sample).

### Compounds

Provided herein are compounds of Formula (I):

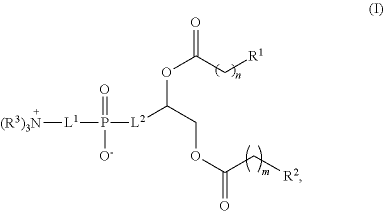

and pharmaceutically acceptable salts, solvates, hydrates, polymorphs, co-crystals, tautomers, stereoisomers, isotopically labeled derivatives, and prodrugs thereof, wherein:

R¹ is branched or unbranched C₁₋₄ alkyl, or C₃₋₇ carbocyclic;

R² is branched or unbranched C₁₋₄ alkyl, or C₃₋₇ carbocyclic;

at least one of R¹ and R² is branched C₁₋₄ alkyl, or C₃₋₇ carbocyclic;

R³ is —H or branched or unbranched C₁₋₆ alkyl;

L¹ is substituted or unsubstituted C₁₋₅ alkylene, substituted or unsubstituted C₁₋₈ heteroalkylene, substituted or unsubstituted carbocyclylene, substituted or unsubstituted heterocyclylene, substituted or unsubstituted arylene, substituted or unsubstituted heteroarylene, or any combination thereof;

L² is unbranched and unsubstituted C₁₋₆ heteroalkylene, or unbranched and unsubstituted C₁₋₆ alkylene;

n is an integer from 7 to 13, inclusive; and

m is an integer from 7 to 13, inclusive.

In certain embodiments, a compound of Formula (I) is not of one of the following formulae:

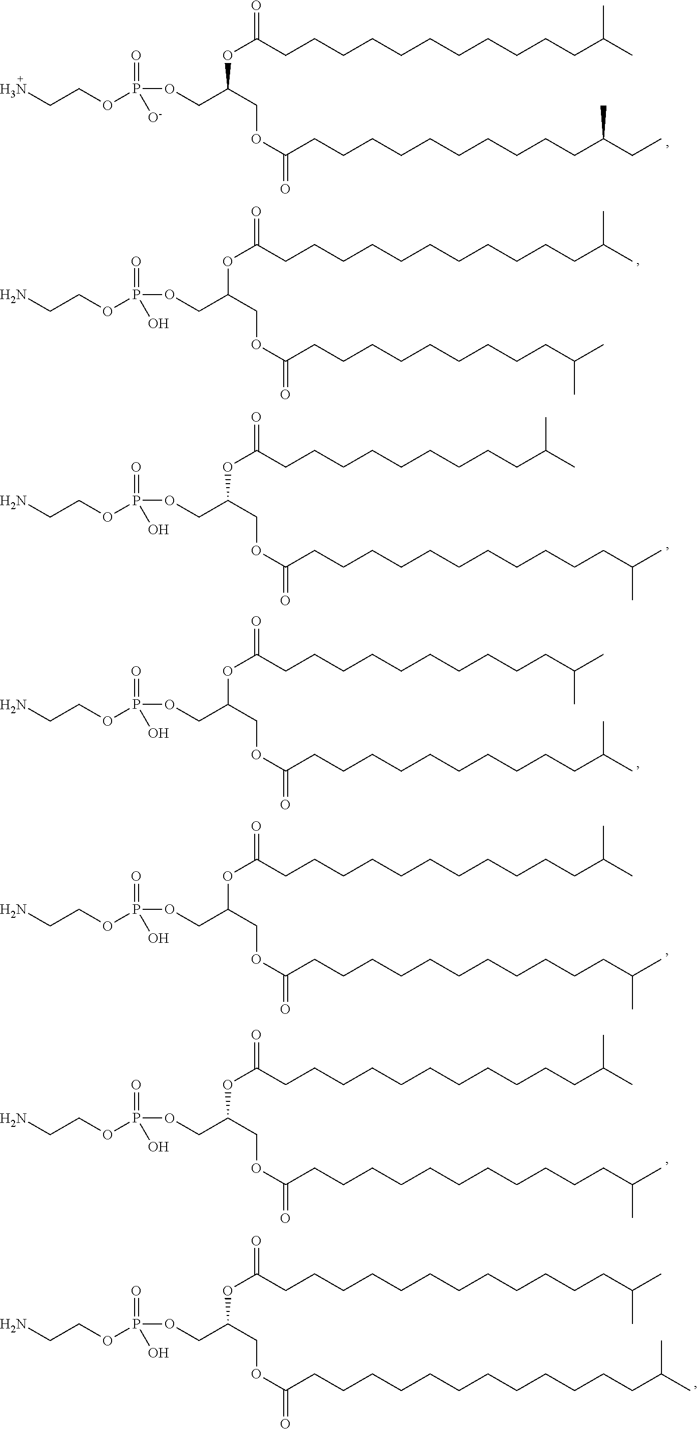

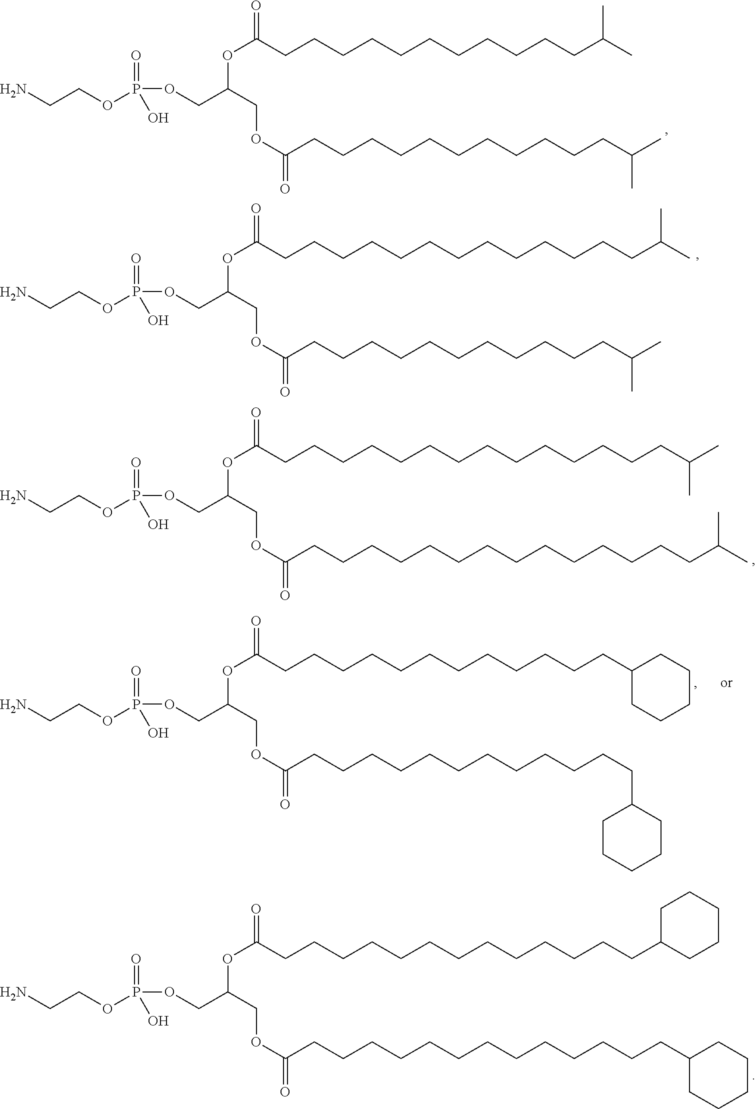

In certain embodiments, the compound of Formula (I) is of one of the foregoing formulae.

In certain embodiments, a compound of Formula (I) is not a natural product.

In certain embodiments, a compound of Formula (I) is of Formula (Ia):

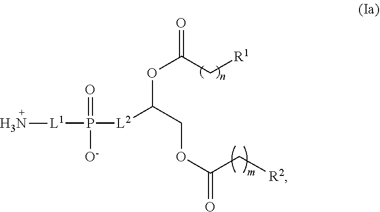

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ib):

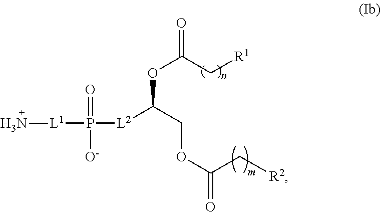

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ic):

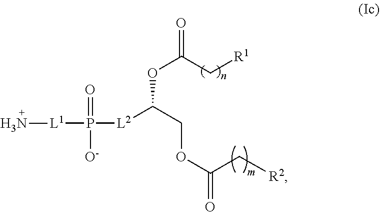

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Id):

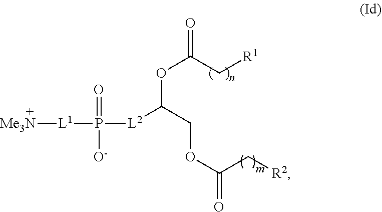

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ie):

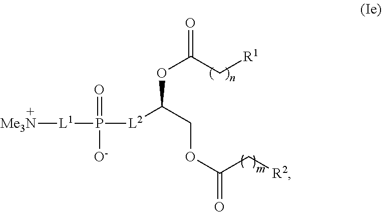

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (If):

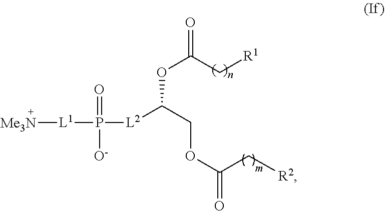

or a pharmaceutically acceptable salt, solvate, hydrate, polymorph, co-crystal, tautomer, stereoisomer, isotopically labeled derivative, or prodrug thereof.

As defined herein, R¹ is branched or unbranched C₁₋₄ alkyl, or C₃₋₇ carbocyclic. In certain embodiments, R¹ is branched or unbranched C₁₋₄ alkyl. In some embodiments, R¹ is branched C₁₋₄ alkyl. In certain embodiments, R¹ is unbranched C₁₋₄ alkyl. In some embodiments, R¹ is branched or unbranched C₃₋₄ alkyl. In certain embodiments, R¹ is branched C₃₋₄ alkyl. In some embodiments, R¹ is unbranched C₃₋₄ alkyl. In certain embodiments, R¹ is unbranched C₁ 2 alkyl. In some embodiments, R¹ is C₁ alkyl. In certain embodiments, R¹ is C₂ alkyl. In some embodiments, R¹ is unbranched C₃ alkyl. In certain embodiments, R¹ is unbranched C₄ alkyl. In some embodiments, R¹ is branched C₃ alkyl. In certain embodiments, R¹ is branched C₄ alkyl. In some embodiments, R¹ is methyl. In certain embodiments, R¹ is ethyl. In some embodiments, R¹ is n-propyl. In certain embodiments, R¹ is n-butyl. In some embodiments, R¹ is isopropyl. In certain embodiments, R¹ is sec-butyl. In some embodiments, R¹ is R-sec-butyl. In certain embodiments, R¹ is S-sec-butyl. In some embodiments, R¹ is isobutyl. In certain embodiments, R¹ is tert-butyl. In some embodiments, R¹ is C₃₋₇ carbocyclic. In certain embodiments, R¹ is C₃₋₄ carbocyclic. In some embodiments, R¹ is C₅₋₇ carbocyclic. In certain embodiments, R¹ is C₃ carbocyclic. In some embodiments, R¹ is C₄ carbocyclic. In certain embodiments, R¹ is C₅ carbocyclic. In some embodiments, R¹ is C₆ carbocyclic. In certain embodiments, R¹ is C₇ carbocyclic. In some embodiments, R¹ is C₃₋₇ saturated carbocyclic. In certain embodiments, R¹ is C₃₋₄ saturated carbocyclic. In some embodiments, R¹ is C₅₋₇ saturated carbocyclic. In some embodiments, R¹ is cyclopropyl. In certain embodiments, R¹ is cyclobutyl. In some embodiments, R¹ is cyclopentyl. In certain embodiments, R¹ is cyclohexyl. In some embodiments, R¹ is cycloheptyl. In certain embodiments, R¹ is C₃₋₇ unsaturated carbocyclic. In some embodiments, R¹ is C₃₋₄ unsaturated carbocyclic. In certain embodiments, R¹ is C₅₋₇ unsaturated carbocyclic. In some embodiments, R¹ is cyclopropenyl. In certain embodiments, R¹ is cyclobutenyl. In some embodiments, R¹ is cyclobutadienyl. In certain embodiments, R¹ is cyclopentenyl. In some embodiments, R¹ is cyclopentadienyl. In certain embodiments, R¹ is cyclohexenyl. In some embodiments, R¹ is cyclohexadienyl. In certain embodiments, R¹ is cycloheptenyl. In some embodiments, R¹ is cycloheptadienyl. In certain embodiments, R¹ is cycloheptatrienyl.

As defined herein, R² is branched or unbranched C₁₋₄ alkyl, or C₃₋₇ carbocyclic. In certain embodiments, R² is branched or unbranched C₁₋₄ alkyl. In some embodiments, R² is branched C₁₋₄ alkyl. In certain embodiments, R² is unbranched C₁₋₄ alkyl. In some embodiments, R² is branched or unbranched C₃₋₄ alkyl. In certain embodiments, R² is branched C₃₋₄ alkyl. In some embodiments, R² is unbranched C₃₋₄ alkyl. In certain embodiments, R² is unbranched C₁₋₂ alkyl. In some embodiments, R² is C₁ alkyl. In certain embodiments, R² is C₂ alkyl. In some embodiments, R² is unbranched C₃ alkyl. In certain embodiments, R² is unbranched C₄ alkyl. In some embodiments, R² is branched C₃ alkyl. In certain embodiments, R² is branched C₄ alkyl. In some embodiments, R² is methyl. In certain embodiments, R² is ethyl. In some embodiments, R² is n-propyl. In certain embodiments, R² is n-butyl. In some embodiments, R² is isopropyl. In certain embodiments, R² is sec-butyl. In some embodiments, R² is R-sec-butyl. In certain embodiments, R² is S-sec-butyl. In some embodiments, R² is isobutyl. In certain embodiments, R² is tert-butyl. In some embodiments, R² is C₃₋₇ carbocyclic. In certain embodiments, R² is C₃₋₄ carbocyclic. In some embodiments, R² is C₅₋₇ carbocyclic. In certain embodiments, R² is C₃ carbocyclic. In some embodiments, R² is C₄ carbocyclic. In certain embodiments, R² is C₅ carbocyclic. In some embodiments, R² is C₆ carbocyclic. In certain embodiments, R² is C₇ carbocyclic. In some embodiments, R² is C₃₋₇ saturated carbocyclic. In certain embodiments, R² is C₃₋₄ saturated carbocyclic. In some embodiments, R² is C₅₋₇ saturated carbocyclic. In some embodiments, R² is cyclopropyl. In certain embodiments, R² is cyclobutyl. In some embodiments, R² is cyclopentyl. In certain embodiments, R² is cyclohexyl. In some embodiments, R² is cycloheptyl. In certain embodiments, R² is C₃₋₇ unsaturated carbocyclic. In some embodiments, R² is C₃₋₄ unsaturated carbocyclic. In certain embodiments, R² is C₅₋₇ unsaturated carbocyclic. In some embodiments, R² is cyclopropenyl. In certain embodiments, R² is cyclobutenyl. In some embodiments, R² is cyclobutadienyl. In certain embodiments, R² is cyclopentenyl. In some embodiments, R² is cyclopentadienyl. In certain embodiments, R² is cyclohexenyl. In some embodiments, R² is cyclohexadienyl. In certain embodiments, R² is cycloheptenyl. In some embodiments, R² is cycloheptadienyl. In certain embodiments, R² is cycloheptatrienyl.

In certain embodiments, at least one of R¹ and R² is branched C₁₋₄ alkyl or C₃₋₇ carbocyclic. In some embodiments, at least one of R¹ and R² is branched C₃₋₄ alkyl or C₃₋₇ carbocyclic. In certain embodiments, at least one of R¹ and R² is branched C₃₋₄ alkyl or C₃₋₄ carbocyclic. In some embodiments, at least one of R¹ and R² is branched C₃₋₄ alkyl or C₃₋₄ saturated carbocyclic. In some embodiments, R¹ and R² are the same. In certain embodiments, R¹ and R² are different.

In certain embodiments, R¹ is branched C₁₋₄ alkyl, and R² is branched C₁₋₄ alkyl. In some embodiments, R¹ is branched C₃₋₄ alkyl, and R² is branched C₁₋₄ alkyl. In certain embodiments, R¹ is branched C₁₋₄ alkyl, and R² is branched C₃₋₄ alkyl. In some embodiments, R¹ is branched C₃₋₄ alkyl, and R² is branched C₃₋₄ alkyl. In certain embodiments, R¹ is unbranched C₁₋₄ alkyl, and R² is branched C₁₋₄ alkyl. In some embodiments, R¹ is unbranched C₃₋₄ alkyl, and R² is branched C₁₋₄ alkyl. In certain embodiments, R¹ is unbranched C₁₋₄ alkyl, and R² is branched C₃₋₄ alkyl. In some embodiments, R¹ is unbranched C₃₋₄ alkyl, and R² is branched C₃₋₄ alkyl. In certain embodiments, R¹ is branched C₁₋₄ alkyl, and R² is unbranched C₁₋₄ alkyl. In some embodiments, R¹ is branched C₃₋₄ alkyl, and R² is unbranched C₁₋₄ alkyl. In certain embodiments, R¹ is branched C₁₋₄ alkyl, and R² is unbranched C₃₋₄ alkyl. In some embodiments, R¹ is branched C₃₋₄ alkyl, and R² is unbranched C₃₋₄ alkyl. In certain embodiments, R¹ is branched C₁₋₄ alkyl, and R² is C₃₋₇ carbocyclic. In some embodiments, R¹ is C₃₋₇ carbocyclic and R² is branched C₁₋₄ alkyl. In certain embodiments, R¹ is unbranched C₁₋₄ alkyl, and R² is C₃₋₇ carbocyclic. In some embodiments, R¹ is C₃₋₇ carbocyclic and R² is unbranched C₁₋₄ alkyl. In certain embodiments, R¹ is branched C₃₋₄ alkyl, and R² is C₃₋₇ carbocyclic. In some embodiments, R¹ is C₃₋₇ carbocyclic, and R² is branched C₃₋₄ alkyl. In certain embodiments, R¹ is unbranched C₃₋₄ alkyl, and R² is C₃₋₇ carbocyclic. In some embodiments, R¹ is C₃₋₇ carbocyclic, and R² is unbranched C₃₋₄ alkyl. In certain embodiments, R¹ is branched C₁₋₄ alkyl, and R² is C₃₋₄ carbocyclic. In some embodiments, R¹ is C₃₋₄ carbocyclic, and R² is branched C₁₋₄ alkyl. In certain embodiments, R¹ is unbranched C₁₋₄ alkyl, and R² is C₃₋₄ carbocyclic. In some embodiments, R¹ is C₃₋₄ carbocyclic, and R² is unbranched C₁₋₄ alkyl. In certain embodiments, R¹ is branched C₃₋₄ alkyl, and R² is C₃₋₄ carbocyclic. In some embodiments, R¹ is C₃₋₄ carbocyclic, and R² is branched C₃₋₄ alkyl. In certain embodiments, R¹ is unbranched C₃₋₄ alkyl, and R² is C₃₋₄ carbocyclic. In some embodiments, R¹ is C₃₋₄ carbocyclic, and R² is unbranched C₃₋₄ alkyl. In certain embodiments, R¹ is C₃₋₇ carbocyclic, and R² is C₃₋₇ carbocyclic. In some embodiments, R¹ is C₃₋₇ carbocyclic, and R² is C₃₋₄ carbocyclic. In certain embodiments, R¹ is C₃₋₄ carbocyclic, and R² is C₃₋₇ carbocyclic. In some embodiments, R¹ is C₃₋₄ carbocyclic, and R² is C₃₋₄ carbocyclic.

As defined herein, m is an integer from 7-13, inclusive. In certain embodiments, m is 7. In some embodiments, m is 8. In certain embodiments, m is 9. In some embodiments, m is 10. In certain embodiments, m is 11. In some embodiments, m is 12. In certain embodiments, m is 13. In some embodiments, m is an integer from 7-12, inclusive. In certain embodiments, m is an integer from 8-13, inclusive. In some embodiments, m is an integer from 8-12, inclusive. In certain embodiments, m is an integer from 7-11, inclusive. In some embodiments, m is an integer from 8-11, inclusive. In certain embodiments, m is an integer from 9-12, inclusive. In some embodiments, m is an integer from 9-13, inclusive. In certain embodiments, m is an integer from 10-13, inclusive. In some embodiments, m is an integer from 10-12, inclusive. In certain embodiments, m is an integer from 11-13, inclusive. In some embodiments, m is an integer from 11-12, inclusive.

As defined herein, n is an integer from 7-13. In certain embodiments, n is 7. In some embodiments, n is 8. In certain embodiments, n is 9. In some embodiments, n is 10. In certain embodiments, n is 11. In some embodiments, n is 12. In certain embodiments, n is 13. In some embodiments, n is an integer from 7-12, inclusive. In certain embodiments, n is an integer from 8-13, inclusive. In some embodiments, n is an integer from 8-12, inclusive. In certain embodiments, m is an integer from 7-11, inclusive. In some embodiments, m is an integer from 8-11, inclusive. In certain embodiments, n is an integer from 9-12, inclusive. In some embodiments, n is an integer from 9-13, inclusive. In certain embodiments, n is an integer from 10-13, inclusive. In some embodiments, n is an integer from 10-12, inclusive. In certain embodiments, n is an integer from 11-13, inclusive. In some embodiments, n is an integer from 11-12, inclusive.

In certain embodiments, m is 10, and n is 10. In some embodiments, m is 10, and n is 11. In certain embodiments, m is 10, and n is 12. In some embodiments, m is 11, and n is 10. In certain embodiments, m is 11, and n is 11. In some embodiments, m is 11, and n is 12. In certain embodiments, m is 12, and n is 10. In some embodiments, m is 12, and n is 11. In certain embodiments, m is 12, and n is 12.

In certain embodiments, n is 11, and R¹ is branched C₃ alkyl or C₃ carbocyclic. In some embodiments, n is 11, and R¹ is branched C₃ alkyl. In certain embodiments, n is 11, and R¹ is C₃ carbocyclic. In some embodiments, n is 11, and R¹ is C₃ saturated carbocyclic. In certain embodiments, n is 11, and R¹ is C₃ unsaturated carbocyclic. In some embodiments, n is 11, and R¹ is isopropyl. In certain embodiments, n is 11, and R¹ is cyclopropyl. In some embodiments, n is 11, and R¹ is cyclopropenyl.

In certain embodiments, n is 10, and R¹ is branched C₄ alkyl or C₄ carbocyclic. In some embodiments, n is 10, and R¹ is branched C₄ alkyl. In certain embodiments, n is 10, and R¹ is C₄ carbocyclic. In some embodiments, n is 10, and R¹ is C₄ saturated carbocyclic. In certain embodiments, n is 10, and R¹ is C₄ unsaturated carbocyclic. In some embodiments, n is 10, and R¹ is sec-butyl. In certain embodiments, n is 10, and R¹ is S-sec-butyl. In some embodiments, n is 10, and R¹ is R-sec-butyl. In certain embodiments, n is 10, and R¹ is tert-butyl. In some embodiments, n is 10, and R¹ is cyclobutyl. In certain embodiments, n is 10, and R¹ is cyclobutenyl. In certain embodiments, n is 10, and R¹ is cyclobutadienyl.

In certain embodiments, m is 11, and R² is branched C₃ alkyl or C₃ carbocyclic. In some embodiments, m is 11, and R² is branched C₃ alkyl. In certain embodiments, m is 11, and R² is C₃ carbocyclic. In some embodiments, m is 11, and R² is C₃ saturated carbocyclic. In certain embodiments, m is 11, and R² is C₃ unsaturated carbocyclic. In some embodiments, m is 11, and R² is isopropyl. In certain embodiments, m is 11, and R² is cyclopropyl. In some embodiments, m is 11, and R² is cyclopropenyl.

In certain embodiments, m is 10, and R² is branched C₄ alkyl or C₄ carbocyclic. In some embodiments, m is 10, and R² is branched C₄ alkyl. In certain embodiments, m is 10, and R² is carbocyclic. In some embodiments, m is 10, and R² is saturated carbocyclic. In certain embodiments, m is 10, and R² is C₄ unsaturated carbocyclic. In some embodiments, m is 10, and R² is sec-butyl. In certain embodiments, m is 10, and R² is S-sec-butyl. In some embodiments, m is 10, and R² is R-sec-butyl. In certain embodiments, m is 10, and R² is tert-butyl. In some embodiments, m is 10, and R² is cyclobutyl. In certain embodiments, m is 10, and R² is cyclobutenyl. In certain embodiments, m is 10, and R² is cyclobutadienyl.

In certain embodiments, n is 10, and R¹ is branched C₃ alkyl or C₃ carbocyclic. In some embodiments, n is 10, and R¹ is branched C₃ alkyl. In certain embodiments, n is 10, and R¹ is C₃ carbocyclic. In some embodiments, n is 10, and R¹ is C₃ saturated carbocyclic. In certain embodiments, n is 10, and R¹ is C₃ unsaturated carbocyclic. In some embodiments, n is 10, and R¹ is isopropyl. In certain embodiments, n is 10, and R¹ is cyclopropyl. In some embodiments, n is 10, and R¹ is cyclopropenyl.

In certain embodiments, n is 9, and R¹ is branched C₄ alkyl or C₄ carbocyclic. In some embodiments, n is 9, and R¹ is branched C₄ alkyl. In certain embodiments, n is 9, and R¹ is C₄ carbocyclic. In some embodiments, n is 9, and R¹ is C₄ saturated carbocyclic. In certain embodiments, n is 9, and R¹ is C₄ unsaturated carbocyclic. In some embodiments, n is 9, and R¹ is sec-butyl. In certain embodiments, n is 9, and R¹ is S-sec-butyl. In some embodiments, n is 9, and R¹ is R-sec-butyl. In certain embodiments, n is 9, and R¹ is tert-butyl. In some embodiments, n is 9, and R¹ is cyclobutyl. In certain embodiments, n is 9, and R¹ is cyclobutenyl. In certain embodiments, n is 9, and R¹ is cyclobutadienyl.

In certain embodiments, m is 10, and R² is branched C₃ alkyl or C₃ carbocyclic. In some embodiments, m is 10, and R² is branched C₃ alkyl. In certain embodiments, m is 10, and R² is C₃ carbocyclic. In some embodiments, m is 10, and R² is C₃ saturated carbocyclic. In certain embodiments, m is 10, and R² is C₃ unsaturated carbocyclic. In some embodiments, m is 10, and R² is isopropyl. In certain embodiments, m is 10, and R² is cyclopropyl. In some embodiments, m is 10, and R² is cyclopropenyl.

In certain embodiments, m is 9, and R² is branched C₄ alkyl or C₄ carbocyclic. In some embodiments, m is 9, and R² is branched C₄ alkyl. In certain embodiments, m is 9, and R² is carbocyclic. In some embodiments, m is 10, and R² is saturated carbocyclic. In certain embodiments, m is 9, and R² is C₄ unsaturated carbocyclic. In some embodiments, m is 9, and R² is sec-butyl. In certain embodiments, m is 9, and R² is S-sec-butyl. In some embodiments, m is 9, and R² is R-sec-butyl. In certain embodiments, m is 9, and R² is tert-butyl. In some embodiments, m is 9, and R² is cyclobutyl. In certain embodiments, m is 9, and R² is cyclobutenyl. In certain embodiments, m is 9, and R² is cyclobutadienyl.

In certain embodiments, n is 12, and R¹ is branched C₃ alkyl or C₃ carbocyclic. In some embodiments, n is 12, and R¹ is branched C₃ alkyl. In certain embodiments, n is 12, and R¹ is C₃ carbocyclic. In some embodiments, n is 12, and R¹ is C₃ saturated carbocyclic. In certain embodiments, n is 12, and R¹ is C₃ unsaturated carbocyclic. In some embodiments, n is 12, and R¹ is isopropyl. In certain embodiments, n is 12, and R¹ is cyclopropyl. In some embodiments, n is 12, and R¹ is cyclopropenyl.

In certain embodiments, n is 11, and R¹ is branched C₄ alkyl or C₄ carbocyclic. In some embodiments, n is 11, and R¹ is branched C₄ alkyl. In certain embodiments, n is 11, and R¹ is C₄ carbocyclic. In some embodiments, n is 11, and R¹ is C₄ saturated carbocyclic. In certain embodiments, n is 11, and R¹ is C₄ unsaturated carbocyclic. In some embodiments, n is 11, and R¹ is sec-butyl. In certain embodiments, n is 11, and R¹ is S-sec-butyl. In some embodiments, n is 11, and R¹ is R-sec-butyl. In certain embodiments, n is 11, and R¹ is tert-butyl. In some embodiments, n is 11, and R¹ is cyclobutyl. In certain embodiments, n is 11 and R¹ is cyclobutenyl. In certain embodiments, n is 11, and R¹ is cyclobutadienyl.

In certain embodiments, m is 12, and R² is branched C₃ alkyl or C₃ carbocyclic. In some embodiments, m is 12, and R² is branched C₃ alkyl. In certain embodiments, m is 12, and R² is C₃ carbocyclic. In some embodiments, m is 12, and R² is C₃ saturated carbocyclic. In certain embodiments, m is 12, and R² is C₃ unsaturated carbocyclic. In some embodiments, m is 12, and R² is isopropyl. In certain embodiments, m is 12, and R² is cyclopropyl. In some embodiments, m is 12, and R² is cyclopropenyl.

In certain embodiments, m is 11, and R² is branched C₄ alkyl or C₄ carbocyclic. In some embodiments, m is 11, and R² is branched C₄ alkyl. In certain embodiments, m is 11, and R² is carbocyclic. In some embodiments, m is 11, and R² is saturated carbocyclic. In certain embodiments, m is 11, and R² is C₄ unsaturated carbocyclic. In some embodiments, m is 11, and R² is sec-butyl. In certain embodiments, m is 11, and R² is S-sec-butyl. In some embodiments, m is 11, and R² is R-sec-butyl. In certain embodiments, m is 11, and R² is tert-butyl. In some embodiments, m is 11, and R² is cyclobutyl. In certain embodiments, m is 11, and R² is cyclobutenyl. In certain embodiments, m is 11, and R² is cyclobutadienyl.

As defined herein, R³ is —H or branched or unbranched C₁₋₆ alkyl. In certain embodiments, R³ is branched C₁₋₆ alkyl. In some embodiments, R³ is unbranched C₁₋₆ alkyl. In some embodiments, R³ is branched C₁₋₄ alkyl. In some embodiments, R³ is unbranched C₁₋₄ alkyl. In certain embodiments, R³ is -n-Bu, -n-Pr, -Et, Me, or —H. In some embodiments, R³ is -n-Bu. In certain embodiments, R³ is -n-Pr. In some embodiments, R³ is -Et. In certain embodiments, R³ is -Me. In some embodiments, R³ is —H.

As defined herein, L¹ is substituted or unsubstituted C₁₋₈ alkylene, substituted or unsubstituted C₁₋₈ heteroalkylene, substituted or unsubstituted carbocyclylene, substituted or unsubstituted heterocyclylene, substituted or unsubstituted arylene, substituted or unsubstituted heteroarylene, or any combination thereof. In certain embodiments, L¹ is substituted or unsubstituted C₁₋₅ alkylene. In some embodiments, L₁ is substituted C₁₋₈ alkylene. In certain embodiments, L₁ is unsubstituted C₁₋₈ alkylene. In some embodiments, L¹ is selected from the group consisting of

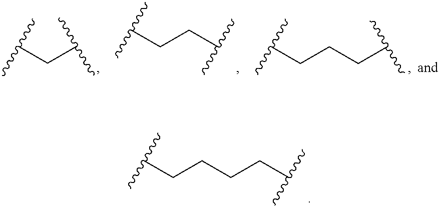

In certain embodiments, L¹ is

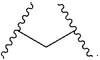 [SMILES: *C*]

In some embodiments, L¹ is

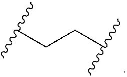 [SMILES: *CC*]

In certain embodiments, L¹ is

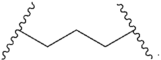 [SMILES: *CCC*]

In some embodiments, L¹ is

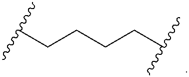 [SMILES: *CCCC*]

In certain embodiments, L¹ is substituted or unsubstituted C₁₋₈ heteroalkylene. In some embodiments, L¹ is substituted C₁₋₈ heteroalkylene. In certain embodiments, L¹ is substituted C₁₋₈ heteroalkylene comprising one or more oxygen atoms. In some embodiments, L¹ is substituted C₂ heteroalkylene comprising one or more oxygen atoms. In certain embodiments, L¹ is substituted C₃ heteroalkylene comprising one or more oxygen atoms. In some embodiments, L¹ is substituted C₄ heteroalkylene comprising one or more oxygen atoms. In certain embodiments, L¹ is substituted C₅ heteroalkylene comprising one or more oxygen atoms. In some embodiments, L¹ is substituted C₆ heteroalkylene comprising one or more oxygen atoms. In certain embodiments, L¹ is substituted C₇ heteroalkylene comprising one or more oxygen atoms. In some embodiments, L¹ is substituted C₈ heteroalkylene comprising one or more oxygen atoms. In certain embodiments, L¹ is unsubstituted C₁₋₈ heteroalkylene. In some embodiments, L¹ is unsubstituted C₁₋₈ heteroalkylene comprising one or more oxygen atoms. In certain embodiments, L¹ is C₂ heteroalkylene comprising one or more oxygen atoms. In some embodiments, L¹ is unsubstituted C₃ heteroalkylene comprising one or more oxygen atoms. In certain embodiments, L¹ is unsubstituted C₄ heteroalkylene comprising one or more oxygen atoms. In some embodiments, L¹ is unsubstituted C₅ heteroalkylene comprising one or more oxygen atoms. In certain embodiments, L¹ is unsubstituted C₆ heteroalkylene comprising one or more oxygen atoms. In some embodiments, L¹ is unsubstituted C₇ heteroalkylene comprising one or more oxygen atoms. In certain embodiments, L¹ is unsubstituted C₈ heteroalkylene comprising one or more oxygen atoms. In some embodiments, L¹ is selected from the group consisting of

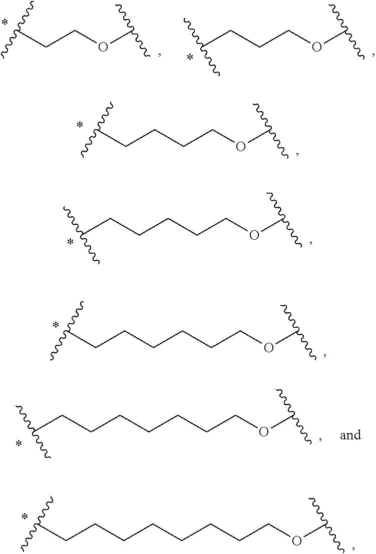

wherein the attachment point labeled with “*” is attached to the nitrogen atom. In some embodiments, L¹ is selected from the group consisting of

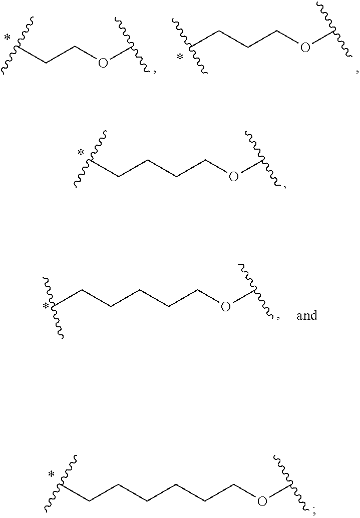 [SMILES: *CCO* ; *CCCO* ; *CCCCO* ; *CCCCCO* ; *CCCCCCO*]

wherein the attachment point labeled with “*” is attached to the nitrogen atom. In certain embodiments, L¹ is

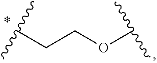 [SMILES: *CCO*]

wherein the attachment point labeled with “*” is attached to the nitrogen atom. In some embodiments, L¹ is

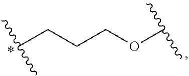 [SMILES: *CCCO*]

wherein the attachment point labeled with “*” is attached to the nitrogen atom. In certain embodiments, L¹ is

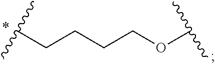 [SMILES: *CCCCO*]

wherein the attachment point labeled with “*” is attached to the nitrogen atom.

In certain embodiments, L¹ comprises substituted or unsubstituted carbocyclylene. In some embodiments, L¹ comprises unsubstituted carbocyclylene. In certain embodiments, L¹ comprises substituted carbocyclylene. In some embodiments, L¹ comprises C₃₋₆ substituted carbocyclylene. In certain embodiments, L¹ comprises C₅₋₆ carbocyclylene.

In certain embodiments, L¹ comprises substituted or unsubstituted heterocyclylene. In some embodiments, L¹ comprises substituted heterocyclylene. In certain embodiments, L¹ comprises unsubstituted heterocyclylene. In some embodiments, L¹ comprises C₅₋₆ substituted heterocyclylene.

In certain embodiments, L¹ comprises substituted or unsubstituted arylene. In some embodiments, L¹ comprises substituted arylene. In certain embodiments, L¹ comprises unsubstituted arylene. In some embodiments, L¹ comprises unsubstituted C₆₋₁₄ arylene. In certain embodiments, L¹ comprises substituted C₆₋₁₄ arylene.

In certain embodiments, L¹ comprises substituted or unsubstituted heteroarylene. In some embodiments, L¹ comprises substituted heteroarylene. In certain embodiments, L¹ comprises unsubstituted heteroarylene. In some embodiments, L¹ comprises substituted 5-6 membered heteroarylene. In certain embodiments, L¹ comprises unsubstituted 5-6 membered heteroarylene.

As defined herein, L² is unbranched and unsubstituted C₁₋₆ heteroalkylene, or unbranched and unsubstituted C₁₋₆ alkylene. In certain embodiments, L² is unbranched and unsubstituted C₁₋₆ heteroalkylene. In some embodiments, L² is unbranched and unsubstituted C₁₋₆ heteroalkylene comprising one or more oxygen atoms. In certain embodiments, L² is unbranched and unsubstituted C₁₋₆ heteroalkylene comprising one oxygen atom. In some embodiments, L² is unbranched and unsubstituted C₁ heteroalkylene comprising one oxygen atom. In certain embodiments, L² is unbranched and unsubstituted C₂ heteroalkylene comprising one oxygen atom. In some embodiments, L² is unbranched and unsubstituted C₁ comprising two oxygen atoms. In certain embodiments, L² is selected from the group consisting of

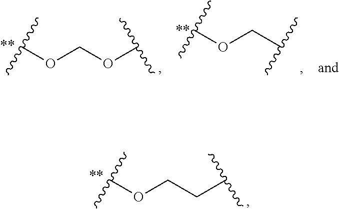

wherein the attachment point labeled with “**” is attached to the phosphorous atom. In some embodiments, L² is

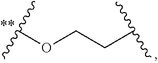 [SMILES: *CCO*]

wherein the attachment point labeled with “**” is attached to the phosphorous atom. In certain embodiments, L² is selected from the group consisting of

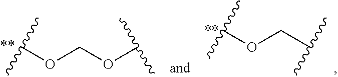

wherein the attachment point labeled with “**” is attached to the phosphorous atom. In some embodiments, L² is

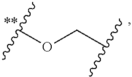 [SMILES: *CO*]

wherein the attachment point labeled with “**” is attached to the phosphorous atom. In certain embodiments, L² is

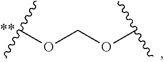 [SMILES: *OCO*]

wherein the attachment point labeled with “**” is attached to the phosphorous atom.

In certain embodiments, L² is unbranched and unsubstituted C₁₋₆ alkylene. In some embodiments, L² is unbranched and unsubstituted C₁₋₄ alkylene. In certain embodiments, L² is unbranched and unsubstituted C₁ alkylene. In some embodiments, L² is unbranched and unsubstituted C₂ alkylene. In certain embodiments, L² is unbranched and unsubstituted C₃ alkylene. In some embodiments, L² is unbranched and unsubstituted C₄ alkylene. In certain embodiments, L² is unbranched and unsubstituted C₅ alkylene. In some embodiments, L² is unbranched and unsubstituted C₆ alkylene.

In certain embodiments, the compound of Formula (I) is of the following formula:

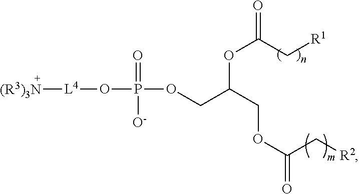

or a pharmaceutically acceptable salt, solvate, hydrate, polymorph, co-crystal, tautomer, stereoisomer, isotopically labeled derivative, or prodrug thereof, wherein:

L⁴ is substituted or unsubstituted C₂₋₈ alkylene, substituted or unsubstituted C₂₋₈ heteroalkylene, substituted or unsubstituted carbocyclylene, substituted or unsubstituted heterocyclylene, substituted or unsubstituted arylene, substituted or unsubstituted heteroarylene, or combinations thereof.

In certain embodiments, the compound of Formula (I) is of the following formula:

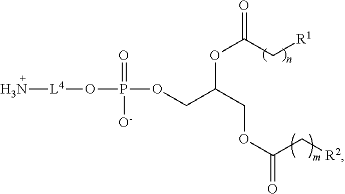

or a pharmaceutically acceptable salt, solvate, hydrate, polymorph, co-crystal, tautomer, stereoisomer, isotopically labeled derivative, or prodrug thereof, wherein:

L⁴ is substituted or unsubstituted C₂₋₈ alkylene, substituted or unsubstituted C₂₋₈ heteroalkylene, substituted or unsubstituted carbocyclylene, substituted or unsubstituted heterocyclylene, substituted or unsubstituted arylene, substituted or unsubstituted heteroarylene, or combinations thereof.

In certain embodiments, the compound of Formula (I) is of the following formula:

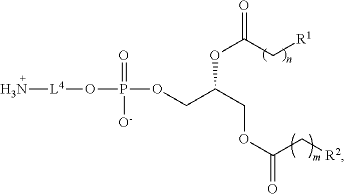

or a pharmaceutically acceptable salt, solvate, hydrate, polymorph, co-crystal, tautomer, stereoisomer, isotopically labeled derivative, or prodrug thereof, wherein:

L⁴ is substituted or unsubstituted C₂₋₈ alkylene, substituted or unsubstituted C₂₋₈ heteroalkylene, substituted or unsubstituted carbocyclylene, substituted or unsubstituted heterocyclylene, substituted or unsubstituted arylene, substituted or unsubstituted heteroarylene, or combinations thereof.

In certain embodiments, the compound of Formula (I) is of the following formula:

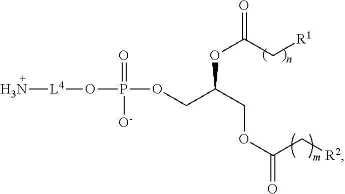

or a pharmaceutically acceptable salt, solvate, hydrate, polymorph, co-crystal, tautomer, stereoisomer, isotopically labeled derivative, or prodrug thereof, wherein:

L⁴ is substituted or unsubstituted C₂₋₈ alkylene, substituted or unsubstituted C₂₋₈ heteroalkylene, substituted or unsubstituted carbocyclylene, substituted or unsubstituted heterocyclylene, substituted or unsubstituted arylene, substituted or unsubstituted heteroarylene, or combinations thereof.

In certain embodiments, the compound of Formula (I) is of the following formula:

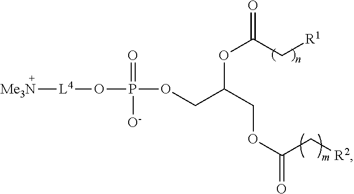

or a pharmaceutically acceptable salt, solvate, hydrate, polymorph, co-crystal, tautomer, stereoisomer, isotopically labeled derivative, or prodrug thereof, wherein:

L⁴ is substituted or unsubstituted C₂₋₈ alkylene, substituted or unsubstituted C₂₋₈ heteroalkylene, substituted or unsubstituted carbocyclylene, substituted or unsubstituted heterocyclylene, substituted or unsubstituted arylene, substituted or unsubstituted heteroarylene, or combinations thereof.

In certain embodiments, the compound of Formula (I) is of the following formula:

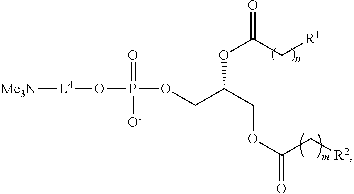

or a pharmaceutically acceptable salt, solvate, hydrate, polymorph, co-crystal, tautomer, stereoisomer, isotopically labeled derivative, or prodrug thereof, wherein:

L⁴ is substituted or unsubstituted C₂₋₈ alkylene, substituted or unsubstituted C₂₋₈ heteroalkylene, substituted or unsubstituted carbocyclylene, substituted or unsubstituted heterocyclylene, substituted or unsubstituted arylene, substituted or unsubstituted heteroarylene, or combinations thereof.

In certain embodiments, the compound of Formula (I) is of the following formula:

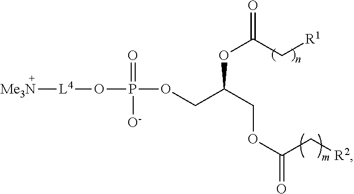

or a pharmaceutically acceptable salt, solvate, hydrate, polymorph, co-crystal, tautomer, stereoisomer, isotopically labeled derivative, or prodrug thereof, wherein:

L⁴ is substituted or unsubstituted C₂₋₈ alkylene, substituted or unsubstituted C₂₋₈ heteroalkylene, substituted or unsubstituted carbocyclylene, substituted or unsubstituted heterocyclylene, substituted or unsubstituted arylene, substituted or unsubstituted heteroarylene, or combinations thereof.

As defined herein, L⁴ is substituted or unsubstituted C₂₋₈ alkylene, substituted or unsubstituted C₂₋₈ heteroalkylene, substituted or unsubstituted carbocyclylene, substituted or unsubstituted heterocyclylene, substituted or unsubstituted arylene, substituted or unsubstituted heteroarylene, or any combination thereof. In certain embodiments, L⁴ is substituted or unsubstituted C₂₋₈ alkylene, substituted or unsubstituted carbocyclylene, substituted or unsubstituted heterocyclylene, substituted or unsubstituted arylene, substituted or unsubstituted heteroarylene, or any combination thereof.

In some embodiments, L⁴ is substituted or unsubstituted C₂₋₈ alkylene. In certain embodiments, L⁴ is unsubstituted C₂₋₈ alkylene. In some embodiments, L⁴ is unsubstituted C₂ alkylene. In certain embodiments, L⁴ is unsubstituted C₃ alkylene. In some embodiments, L⁴ is unsubstituted C₄ alkylene. In certain embodiments, L⁴ is unsubstituted C₅ alkylene. In some embodiments, L⁴ is unsubstituted C₆ alkylene. In certain embodiments, L⁴ is unsubstituted C₇ alkylene. In some embodiments, L⁴ is unsubstituted C₈ alkylene. In certain embodiments, L⁴ is selected from the group consisting of

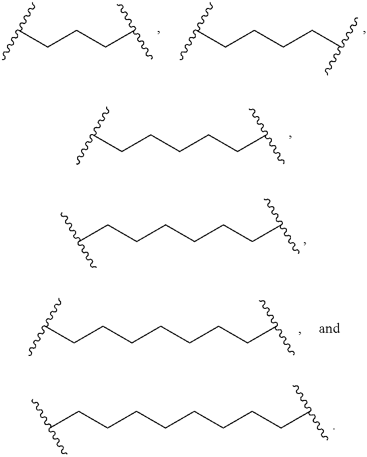 [SMILES: *CCC* ; *CCCC* ; *CCCCC* ; *CCCCCC* ; *CCCCCCC* ; *CCCCCCCC*]

In some embodiments, L⁴ is selected from the group consisting of

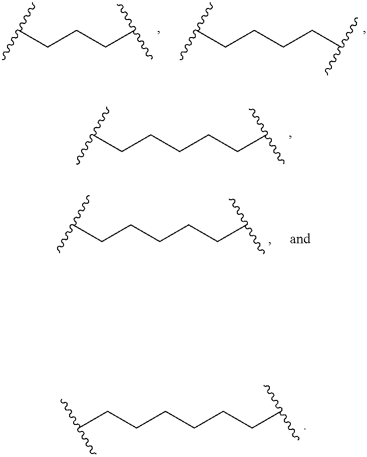 [SMILES: *CCC* ; *CCCC* ; *CCCCC* ; *CCCCC* ; *CCCCCC*]

In certain embodiments, L⁴ is

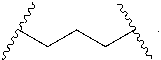 [SMILES: *CCC*]

In some embodiments, L⁴ is

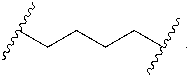 [SMILES: *CCCC*]

In certain embodiments, L⁴ is

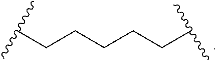 [SMILES: *CCCCC*]

In some embodiments, L⁴ is

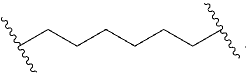 [SMILES: *CCCCCC*]

In certain embodiments, L⁴ is substituted C₂₋₈ alkylene. In some embodiments, L⁴ is substituted C₂ alkylene. In certain embodiments, L⁴ is substituted C₃ alkylene. In some embodiments, L⁴ is substituted C₄ alkylene. In certain embodiments, L⁴ is substituted C₅ alkylene. In some embodiments, L⁴ is substituted C₆ alkylene. In certain embodiments, L⁴ is substituted C₇ alkylene. In certain embodiments, L⁴ is substituted C₈ alkylene.

In some embodiments, L⁴ is substituted or unsubstituted C₂₋₈ heteroalkylene. In certain embodiments, L⁴ is unsubstituted C₂₋₈ heteroalkylene. In some embodiments, L⁴ is unsubstituted C₂ heteroalkylene. In certain embodiments, L⁴ is unsubstituted C₃ heteroalkylene. In some embodiments, L⁴ is unsubstituted C₄ heteroalkylene. In certain embodiments, L⁴ is unsubstituted C₅ heteroalkylene. In some embodiments, L⁴ is unsubstituted C₆ heteroalkylene. In certain embodiments, L⁴ is unsubstituted C₇ heteroalkylene. In certain embodiments, L⁴ is unsubstituted C₈ heteroalkylene.

In certain embodiments, L⁴ is substituted C₂₋₈ heteroalkylene. In some embodiments, L⁴ is substituted C₂ heteroalkylene. In certain embodiments, L⁴ is substituted C₃ heteroalkylene. In some embodiments, L⁴ is substituted C₄ heteroalkylene. In certain embodiments, L⁴ is substituted C₅ heteroalkylene. In some embodiments, L⁴ is substituted C₆ heteroalkylene. In certain embodiments, L⁴ is substituted C₇ heteroalkylene. In certain embodiments, L⁴ is substituted C₈ heteroalkylene.

In certain embodiments, L⁴ comprises substituted or unsubstituted carbocyclylene. In some embodiments, L⁴ comprises substituted carbocyclylene. In certain embodiments, L⁴ comprises unsubstituted carbocyclylene. In some embodiments, L⁴ comprises C₅₋₆ substituted carbocyclylene. In certain embodiments, L⁴ comprises C₅₋₆ unsubstituted carbocyclylene.

In certain embodiments, L⁴ comprises substituted or unsubstituted heterocyclylene. In some embodiments, L⁴ comprises substituted heterocyclylene. In certain embodiments, L⁴ comprises unsubstituted heterocyclylene. In some embodiments, L⁴ comprises C₅₋₆ substituted heterocyclylene.

In certain embodiments, L⁴ comprises substituted or unsubstituted arylene. In some embodiments, L⁴ comprises substituted arylene. In certain embodiments, L⁴ comprises unsubstituted arylene. In some embodiments, L⁴ comprises unsubstituted C₆₋₁₄ arylene. In certain embodiments, L⁴ comprises substituted C₆₋₁₄ arylene.

In some embodiments, L⁴ comprises substituted or unsubstituted heteroarylene. In some embodiments, L⁴ comprises substituted heteroarylene. In certain embodiments, L⁴ comprises substituted 5-6 membered heteroarylene. In some embodiments, L⁴ comprises unsubstituted heteroarylene. In certain embodiments, L⁴ comprises unsubstituted 5-6 membered heteroarylene.

In certain embodiments, the compound of Formula (I) is of the following formula:

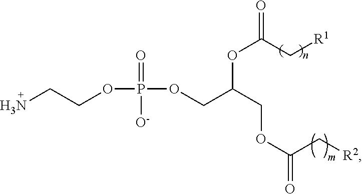

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

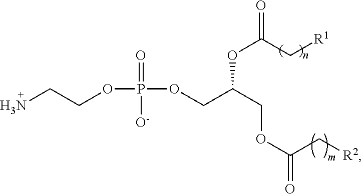

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

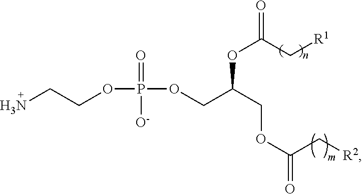

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

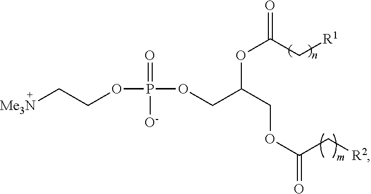

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

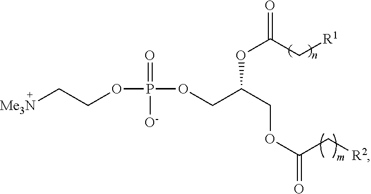

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

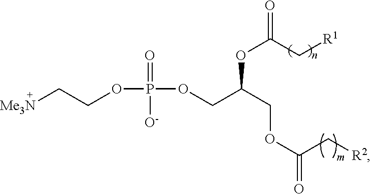

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

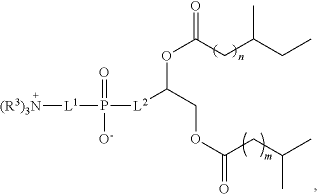

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

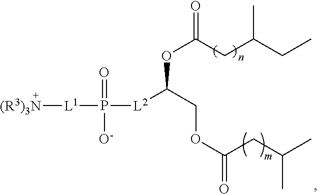

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

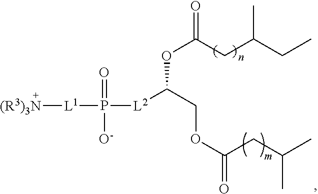

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

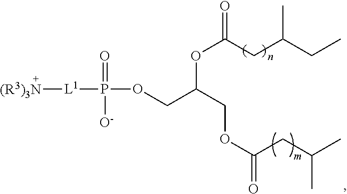

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

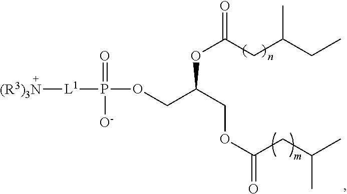

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the following formula:

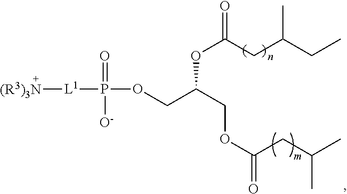

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of one of the following formulae:

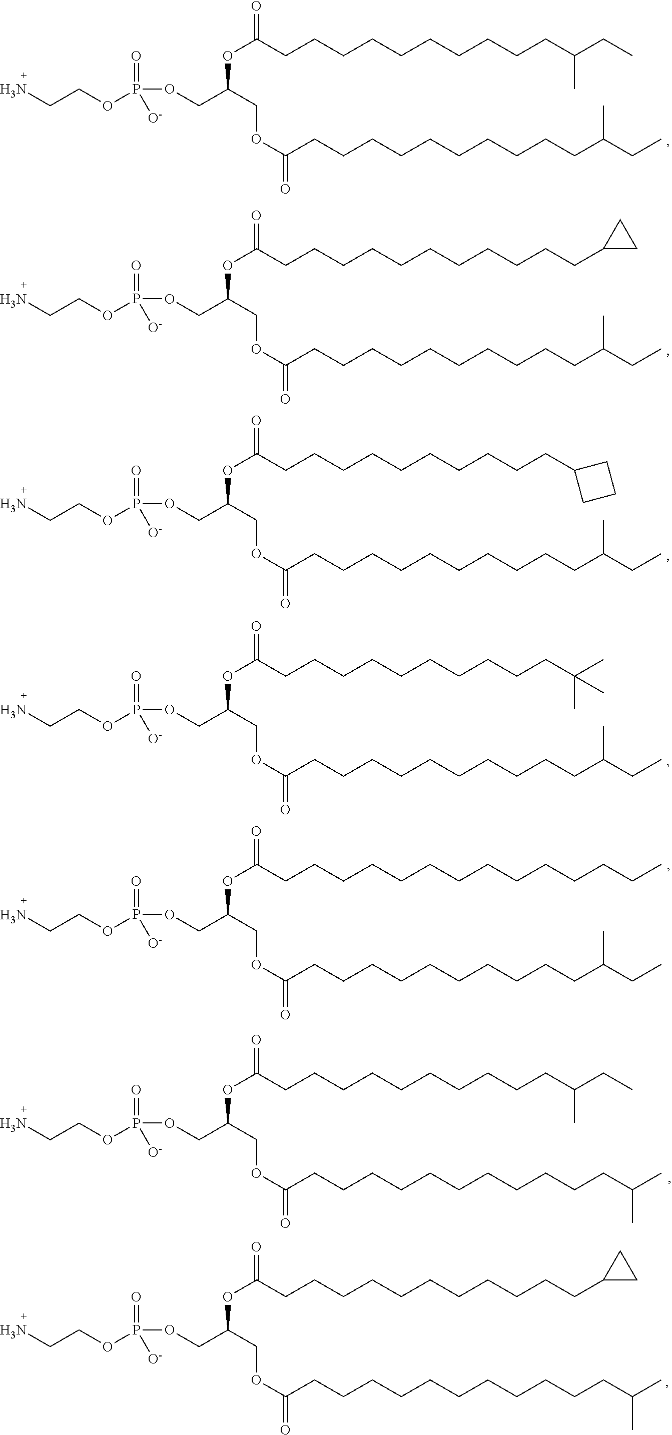

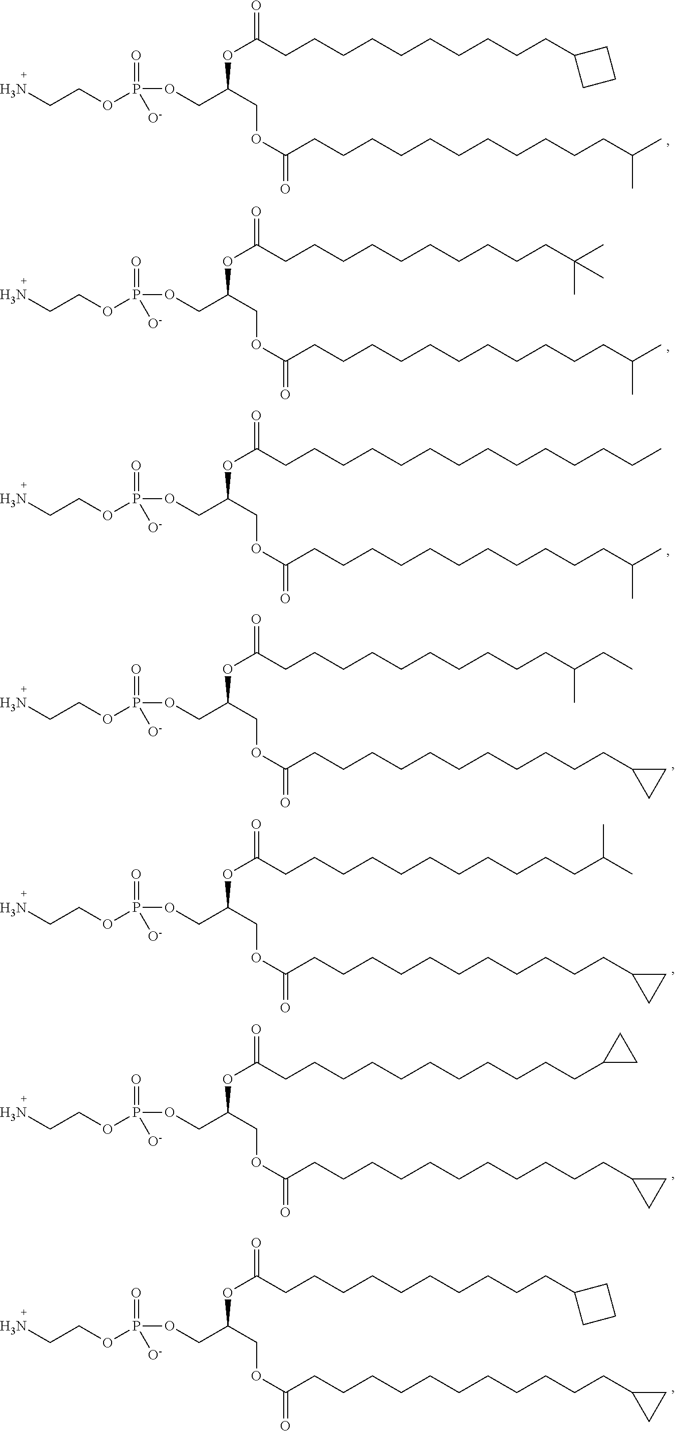

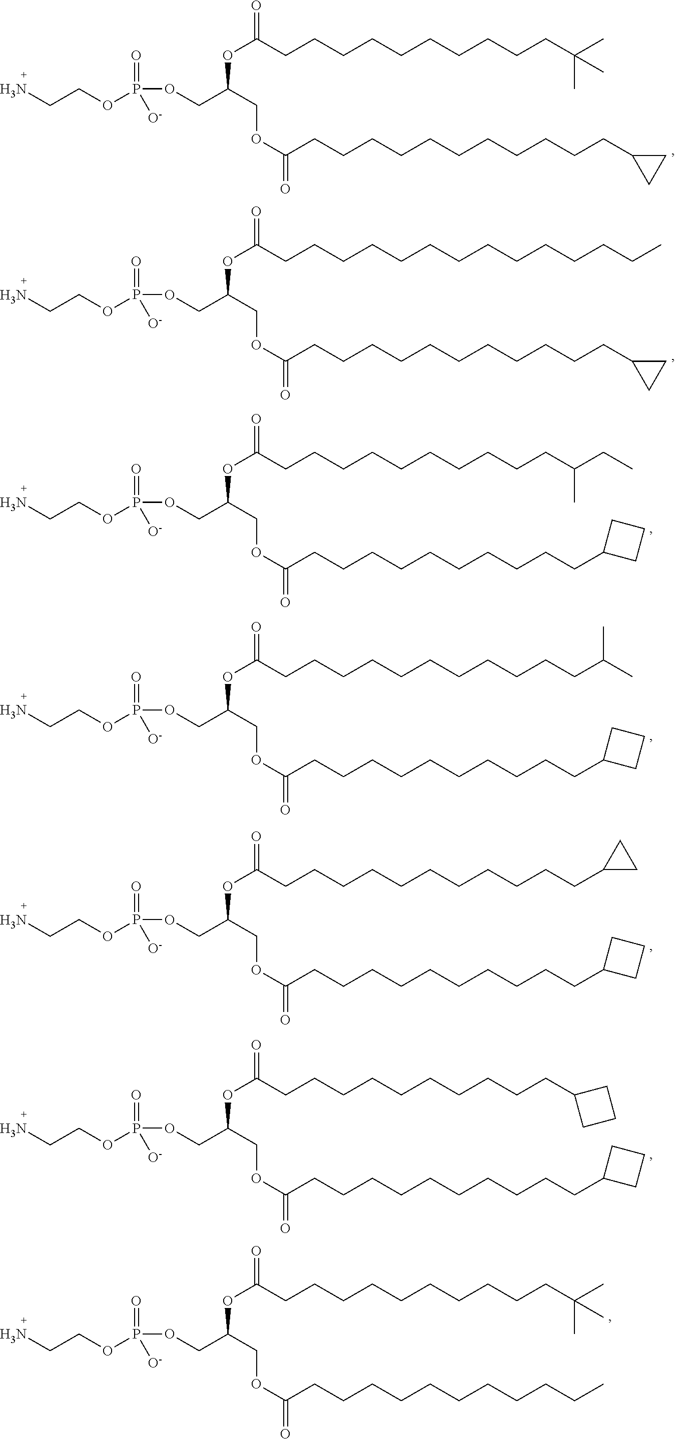

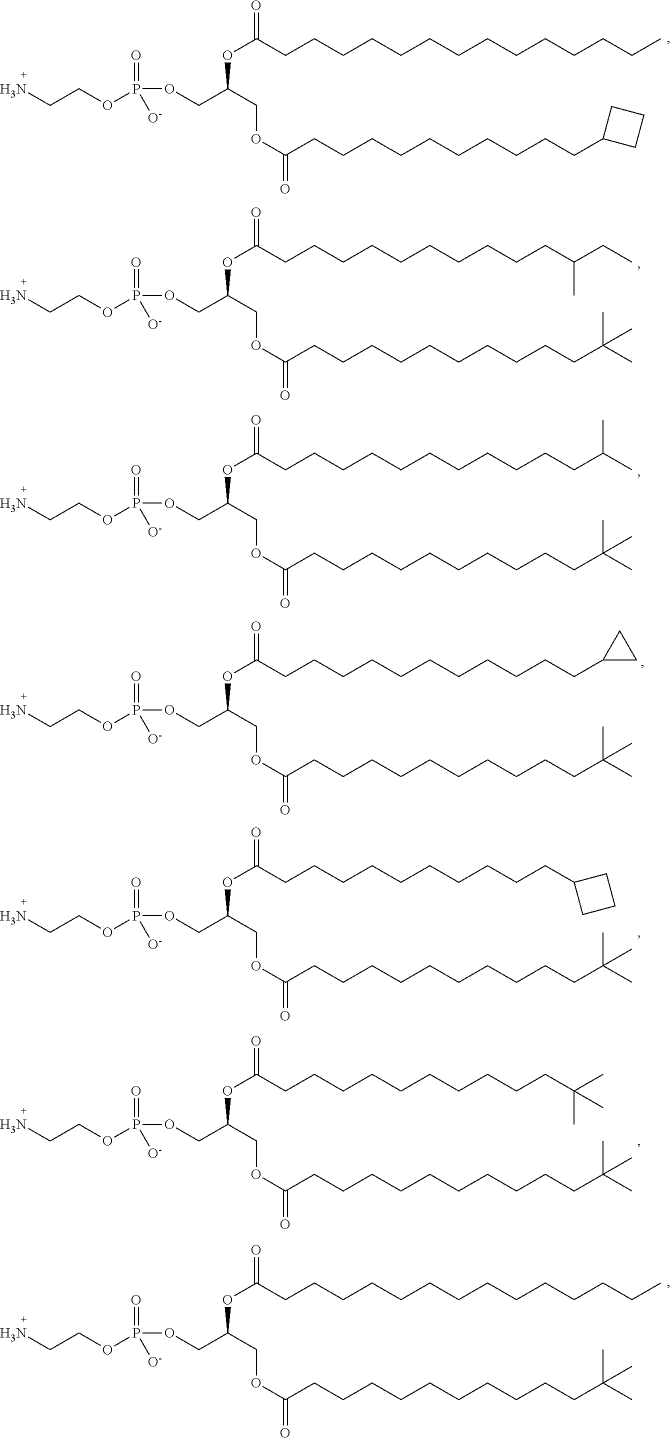

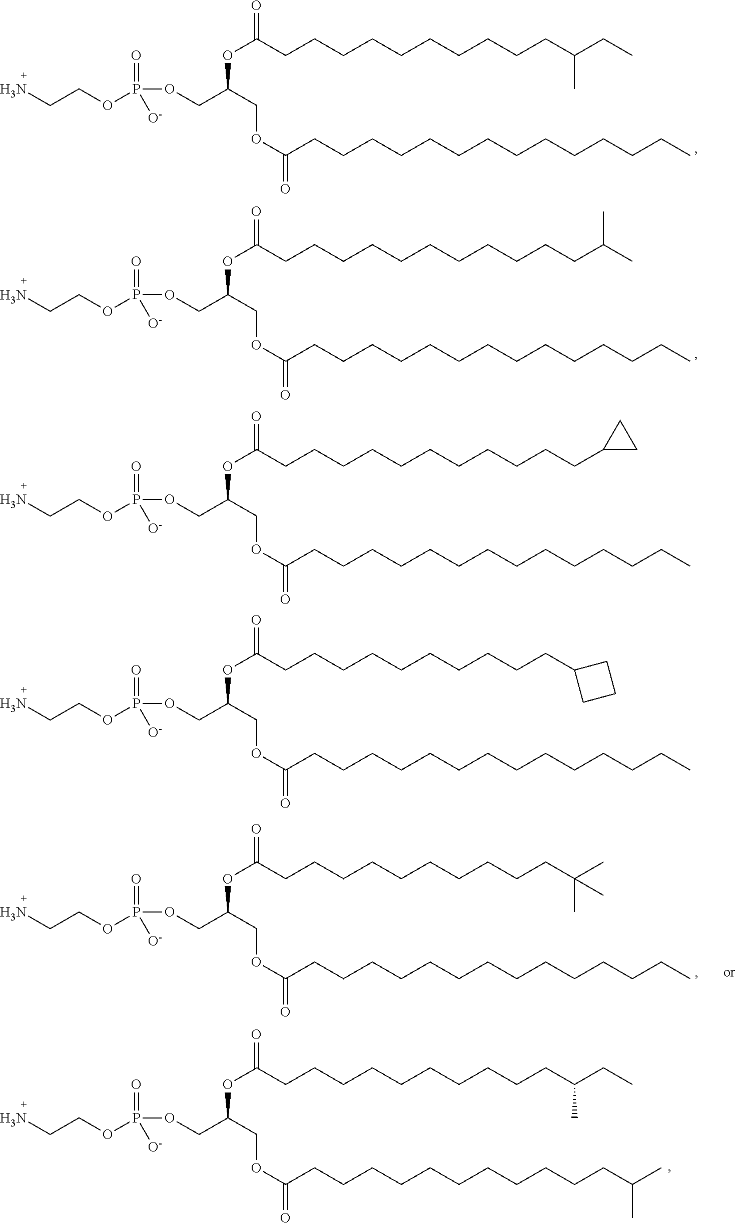 [SMILES: CCCCCCCCCCCCCCC(=O)OC[C@@H](COP(=O)([O-])OCCN)OC(=O)CCCCCCCCCCC(C)CC ; CCCCCCCCCCCCCCC(=O)OC[C@@H](COP(=O)([O-])OCCN)OC(=O)CCCCCCCCCCCC(C)C ; CCCCCCCCCCCCCCC(=O)OC[C@@H](COP(=O)([O-])OCCN)OC(=O)CCCCCCCCCCCC1CC1 ; CCCCCCCCCCCCCCC(=O)OC[C@@H](COP(=O)([O-])OCCN)OC(=O)CCCCCCCCCCC1CCC1 ; CCCCCCCCCCCCCCC(=O)OC[C@@H](COP(=O)([O-])OCCN)OC(=O)CCCCCCCCCCC(C)(C)C ; CC[C@H](C)CCCCCCCCCCC(=O)O[C@@H](COC(=O)CCCCCCCCCCCC(C)C)COP(=O)([O-])OCCN]

or a pharmaceutically acceptable salt, hydrate, polymorph, co-crystal, tautomer, stereoisomer, isotopically labeled derivative, or prodrug thereof.

Also provided herein are compounds of Formula (II):

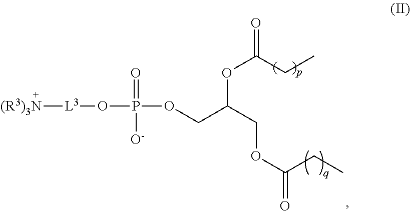

or a pharmaceutically acceptable salt, solvate, hydrate, polymorph, co-crystal, tautomer, stereoisomer, isotopically labeled derivative, or prodrug thereof, wherein:

L³ is substituted or unsubstituted C₃₋₈ alkylene, substituted or unsubstituted C₃₋₈ heteroalkylene, substituted or unsubstituted carbocyclylene, substituted or unsubstituted heterocyclylene, substituted or unsubstituted arylene, substituted or unsubstituted heteroarylene, or any combination thereof;

R³ is —H or branched or unbranched C₁₋₆ alkyl;

p is an integer from 12 to 14, inclusive; and

q is an integer from 12 to 14, inclusive.

In certain embodiments, a compound of Formula (II) is not of one of the following formulae:

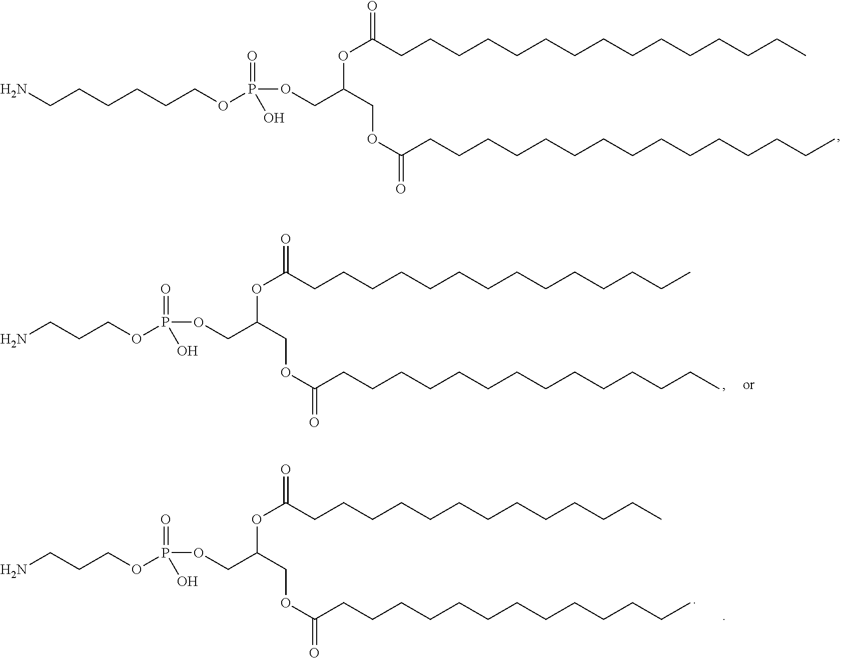 [SMILES: CCCCCCCCCCCCCCCC(=O)OCC(COP(=O)(O)OCCCCCCN)OC(=O)CCCCCCCCCCCCCCC ; CCCCCCCCCCCCCCC(=O)OCC(COP(=O)(O)OCCCN)OC(=O)CCCCCCCCCCCCCC ; CCCCCCCCCCCCCC(=O)OCC(COP(=O)(O)OCCCN)OC(=O)CCCCCCCCCCCCC]

In certain embodiments, the compound of Formula (II) is of one of the foregoing formulae.

In certain embodiments, a compound of Formula (II) is not a natural product.

In certain embodiments, a compound of Formula (II) is of Formula (IIa):

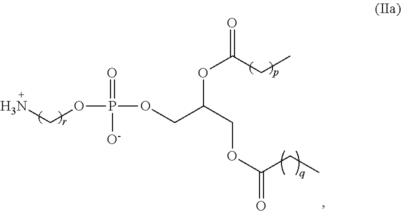

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIb):

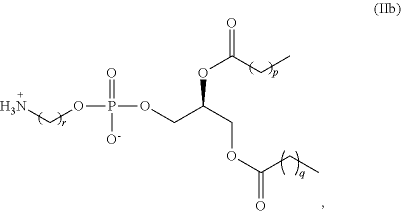

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIc):

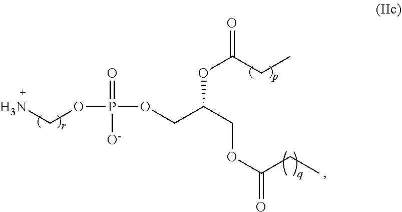

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IId):

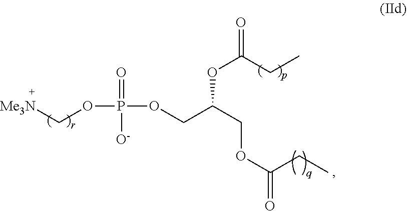

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIe):

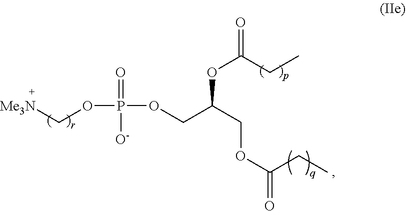

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIf):

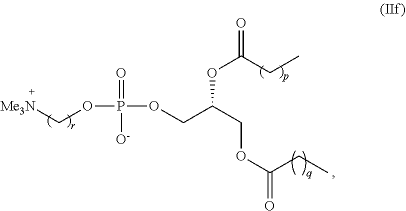

or a pharmaceutically acceptable salt, solvate, hydrate, polymorph, co-crystal, tautomer, stereoisomer, isotopically labeled derivative, or prodrug thereof.

As defined herein, p is an integer from 12 to 14, inclusive. In certain embodiments, p is 12. In some embodiments, p is 13. In certain embodiments, p is 14.

As defined herein, q is an integer from 12 to 14, inclusive. In certain embodiments, q is 12. In some embodiments, q is 13. In certain embodiments, q is 14.

In certain embodiments, p is 12, and q is 12. In some embodiments, p is 12, and q is 13. In certain embodiments, p is 12, and q is 14. In some embodiments, p is 13, and q is 12. In certain embodiments, p is 13, and q is 13. In some embodiments, p is 13, and q is 14. In certain embodiments, p is 14, and q is 12. In some embodiments, p is 14, and q is 13. In certain embodiments, p is 14, and q is 14.

As defined herein, L³ is substituted or unsubstituted C₃₋₈ alkylene, substituted or unsubstituted C₃₋₈ heteroalkylene, substituted or unsubstituted carbocyclylene, substituted or unsubstituted heterocyclylene, substituted or unsubstituted arylene, substituted or unsubstituted heteroarylene, or any combination thereof. In certain embodiments, L³ is substituted or unsubstituted C₃₋₈ alkylene, substituted or unsubstituted carbocyclylene, substituted or unsubstituted heterocyclylene, substituted or unsubstituted arylene, substituted or unsubstituted heteroarylene, or any combination thereof.

In some embodiments, L³ is substituted or unsubstituted C₃₋₈ alkylene. In certain embodiments, L³ is unsubstituted C₃₋₈ alkylene. In some embodiments, L³ is unsubstituted C₃ alkylene. In certain embodiments, L³ is unsubstituted C₄ alkylene. In some embodiments, L³ is unsubstituted C₅ alkylene. In certain embodiments, L³ is unsubstituted C₆ alkylene. In some embodiments, L³ is unsubstituted C₇ alkylene. In certain embodiments, L³ is unsubstituted C₈ alkylene. In some embodiments, L³ is selected from the group consisting of

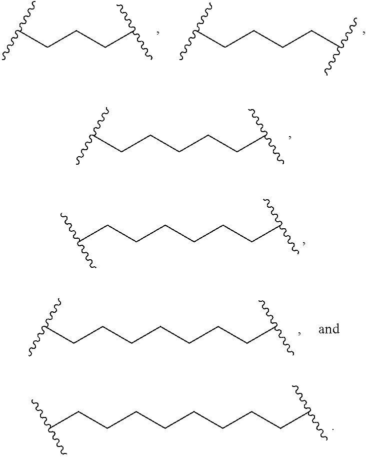 [SMILES: *CCC* ; *CCCC* ; *CCCCC* ; *CCCCCC* ; *CCCCCCC* ; *CCCCCCCC*]

In some embodiments, L³ is selected from the group consisting of

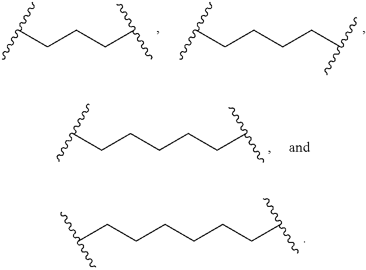 [SMILES: *CCC* ; *CCCC* ; *CCCCC* ; *CCCCCC*]

In certain embodiments, L³ is

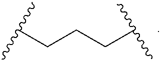 [SMILES: *CCC*]

In some embodiments, L³ in

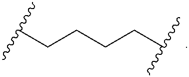 [SMILES: *CCCC*]

In certain embodiments, L³ is

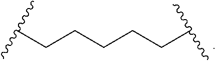 [SMILES: *CCCCC*]

In some embodiments, L³ is

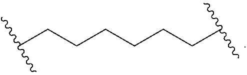 [SMILES: *CCCCCC*]

In some embodiments, L³ is substituted C₃₋₈ alkylene. In certain embodiments, L³ is substituted C₃ alkylene. In some embodiments, L³ is substituted C₄ alkylene. In certain embodiments, L³ is substituted C₅ alkylene. In some embodiments, L³ is substituted C₅ alkylene. In certain embodiments, L³ is substituted C₇ alkylene. In certain embodiments, L³ is substituted C₈ alkylene.

In some embodiments, L³ is substituted or unsubstituted C₃ s heteroalkylene. In certain embodiments, L³ is unsubstituted C₃₋₈ heteroalkylene. In some embodiments, L³ is unsubstituted C₃ heteroalkylene. In certain embodiments, L³ is unsubstituted C₄ heteroalkylene. In some embodiments, L³ is unsubstituted C₅ heteroalkylene. In certain embodiments, L³ is unsubstituted C₆ heteroalkylene. In some embodiments, L³ is unsubstituted C₇ heteroalkylene. In certain embodiments, L³ is unsubstituted C₈ heteroalkylene. In some embodiments, L³ is substituted C_{3 8} heteroalkylene. In certain embodiments, L³ is substituted C₃ heteroalkylene. In some embodiments, L³ is substituted C₄ heteroalkylene. In certain embodiments, L³ is substituted C₅ heteroalkylene. In some embodiments, L³ is substituted C₆ heteroalkylene. In certain embodiments, L³ is substituted C₇ heteroalkylene. In certain embodiments, L³ is substituted C₈ heteroalkylene.

In certain embodiments, L³ comprises substituted or unsubstituted carbocyclylene. In some embodiments, L³ comprises substituted carbocyclylene. In certain embodiments, L³ comprises unsubstituted carbocyclylene. In some embodiments, L³ comprises C₅₋₆ substituted carbocyclylene. In certain embodiments, L³ comprises C₅₋₆ unsubstituted carbocyclylene.

In certain embodiments, L³ comprises substituted or unsubstituted heterocyclylene. In some embodiments, L³ comprises substituted heterocyclylene. In certain embodiments, L³ comprises unsubstituted heterocyclylene. In some embodiments, L³ comprises C₅₋₆ substituted heterocyclylene.

In certain embodiments, L³ comprises substituted or unsubstituted arylene. In some embodiments, L³ comprises substituted arylene. In certain embodiments, L³ comprises unsubstituted arylene. In some embodiments, L³ comprises unsubstituted C₆₋₁₄ arylene. In certain embodiments, L³ comprises substituted C₆₋₁₄ arylene.

In some embodiments, L³ comprises substituted or unsubstituted heteroarylene. In some embodiments, L³ comprises substituted heteroarylene. In certain embodiments, L³ comprises substituted 5-6 membered heteroarylene. In some embodiments, L³ comprises unsubstituted heteroarylene. In certain embodiments, L³ comprises unsubstituted 5-6 membered heteroarylene.

As defined herein, r is an integer from 3 to 8, inclusive. In certain embodiments, r is 3. In some embodiments, r is 4. In certain embodiments, r is 5. In some embodiments, r is 6. In certain embodiments, r is 7. In some embodiments, r is 8.

In certain embodiments, p is 12, q is 12, and r is 3. In some embodiments, p is 12, q is 13, and r is 3. In certain embodiments, p is 12, q is 14, and r is 3. In some embodiments, p is 13, q is 12, and r is 3. In certain embodiments, p is 13, q is 13, and r is 3. In some embodiments, p is 13, q is 14, and r is 3. In certain embodiments, p is 14, q is 12, and r is 3. In some embodiments, p is 14, q is 13, and r is 3. In certain embodiments, p is 14, q is 14, and r is 3.

In certain embodiments, p is 12, q is 12, and r is 4. In some embodiments, p is 12, q is 13, and r is 4. In certain embodiments, p is 12, q is 14, and r is 4. In some embodiments, p is 13, q is 12, and r is 4. In certain embodiments, p is 13, q is 13, and r is 4. In some embodiments, p is 13, q is 14, and r is 4. In certain embodiments, p is 14, q is 12, and r is 4. In some embodiments, p is 14, q is 13, and r is 4. In certain embodiments, p is 14, q is 14, and r is 4.

In certain embodiments, p is 12, q is 12, and r is 5. In some embodiments, p is 12, q is 13, and r is 5. In certain embodiments, p is 12, q is 14, and r is 5. In some embodiments, p is 13, q is 12, and r is 5. In certain embodiments, p is 13, q is 13, and r is 5. In some embodiments, p is 13, q is 14, and r is 5. In certain embodiments, p is 14, q is 12, and r is 5. In some embodiments, p is 14, q is 13, and r is 5. In certain embodiments, p is 14, q is 14, and r is 5.

In certain embodiments, p is 12, q is 12, and r is 6. In some embodiments, p is 12, q is 13, and r is 6. In certain embodiments, p is 12, q is 14, and r is 6. In some embodiments, p is 13, q is 12, and r is 6. In certain embodiments, p is 13, q is 13, and r is 6. In some embodiments, p is 13, q is 14, and r is 6. In certain embodiments, p is 14, q is 12, and r is 6. In some embodiments, p is 14, q is 13, and r is 6. In certain embodiments, p is 14, q is 14, and r is 6.

In certain embodiments, p is 12, q is 12, and r is 7. In some embodiments, p is 12, q is 13, and r is 7. In certain embodiments, p is 12, q is 14, and r is 7. In some embodiments, p is 13, q is 12, and r is 7. In certain embodiments, p is 13, q is 13, and r is 7. In some embodiments, p is 13, q is 14, and r is 7. In certain embodiments, p is 14, q is 12, and r is 7. In some embodiments, p is 14, q is 13, and r is 7. In certain embodiments, p is 14, q is 14, and r is 7.

In certain embodiments, p is 12, q is 12, and r is 8. In some embodiments, p is 12, q is 13, and r is 8. In certain embodiments, p is 12, q is 14, and r is 8. In some embodiments, p is 13, q is 12, and r is 8. In certain embodiments, p is 13, q is 13, and r is 8. In some embodiments, p is 13, q is 14, and r is 8. In certain embodiments, p is 14, q is 12, and r is 8. In some embodiments, p is 14, q is 13, and r is 8. In certain embodiments, p is 14, q is 14, and r is 8.

In certain embodiments, a compound of Formula (II) is of one of the the following formulae:

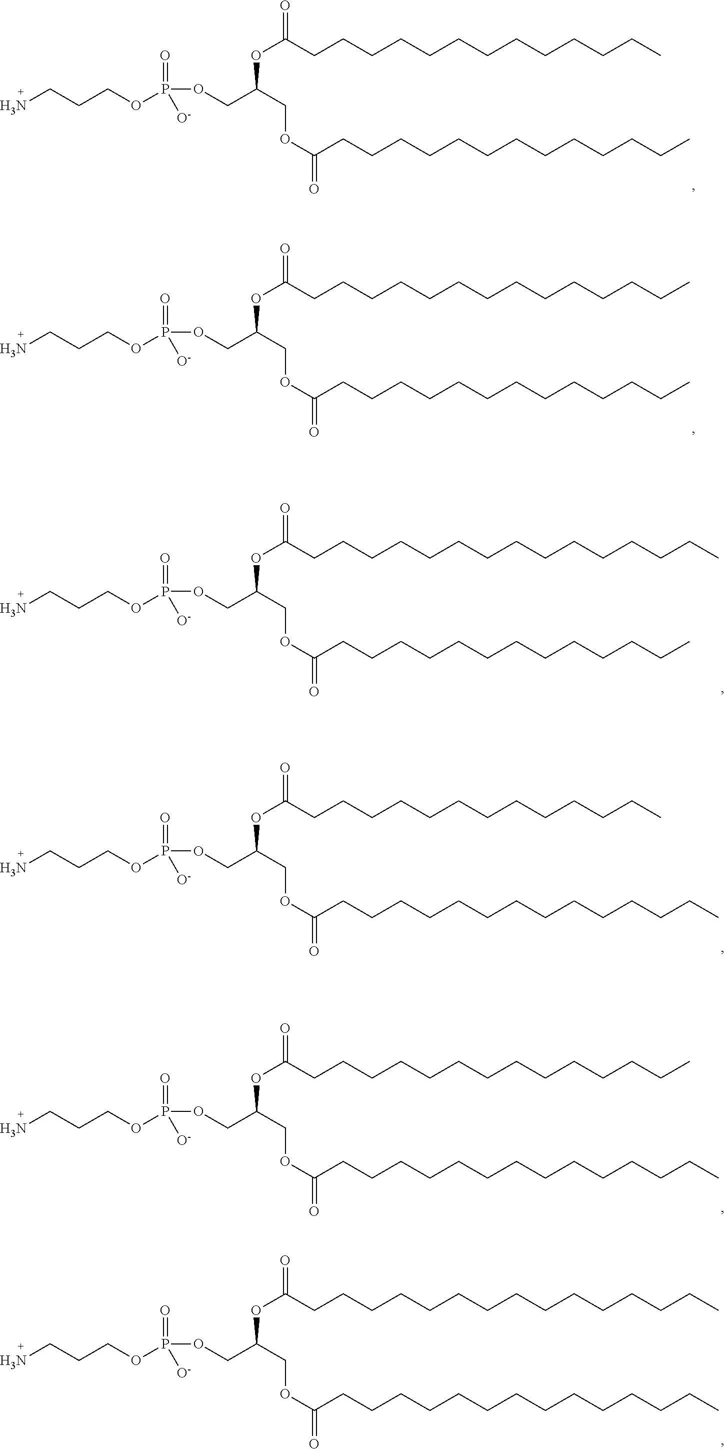 [SMILES: CCCCCCCCCCCCCC(=O)OC[C@@H](COP(=O)([O-])OCCCN)OC(=O)CCCCCCCCCCCCC ; CCCCCCCCCCCCCCC(=O)O[C@@H](COC(=O)CCCCCCCCCCCCC)COP(=O)([O-])OCCCN ; CCCCCCCCCCCCCCCC(=O)O[C@@H](COC(=O)CCCCCCCCCCCCC)COP(=O)([O-])OCCCN ; CCCCCCCCCCCCCCC(=O)OC[C@@H](COP(=O)([O-])OCCCN)OC(=O)CCCCCCCCCCCCC ; CCCCCCCCCCCCCCC(=O)OC[C@@H](COP(=O)([O-])OCCCN)OC(=O)CCCCCCCCCCCCCC ; CCCCCCCCCCCCCCCC(=O)O[C@@H](COC(=O)CCCCCCCCCCCCCC)COP(=O)([O-])OCCCN]

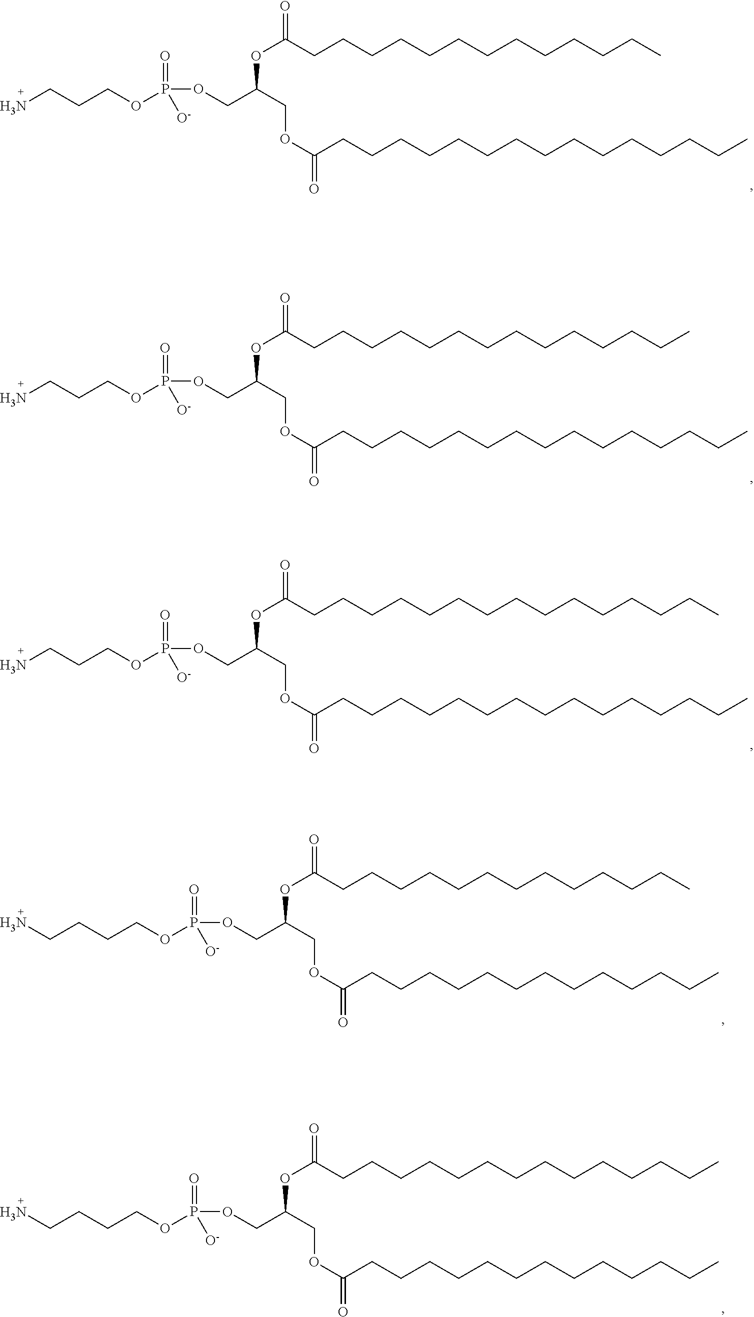 [SMILES: CCCCCCCCCCCCCCCC(=O)OC[C@@H](COP(=O)([O-])OCCCN)OC(=O)CCCCCCCCCCCCC ; CCCCCCCCCCCCCCCC(=O)OC[C@@H](COP(=O)([O-])OCCCN)OC(=O)CCCCCCCCCCCCCC ; CCCCCCCCCCCCCCCC(=O)OC[C@@H](COP(=O)([O-])OCCCN)OC(=O)CCCCCCCCCCCCCCC ; CCCCCCCCCCCCCC(=O)OC[C@@H](COP(=O)([O-])OCCCCN)OC(=O)CCCCCCCCCCCCC ; CCCCCCCCCCCCCCC(=O)O[C@@H](COC(=O)CCCCCCCCCCCCC)COP(=O)([O-])OCCCCN]

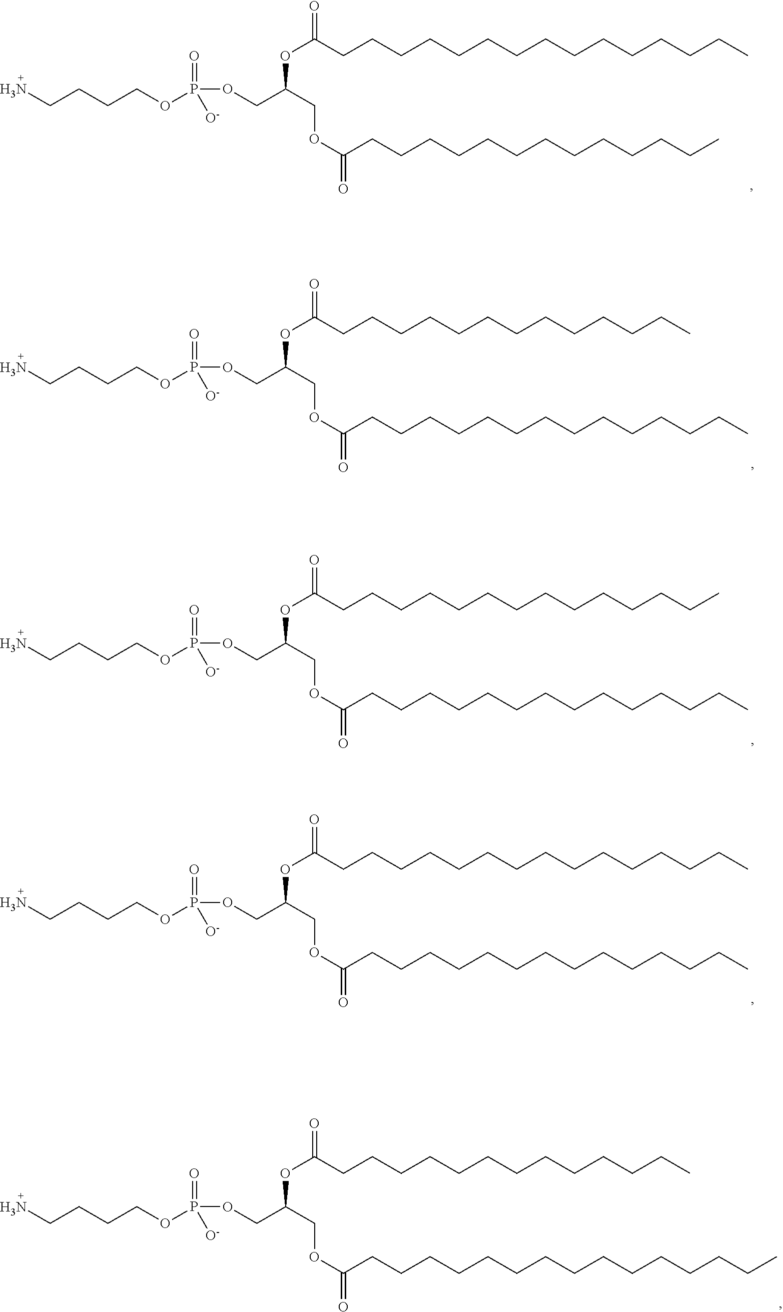 [SMILES: CCCCCCCCCCCCCCCC(=O)O[C@@H](COC(=O)CCCCCCCCCCCCC)COP(=O)([O-])OCCCCN ; CCCCCCCCCCCCCCC(=O)OC[C@@H](COP(=O)([O-])OCCCCN)OC(=O)CCCCCCCCCCCCC ; CCCCCCCCCCCCCCC(=O)OC[C@@H](COP(=O)([O-])OCCCCN)OC(=O)CCCCCCCCCCCCCC ; CCCCCCCCCCCCCCCC(=O)O[C@@H](COC(=O)CCCCCCCCCCCCCC)COP(=O)([O-])OCCCCN ; CCCCCCCCCCCCCCCC(=O)OC[C@@H](COP(=O)([O-])OCCCCN)OC(=O)CCCCCCCCCCCCC]

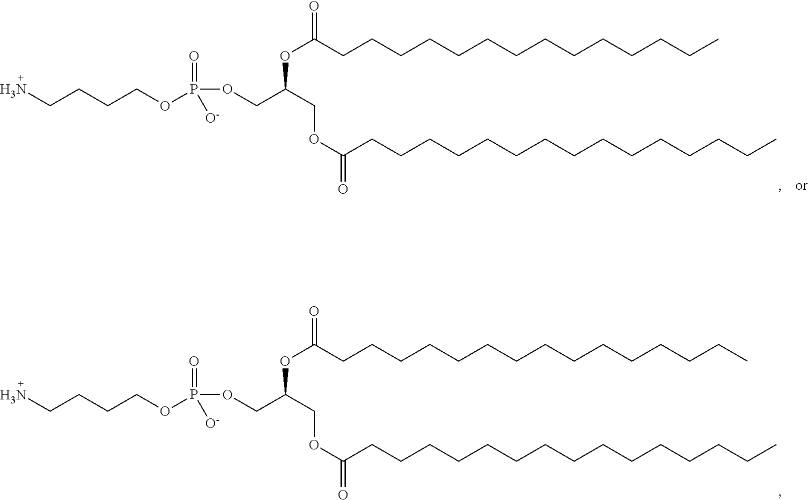 [SMILES: CCCCCCCCCCCCCCCC(=O)OC[C@@H](COP(=O)([O-])OCCCCN)OC(=O)CCCCCCCCCCCCCC ; CCCCCCCCCCCCCCCC(=O)OC[C@@H](COP(=O)([O-])OCCCCN)OC(=O)CCCCCCCCCCCCCCC]

or a pharmaceutically acceptable salt, hydrate, polymorph, co-crystal, tautomer, stereoisomer, isotopically labeled derivative, or prodrug thereof.

In some instances, compounds and generic formulae provided herein are drawn in zwitterionic form. Any compound or generic formula drawn as a zwitterion herein is understood to also encompass the corresponding non-zwitterionic form. For example, compounds of Formula (I) wherein R³ is —H (drawn in zwitterionic form herein) also encompass compounds of the following formula (non-zwitterionic form):

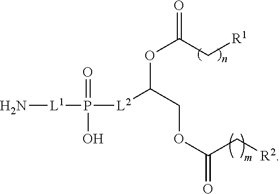

Similarly, for example, compounds of Formula (II) wherein R³ is —H (drawn in zwitterionic form herein) also encompass compounds of the following formula (non-zwitterionic form):

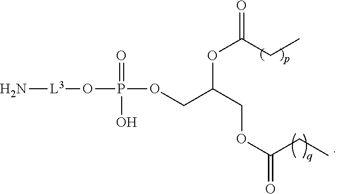

Compounds excluded by proviso in their non-zwitterionic form are also understood to be excluded in their zwitterionic form.

### Pharmaceutical Compositions, Kits, and Administration

The present disclosure provides pharmaceutical compositions comprising a compound described herein (e.g., a compound of Formula (I) or (II)), or a pharmaceutically acceptable salt, solvate, hydrate, polymorph, co-crystal, tautomer, stereoisomer, isotopically labeled derivative, or prodrug thereof, and optionally a pharmaceutically acceptable excipient. In certain embodiments, the pharmaceutical composition described herein comprises a compound of Formula (I) or (II), or a pharmaceutically acceptable salt, solvate, hydrate, polymorph, co-crystal, tautomer, stereoisomer, isotopically labeled derivative, or prodrug thereof, and a pharmaceutically acceptable excipient.

In certain embodiments, the compound described herein, or a pharmaceutically acceptable salt, solvate, hydrate, polymorph, co-crystal, tautomer, stereoisomer, isotopically labeled derivative, or prodrug thereof, is provided in an effective amount in the pharmaceutical composition. In certain embodiments, the effective amount is a therapeutically or prophylactically effective amount. In certain embodiments, the effective amount is an amount effective for treating a disease (e.g., proliferative disease, metabolic disorder, or inflammatory disease) in a subject in need thereof. In certain embodiments, the effective amount is an amount effective for preventing a disease (e.g., proliferative disease, metabolic disorder, or inflammatory disease) in a subject in need thereof. In certain embodiments, the effective amount is an amount effective for reducing the risk of developing a disease (e.g., proliferative disease, metabolic disorder, or inflammatory disease) in a subject in need thereof.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit. In certain embodiments, a pharmaceutical composition described herein could be prepared according to the known method such as a method described in the general rules for preparations of the Japanese Pharmacopoeia, 16th edition, the United States Pharmacopoeia, and the European Pharmacopoeia, 9th edition. A pharmaceutical composition of the invention could be administered to patients appropriately depending on the dosage form.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the compound of the disclosure, pharmaceutically acceptable excipient, agent, and/or any additional ingredients in a composition described herein will vary, depending upon the identity, size, and/or condition of the subject treated and further depending upon the route by which the composition is to be administered. The composition may comprise between 0.1% and 100% (w/w) agent.

Pharmaceutically acceptable excipients used in manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter and suppository waxes, coloring agents, coating agents, sweetening, flavoring, and perfuming agents, may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound of the disclosure required to achieve an effective amount will vary from subject to subject, depending, for example, on species, age, and general condition of a subject, severity of the side effects or disorder, identity of the particular compound of the disclosure, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of an agent described herein.

In certain embodiments, when multiple doses are administered to a subject or applied to a tissue or cell, the frequency of administering the multiple doses to the subject or applying the multiple doses to the tissue or cell may be, in non-limiting examples, three doses a day, two doses a day, one dose a day, one dose every other day, one dose every third day, one dose every week, one dose every two weeks, one dose every three weeks, or one dose every four weeks, or even slow dose controlled delivery over a selected period of time using a drug delivery device. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided compounds or compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., potency and/or efficacy) in treating a disease in a subject in need thereof, in preventing a disease in a subject in need thereof, in reducing the risk to develop a disease in a subject in need thereof, improving bioavailability, improving safety, reducing drug resistance, reducing and/or modifying metabolism, inhibiting excretion, and/or modifying distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent.

Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

In certain embodiments, the compounds or pharmaceutical compositions described herein can be administered in combination with an anti-cancer therapy including, but not limited to, surgery, radiation therapy, transplantation (e.g., stem cell transplantation, bone marrow transplantation), immunotherapy, and chemotherapy.

Also encompassed by the disclosure are kits (e.g., pharmaceutical packs). The kits provided may comprise a pharmaceutical composition or compound provided herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound provided herein. In some embodiments, the pharmaceutical composition or compound provided in the first container and the second container are combined to form one unit dosage form. A kit described herein may include one or more additional pharmaceutical agents described herein as a separate composition.

Thus, in one aspect, provided are kits including a first container comprising a compound or pharmaceutical composition described herein. In certain embodiments, the kits are useful for treating a disease (e.g., proliferative disease, metabolic disorder, or inflammatory disease) in a subject in need thereof. In certain embodiments, the kits are useful for preventing a disease (e.g., proliferative disease, metabolic disorder, or inflammatory disease) in a subject in need thereof. In certain embodiments, the kits are useful for reducing the risk of developing a disease (e.g., proliferative disease, metabolic disorder, or inflammatory disease) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, metabolic disorder, or inflammatory disease) in a subject in need thereof. In certain embodiments, the kits and instructions provide for preventing a disease (e.g., proliferative disease, metabolic disorder, or inflammatory disease) in a subject in need thereof. In certain embodiments, the kits and instructions provide for reducing the risk of developing a disease (e.g., proliferative disease, metabolic disorder, or inflammatory disease) in a subject in need thereof. A kit described herein may include one or more additional pharmaceutical agents described herein as a separate composition.

### Methods of Treatment and Uses

Provided herein are methods of using the compounds provided herein (e.g., compounds of Formulae (I) and (II)), and pharmaceutically acceptable salts, solvates, hydrates, polymorphs, co-crystals, tautomers, stereoisomers, isotopically labeled derivatives, and prodrugs thereof, and pharmaceutical compositions thereof.

Provided herein are methods of treating and/or preventing a disease in a subject, the methods comprising administering to the subject a compound of Formula (I) or (II), or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the disease is a proliferative disease (e.g., cancer), a metabolic disease, immune disorder, or an inflammatory disease.

Also provided herein are uses of the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, for the preparation of a medicament for treating and/or preventing a disease in a subject. In certain embodiments, the disease is a proliferative disease (e.g., cancer), a metabolic disease, immune disorder, or an inflammatory disease.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, for use in treating and/or preventing a disease in a subject. In certain embodiments, the disease is a proliferative disease (e.g., cancer), a metabolic disease, immune disorder, or an inflammatory disease.

Provided herein are methods of treating and/or preventing a disease in a subject, the methods comprising administering to the subject a compound of the formula:

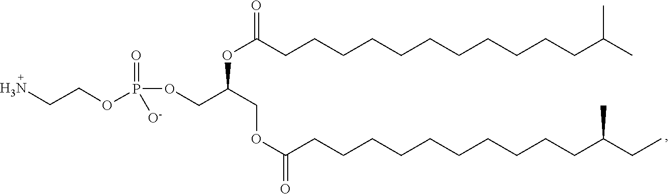 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the disease is a proliferative disease (e.g., cancer), a metabolic disease, immune disorder, or an inflammatory disease.

Also provided herein are uses of the compound of the formula:

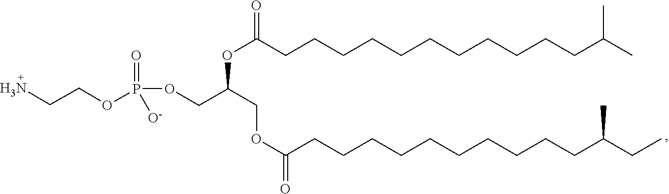 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labeled derivative, or prodrug thereof, or a pharmaceutical composition thereof, for the preparation of a medicament for treating a disease in a subject. In certain embodiments, the disease is a proliferative disease (e.g., cancer), a metabolic disease, immune disorder, or an inflammatory disease.

Also provided herein is a compound of the formula:

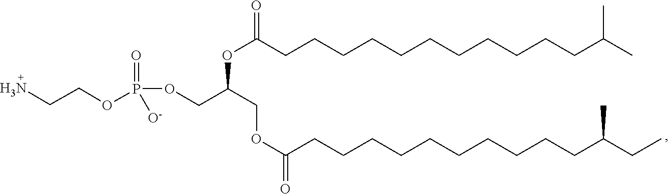 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labeled derivative, or prodrug thereof, or a pharmaceutical composition thereof, for use in treating and/or preventing a disease in a subject. In certain embodiments, the disease is a proliferative disease (e.g., cancer), a metabolic disease, immune disorder, or an inflammatory disease.

Also provided herein are methods of treating and/or preventing a disease in a subject, the methods comprising administering to the subject a low dose of a TLR2 agonist. In some embodiments, the TLR2 agonist is a compound of Formula (I) or Formula (II), or a pharmaceutically acceptable salt, solvate, hydrate, polymorph, co-crystal, tautomer, stereoisomer, isotopically labelled derivative, or prodrug thereof. In certain embodiments, the disease is a proliferative disease (e.g., cancer), a metabolic disease, immune disorder, or an inflammatory disease.

Also provided herein are uses of a TLR2 agonist for the preparation of a medicament for treating and/or preventing a disease in a subject comprising a low dose of the TLR2 agonist. In some embodiments, the TLR2 agonist is a compound of Formula (I) or Formula (II), or a pharmaceutically acceptable salt, solvate, hydrate, polymorph, co-crystal, tautomer, stereoisomer, isotopically labelled derivative, or prodrug thereof. In certain embodiments, the disease is a proliferative disease (e.g., cancer), a metabolic disease, immune disorder, or an inflammatory disease.

Also provided herein are TLR2 agonists and pharmaceutical compositions thereof comprising a low dose of the TLR2 agonist, for use in treating and/or preventing a disease in a subject. In some embodiments, the TLR2 agonist is a compound of Formula (I) or Formula (II), or a pharmaceutically acceptable salt, solvate, hydrate, polymorph, co-crystal, tautomer, stereoisomer, isotopically labelled derivative, or prodrug thereof. In certain embodiments, the disease is a proliferative disease (e.g., cancer), a metabolic disease, immune disorder, or an inflammatory disease.

Also provided herein are methods comprising administering to a subject an effective amount of a TLR2 agonist, wherein the effective amount of the TLR2 agonist is a low dose as described herein.

In certain embodiments, the “low dose” of the TLR2 agonist is approximately 0.01-10% of the TLR2 agonist's EC₅₀. In certain embodiments, the low dose of the TLR2 agonist is approximately 0.1-5% of the TLR2 agonist's EC₅₀. In certain embodiments, the low dose of the TLR2 agonist is approximately 0.5-2% of the TLR2 agonist's EC₅₀. In some embodiments, the low dose of the TLR2 agonist is approximately 0.5%, approximately 0.6% approximately 0.7%, approximately 0.8%, approximately 0.9%, approximately 1.0%, approximately 1.1%, approximately 1.2%, approximately 1.3%, approximately 1.4%, approximately 1.5%, approximately 1.6%, approximately 1.7%, approximately 1.8%, approximately 1.9%, or approximately 2.0%. In certain embodiments, the low dose of the TLR2 agonist is approximately 0.75-1.25% of the TLR2 agonist's EC₅₀. In certain embodiments, the low dose of the TLR2 agonist is approximately 0.75%, approximately 0.80%, approximately 0.85%, approximately 0.90%, approximately 0.95%, approximately 1%, approximately 1.05%, approximately 1.10%, approximately 1.15%, approximately 1.20%, or approximately 1.25% of the TLR2 agonist's EC₅₀. In certain embodiments, the low dose of the TLR2 agonist is approximately 1% of the TLR2 agonist's EC₅₀.

In certain embodiments, the disease is a metabolic disease. In some embodiments, the metabolic disease is metabolic syndrome, type 1 diabetes, type 2 diabetes, gestational diabetes, hyperglycemia, hyperinsulinemia, insulin resistance, or obesity. In certain embodiments, the metabolic disease is metabolic syndrome. In some embodiments, the metabolic disease is type 2 diabetes.

In certain embodiments, the disease is an inflammatory disease. In some embodiments, the inflammatory disorder and/or the immune disorder is a gastrointestinal disorder. In certain embodiments, the inflammatory disease is inflammatory bowel disease. Other non-limiting examples of inflammatory diseases are provided herein.

In certain embodiments, the disease is a proliferative disease. In some embodiments, the proliferative disease is cancer. Non-limiting examples of cancers are provided herein.

In certain embodiments, the compound of Formula (I) or (II), or a pharmaceutically acceptable salt, solvate, hydrate, polymorph, co-crystal, tautomer, stereoisomer, isotopically labeled derivative, or prodrugs thereof, or a pharmaceutical composition thereof, is administered orally, parenterally, intramuscularly, subcutaneously, intravenously, or transdermally. In some embodiments, the compound of Formula (I) or (II), or a pharmaceutically acceptable salt, solvate, hydrate, polymorph, co-crystal, tautomer, stereoisomer, isotopically labeled derivative, or prodrugs thereof, or a pharmaceutical composition thereof, is administered orally. In certain embodiments, the compound of Formula (I) or (II), or a pharmaceutically acceptable salt, solvate, hydrate, polymorph, co-crystal, tautomer, stereoisomer, isotopically labeled derivative, or prodrugs thereof, or a pharmaceutical composition thereof, is administered parenterally. In some embodiments, the compound of Formula (I) or (II), or a pharmaceutically acceptable salt, solvate, hydrate, polymorph, co-crystal, tautomer, stereoisomer, isotopically labeled derivative, or prodrugs thereof, or a pharmaceutical composition thereof, is administered intramuscularly. In certain embodiments, the compound of Formula (I) or (II), or a pharmaceutically acceptable salt, solvate, hydrate, polymorph, co-crystal, tautomer, stereoisomer, isotopically labeled derivative, or prodrugs thereof, or a pharmaceutical composition thereof, is administered subcutaneously. In some embodiments, the compound of Formula (I) or (II), or a pharmaceutically acceptable salt, solvate, hydrate, polymorph, co-crystal, tautomer, stereoisomer, isotopically labeled derivative, or prodrugs thereof, or a pharmaceutical composition thereof, is administered intravenously. In certain embodiments, the compound of Formula (I) or (II), or a pharmaceutically acceptable salt, solvate, hydrate, polymorph, co-crystal, tautomer, stereoisomer, isotopically labeled derivative, or prodrugs thereof, or a pharmaceutical composition thereof, is administered transdermally.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, for use as a vaccine adjuvant.

Also provided herein are methods of modulating an immune response, the methods comprising administering to the subject a compound of Formula (I) or (II), or a pharmaceutically acceptable salt, solvate, hydrate, polymorph, co-crystal, tautomer, stereoisomer, isotopically labeled derivative, or prodrug thereof, or a pharmaceutical composition thereof. In some embodiments, the method is for inhibiting an immune response. In certain embodiments, the method is for inducing immune tolerance. In some embodiments, the method is for stimulating an immune response. In certain embodiments, the method is for reducing an immune response.

Also provided herein are methods of inducing cytokine release in a subject, the methods comprising administering to the subject a compound of Formula (I) or (II), or a pharmaceutically acceptable salt, solvate, hydrate, polymorph, co-crystal, tautomer, stereoisomer, isotopically labeled derivative, or prodrug thereof, or a pharmaceutical composition thereof. In some embodiments, the method is for inducing release or production of type 1 cytokines. In certain embodiments, the method is for inducing release or production of type 2 cytokines. In some embodiments, the method is for inducing release or production of IL-2, IFNγ, IL-12, TNFα, TNF-β, MCP-1, IL-4, IL-5, IL-6, IL-10, IL-13, IL-17A, TGF-β, IL-12B, or IL-23A, or combinations thereof. In certain embodiments, the method is for inducing release or production of TNFα, IL-6, IL-10, MCP-1, or combinations thereof. In certain embodiments, the method is for inducing release or production of TNFα, IL-6, IL-23A, IL-12B, or combinations thereof. In some embodiments, the method is for inducing release or production of TNFα. In certain embodiments, the method is for inducing release or production of IL-6. In some embodiments, the method is for inducing release or production of IL-10. In certain embodiments, the method is for inducing release or production of MCP-1. In certain embodiments, the method is for inducing release or production of IL-23A. In certain embodiments, the method is for inducing release or production of IL-12B. In some embodiments, the method is for inducing release or production of IFNγ, IL-17A, IL-2, IL-4, or combinations thereof. In certain embodiments, the method is for inducing release or production of IFNγ. In some embodiments, the method is for inducing release or production of IL-17A. In certain embodiments, the method is for inducing release or production of IL-2. In some embodiments, the method is for inducing release or production of IL-4.

Also provided herein are methods of activating a TLR2 receptor, the methods comprising administering to the subject a compound of Formula (I) or (II), or a pharmaceutically acceptable salt, solvate, hydrate, polymorph, co-crystal, tautomer, stereoisomer, isotopically labeled derivative, or prodrug thereof, or a pharmaceutical composition thereof.

Also provided herein are uses of the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, for the preparation of a medicament for modulating an immune response in a subject. In some embodiments, the medicament is for inhibiting an immune response. In certain embodiments, the medicament is for inducing immune tolerance. In some embodiments, the medicament is for stimulating an immune response. In certain embodiments, the medicament is for reducing an immune response.

Also provided herein are uses of the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, for the preparation of a medicament for inducing cytokine release in a subject. In some embodiments, the medicament is for inducing release or production of type 1 cytokines. In certain embodiments, the medicament is for inducing release or production of type 2 cytokines. In some embodiments, the medicament is for inducing release or production of IL-2, IFNγ, IL-12, TNFα, TNF-β, MCP-1, IL-4, IL-5, IL-6, IL-10, IL-13, IL-17A, TGF-β, IL-12B, or IL-23A, or combinations thereof. In certain embodiments, the medicament is for inducing release or production of TNFα, IL-6, IL-23A, IL-12B, or combinations thereof. In certain embodiments, the medicament is for inducing release or production of TNFα, IL-6, IL-10, MCP-1, or combinations thereof. In some embodiments, the medicament is for inducing release or production of TNFα. In certain embodiments, the medicament is for inducing release or production of IL-6. In some embodiments, the medicament is for inducing release or production of IL-10. In certain embodiments, the medicament is for inducing release or production of MCP-1. In certain embodiments, the medicament is for inducing release or production of IL-23A. In certain embodiments, the medicament is for inducing release or production of IL-12B. In some embodiments, the medicament is for inducing release or production of IFNγ, IL-17A, IL-2, IL-4, or combinations thereof. In certain embodiments, the medicament is for inducing release or production of IFNγ. In some embodiments, the medicament is for inducing release or production of IL-17A. In certain embodiments, the medicament is for inducing release or production of IL-2. In some embodiments, the medicament is for inducing release or production of IL-4.

Also provided herein are uses of the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, for the preparation of a medicament for activating a TLR2 receptor in a subject.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, for use in modulating an immune response in a subject. In some embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inhibiting an immune response. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing immune tolerance. In some embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in stimulating an immune response. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in reducing an immune response.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, for use in inducing a cytokine release in a subject. In some embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of type 1 cytokines. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of type 2 cytokines. In some embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of IL-2, IFNγ, IL-12, TNFα, TNF-β, MCP-1, IL-4, IL-5, IL-6, IL-10, IL-13, IL-17A, TGF-β, IL-12B, or IL-23A, or combinations thereof. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of TNFα, IL-6, IL-23A, IL-12B, or combinations thereof. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of TNFα, IL-6, IL-10, MCP-1, or combinations thereof. In some embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of TNFα. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of IL-6. In some embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of IL-10. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of MCP-1. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of IL-23A. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of IL-12B. In some embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of IFNγ, IL-17A, IL-2, IL-4, or combinations thereof. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of IFNγ. In some embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of IL-17A. In certain embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of IL-2. In some embodiments, the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, are for use in inducing release or production of IL-4.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical compositions thereof, for use in activating a TLR2 receptor in a subject.

Also provided herein are methods of modulating an immune response in a subject, the methods comprising administering to the subject a compound of the formula:

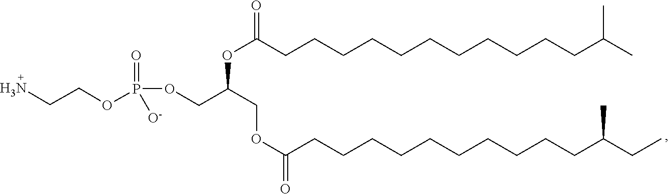 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labeled derivative, or prodrug thereof, or a pharmaceutical composition thereof. In some embodiments, the method is for inhibiting an immune response. In certain embodiments, the method is for inducing immune tolerance. In some embodiments, the method is for stimulating an immune response. In certain embodiments, the method is for reducing an immune response.

Also provided herein are methods of inducing a cytokine release in a subject, the methods comprising administering to the subject a a compound of the formula:

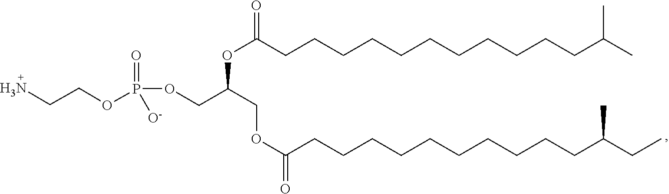 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labeled derivative, or prodrug thereof, or a pharmaceutical composition thereof. In some embodiments, the method is for inducing release or production of type 1 cytokines. In certain embodiments, the method is for inducing release or production of type 2 cytokines. In some embodiments, the method is for inducing release or production of IL-2, IFNγ, IL-12, TNFα, TNF-β, MCP-1, IL-4, IL-5, IL-6, IL-10, IL-13, IL-17A, TGF-β, IL-12B, or IL-23A, or combinations thereof. In certain embodiments, the method is for inducing release or production of TNFα, IL-6, IL-23A, IL-12B, or combinations thereof. In certain embodiments, the method is for inducing release or production of TNFα, IL-6, IL-10, MCP-1, or combinations thereof. In some embodiments, the method is for inducing release or production of TNFα. In certain embodiments, the method is for inducing release or production of IL-6. In some embodiments, the method is for inducing release or production of IL-10. In certain embodiments, the method is for inducing release or production of MCP-1. In certain embodiments, the method is for inducing release or production of IL-23A. In certain embodiments, the method is for inducing release or production of IL-12B. In some embodiments, the method is for inducing release or production of IFNγ, IL-17A, IL-2, IL-4, or combinations thereof. In certain embodiments, the method is for inducing release or production of IFNγ. In some embodiments, the method is for inducing release or production of IL-17A. In certain embodiments, the method is for inducing release or production of IL-2. In some embodiments, the method is for inducing release or production of IL-4.

Also provided herein are methods of activating a TLR2 receptor in a subject, the methods comprising administering to the subject a a compound of the formula:

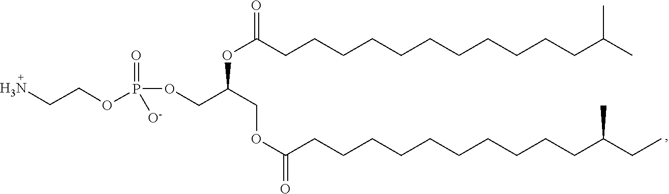 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labeled derivative, or prodrug thereof, or a pharmaceutical composition thereof.

Also provided herein are uses of a compound of the formula:

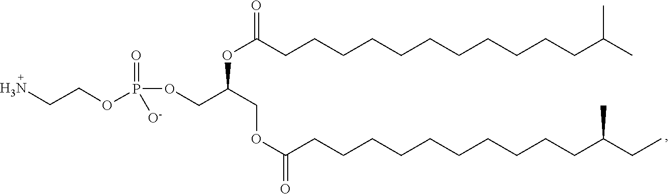 [SMILES: CC[C@H](C)CCCCCCCCCCC(=O)OC[C@@H](COP(=O)([O-])OCCN)OC(=O)CCCCCCCCCCCC(C)C]

and pharmaceutically acceptable salts, solvates, hydrates, polymorphs, co-crystals, tautomers, stereoisomers, isotopically labeled derivatives, and prodrugs thereof, and pharmaceutical compositions thereof, for the preparation of a medicament for modulating an immune response in a subject. In some embodiments, the medicament is for inhibiting an immune response. In certain embodiments, the medicament is for inducing immune tolerance. In some embodiments, the medicament is for stimulating an immune response. In certain embodiments, the medicament is for reducing an immune response.

Also provided herein are uses of a compound of the formula:

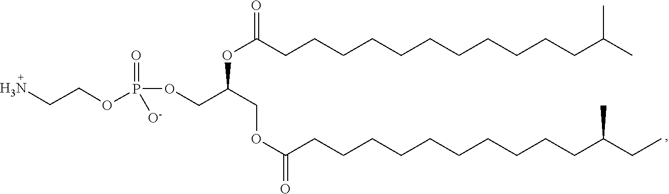 [SMILES: CC[C@H](C)CCCCCCCCCCC(=O)OC[C@@H](COP(=O)([O-])OCCN)OC(=O)CCCCCCCCCCCC(C)C]

and pharmaceutically acceptable salts, solvates, hydrates, polymorphs, co-crystals, tautomers, stereoisomers, isotopically labeled derivatives, and prodrugs thereof, and pharmaceutical compositions thereof, for the preparation of a medicament for inducing a cytokine release in a subject. In some embodiments, the medicament is for inducing release or production of type 1 cytokines. In certain embodiments, the medicament is for inducing release or production of type 2 cytokines. In some embodiments, the medicament is for inducing release or production of IL-2, IFNγ, IL-12, TNFα, TNF-β, MCP-1, IL-4, IL-5, IL-6, IL-10, IL-13, IL-17A, TGF-β, IL-12B, or IL-23A, or combinations thereof. In certain embodiments, the medicament is for inducing release or production of TNFα, IL-6, IL-23A, IL-12B, or combinations thereof. In certain embodiments, the medicament is for inducing release or production of TNFα, IL-6, IL-10, MCP-1, or combinations thereof. In some embodiments, the medicament is for inducing release or production of TNFα. In certain embodiments, the medicament is for inducing release or production of IL-6. In some embodiments, the medicament is for inducing release or production of IL-10. In certain embodiments, the medicament is for inducing release or production of MCP-1. In certain embodiments, the medicament is for inducing release or production of IL-23A. In certain embodiments, the medicament is for inducing release or production of IL-12B. In some embodiments, the medicament is for inducing release or production of IFNγ, IL-17A, IL-2, IL-4, or combinations thereof. In certain embodiments, the medicament is for inducing release or production of IFNγ. In some embodiments, the medicament is for inducing release or production of IL-17A. In certain embodiments, the medicament is for inducing release or production of IL-2. In some embodiments, the medicament is for inducing release or production of IL-4.

Also provided herein are uses of a compound of the formula:

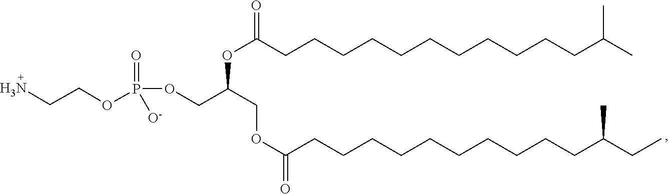 [SMILES: CC[C@H](C)CCCCCCCCCCC(=O)OC[C@@H](COP(=O)([O-])OCCN)OC(=O)CCCCCCCCCCCC(C)C]

and pharmaceutically acceptable salts, solvates, hydrates, polymorphs, co-crystals, tautomers, stereoisomers, isotopically labeled derivatives, and prodrugs thereof, and pharmaceutical compositions thereof, for the preparation of a medicament for activating a TLR2 receptor in a subject.

In certain embodiments, the compound of the formula:

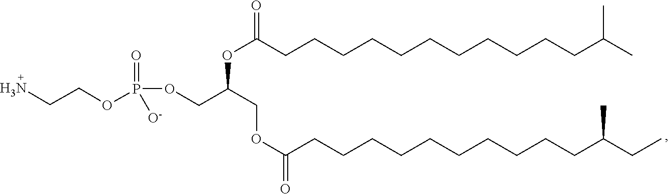 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labeled derivative, or prodrugs thereof, or a pharmaceutical composition thereof, is administered orally, parenterally, intramuscularly, subcutaneously, intravenously, or transdermally.

### Additional Methods and Uses

The present disclosure also provides methods comprising contacting a cell, tissue, or biological sample with an effective amount of a compound provided herein, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, these methods modulate an immune response. In some embodiments, these methods inhibit an immune response. In certain embodiments, these methods induce immune tolerance. In some embodiments, these methods stimulate an immune response. In certain embodiments, these methods reduce an immune response. In some embodiments, these methods induce a cytokine release. In certain embodiments, these methods activate a TLR2 receptor.

Provided herein are methods comprising contacting a cell, tissue, or biological sample with a compound of Formula (I) or (II), or a compound of the formula:

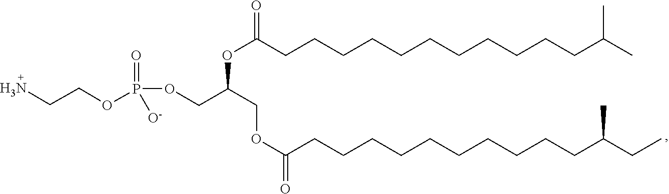 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the contacting modulates an immune response. In some embodiments, the contacting inhibits an immune response. In certain embodiments, the contacting induces immune tolerance. In some embodiments, the contacting stimulates an immune response. In certain embodiments, the contacting reduces an immune response. In some embodiments, these methods induce cytokine release. In certain embodiments, these methods activate a TLR2 receptor.

In some embodiments, any of the compounds or compositions described herein are contacted with a cell in vivo, e.g., in an organism. In some embodiments, any of the compounds or compositions described herein are contacted with a cell in vitro, e.g., in cell culture. In some embodiments, any of the compounds or compositions described herein are contacted with a cell ex vivo, meaning the cell is removed from an organism prior to the contacting. As will be evident to one of skill in the art, the term cell may be used to refer to a single cell as well as a population of cells. In some embodiments, the populations cells are contacted with any of the compounds described herein for use in personalized medicine, for example, for diagnostic and/or therapeutic purposes.

In general, any cells known in the art may be used in the methods and uses described herein. In certain embodiments, the cell is derived from a cell line. In some embodiments, the cell is obtained from an organism, such as a subject. In certain embodiments, the cell is a dendritic cell. In some embodiments, the cell is a bone marrow-derived dendritic cell. In certain embodiments, the cell is a blood cell. In some embodiments, the cell is a white blood cell. In certain embodiments, the cell is an immune cell. In some embodiments, the cell is an innate immune cell. In certain embodiments, the cell is a T cell. In some embodiments, the cell is a mouse bone marrow-derived dendritic cell (mBMDC). In certain embodiments, the cell is an OVA-specific CD4⁺ T cell. In some embodiments, the cell is an IL-17A producing cell. In certain embodiments, the cell is an IFNγ producing cell. In some embodiments, innate immune cells are co-cultured with OVA-specific CD4⁺ T cells and OT-II peptides. In certain embodiments, BMDCs are co-cultured with OVA-specific CD4⁺ T cells and OT-II peptides. In some embodiments, the cell is a myeloid cell. In certain embodiments, the cell is a human myeloid cell.

In certain embodiments, these methods modulate an immune response. In some embodiments, these methods inhibit an immune response. In certain embodiments, these methods induce immune tolerance. In some embodiments, these methods stimulate an immune response. In certain embodiments, these methods reduce an immune response. In some embodiments, these methods induce a cytokine release. In certain embodiments, these methods activate a TLR2 receptor. In some embodiments, these methods induce release or production of type 1 cytokines. In certain embodiments, these methods induce release or production of type 2 cytokines. In some embodiments, these methods induce release or production of IL-2, IFNγ, IL-12, TNFα, TNF-β, MCP-1, IL-4, IL-5, IL-6, IL-10, IL-13, IL-17A, TGF-β, IL-12B, or IL-23A, or combinations thereof. In certain embodiments, these methods induce release or production of TNFα, IL-6, IL-23A, IL-12B, or combinations thereof. In certain embodiments, these methods induce release or production of TNFα, IL-6, IL-10, MCP-1, or combinations thereof. In some embodiments, these methods induce release or production of TNFα. In certain embodiments, these methods induce release or production of IL-6. In some embodiments, these methods induce release or production of IL-10. In certain embodiments, these methods induce release or production of MCP-1. In certain embodiments, these methods induce release or production of IL-23A. In certain embodiments, these methods induce release or production of IL-12B. In some embodiments, these methods induce release or production of IFNγ, IL-17A, IL-2, IL-4, or combinations thereof. In certain embodiments, these methods induce release or production of IFNγ. In some embodiments, these methods induce release or production of IL-17A. In certain embodiments, these methods induce release or production of IL-2. In some embodiments, these methods induce release or production of IL-4.

In some embodiments, the methods further comprise measuring or assessing the level of one or more properties of the cell. In some embodiments, the level of one or more properties of the cell is assessed following contacting the cell with any of the compounds or compositions described herein. In some embodiments, the level of one or more properties following contacting the cell with any of the compounds or compositions described herein is compared to the level of one or more properties in a reference sample or prior to contacting the cell with the compounds or composition. In some embodiments, the contacting the cell with any of the compounds or compositions described herein increases one or more properties of the cell. In some aspects, the methods described herein may be used to determine whether a cell is susceptible to treatment with the compounds or compositions described herein. In some embodiments, if the level of one or more properties is increased following contacting the cell with any of the compounds or compositions described herein, the cell is determined to be susceptible to treatment with the compound or composition. In some embodiments, if the level of one or more properties is increased following contacting the cell with any of the compounds or compositions described herein, the compound or composition is determined to be a candidate for a disease or disorder associated with the cell.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Development of Phosphatidylalcoholamine Compounds

The human gut bacteria Akkermansia muciniphila frequently has inverse correlations with human disease severity, but the underlying molecular mechanisms have not been understood.(22-24) Identifying these molecular signals has broad significance because A. muciniphila is one of the few bacterial species that induces antigen-specific T cell responses during homeostasis. Akkermansia muciniphila has been associated with positive systemic effects on host metabolism, favorable outcomes to checkpoint blockade in cancer immunotherapy, and homeostatic immunity (5, 6, 25, 26).

Here it is reported that compounds such as phosphatidylethanolamines (PE) in which both acyl groups have a fifteen-carbon chain containing a methyl branch are responsible for immunomodulatory signaling through the TLR2 receptor. Synthetic compounds recapitulate the activity of the natural material. Identifying the lipid signal and its cognate receptor reveals a surprising aspect of immune signaling and suggests potential therapeutic approaches. In particular, the identification of a lipid from A. muciniphila's cell membrane that recapitulates the immunomodulatory activity of A. muciniphila in cell-based assays (7). The isolated immunogen, a diacyl phosphatidylethanolamine with two branched chains (a15:0-i15:0 PE), was characterized through both spectroscopic analysis and chemical synthesis. The immunogenic activity of a15:0-i15:0 PE has a highly restricted structure-activity relationship, and its immune signaling requires an unexpected toll-like receptor 2/1 heterodimer (16, 27). Certain features of the phospholipid's activity are noteworthy: it is significantly less potent than known natural and synthetic toll-like receptor 2 agonists; it preferentially induces some inflammatory cytokines but not others; and at low doses (1% of EC₅₀) it resets activation thresholds and responses for immune signaling. Identifying both the molecule and an equipotent synthetic analog, its non-canonical toll-like receptor 2/1 signaling pathway, its immunomodulatory selectivity, and its low dose immunoregulatory effects provides a molecular mechanism for a model of A. muciniphila's ability to set immunological tone and its varied roles in health and disease.

Previously, to identify immunoregulatory small molecules from gut microbes, an unbiased functional assay was employed using cytokine release from murine bone marrow-derived dendritic cells (mBMDCs) in response to fractionated bacterial extracts (7). Dendritic cells, which are part of the innate immune system, detect pathogen-associated molecules and relay information to the adaptive immune system through the release of cytokines. It was reasoned that the same approach would identify immunomodulatory molecules produced by A. muciniphila.

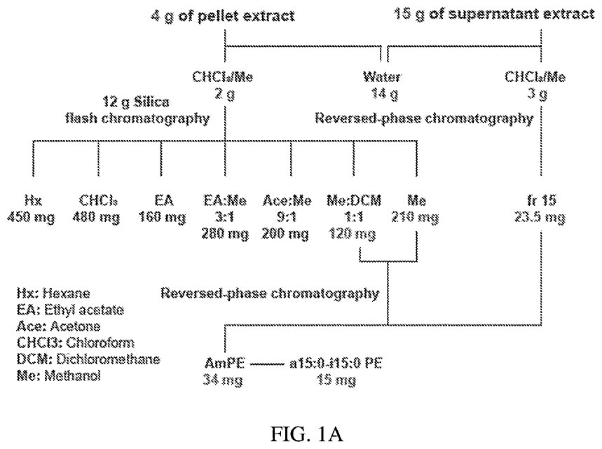

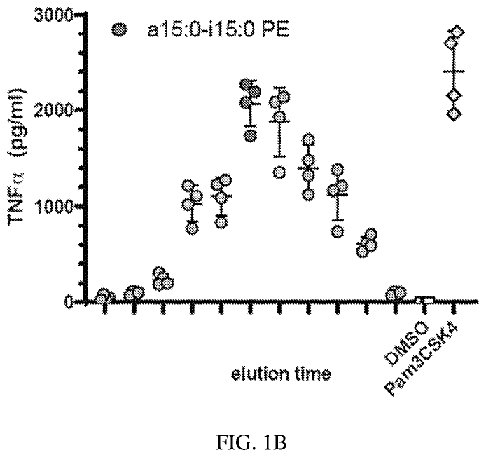

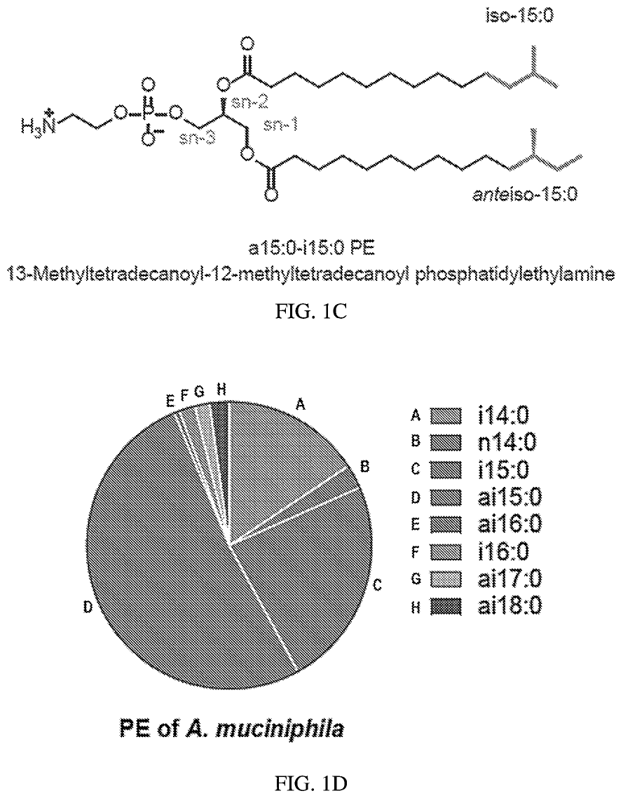

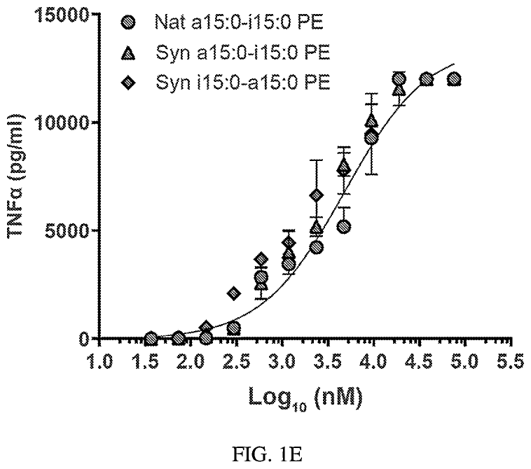

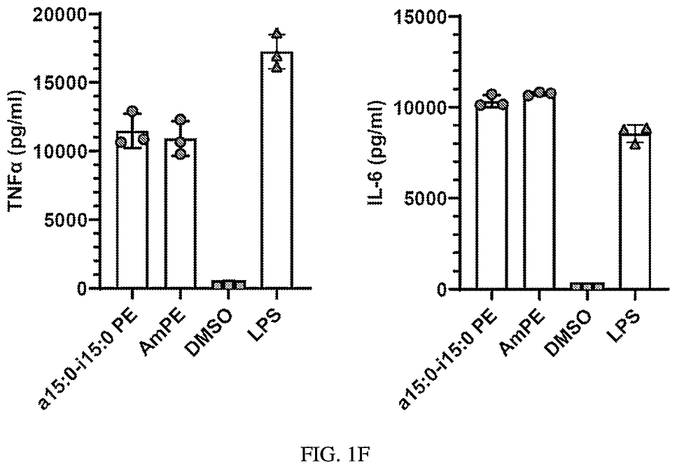

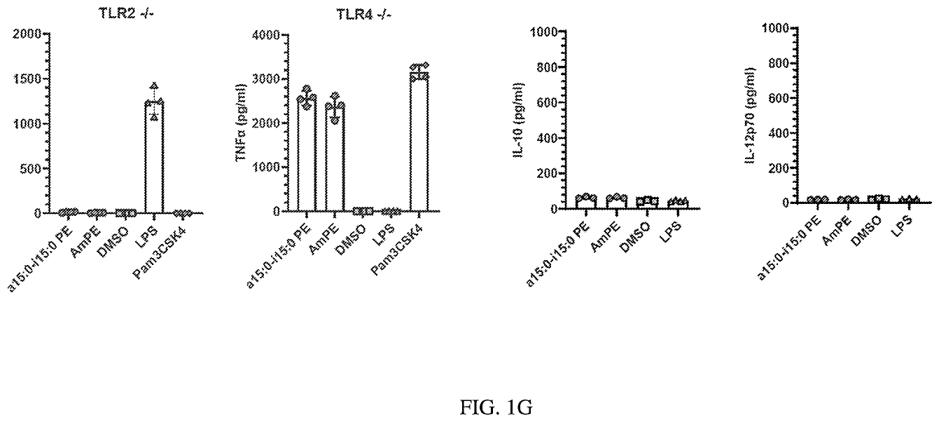

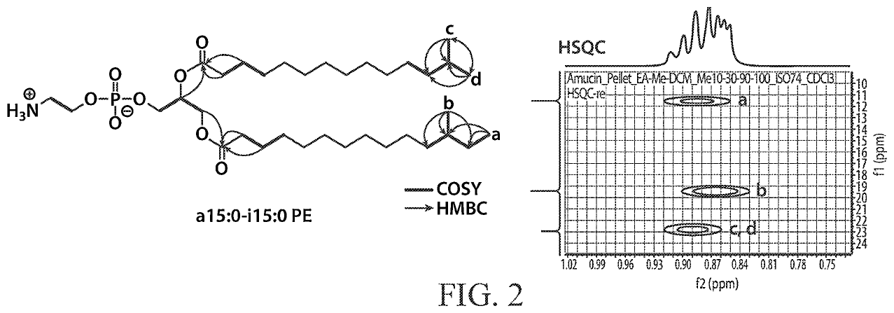

Both the cell pellet and supernatant from A. muciniphila BAA-835 cultures were assayed for cytokine induction (TNFα) from murine bone marrow derived dendritic cells (mBMDCs) (7). An initial culture had a prominent signal in the crude lipid extract (FIG. 1B). Large-scale culturing (128 L) led to a combined lipid extract (19 g), which upon further chromatographic separations with normal and reverse phase chromatography, led to a single active fraction that induced TNFα release (FIG. 1A). The active fraction was a mixture of closely related molecules, and the major component (15 mg) was focused on (FIG. 1A). Mass spectroscopic analysis indicated a molecular formula of C₃₅H₇₁NO₈P, suggesting a phospholipid, and preliminary ¹³C and ¹H NMR analysis identified a phosphatidylethanolamine (PE), the dominant membrane phospholipid in many bacteria (8). PEs often have a glycerol core, a polar phosphoethanolamine head group at the sn-3 position, and two fatty acid esters at the sn-2 and sn-1 positions (FIG. 1C). Additional NMR analysis revealed that both chains had methyl branches (FIG. 1C, FIG. 2). One acyl chain had a terminal iso branch, and the other had a terminal anteiso branch, indicating that the methyl groups were on positions 12 and 13 of a 14-carbon fatty acid (FA) (FIG. 1C). The order of the acyl groups was determined by selective hydrolysis to liberate the FA attached at the sn-2 position. The active molecule's chemical name is 12-methyltetradecanoyl-13-methyltetradecanoyl-sn-glycero-3-phosphoethanolamine, which is commonly abbreviated a15:0-i15:0 PE (FIG. 1C). Producers of a15:0-i15:0 PE were not identified in frequently encountered gut microbes nor in gut microbes with reported immunomodulatory effects (23). Membrane lipids reflect both evolutionary history and current environment. A. muciniphila, the only member of Verrucomicrobia in the gut microbiota, is a phylogenetic outlier specialized for life in the mucin layer. Metabolomic analysis, phylogenetic placement, and a distinctive microenvironment all support a singular association of A. muciniphila with a15:0-i15:0 PE.

The active fraction contained all of the PE produced by A. muciniphila, the later eluting fractions were triglycerides, and the earlier eluting fractions were diacylglycerides with different head groups. The PE FAs were dominated (92%) by relatively short, branched-chain FAs (BCFAs): a15:0 (52%), i15:0 (24%), and i14:0 (16%) (FIG. 1D). Small amounts of a17:0 and i16:0 are also present.

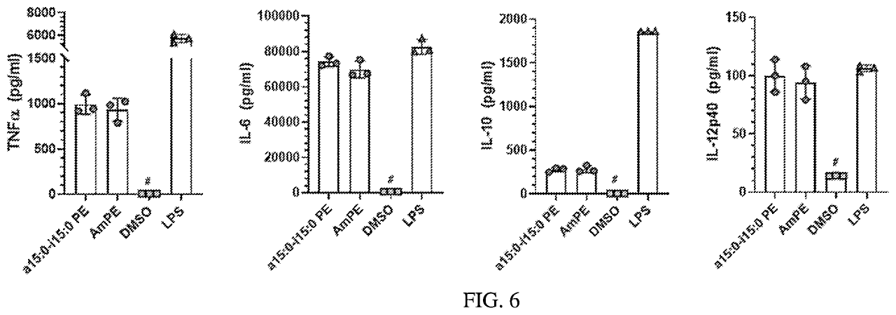

In the assay discussed herein, the major component compound (˜50%), a15:0-i15:0 PE, of A. muciniphila's lipid membrane had a robust dose-response curve for induction of TNFα (FIG. 1E). In addition to dramatically upregulating TNFα release, it promoted the release of IL-6, but not IL-10 or IL-12p70 (FIG. 1F, FIG. 6). Dendritic cells typically respond to bacterial metabolites through the pathogen-associated molecular pattern (PAMP) receptors toll-like receptor 2 (TLR2) and TLR4 (13). Receptor specificity was established by generating mBMDCs from both tlr2^{-/-} and tlr4^{-/-} mice and using them along with wild-type cells in the cytokine induction assay (7). PEs active in wild-type cell assays produced no TNFα induction in mBMDCs from tlr2^{-/-} mice, while mBMDCs generated from the tlr4^{-/-} mice showed robust TNFα induction (FIG. 1G). Previous reports attributed A. muciniphila's immunomodulatory activity to a membrane-associated protein (Amuc_1100) signaling through TLR2 (4, 32). Additional publications supporting the protein's role in maintaining the intestinal mucosal barrier have also appeared (33).

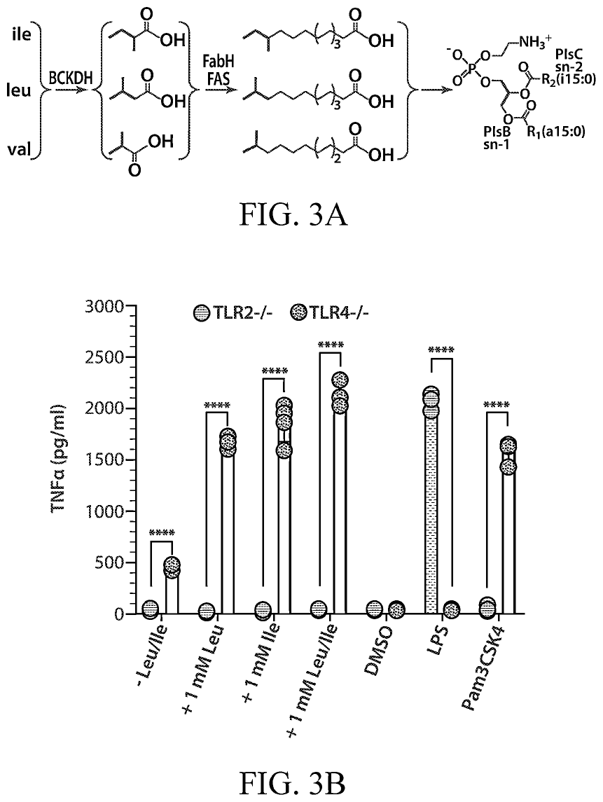

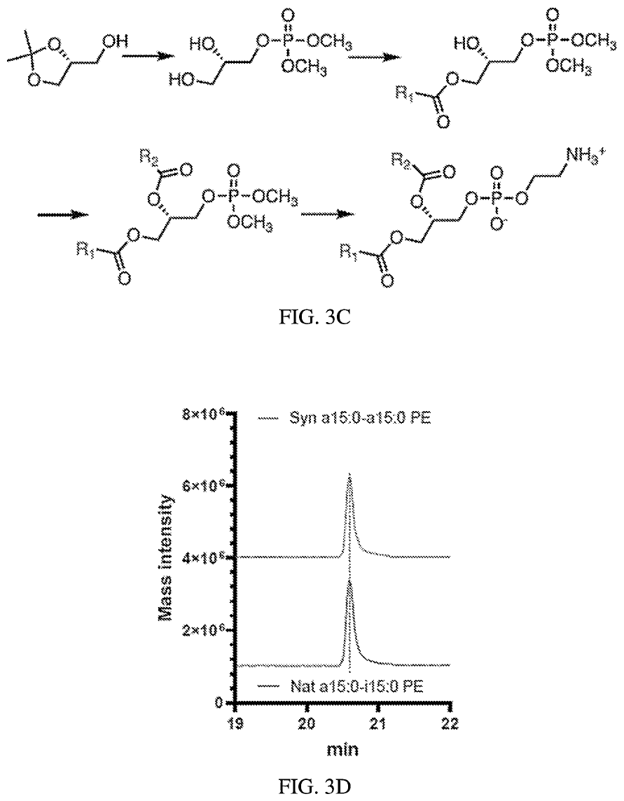

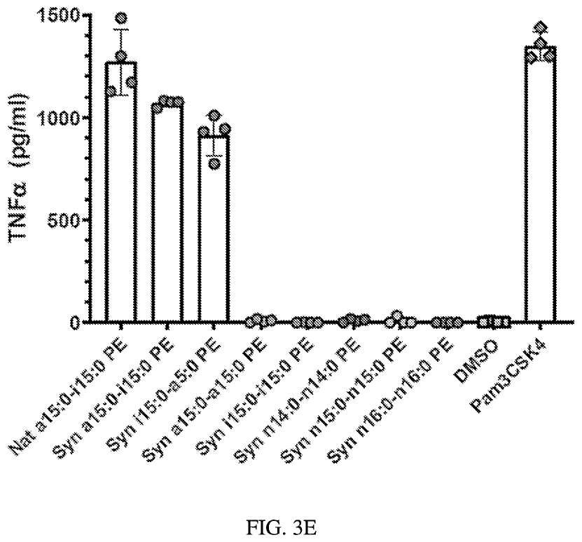

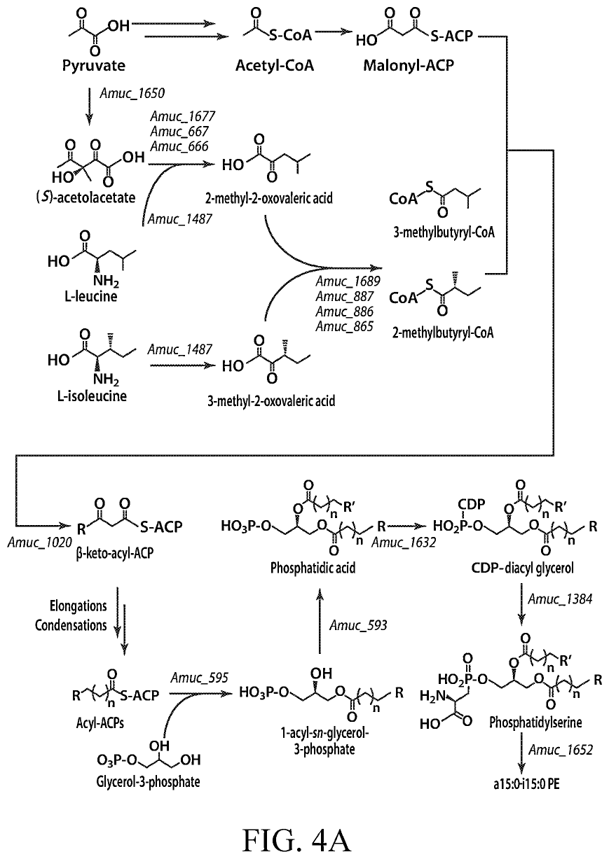

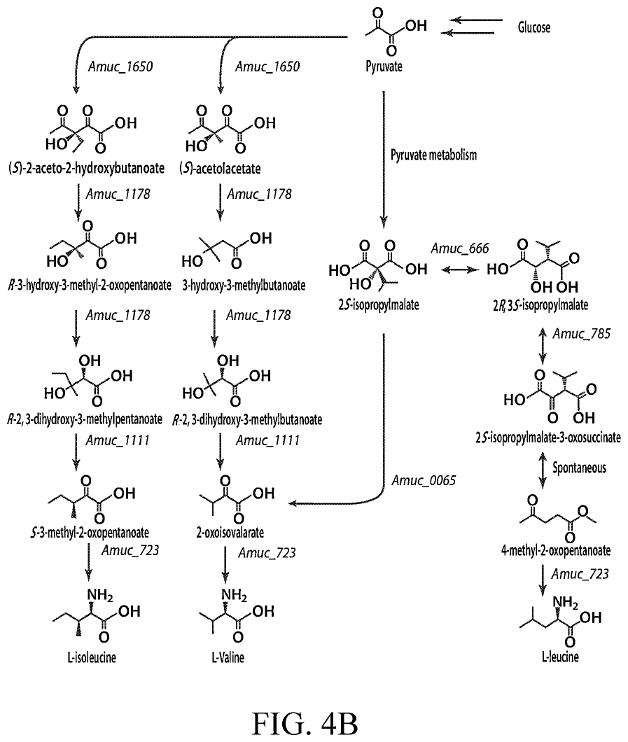

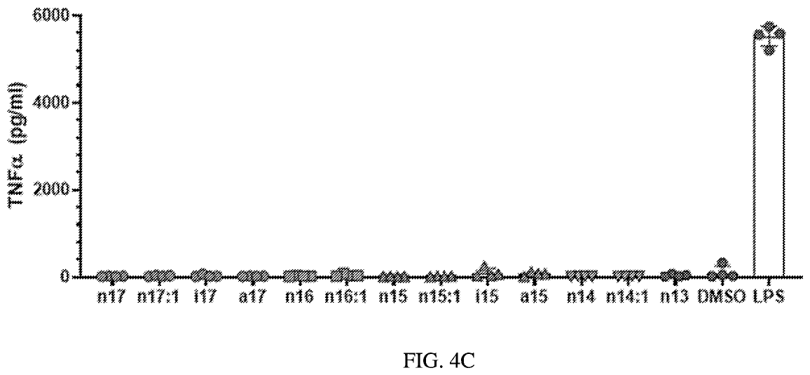

With the identification of a15:0-i15:0 PE as an immunomodulatory signaling molecule and its cognate receptor as TLR2, the pathway and regulation of PE biosynthesis by A. muciniphila and the laboratory synthesis of a15:0-i15:0 PE analogs were examined (FIGS. 3A-3E). Bacterial PE biosynthesis has three distinct stages (FIG. 3A) (14). In the first stage, the branched-chain amino acids (BCAAs) isoleucine (ile), leucine (leu), and valine (val) are converted to branched-chain carboxylic acids by the branched-chain alpha-keto acid dehydrogenase complex (BCKDH) (14). These acids are shuttled into the fatty acid synthase (FAS) cycle by FabH where they become the tail end of a FA chain through the repetitive addition of two-carbon units (14). After elongation, the BCFAs are added to the sn-1 and sn-2 positions of glycerol-3-phosphate by the enzymes PlsB and PlsC, respectively. Finally, the phosphate head group is elaborated into a PE head group (14). The A. muciniphila genome has genes that encode the enzymes for every step of the BCAA to PE pathway just described (FIG. 4A). In addition, A. muciniphila has the genes for the de novo synthesis of branched chain AAs from glucose (FIG. 4B). The composition of PE is regulated by a number of factors from the availability of BCAAs to the selectivity of PlsB and PlsC, the enzymes attaching the acyl chains. The general pathway outlined above is supported by feeding experiments (FIG. 3B). Lipid extracts from A. muciniphila grown in minimal media supplemented with leu and/or ile led to significant increases in TNFα release in a TLR2-dependent fashion from treated mBMDCs. Experiments in rich media (Brain Heart Infusion, BHI) show similar but smaller increases.

Candidate PEs were synthesized to independently confirm the identification, to provide additional material for biological testing, to eliminate the possibility of natural contaminants, and to explore structure-activity relationships (SAR). The synthesis, which is outlined in FIG. 3C, began with a commercially available protected chiral glycerol. With the future sn-2 and sn-3 positions blocked, the future sn-3 position was converted to a protected phosphate (FIG. 3C). The hydroxyl groups at sn-2 and sn-3 were deprotected and the acyl groups were added in a stepwise fashion taking advantage of the greater reactivity at the sn-1 position to install this acyl chain first. The a15:0 carboxylic acid used in the synthesis had the stereochemistry appropriate for natural isoleucine. The synthetic a15:0-i15:0 PE had identical spectroscopic, chromatographic, and biological properties to the natural product (FIG. 1E, FIG. 3D, FIG. 3E).

In addition to confirming the order of the acyl chains and two stereochemical issues, the synthetic scheme allowed a small library of natural and synthetic PEs to be assembled. The library's SAR was assayed to establish a preliminary SAR for the A. muciniphila lipids and their component parts and revealed surprising results (FIG. 3E). First, it was established that FAs activate TLR2 in the context of a diacyl PE, as none has detectable activity on its own (FIG. 4C). This result is consistent with the tlr2^{-/-} mBMDC analysis (FIG. 1G). 1) Compounds with methyl branches showed TNFα induction, whereas the three analogs where both acyl groups have straight chains (n14:0, n15:0, or n16:00) had no detectable activity. 2) Compounds wherein the two acyl chains are the same (a15:0-a15:0 PE and i15:0-i15:0 PE) had no detectable activity. 3) Positional asymmetry appears to play a minor role as a15:0-i15:0 PE and i15:0-a15:0 PE had only subtle differences in activity. The compound i15:0-a15:0 PE has not been detected in natural samples.

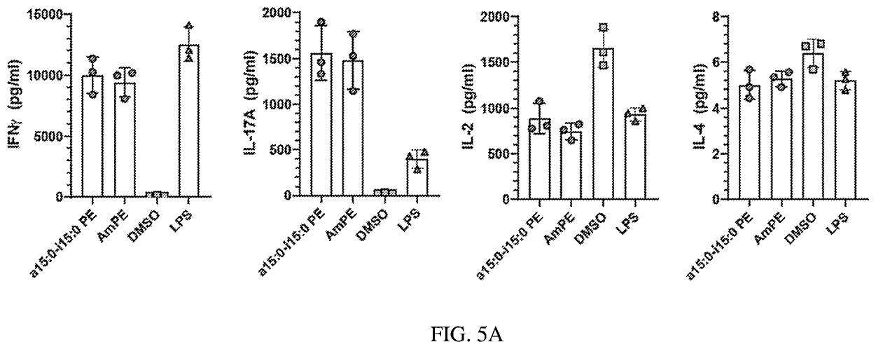

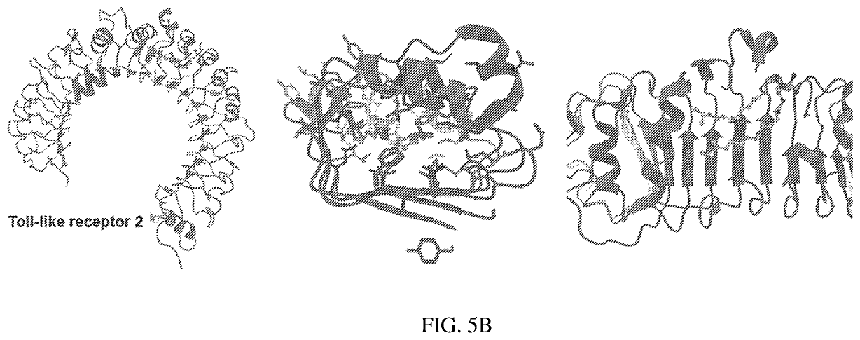

It was sought to connect the active lipids from A. muciniphila to the selective cytokine responses of specific T cell lineages. There is widespread agreement that homeostatic immunity uses modules of both innate and adaptive immunity (15). It was found that when the innate immune cells (mBMDCs) used in this study are co-cultured with OVA-specific CD4⁺ T cells and OT-II peptides, an adaptive response is triggered including selective T cell effector fates for IL-17A and IFNγ producing cells (FIG. 5A). This selectivity argues that A. muciniphila can control the fate of T cells, pushing them toward Th1 and Th17 polarization. In this way, the results both support previous research reports on the immunomodulatory nature of A. muciniphila and identify the lipid signal that initiates it (6).

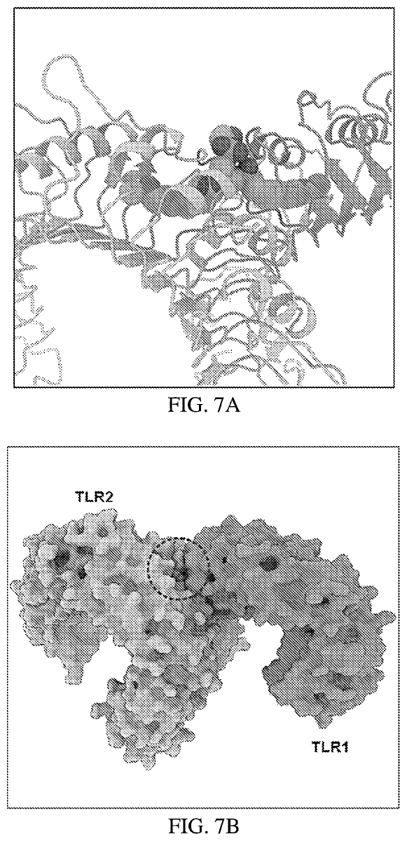

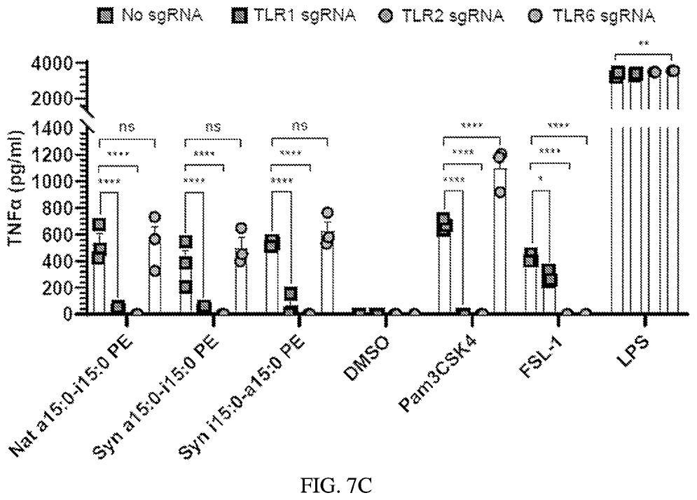

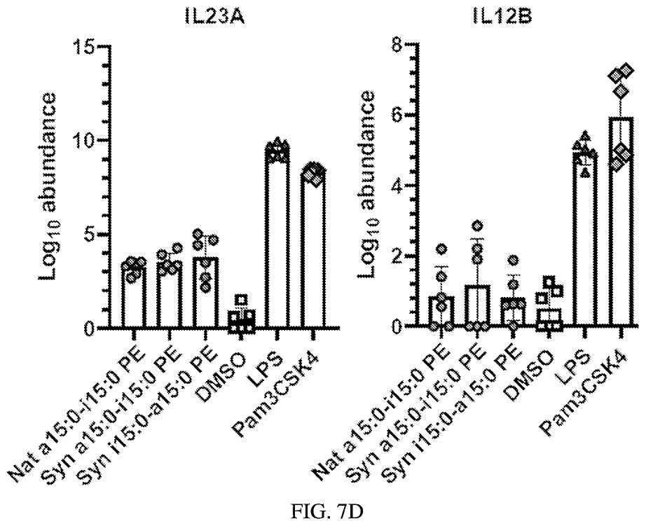

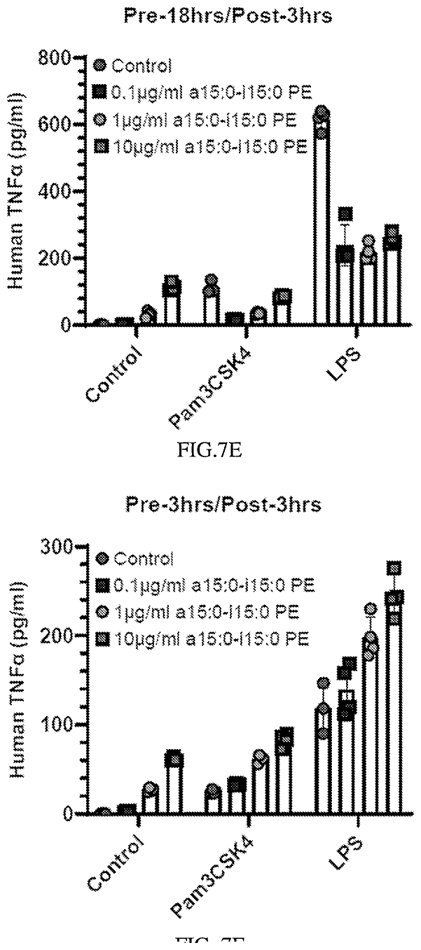

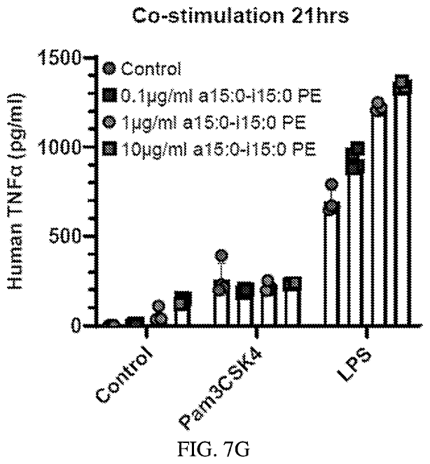

SAR studies on TLR2 ligands invariably focus on the head group that protrudes from the membrane bound receptor. The conventional view of TLR2 signaling relegates the lipid chains to providing hydrophobic anchors for a protruding head group that regulates receptor activation (16, 17, 35). This view is supported by several structural studies on TLR2 receptors with bound ligand and SAR studies (17, 36, 37). The extracellular part of TLR2 is a horseshoe shaped, leucine-rich repeat with a long hydrophobic tunnel that binds two acyl chains (FIGS. 5B, 7A, and 7B). TLR2 typically requires formation of a heterodimer with either TLR1 or TLR6 for immune signaling (16, 36, 37). CRISPR-Cas knockdowns of TLR6 and TLR1 showed that a TLR2-TLR1 heterodimer is required for TNFα induction, a surprising result for a diacyl lipid (FIGS. 7A and 7C) (16, 36, 37). The requirement for a non-canonical TLR2-TLR1 heterodimer suggests that a15:0-i15:0 PE's two acyl chains occupy binding pockets in two different proteins, one in TLR2 and one in TLR1, forming an atypical signaling heterodimer with a buried head group (FIG. 7B). There are other TLR2 agonists that form TLR2-TLR1 heterodimers, and at least two of them (the synthetic molecules diprovocim and CU-T12-9) were developed as adjuvants for cancer immunotherapy, while another (polysaccharide A from Bacteroides fragilis) is produced by a member of the gut microbiome and associated with IL-10 production (38-41). There is an important difference in potency between the synthetic agonists and a15:0-i15:0 PE:EC₅₀ values of pM vs. μM. A similar difference in immunogenicity has been noted in a study of immunomodulatory sphingolipids from B. fragilis (34).

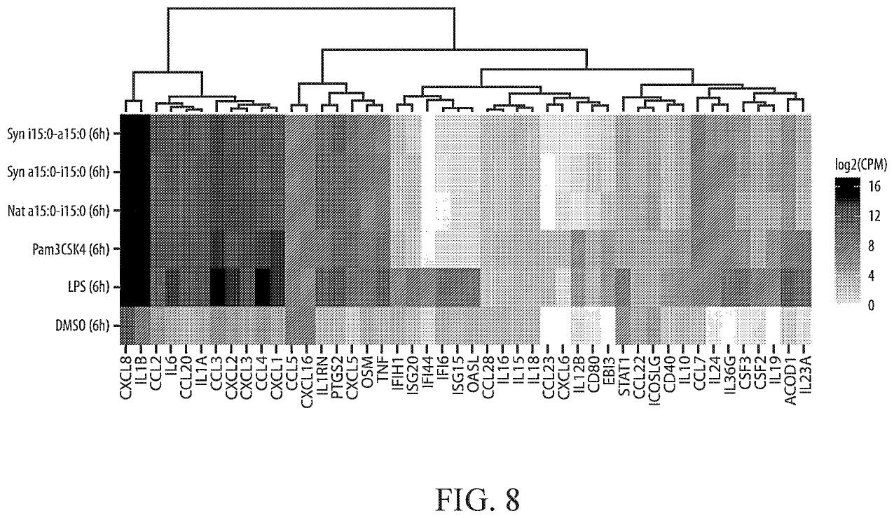

A further objective of the study was to connect the active lipids from A. muciniphila to the selective cytokine responses of specific human immune cell lineages (6). Human monocytes purified from peripheral blood were cultured and stimulated with natural and synthetic TLR2 agonists for 6 hours, after which mRNA was extracted and sequenced (FIG. 8). The a15:0-i15:0 PE induced pro-inflammatory cytokines such as TNFα and IL-6 comparably to lipopolysaccharide (LPS) and Pam3CSK4, albeit at higher doses, but was significantly less effective at inducing IL-23, a heterodimer of IL-23A and IL-12B (FIGS. 1E, 6, 7D, and 8). The IL-23/Th17 immune axis is a major inflammatory pathway, and its therapeutic regulation is an important research target (42).

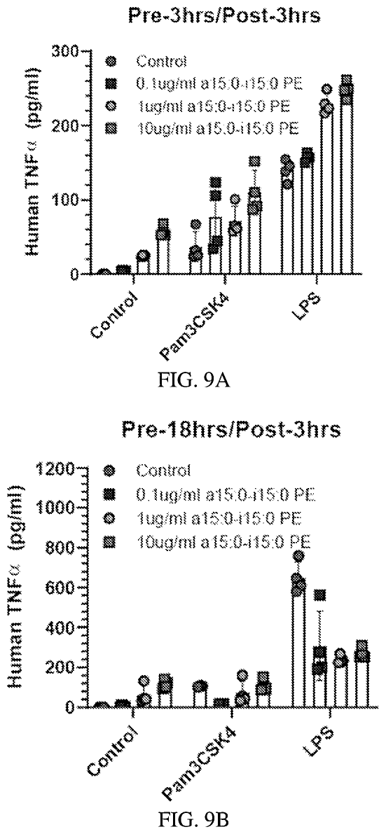

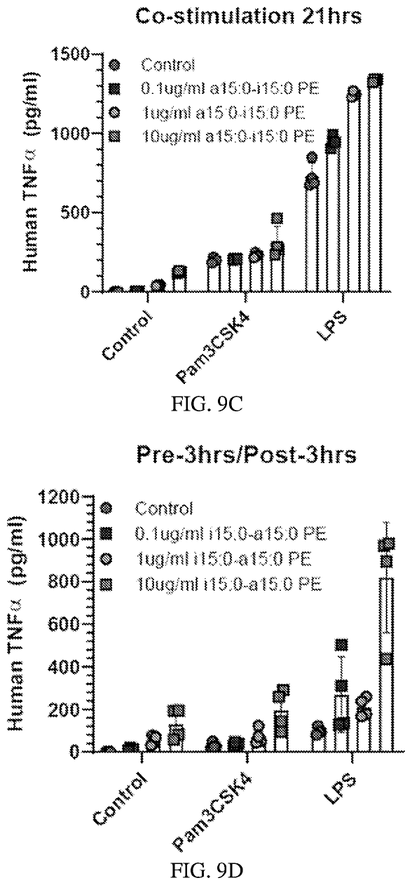

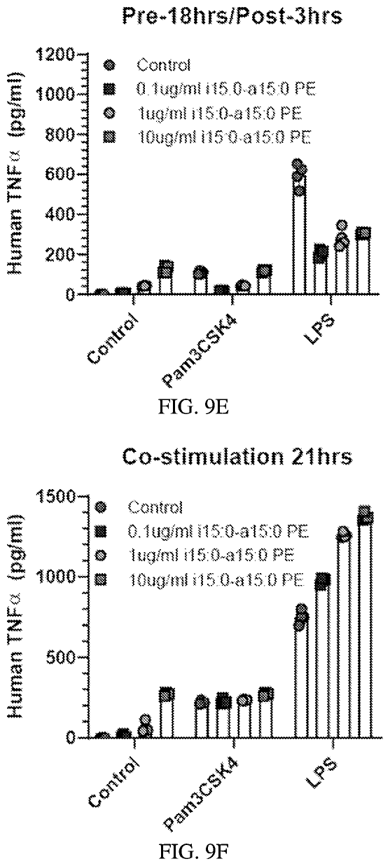

Next, the effects of a15:0-i15:0 PE on other immunogens were investigated by treating human monocyte-derived dendritic cells (which were differentiated by GM-CSF and IL-4) with a15:0-i15:0 PE and the model agonists Pam3CSK4 (to stimulate TLR2/1) and LPS (to stimulate TLR4). Variables were dose, duration (6 or 21 hours), and timing (co-treatment or sequential treatment). In the 21-hour sequential treatment study, a low dose of a15:0-i15:0 PE (0.15 μM, ˜1% of EC₅₀) was followed 18 hours later by the addition of known agonists. This treatment regimen completely suppressed TNFα release (FIGS. 7E and 9A-9F). The suppressive effect was not seen with shorter periods between the lipid and agonist treatments (6-hour and co-stimulation studies) nor with higher doses of lipid (FIGS. 7F, 7G, and 9A-9F). These results support a model in which low doses of a15:0-i15:0 PE and delayed stimulation reset the cellular activation threshold and moderate other cellular immune responses, as indicated by the LPS response, which was reduced but not in a lipid-dependent fashion (FIG. 7E). Low-dose and delayed stimulation reflect likely in vivo conditions. Larger doses and shorter times produce the expected dose-dependent response (FIGS. 1E, 7F, 7G, and 9A-9F).

Since its discovery, multiple lines of investigation have indicated that A. muciniphila plays an outsize role in regulating human immune responses in a variety of contexts (5, 6, 25, 26). Its connections with metabolic syndrome, type 2 diabetes, inflammatory bowel disease, and responses to cancer immunotherapy have attracted the most attention, and a more recent study linking it to homeostatic immunity projects additional roles (6, 15). The study demonstrates that a single lipid forming the majority of A. muciniphila's PE membrane, a15:0-i15:0 PE, activates TLR2 to produce immune responses. A. muciniphila's immunomodulatory activity can be replicated by this diacyl-phosphatidylethanolamine, a15:0-i15:0 PE, a lipid that is not noticeably different from other diacyl-PEs forming the cell membranes of most bacteria found in the human gut (8). Because of its generic structure, its remarkable activity would not have been easily identified by genomic or metabolomic analyses. It agonizes a noncanonical TLR2-TLR1 heterodimer to release a subset of inflammatory cytokines (16, 17, 43): it induces cytokine release in murine dendritic cells and triggers the production of specific T cell lineages characteristic of homeostatic immunity. The potency of TLR2 heterodimers is conventionally thought to be governed by a peptide, peptide-like, or (poly)saccharide moiety emerging from the dimer interface, and the absence of this chain in a15:0-i15:0 PE might be responsible for the molecule's unusual immunomodulatory effects (FIGS. 1E and 7B) (17, 28, 40). The existing data support a model in which repeated low-level stimulation of the TLR2-TLR1 signaling pathway resets the activation threshold so that weak signals are ignored and strong signals are moderated, thereby contributing to homeostatic immunity (15, 44). While it is acknowledged that TLR2 receptors on innate immune cells are activated by bacterial membrane lipids, the study reveals an unexpected and highly specific origin of the signal. Structural modifications, such as relocating a methyl group to an adjacent carbon atom on the part of a buried lipid chain at greatest remove from the signal recognition site, regulates cytokine release and immune activation. Overall, this study describes the molecular mechanism of a druggable pathway that recapitulates in cellular assays the immunomodulatory effects associated with a prominent member of the gut microbiota.

### Materials and Methods

### General Experimental Procedures and Instruments

UV spectra were recorded on an Ultrospec 5300-pro UV/Visible spectrophotometer (Amersham Biosciences, Little Chalfont, UK). Optical rotations were recorded using a JASCO P-2000 polarimeter (sodium light source, JASCO, Easton, PA, USA) with a 1 cm cell. ¹H, ¹³C, and 2D NMR spectra were recorded on a Bruker Advance 500 MHz spectrometer (Bruker, Billerica, MA, USA). GC/MS analysis was performed with Agilent 7890B series gas chromatograph (Agilent Technologies, Santa Clara, CA, USA) using a HP-5 ms Ultra Inert column and a flame ionization detector. Electrospray ionization (ESI) low-resolution LC/MS data were obtained on an Agilent Technologies 6130 quadrupole mass spectrometer (Agilent Technologies, Santa Clara, CA, USA) coupled with an Agilent Technologies 1200-series HPLC. High-resolution electrospray ionization (HR-ESI) mass spectra were acquired using Agilent LC-q-TOF Mass Spectrometer 6530-equipped with a 1290 uHPLC system. HPLC purifications were performed using Agilent 1100 or 1200 series HPLC systems equipped with a photo-diode array detector. All solvents were HPLC quality.

### Bacterial Cultivation and Extraction of Metabolites

Akkermansia muciniphila BAA-835 was inoculated in 3 mL of Brain-Heart Infusion (BHI) medium in a 5-mL Falcon® tube and incubated under anaerobic conditions at 37° C. for 4 days. Then, 3 mL of the culture was used to inoculate 1 L of BHI medium with 1.5 g of mucin from porcine stomach (Sigma-Aldrich, Natick, MA, USA), in a 1-L Pyrex storage bottle (16 bottles×1 L each, total volume 16 L) and the cultures were incubated for 12 days under anaerobic conditions at 37° C. After 12 days of static growth, bacterial cultures were centrifuged to separate cell pellets and supernatants (8000 rpm, 30 min). The cell pellets were extracted with chloroform and methanol (1:1) by stirring for 24 h at room temperature. The solvent mixture was filtered through Whatman® qualitative filter paper (Grade 3, circle, diam. 125 mm) and dried under vacuum.

For the extraction of supernatants, 100 g of hydrophobic resin mixture (Amberlite® XAD4HP and XAD7HP, 20-60 mesh) was added directly to spent media to allow secreted metabolites to adsorb to the resins. Then, the resin mixture containing bacterial metabolites was washed with acetone and methanol (1:1) and stirred for 24 h at room temperature. The solvent mixture with resin mixture was filtered through Whatman® qualitative filter paper (Grade 3, circle, diam. 125 mm) and concentrated on a rotary evaporator. The cultivation and extraction procedures were repeated 8 times (total culture volume: 128 L), yielding 4 g of dry extract from the cell pellets and 15 g of crude extract from the supernatants.

Bioassay-Guided Fractionation and Purification of a15:0-i15:0 PE

The crude extract from the cell pellets (4 g) was dissolved in chloroform and fractionated by normal phase chromatography using 7 different solvent systems (A: 100% hexane, B: 100% chloroform, C: 100% ethyl acetate, D: 75% ethyl acetate/25% methanol, E: 90% acetone/10% methanol, F: 50% methanol/50% dichloromethane, and G: 100% methanol) with a silica column (Teledyne Isco, RediSep RF Gold® Silica 12 g). The pro-inflammatory activity was highly detected in fractions F and G. The mixture of fractions F and G (120 mg and 210 mg, respectively, and 8.3% of total yield) was then subjected to reversed-phase semi-prep HPLC (Luna® C₈ (2): 250×10 mm, 5 μm) using the following gradient solvent system: 10% methanol/90% water isocratic for 10 min, gradient to 30% methanol/70% water for 10 min, then, 30% methanol/70% water to 90% methanol/10% water for 20 min, 90% methanol isocratic for 10 min, gradient to 100% methanol for 25 min, flow rate: 2 mL/min). Fractions were collected every 1 min between 5 min to 75 min, generating 70 fractions. Fractions able to stimulate pro-inflammatory cytokine production from mBMDCs were combined and identified as bacterial phosphatidylethanolamine with branched chain fatty acids (22 mg, yield=0.55%). An essentially pure compound, later identified as a15:0-i15:0 PE, was acquired at a retention time of 63 min (14 mg, yield=0.35%).

The crude extract from the supernatants (15 g) was dissolved in methanol and filtered through a syringe filter (PTFE, 0.2 μm). The filtered extract was directly injected onto a reversed-phase prep-HPLC column (Luna® C₁₈ (2): 250×21.2 mm, 5 μm) with a gradient mobile solution (30% methanol/70% water to 100% methanol for 30 min, 100% methanol isocratic for 30 min, flow rate: 10 mL/min). Fractions were collected every 2 min from 5 min to 55 min, generating 25 fractions. Fractions able to stimulate pro-inflammatory cytokine production from mBMDCs were collected at 50 min (23.5 mg, 0.16%) and further purified as described above, resulting in additional a15:0-i15:0 (1.2 mg, yield=0.008%). Overall, 15.2 mg of a15:0-i15:0 was isolated from 19 g of crude extract (0.08%).

a15:0-i15:0 PE: White powder; UV (MeOH) λₘₐₓ (log ε) 210 nm; ¹H (500 MHz) and ¹³C (125 MHz) NMR data, see Table 1; HR-ESI-QTOF-MS (positive-ion mode) m/z 664.4922 [M+H]⁺ (calcd for C₃₅H₇₁NO₈P, 664.4917).

Identification of a15:0-i15:0

The active compound was purified as described above as a white powder and preliminary MS analysis showed a [M+H]⁺ ion at m/z 664.4922 (HR-ESI-QTOF-MS). The recorded exact mass was consistent with the molecular formula C₃₅H₇₀NO₈P (calcd at [M+H]⁺ at m/z).

Combined analysis of ¹H, ¹³C, and HSQC NMR data (Table 1) of the active molecule in CDCl3 identified two amide/ester carbonyl signals (δ_{C} 173.6 and 173.3), five oxygen/nitrogen bound sp3 methine groups (δ_{C}/δ_{H}: 70.5/5.21, 62.8/4.38, 62.7/4.14, 62.4/4.07, 64.0/3.99, 40.5/3.15), four diastereotopic methylene groups (δ_{C}/δ_{H}: 39.1/1.12, 34.5/2.28, 34.4/2.30, 25.2/1.59, and 25.1/1.58), largely overlapped aliphatic methylene groups (δ_{C}/δ_{H}: 34.6-27.3/1.25), and four methyl groups (δ_{C}/δ_{H}: 22.9/0.89, 22.8/0.88, 19.4/0.86, and 14.3/0.87). On the basis of NMR and UV spectroscopic data, the molecule has two fatty acid chains associated with a phosphoryl group.

Interpretation of COSY NMR correlations identified four discrete spin systems. The COSY correlations between H₂-13′ (δ_{H} 1.27) and terminal methyl group (H₃-14′, δ_{H} 0.88) connected C-13′ (δ_{C} 27.3) with C-14′ (δ_{C} 11.6). Further COSY analysis identified H-12′ (δ_{H} 1.32) and H₃-15′ (δ_{H} 0.87) as having correlations, connecting C-12′ (δ_{C} 34.3) and methyl carbon (δ_{C} 19.4). Then, the COSY correlations between H₂-2′ (δ_{H} 2.28), H₂-3′ (δ_{H} 1.60), H-12′ and a largely overlapped aliphatic signals at δ_{H} 1.25 indicated connectivity from C-12′ to C-2′ (δ_{C} 27.6), establishing the first spin system as an anteiso-branched fatty acid. Additional COSY relationship between overlapped two methyl groups (H₃-13″ and H₃-14″, δ_{H} 0.89) and H-12″ (δ_{H} 0.90) placed terminal dimethyl groups at C-12″ (δ_{C} 14.4). As a first partial structure, H₂-2″, H₂-3″, H-12″ and large methylene envelope (H₂-4″ to H₂-11″, δ_{H} 1.25) showed clear COSY correlations, deducing the second spin system as an iso-branched fatty acid. The glycerol fragment as a third partial structure was established based on the sequential COSY correlations between H₂-1 (δ_{H} 4.39 and 4.15)/H-2 (δ_{H} 5.21)/H₂-3 (δ_{H} 3.99). The HMBC correlates from H₂-1/H₂-2″ and H-2/H₂-2″ to carbonyl carbon signals at δ_{C} 173.7 and 173.3, indicating a diacylglycerol. The remaining COSY correlations between H₂-4 (δ_{H} 4.07) and H₂-5 (δ_{H}3.15) were analyzed as an ethanolamine moiety. Each of the partial structures, diacylglycerol and ethanolamine, were connected by phosphoryl group based on the molecular weight and biosynthetic logic, completing structural characterization of the active molecules as a phosphatidylethanolamine bearing branched chain fatty acid.

TABLE 1
¹H and ¹³C NMR data (500 MHz) of a15:0-i15:0 PE in CDCl₃.

| no. | δ_{H}, mult (J in Hz)^{a} | δ^{b} | |
|---|---|---|---|
| 1a | 4.37, dd (11.5, 2.5) | 62.8 | CH₂ |
| 1b | 4.14, dd (11.5, 6.5) | | |
| 2 | 5.21, m | 70.6 | CH |
| 3 | 3.93, br | 64.1 | CH₂ |
| 4 | 4.07, br | 62.4 | CH₂ |
| 5 | 3.15, br | 40.6 | CH₂ |
| 1′ | | 173.6 | C |
| 2′ | 2.29, m | 34.6 | CH₂ |
| 3′ | 1.58, m | 25.2 | CH₂ |
| 4′-10′ | 1.31-1.21 | 30.3- | CH₂ |
| | | 27.4 | |
| 11′a | 1.26, m | 36.7 | CH₂ |
| 11′b | 1.07 | | |
| 12′ | 1.27, m | 34.5 | CH |
| 13′ | 1.13 | 29.5 | CH₂ |
| 14′ | 0.83, t (6.5) | 11.6 | CH₃ |
| 15′ | 0.87, d (6.5) | 19.4 | CH₃ |
| 1″ | | 173.3 | C |
| 2″ | 2.29, m | 34.3 | CH₂ |
| 3′ | 1.58, m | 25.1 | CH₂ |
| 4″-11″ | 1.31-1.21 | 30.3- | CH₂ |
| | | 27.4 | |
| 12″ | 1.16, m | 39.3 | CH |
| 13″ | 1.51, m | 28.2 | CH |
| 14″ | 0.85, d (6.5) | 22.9 | CH₃ |
| 15″ | 0.86, d (6.5) | 22.8 | CH₃ |

^{a1}H: 500 MHz,
^{b13}C: 125 MHz

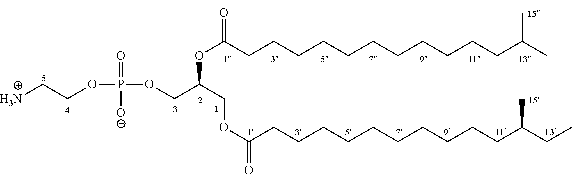 [SMILES: CC[C@H](C)CCCCCCCCCCC(=O)OC[C@@H](COP(=O)([O-])OCC[NH3+])OC(=O)CCCCCCCCCCCC(C)C]

### NMR Spectroscopy

All ¹H NMR spectra were acquired at 500 MHz at 30° C. and chemical shifts are represented in δ scale. Residual protium in the NMR solvent (CDCl₃, δ 7.26) was used to reference chemical shifts. Data are represented as follows: assignment, chemical shift, integration, multiplicity (s, singlet; d, doublet; t, triplet; q, quartet; m, multiplet; br, broad), and coupling constant in Hertz. All ¹³C NMR spectra were obtained at 125 MHz at 30° C. and chemical shifts are represented in δ scale. The carbon resonances of the NMR solvent (CDCl₃, δ 77.17) are used to reference chemical shifts. Full assignment of protons and carbons were completed on the basis of the following two-dimensional NMR spectroscopy experiments: Gradient ¹H-¹H correlation spectroscopy (gCOSY), gradient ¹H-¹³C heteronuclear single quantum coherence (gHSQC), gradient ¹H-¹³C heteronuclear multiple bond connectivity (gHMBC).

HRMS for a15:0-i15:0 PE and Other Family Members

High-resolution mass spectrometry (HRMS) data were recorded on using Mass Hunter Work Station LC/MS Data Acquisition 10.1 and Agilent LC-q-TOF Mass Spectrometer 6530-equipped with a 1290 uHPLC system and electrospray ionization (ESI) detector scanning from m/z 50-3200. 5 μL aliquots of a15:0-i15:0 PE and its family members were injected into a reversed-phase analytical column (Luna® C₈: 100 mm×2.1 mm, 5 μm) using a gradient solvent system with 0.1% formic acid (10% methanol/water to 90% methanol/water for 10 min, 90% methanol/water isocratic for 10 min, then gradient to 100% for 10 min, flow rate: 0.3 mL). Agilent Mass Hunter Qualitative Analysis B.07.00 was used to analyze the data.

Fatty Acid Methyl Esterification and GC-MS Analysis of A. muciniphila PE

A 0.1 mg sample of both a15:0-i15:0 PE and complete A. muciniphila PE were dissolved in 200 μL of methanol and 1.4 mg of sodium methoxide was added to prepare 0.5 M sodium methoxide solution. The reaction mixture was stirred at room temperature for 3 h then quenched by addition of 1 N HCl. The methanolysis products were dried under vacuum and extracted with ethyl acetate and water (300 μL, v/v=2:1). The water layers were removed and each of ethyl acetate layers containing fatty acid methyl esters (FAME) were injected into a gas chromatograph (GC, Agilent Mass Hunter GC/MS Acquisition B.07.05.2479) combined with HP-5 ms Ultra Inert column (0.25 mm×30 m). The temperature of the injector and the detector in the GC was maintained at 150° C. During analysis, the temperature of the GC column was controlled (150° C. for 3 min, 150-250° C. at 6° C./min, and 250° C. for 3 min). The FAME derivatives of a15:0-i15:0 PE were composed of i15:0 and a15:0 (1:1 ratio) having retention times at 10.2 min and 9.7 min, respectively. The GC-MS analysis of FAME derivatives of AmPE displayed i14:0 (15.7%), n14:0 (2.7%), a15:0 (51.7%), i15:0 (23.6%), a16:0 (0.6%), i16:0 (1.8%), a17:0 (1.7%), and a18:0 (2.2%), having retention times at 8.0, 8.6, 9.7, 10.2, 11.3, 11.9, 13.4, and 15.0 min, respectively (FIG. 1D). Agilent Mass Hunter Qualitative Analysis B.07.00 was used to analyze GC-MS data.

### O-Deacylation for Determination of a15:0 Connected to Sn-1

A 5 mg sample of a15:0-i15:0 PE was prepared and lyophilized for 24 h. 1 mg/mL of NaOMe solution was prepared and the mixture was dissolved in 500 μL of NaOMe solution at room temperature. The solution was stirred under argon for 30 min. After 30 min, the reaction was quenched by addition of 1N HCl and dried under vacuum. The O-deacylated product, a15:0 PE was purified by reversed-phase HPLC (Luna® C₈(2): 250×10 mm, 5 μm) with an isocratic solvent system (45% acetonitrile/water over 30 min, UV 210 nm detection, flow rate: 2 mL/min). The O-deacylated product (1.8 mg) was eluted at 12.5 min, and its structure was determined by 1D, 2D NMR spectroscopy, and by low-resolution ESI-MS ([M+H]⁺ m/z at 440, molecular formula; C₂OH₄₃NO₇P).

TABLE 2
¹H and ¹³C NMR data (500 MHz) of a15:0 PE in CD₃OD.

| no. | δ_{H}, mult (J in Hz)^{a} | δ^{b} | |
|---|---|---|---|
| 1a | 4.16, dd (11.5, 4.5) | 63.4 | CH₂ |
| 1b | 4.10, dd (11.5, 6.5) | | |
| 2 | 3.96, m | 70.0 | CH |
| 3 | 3.90, m | 68.0 | CH₂ |
| 4 | 4.05, m | 63.41 | CH₂ |
| 5 | 3.14, br | 42.0 | CH₂ |
| 1′ | | 175.6 | C |
| 2′ | 2.35, t (7.5) | 35.1 | CH₂ |
| 3′ | 1.61, m | 26.1 | CH₂ |
| 4′-10′ | 1.31-1.21 | 33.2- | CH₂ |
| | | 28.3 | |
| 11′a | 1.26, m | 37.9 | CH₂ |
| 11′b | 1.10 | | |
| 12′ | 1.27, m | 35.8 | CH |
| 13′ | 1.13 | 30.7 | CH₂ |
| 14′ | 0.83, t (6.5) | 11.8 | CH₃ |
| 15′ | 0.87, d (6.5) | 19.8 | CH₃ |

^{a1}H: 500 MHz,
^{b13}C: 125 MHz

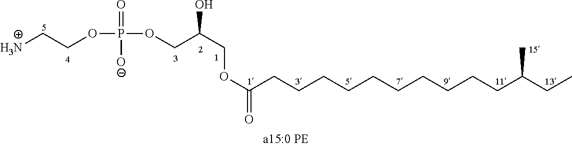 [SMILES: CC[C@H](C)CCCCCCCCCCC(=O)OC[C@H](O)COP(=O)([O-])OCC[NH3+]]

### Amino Acid Feeding Experiment

5 mL of A. muciniphila BAA-835 grown in BHI was inoculated into three 1 L bottles of M9 medium supplemented with 1.5 g of mucin from porcine stomach (Sigma-Aldric, Natick, MA, USA), and either 1 mM of 1-leucine, 1-isoleucine, 1-leucine/1-isoleucine mixture (1:1 ratio), or nothing as a control. The cultures were grown under anaerobic conditions at 37° C. for 12 days. The cell pellets from these cultures were centrifuged and extracted with 40 mL of chloroform and methanol (1:1). The extract was dried under vacuum and dissolved in DMSO at a 10 mg/mL concentration and tested for activity in the mBMDC cytokine assay. Statistical significances were determined using an unpaired two-tailed Student's t-test.

a15:0-i15:0 PE Biosynthetic Gene Identification and Analysis

Sequence comparison and analysis of the a15:0-i15:0 PE biosynthetic pathway to the previously reported branched-chain fatty acid biosynthetic pathway and de novo biosynthetic pathway of leucine, isoleucine, and valine were performed using blastp (NCBI refseq database, updated Aug. 9, 2015), Kyoto Encyclopedia of Genes and Genomes (KEGG), and Geneious 11.1.4 for pairwise sequence alignments that were previously reported. The accession number for the genes used in this analysis is CP001071.1.

### Total Synthesis for a Small Library of PEs

The total synthesis of a15:0-i15:0 PE, i15:0-a15:0 PE, a15:0-a15:0 PE, i15:0-i15:0 PE, and n15:0-n15:0 PE were performed by previously reported methods (18-20).

### Animal and Human Cell Studies

Mouse experimental procedures complied with all relevant ethical regulations and were conducted according to protocol 2003N000158 approved by the Institutional Animal Care and Use Committee (IACUC) at Massachusetts General Hospital. Appropriate sample sizes were estimated based on the effect size and variance of cytokine measurements in myeloid cells stimulated with canonical TLR ligands. In all mouse experiments, animals were allocated into experimental groups based on genotype and/or age- and sex-matched. Male or female wild-type, TLR2^{-/-}, or TLR4^{-/-} C57BL/6 mice at least 3-4 weeks old and preferably 7-12 weeks of age were used. Mice were housed with a 12-hour light/dark cycle at an ambient temperature between 65-75° F. and 30-70% relative humidity.

Human monocytes were isolated from buffy coats collected from healthy donors at the Blood Donor Center at Massachusetts General Hospital in compliance with all relevant ethical regulations and according to protocol 2018P001504 approved by the Mass General Brigham Institutional Review Board (IRB). Donors provided informed written consent.

mBMDC Cytokine Assays

These assays were done as described previously (21). Femurs and tibias were collected from male or female wild-type, TLR2^{-/-}, or TLR4^{-/-} C57BL/6 mice that were at least 3-4 weeks old and preferably 7-12 weeks of age. The bone marrow was pushed from the bones using a needle and syringe of complete Dulbecco's Modified Eagle Medium (DMEM) (supplemented with Gibco GlutaMAX Supplement (35050061), Gibco Penicillin-Streptomycin (15140122), and 10% heat-inactivated fetal bovine serum (FBS)) and strained through a 70 μm nylon filter. The collected bone marrow was then centrifuged and red blood cells were lysed using Invitrogen eBioscience 1×RBC Lysis Buffer (00-4333-57). The cells were then centrifuged and strained through a 70 μm nylon filter again, and resuspended in complete DMEM. Cells were counted and then plated at approximately 5 million cells per plate with ˜20-40 ng/ml RmGM-CSF (PreProTech 315-03). They were allowed to grow for seven days, sometimes with additional feeding of 20-40 ng/ml RmGM-CSF at day three. The resulting bone marrow derived dendritic cells (mBMDCs), were then scraped and counted again. They were plated in 96 well tissue culture-treated microplates (Corning CLS3599) from 50,000-90,000 cells/well and allowed to adhere for at least three hours. The cells were then treated with chromatographic fractions or purified compounds at a final concentration of 50 μg/ml, or 3-625 ng/ml final concentration of LPS (InvivoGen tlrl-b5lps), or 0.25-1.562 μg/ml final concentration of Pam3CSK4 (InvivoGen tlrl-pms) and incubated overnight. The following morning, supernatant was removed and an enzyme-linked immunosorbent assay (ELISA) was performed to measure TNFα using an Invitrogen Mouse ELISA kit (88-7324-77) per the manufacturer's instructions. Gen5 3.03 or SoftMax Pro 6.2.1 (SpectraMax, Molecular Devices) was used to analyze ELISA plates. For cytokine detection using flow-cytometry, the cytometric bead array mouse inflammation kit from BD Biosciences (552364) was utilized per the manufacturer's instructions. Data was collected with NovoExpress 1.4.1 and analyzed using FlowJo v10.7.

### PBMC Cytokine Assay

Peripheral blood mononuclear cells (PBMCs) were enriched for monocytes using RosetteSep™ Human Monocyte Enrichment Cocktail (Stem Cell Technologies #15028). Briefly, the buffy coats, which were obtained from Massachusetts General Hospital Blood Donor center, were incubated with monocyte enrichment cocktail for 20 min at room temperature while rocking. They were then diluted with 1×PBS and layered over the Ficoll-Paque™ PLUS medium (GE Healthcare #17-1440-02) medium and centrifuged for 20 min at 1200×g. Enriched monocytes were collected and cultured/treated with chromatographic fractions or purified compounds at 50 μg/ml in DMEM media containing 10% FBS and 1% penicillin-streptomycin. LPS and Pam3CSK4 at a final concentration of 100 ng/ml were used as controls. After overnight incubation, supernatant was collected and analyzed for IL-6, IL-10, IL-12/IL-23p40 and TNFα cytokines using Human Flex Set Kits (BD™ CBA, 558276, 558274, 560154, 560112).

mBMDC+ T Cells Co-Culture Assay

To assess the ability of different lipids of A. muciniphila to preferentially regulate T cell lineages, cocultures of dendritic cells and T cells were treated with chromatographic fractions or purified compounds. OT-II specific CD4+ T cells were purified from splenocytes of OT-II mice (JAX 004194) with EasySep mouse CD4+ T cell isolation kit (StemCell 19852). mBMDC were produced from C57BL/6 bone marrow cultured with 20 ng/ml GM-CSF for 7 days. 25,000 mBMDCs and 50,000 purified CD4+ T cells were added in each well of a 96-well plate with 100 nM OT-II peptide, 1 μL of the specified lipid and 1 mM aminoguanidine hemisulfate. LPS at a final concentration of 0.4 μg/ml was used as a control. After 3 days co-culturing, supernatants were assayed for IL-17A, IFNγ, IL-2, and IL-4 cytokines by mouse IL-17A, IFNγ, IL-2, and IL-4 Flex set kit (BD cytometric bead assay, 560283, 558296, 555148, and 558298).

### RNA Sequencing

Monocytes were isolated from PBMCs as described previously (45). Bulk RNA sequencing libraries were prepared using Smart-seq2. Libraries were sequenced on a NextSeq (Illumina). FastQC v0.11.5 and MultiQC v1.8 were used to confirm the quality of the sequenced libraries (46, 47). Next, kallisto v0.46.1 was used with a GRCh38 reference to generate the counts of reads mapped to each gene (48, 49). The matrix of counts was used for the calculation of counts per million (CPM) values, and the generated CPM matrix was treated with log₂(CPM+1) to obtain a log expression matrix. A gene with a CPM value greater than 1 was considered as expressed. Samples obtained after the above steps were then used to detect differentially expressed genes (DEGs) via EdgeR v3.35.1 (48). The lists of DEGs were generated from likelihood ratio tests (LRT) based on the generative linear model (GLM) framework, following the prerequisite gene filtering, normalization, and dispersion estimation steps of the software.

### CRISPR Targeting

PBMCs were isolated from buffy coats using Sepmate™ tubes (STEMCELL Technologies) and ammonium-chloride-potassium (ACK) lysis buffer following the manufacturer's protocol. Human monocytes were harvested from PBMCs by negative selection using RosetteSep™ human Monocyte Enriched Cocktail (STEMCELL Technologies) according to the manufacturer's protocol. Alt-sgRNAs were purchased from IDT and reconstituted to 100 μM with Nuclease-Free Duplex Buffer (IDT). In a sterile PCR strip, the sgRNAs were mixed with Cas9 (IDT, Alt-R S.p. Cas9 Nuclease V3) at a molar ratio of 2:1 (2 μL sgRNA of 100 μM+2 μL Cas9 5 mg/mL) for each reaction and incubated at room temperature for over 20 min. Monocytes were washed twice with 5 mL of PBS and counted. 2×10⁶ cells per reaction were resuspended in 16 μL of P3 primary nucleofection solution (Lonza). The 16 μL of cells in P3 buffer was added to each Cas9-RNP complex. The cell/RNP mix was then immediately loaded into the supplied nucleofector cassette strip (Lonza) and nucleofected using 4D-Nucleofector with CM-137 program. 180 μL of prewarmed medium was immediately added into each cassette well. 1×10⁵ cells were seeded into a 96-well plate with medium (RPMI-1640 with 10% FBS, 2 mM Glutamax, 55 μM β-mercaptoethanol, 100 U/mL penicillin, 100 μg/mL streptomycin, GM-CSF 800 U/mL, and IL-4 500 U/mL). Medium was changed every 2-3 days. At day 5, MDDCs were stimulated with 10 μg/mL of Akkermansia lipids, 100 ng/mL of Pam3CSK4, 100 ng/mL of FSL-1, or 100 ng/mL of LPS for 18 hours or as indicated. Cell supernatants were collected for human TNFα measurements by ELISA (Invitrogen) following the manufacturer's protocol. SoftMax Pro 6.2.1 (SpectraMax, Molecular Devices) was used to analyze ELISA plates. The sgRNA sequences used were as follows:

Human TLR1:
(SEQ ID NO: 1)
GGTCTTAGGAGAGACTTATG
Human TLR2:
(SEQ ID NO: 2)
GACCGCAATGGTATCTGCAA
Human TLR6:
(SEQ ID NO: 3)
ATTCATTTCCGTCGGAGAAC

TLR2-TLR1-a15:0-i15:0 PE Complex Modeling

Modelling of the a15:0-i15:0 PE ligand complex was based on the crystal structure of the TLR2-TLR1-Pam3CSK4 complex from Protein Data Bank (PDB ID: 2z7x) (36). The Pam3CSK4 ligand was removed from the crystal structure coordinates, and an a15:0-i15:0 PE ligand was prepared using Lidia and AceDRG in Coot 0.9 (50, 51). The a15:0i15:0 PE ligand placement in the ligand-binding pockets of TLR2 and TLR1 was guided by the electron density belonging to the acyl chains of the Pam3CSK4 ligand in the crystal structure. Structural figures and movies were generated using ChimeraX 1.0 (52). Structural biology software was compiled and configured by SBGrid consortium (53).

TABLE 3
Proposed a15:0-i15:0 PE biosynthetic genes,
protein size, function and closest homologs.

| | | | | Sequence similarity |
|---|---|---|---|---|
| | Deduced function of the | | | (origin/ protein |
| Gene | encoded protein | size | Similarity/identity | accession number) |
| Amuc_1689 | dihydrolipoamide | 462 | 100/100 | Akkermansia |
| | dehydrogenase | | | muciniphila |
| | | | | (WP_012420722.1) |
| Amuc_1677 | DEAD/DEAH box helicase | 1110 | 100/100 | Akkermansia |
| | family protein | | | muciniphila |
| | | | | (WP_012420710.1) |
| Amuc_1652 | phosphatidylserine | 298 | 100/100 | Akkermansia |
| | decarboxylase | | | muciniphila |
| | | | | (WP_012420686.1) |
| Amuc_1650 | acetolactate synthase | 617 | 100/99.8 | Akkermansia |
| | | | | muciniphila |
| | | | | (WP_123044219.1) |
| Amuc_1634 | Inorganic phosphate | 746 | 100/100 | Akkermansia |
| | transporter | | | muciniphila |
| | | | | (WP_012420669.1) |
| Amuc_1632 | phosphatidate | 306 | 100/100 | Akkermansia |
| | cytidylyltransferase | | | muciniphila |
| | | | | (WP_031931152.1) |
| Amuc_1487 | Valine-pyruvate transaminase | 424 | 100/100 | Akkermansia |
| | | | | muciniphila |
| | | | | (WP_012420524.1) |
| Amuc_1178 | Ketol-acid reductoisomerase | 326 | 100/100 | Akkermansia |
| | | | | muciniphila |
| | | | | (WP_012420218.1) |
| Amuc_1111 | dihydroxy-acid dehydratase | 558 | 100/100 | Akkermansia |
| | | | | muciniphila |
| | | | | (WP_012420152.1) |
| Amuc_1020 | 3-oxoacyl-[acyl carrier | 546 | 100/100 | Akkermansia |
| | protein] synthase 3 | | | muciniphila |
| | | | | (WP_012420061.1) |
| Amuc_887 | thiamine pyrophosphate- | 312 | 100/100 | Akkermansia |
| | dependent dehydrogenase E1 | | | muciniphila |
| | component subunit alpha | | | (WP_012419935.1) |
| Amuc_886 | transketolase | 326 | 100/100 | Akkermansia |
| | | | | muciniphila |
| | | | | (WP_012419934.1) |
| Amuc_885 | 2-oxo acid dehydrogenase | 424 | 100/100 | Akkermansia |
| | subunit E2 | | | muciniphila |
| | | | | (WP_012419933.1) |
| Amuc_785 | 3-isopropylmalate | 369 | 100/100 | Akkermansia |
| | dehydrogenase | | | muciniphila |
| | | | | (WP_012419834.1) |
| Amuc_723 | branched chain amino acid | 288 | 100/100 | Akkermansia |
| | transaminase | | | muciniphila |
| | | | | (WP_012419775.1) |
| Amuc_667 | 3-isopropylmalate dehydratase | 202 | 100/100 | Akkermansia |
| | small subunit | | | muciniphila |
| | | | | (WP_012419719.1) |
| Amuc_666 | 3-isopropylmalate dehydratase | 470 | 100/100 | Akkermansia |
| | large subunit | | | muciniphila |
| | | | | (WP_012419718.1) |
| Amuc_595 | 1-acyl-sn-glycerol-3- | 234 | 100/100 | Akkermansia |
| | phosphate acyltransferase | | | muciniphila |
| | | | | (WP_012419647.1) |
| Amuc_593 | lysophospholipid | 826 | 100/100 | Akkermansia |
| | acyltransferase family protein | | | muciniphila |
| | | | | (WP_012419645.1) |
| Amuc_65 | 2-isopropylmalate synthase | 1087 | 100/100 | Akkermansia |
| | | | | muciniphila |
| | | | | (WP_012419125.1) |

### Experimental Procedures for Synthesis

Total synthesis of a15:0-i15:0 PE, i15:0-a15:0 PE, a15:0-a15:0 PE, i15:0-i15:0 PE, and n15:0-n15:0 PE

### Reagents, Instruments, and Techniques

All commercially available starting materials were purchased from Sigma Aldrich, Fisher Scientific, Oakwood Chemical, Combi-Blocks, and Avanti Polar Lipids. All reagents were used as received without further purification. Some intermediates were synthesized following reported procedures and any modifications are noted. Anhydrous solvents, such as diethyl ether (Et₂O), dichloromethane (DCM), pyridine, acetonitrile (MeCN) were purchased from Fisher Scientific or Sigma Aldrich, and used as received. If necessary, air- or moisture-sensitive reactions were carried out under an inert atmosphere of nitrogen.

Removal of solvents was accomplished on a Buchi R-300 rotary evaporator and further concentration was done under a Welch 1400B-01 vacuum line and Labconco FreeZone 6 plus system. Purification of compounds was performed by normal phase column chromatography using a Teledyne CombiFlash chromatography system, and/or reversed phase chromatography on a Waters Micromass ZQ preparative system with SunFire® Prep C₁₈ OBD™ 5 μM column. Analytical thin layer chromatography (TLC) plates were purchased from Fisher Scientific (EMD Millipore TLC Silica Gel60 F254). Visualization was accomplished using CAM stain (cerium sulfate (5.0 g), ammonium molybdate (25.0 g), H₂SO₄ (50 mL), H₂O (450 mL)).

All ¹H-NMR spectra were recorded at 24.85° C. on a Bruker ARX 500 (500 MHz) spectrometer. ¹³C-NMR spectra were recorded on a Bruker ARX 500 (126 MHz) spectrometer. Samples were dissolved in CDCl₃, DMSO-d6, or CD₃OD. The spectra were referenced to the residual solvent peak (chlorofrom-d: 7.26 ppm for ¹H-NMR and 77.16 ppm for ¹³C-NMR; DMSO-d₆: 2.50 ppm for ¹H-NMR and 39.25 ppm for ¹³C-NMR, CD₃OD: 3.31 ppm for ¹H NMR and 49.00 ppm for ¹³C NMR or tetramethylsilane (TMS) as the internal standard. Chemical shift, multiplicity (s=singlet, d=doublet, t=triplet, q=quartet, m=multiplet, br=broad peak), coupling constants (Hz), and number of protons. Mass spectrometry (LCMS) data were obtained on Waters Acquity UPLC system in positive ESI mode.

### Abbreviations

| | |
|---|---|
| Dichloromethane | DCM |
| Diethyl ether | Et₂O |
| Acetonitrile | MeCN |
| Trifluoroacetic acid | TFA |
| N,N′-Dicyclohexylcarbodimide | DCC |
| 4-Dimethylaminopyridine | DMAP |
| Bromotrimethylsilane | TMSBr |
| Pentadecanoic acid | n15:0 fatty acid |
| 13-Methylteradecanoic acid | i15:0 fatty acid |
| (S)-12-Methyltetradecanoic acid | (S)-a15:0 fatty acid |
| 2,4,6-Triisopropylbenzenesulfonyl chloride | TPSCl |
| Overnight | o/n |
| Room temperature | RT |

### Compound Experimentals

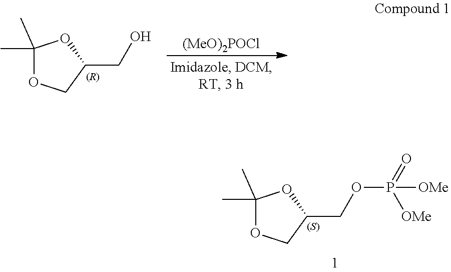

(R)-isopropylidine glycerol (1.0 g, 7.6 mmol) was dissolved in anhydrous DCM (25 mL) at 0° C. Into the solution imidazole (0.77 g, 11.4 mmol) was added in one portion, followed by dropwise addition of dimethylchlorophosphate (1.36 g, 9.5 mmol). The reaction mixture was then warmed up to room temperature, and stirred for 3.5 h. After completion, the crude was diluted with DCM (50 mL) and washed with brine (50 mL×2). The combined aqueous layers were then extracted with DCM (50 mL×2). The combined organic layer was dried over anhydrous sodium sulfate, filtered, and concentrated under reduced pressure. The crude material was then purified by flash chromatography (80% EtOAc in hexanes to 100% EtOAc to 10% MeOH in EtOAc) to afford the desired product (0.36 g, 20%) (18) and deprotected diol (0.95 g, 63%).

¹H NMR of 1 (500 MHz, CDCl₃) δ 4.38-4.24 (m, 1H), 4.05 (m, 3H), 3.83 (dd, J=8.6, 5.5 Hz, 1H), 3.79 (d, J=5.5 Hz, 3H), 3.77 (d, J=5.5 Hz, 3H), 1.43 (s, 3H), 1.36 (s, 3H).

¹³C NMR of 1 (125 MHz, CDCl₃) δ 109.65 (s), 73.87 (d, J=7.8 Hz), 67.38 (d, J=5.7 Hz), 65.77 (s), 54.25 (d, J=2.1 Hz), 54.20 (d, J=1.9 Hz), 26.47 (s), 25.03 (s).

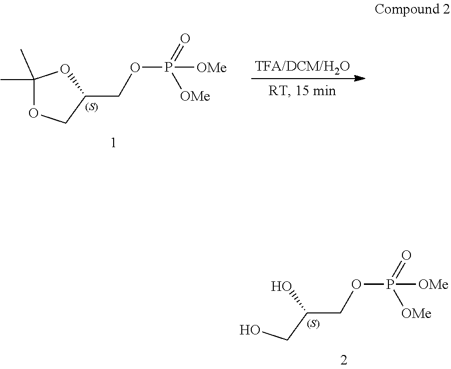

(S)-(2,2-dimethyl-1,3-dioxolan-4-yl) methyl dimethyl phosphate (0.16 g, 0.67 mmol) was dissolved in 0.2 mL DCM and 1.8 mL H₂O. Into the mixture was added 0.2 mL TFA. The reaction was stirred at room temperature for 15 min before concentrated under reduced pressure. The crude material was purified by flash chromatography (100% EtOAc to 20% MeOH in EtOAc with 0.5% Et₃N) to afford desired product (0.10 g, 77%).

¹H NMR of 2 (500 MHz, CDCl₃) δ 4.09 (dd, J=8.6, 5.3 Hz, 2H), 3.91 (m, 2H), 3.78 (d, J=1.9 Hz, 3H), 3.76 (d, J=1.9 Hz, 3H), 3.72-3.67 (dd, J=11.7, 5.8 Hz, 1H), 3.63 (dd, J=11.6, 5.5 Hz, 1H).

¹³C NMR of 2 (125 MHz, CDCl₃) δ 70.60 (d, J=6.4 Hz), 68.68 (d, J=6.1 Hz), 62.76 (s), 54.67 (d, J=6.2 Hz).

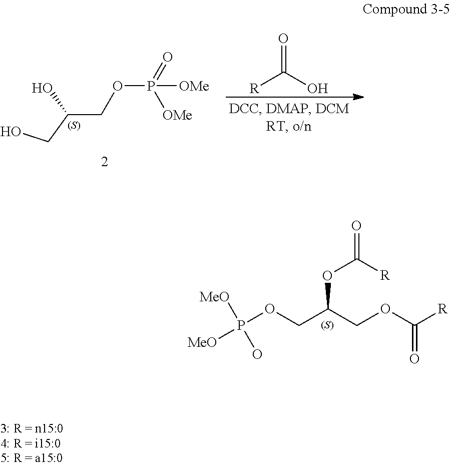

(S)-2,3-dihydroxypropyl dimethyl phosphate (1.0 eq.) and fatty acids (2.0 eq.) were dissolved in anhydrous DCM (1-3 mL). DMAP (2.0 eq.) and DCC (5.0 eq.) were sequentially added into the stirred solution. The mixture was stirred in a sealed vial at room temperature overnight. The mixture was then filtered through a cotton-ball pipette, and concentrated under reduced pressure. The crude material was then purified using flash chromatography (20% to 50% EtOAc in hexanes) to afford desired products.

| | (S)-2,3-dihydroxypropyl | | | Yield |
|---|---|---|---|---|
| | dimethyl phosphate | DCC | DMAP | (%) |
| n15:0 fatty acid | 50 mg | 250 mg | 60 mg | 110 mg |
| (125 mg, 0.5 mmol) | (0.25 mmol) | (1.25 mmol) | (0.5 mmol) | (70%) |
| i15:0 fatty acid | 10 mg | 52 mg | 12 mg | 28 mg |
| (25 mg, 0.1 mmol) | (0.05 mmol) | (0.25 mmol) | (0.1 mmol) | (86%) |
| (S)-a15:0 fatty acid | 10 mg | 52 mg | 12 mg | 29 g |
| (25 mg, 0.1 mmol) | (0.05 mmol) | (0.25 mmol) | (0.1 mmol) | (89%) |

When R—COOH=n15:0 fatty acid:

¹H NMR of 3 (500 MHz, CDCl₃) δ 5.30-5.21 (m, 1H), 4.35 (dd, J=11.9, 4.4 Hz, 1H), 4.26-4.16 (m, 3H), 3.81 (d, J=4.1 Hz, 3H), 3.79 (d, J=4.1 Hz, 3H), 2.35 (dt, J=11.4, 7.6 Hz, 4H), 1.65 (dd, J=14.3, 7.4 Hz, 4H), 1.37-1.21 (m, 44H), 0.94-0.87 (m, 6H).

¹³C NMR of 3 (125 MHz, CDCl₃) δ 172.95, 172.61, 69.69, 69.63, 65.79, 65.75, 61.54, 54.07, 54.03, 33.70, 33.56, 31.89, 29.68, 29.64, 29.50, 29.34, 29.30, 29.02, 28.96, 24.72, 22.55, 13.45, 13.43.

When R—COOH=i15:0 fatty acid:

¹H NMR of 4 (500 MHz, CDCl₃) δ 5.29-5.22 (m, 1H), 4.34 (dd, J=15.2, 5.8 Hz, 1H), 4.26-4.13 (m, 3H), 3.80 (d, J=4.1 Hz, 3H), 3.78 (d, J=4.1 Hz, 3H), 2.34 (dt, J=11.4, 7.6 Hz, 4H), 1.67-1.58 (m, 4H), 1.58-1.48 (m, 2H), 1.29 (m, 32H), 1.16 (m, 4H), 0.88 (d, J=6.6 Hz, 12H).

¹³C NMR of 4 (125 MHz, CDCl₃) δ 173.39, 172.99, 69.55, 69.49, 65.63, 65.58, 61.74, 54.64, 54.59, 39.19, 34.30, 34.16, 30.08, 29.85, 29.80, 29.76, 29.61, 29.42, 29.26, 29.22, 28.10, 27.56, 24.98, 22.82, 22.79, 22.76.

When R—COOH=a15:0 fatty acid:

¹H NMR of 5 (500 MHz, CDCl₃) δ 5.28-5.16 (m, 1H), 4.31 (dd, J=11.7, 5.9 Hz, 1H), 4.24-4.06 (m, 3H), 3.76 (d, J=3.9 Hz, 3H), 3.75 (d, J=4.1 Hz, 3H), 2.31 (dt, J=11.4, 7.6 Hz, 4H), 1.65-1.53 (m, 4H), 1.34-1.17 (m, 34H), 1.16-1.01 (m, 4H), 0.87-0.78 (m, 12H).

¹³C NMR of 5 (125 MHz, CDCl₃) δ 173.33, 172.94, 69.52, 69.46, 65.60, 65.55, 61.70, 54.60, 54.55, 36.74, 34.49, 34.26, 34.12, 30.12, 29.80, 29.73, 29.63, 29.59, 29.38, 29.22, 29.18, 27.21, 24.94, 19.31, 11.50.

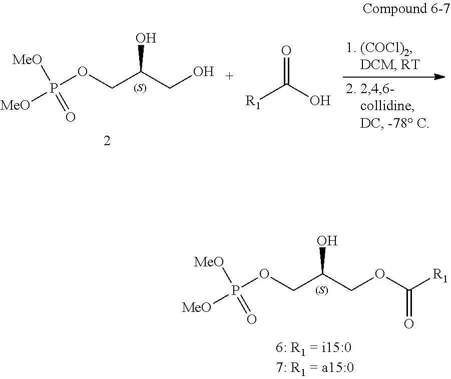

13-methyltetradecanoic acid (0.02 g, 0.08 mmol) or (S)-12-methyltetradecanoic acid (0.02 g, 0.08 mmol) was dissolved in anhydrous DCM (2 mL) under N₂. Oxalyl chloride (0.05 g, 0.4 mmol) and 2 drops of anhydrous DMF were added sequentially. The solution was stirred at room temperature under N₂ for 3 h. Then the solvent was removed under vacuum. The residual material was re-dissolved in anhydrous DCM (2 mL), and added dropwise into the solution of (S)-2,3-dihydroxypropyl dimethyl phosphate (0.025 g, 0.13 mmol) and 2,4,6-collidine (0.05 g, 0.4 mmol) at −78° C. under N₂. The mixture was stirred for 1 hour, quenched by addition of MeOH (0.2 mL), and concentrated under reduced pressure. The crude material was purified by column chromatography (20% to 100% EtOAc in hexanes) to afford the desired product (19).

When R₁—COOH=i15:0 fatty acid (6 mg, 14%):

¹H NMR of 6 (500 MHz, CDCl₃) δ 4.23-4.07 (m, 5H), 3.84 (s, 3H), 3.83-3.80 (s, 3H), 2.37 (t, J=7.6 Hz, 2H), 1.70-1.59 (m, 2H), 1.53 (tp, J=13.2, 6.7 Hz, 1H), 1.29 (m, 16H), 1.17 (m, 2H), 0.89 (d, J=6.3 Hz, 6H).

¹³C NMR of 6 (126 MHz, CDCl₃) δ 174.00, 69.07, 69.01, 68.96, 64.47, 54.82, 39.20, 34.24, 30.08, 29.85, 29.79, 29.75, 29.60, 29.40, 29.27, 28.12, 27.56, 25.03, 22.81.

When R₁—COOH=a15:0 fatty acid (10 mg, 28%):

¹H NMR of 7 (500 MHz, CDCl₃) δ 4.28-4.01 (m, 5H), 3.83 (d, J=0.85 Hz, 3H), 3.81 (d, J=1.0 Hz, 3H), 2.37 (dd, J=13.1, 5.6 Hz, 2H), 1.69-1.58 (m, 2H), 1.39-1.19 (m, 17H), 1.19-1.01 (m, 2H), 0.93-0.80 (m, 6H).

¹³C NMR of 7 (126 MHz, CDCl₃) δ 173.98, 69.03, 68.99, 68.94, 64.46, 54.80, 36.77, 34.54, 34.23, 30.15, 29.82, 29.75, 29.64, 29.60, 29.39, 29.27, 27.24, 25.02, 19.36, 11.55.

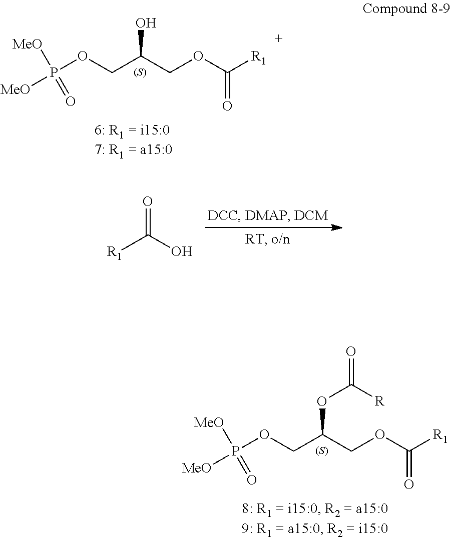

(S)-3-((dimethoxyphosphoryl)oxy)-2-hydroxypropyl 13-methyltetradecanoate (6 mg, 0.014 mmol) or (S)-3-((dimethoxyphosphoryl)oxy)-2-hydroxypropyl (S)-12-methyltetradecanoate (10 mg, 0.024 mmol) and fatty acids (1.0 eq.) were dissolved in anhydrous DCM. DMAP (2.0 eq.) and DCC (3.0 eq.) were added to the solution sequentially. The mixture was stirred in a sealed vial at room temperature overnight. The mixture was then filtered through a cotton-ball pipette, and concentrated under reduced pressure. The crude material was then purified using flash chromatography (20% to 50% EtOAc in hexanes) to afford desired products.

| | (S)-a15:0 | i15:0 | | | Yield |
|---|---|---|---|---|---|
| | fatty acid | fatty acid | DCC | DMAP | (%) |
| (S)-3- | 3.4 mg | — | 8.7 mg | 3.4 mg | 5.7 mg |
| ((dimethoxyphosphoryl)oxy)- | (0.014 mmol) | | (0.042 mmol) | (0.028 mmol) | (63%) |
| 2-hydroxypropyl 13- | | | | | |
| methyltetradecanoate | | | | | |
| (6 mg, 0.014 mmol) | | | | | |
| (S)-3- | — | 5.8 mg | 15 mg | 5.9 mg | 10.0 mg |
| ((dimethoxyphosphoryl)oxy)- | | (0.024 mmol) | (0.072 mmol) | (0.048 mmol) | (64%) |
| 2-hydroxypropyl (S)-12- | | | | | |
| methyltetradecanoate | | | | | |
| (10 mg, 0.024 mmol) | | | | | |

When R₂—COOH=a15:0 fatty acid:

¹H NMR of 8 (500 MHz, CDCl₃) δ 5.28-5.20 (m, 1H), 4.32 (dd, J=11.6, 5.8 Hz, 1H), 4.25-4.12 (m, 3H), 3.78 (d, J=4.1 Hz, 3H), 3.76 (d, J=4.1 Hz, 3H), 2.32 (dt, J=11.4, 7.6 Hz, 4H), 1.61 (dq, J=14.4, 7.3 Hz, 4H), 1.51 (tt, J=13.2, 6.6 Hz, 1H), 1.35-1.19 (m, 33H), 1.19-1.02 (m, 4H), 0.91-0.79 (m, 12H).

¹³C NMR of 8 (125 MHz, CDCl₃) δ 173.41, 173.01, 69.56, 69.50, 65.64, 65.60, 61.76, 54.66, 54.61, 39.21, 36.79, 34.55, 34.32, 34.18, 30.17, 30.10, 29.86, 29.81, 29.78, 29.63, 29.43, 29.27, 29.24, 28.12, 27.57, 27.26, 25.00, 22.81, 19.37, 11.56.

When R₂—COOH=i15:0 fatty acid:

¹H NMR of 9 (500 MHz, CDCl₃) δ 5.28-5.19 (m, 1H), 4.32 (dd, J=11.5, 5.7 Hz, 1H), 4.24-4.11 (m, 3H), 3.78 (d, J=4.1 Hz, 3H), 3.76 (d, J=4.1 Hz, 3H), 2.32 (dt, J=11.2, 7.6 Hz, 4H), 1.61 (dq, J=14.3, 7.3 Hz, 4H), 1.56-1.44 (m, 1H), 1.35-1.19 (m, 33H), 1.19-1.02 (m, 4H), 0.91-0.79 (m, 12H).

¹³C NMR of 9 (125 MHz, CDCl₃) δ 173.41, 173.01, 69.56, 69.50, 65.64, 65.60, 61.75, 54.66, 54.61, 39.20, 36.79, 34.55, 34.32, 34.18, 30.17, 30.09, 29.86, 29.85, 29.81, 29.78, 29.63, 29.43, 29.27, 29.23, 28.12, 27.57, 27.26, 24.99, 22.80, 19.37, 11.55.

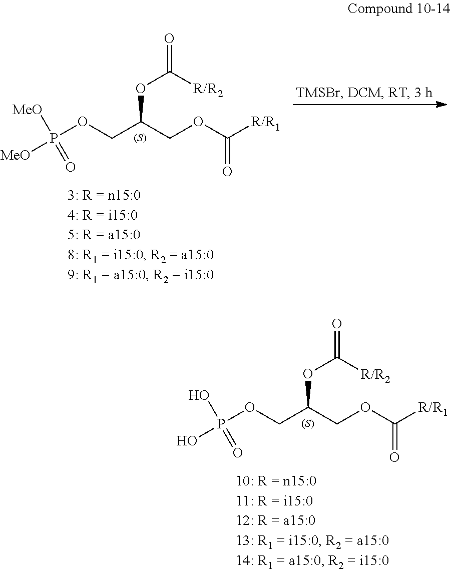

Diacyl-(S)-2,3-dihydroxypropyl dimethyl phosphate compounds were dissolved in anhydrous DCM (1-2 mL). TMSBr (3.0 eq.) was added dropwise. The solution was stirred at room temperature in a sealed vial then concentrated under reduced pressure. The crude material was then dissolved in 95% MeOH, and stirred for 1 h at room temperature. The solution was concentrated again. The crude material was re-dissolved in acetonitrile at 80° C., which was then cooled to 0° C. or −20° C. depending on the material being purified. The precipitated solid was then collected by filtration to afford the desired product.

| R | R₁ | R₂ | Mass/Mol. | TMSBr | Yield (%) |
|---|---|---|---|---|---|
| n15:0 | — | — | 58 mg | 41 mg | 36 mg |
| | | | (0.09 mmol) | (0.27 mmol) | (65%) |
| i15:0 | — | — | 28 mg | 19.8 mg | 22 mg |
| | | | (0.043 mmol) | (0.13 mmol) | (83%) |
| (S)- | — | — | 28 mg | 19.8 mg | 24 mg |
| a15:0 | | | (0.043 mmol) | (0.13 mmol) | (85%) |
| — | i15:0 | (S)- | 6 mg | 4.3 mg | 5 mg |
| | | a15:0 | (0.009 mmol) | (0.027 mmol) | (90%) |
| — | (S)- | i15:0 | 10 mg | 7 mg | 8.8 mg |
| | a15:0 | | (0.015 mmol) | (0.045 mmol) | (95%) |

When R=n15:0 fatty acid:

¹H NMR of 10 (500 MHz, CDCl₃) δ 9.37 (s, 2H), 5.24 (dt, J=10.3, 5.1 Hz, 1H), 4.36 (dd, J=12.0, 3.5 Hz, 1H), 4.19 (dd, J=12.0, 6.4 Hz, 1H), 4.11 (m, 2H), 2.32 (dt, J=13.8, 7.6 Hz, 4H), 1.59 (dt, J=13.7, 6.7 Hz, 4H), 1.37-1.18 (m, 44H), 0.87 (t, J=6.9 Hz, 6H).

¹³C NMR of 10 (125 MHz, CDCl₃) δ 174.00, 173.65, 69.83, 69.77, 64.99, 64.95, 62.30, 46.54, 34.32, 34.20, 32.08, 29.88, 29.86, 29.84, 29.72, 29.53, 29.51, 29.49, 29.32, 29.28, 24.98, 24.97, 22.84, 14.25, 8.70.

When R=i15:0 fatty acid:

¹H NMR of 11 (500 MHz, CDCl₃) δ 8.03 (s, 2H), 5.34-5.14 (m, 1H), 4.35 (dd, J=10.9, 5.5 Hz, 1H), 4.24-4.18 (dd, J=12.2, 6.2 Hz, 1H), 4.18-4.09 (m, 2H), 2.33 (dt, J=13.7, 7.6 Hz, 4H) 1.59 (dt, J=13.9, 6.8 Hz, 4H), 1.56-1.45 (m, 2H), 1.27 (m, 32H), 1.15 (m, 4H), 0.86 (d, J=6.6 Hz, 12H).

¹³C NMR of 11 (125 MHz, CDCl₃) δ 174.27, 173.90, 69.83, 69.77, 65.15, 62.28, 39.22, 34.35, 34.23, 30.13, 29.92, 29.87, 29.70, 29.48, 29.31, 29.26, 28.12, 27.60, 24.97, 24.96, 22.81.

When R=a15:0 fatty acid:

¹H NMR of 12 (500 MHz, CDCl₃) δ 7.79 (s, 2H), 5.30-5.19 (m, 1H), 4.36 (dd, J=12.0, 3.6 Hz, 1H), 4.25-4.17 (m, 1H), 4.17-4.07 (m, 2H), 2.39-2.27 (dt, J=14.5, 7.5 Hz, 4H), 1.60 (dd, J=13.4, 6.7 Hz, 4H), 1.35-1.20 (m, 34H), 1.17-1.01 (m, 4H), 0.85 (m, 12H).

¹³C NMR of 12 (125 MHz, CDCl₃) δ 174.24, 173.88, 69.83, 69.77, 65.07, 62.30, 36.82, 34.56, 34.34, 34.22, 30.22, 29.90, 29.86, 29.85, 29.71, 29.65, 29.49, 29.48, 29.31, 29.26, 27.29, 24.97, 24.96, 19.36, 11.55.

When R₁=i15:0, R₂=a15:0 fatty acid:

¹H NMR of 13 (500 MHz, CDCl₃) δ 5.26 (m, 1H), 4.42-4.28 (m, 1H), 4.20 (dd, J=11.8, 6.2 Hz, 1H), 4.13 (m, 2H), 2.42-2.24 (m, 4H), 1.58 (m, 4H), 1.50 (dq, J=19.9, 6.6 Hz, 1H), 1.38-1.18 (m, 33H), 1.11 (m, 4H), 0.93-0.78 (m, 12H).

¹³C NMR of 13 (125 MHz, CDCl₃) δ 174.28, 173.94, 69.93, 65.12, 62.41, 39.32, 36.93, 34.66, 34.46, 34.33, 30.33, 30.23, 30.02, 29.97, 29.95, 29.82, 29.80, 29.75, 29.60, 29.42, 29.38, 28.23, 27.70, 27.40, 25.07, 22.91, 19.47, 11.66.

When R₁=a15:0, R₂=i15:0 fatty acid:

¹H NMR of 14 (500 MHz, CDCl₃) δ 5.25 (m, 1H), 4.36 (d, J=9.2 Hz, 1H), 4.26-3.99 (m, 3H), 2.48-2.17 (m, 4H), 1.58 (m, 4H), 1.50 (dq, J=19.8, 6.6 Hz, 1H), 1.38-1.18 (m, 33H), 1.18-1.01 (m, 4H), 0.92-0.77 (m, 12H).

¹³C NMR of 14 (126 MHz, CDCl₃) δ 174.13, 173.83, 69.86, 64.95, 62.35, 39.23, 36.82, 34.56, 34.35, 34.23, 30.23, 30.15, 29.94, 29.92, 29.90, 29.88, 29.73, 29.65, 29.52, 29.51, 29.34, 29.29, 28.13, 27.61, 27.30, 24.97, 22.81, 19.37, 11.56.

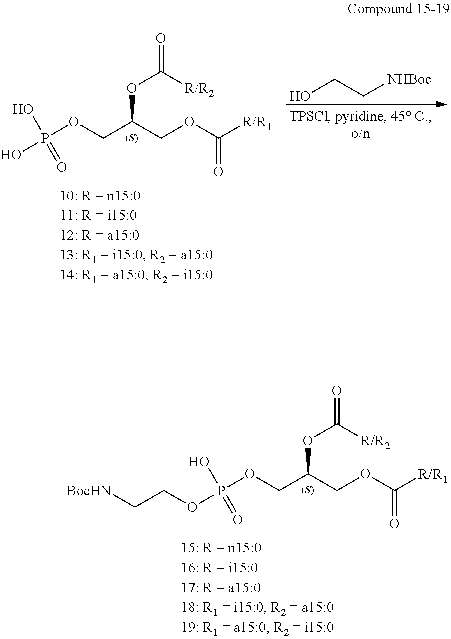

Diacyl-(S)-2,3-dihydroxypropyl dihydrogen phosphate (1.0 eq.) and N-Bocethanolamine (2.0 eq.) were dissolved in anhydrous pyridine (0.5 mL-1.0 mL). 2,4,6-triisopropylbenzenesulfonyl chloride (3.0 eq.) in anhydrous pyridine (0.5 mL-1.0 mL) was added to the stirred solution. The mixture was stirred in a sealed reaction vial at 45° C. overnight. Then the mixture was cooled down to room temperature. 200 μL of H₂O was added to quench the reaction. After stirring for 1 h at room temperature, the mixture was concentrated under reduced pressure. Et₂O was added into the residue from which precipitate was removed by filtration. The filtrate was concentrated under reduced pressure, and subjected to further purification by column chromatography (0% to 20% MeOH in DCM) to afford desired products (20).

| | | | | N-Boc- | | |
|---|---|---|---|---|---|---|
| R | R₁ | R₂ | Mass/Mol. | ethanolamine | TPSCl | Yield (%) |
| n15:0 | — | — | 36 mg | 19 mg | 53 mg | 16 mg |
| | | | (0.058 mmol) | (0.12 mmol) | (0.18 mmol) | (40 %) |
| i15:0 | — | — | 22 mg | 12 mg | 31 mg | 20 mg |
| | | | (0.035 mmol) | (0.07 mmol) | (0.1 mmol) | (75 %) |
| (S)- | — | — | 24 mg | 12 mg | 31 mg | 20 mg |
| a15:0 | | | (0.038 mmol) | (0.07 mmol) | (0.1 mmol) | (69 %) |
| — | i15:0 | (S)- | 5 mg | 2.6 mg | 7.3 mg | 6.8 mg |
| | | a15:0 | (0.008 mmol) | (0.016 mmol) | (0.024 mmol) | (not pure) |
| — | (S)- | i15:0 | 8.8 mg | 4.5 mg | 0.013 g | 10.0 mg |
| | a15:0 | | (0.014 mmol) | (0.028 mmol) | (0.042 mmol) | (not pure) |

When R=n15:0 fatty acid:

¹H NMR of 15 (500 MHz, CD₃OD/CDCl₃=1:3) δ 5.18 (td, J=8.5, 5.4 Hz, 1H), 4.34 (dd, J=10.9, 5.4 Hz, 1H), 4.12 (dd, J=12.0, 6.9 Hz, 1H), 3.95-3.87 (m, 2H), 3.82 (dd, J=13.0, 5.5 Hz, 2H), 3.27 (m, 2H), 2.32-2.21 (m, 4H), 1.56 (d, J=5.8 Hz, 4H), 1.40 (s, 9H), 1.30-1.21 (m, 44H), 0.84 (t, J=6.9 Hz, 6H).

¹³C NMR of 15 (125 MHz, CD₃OD/CDCl₃=1:3) δ 173.77, 173.39, 156.63, 70.28, 70.21, 64.40, 63.43, 62.46, 53.28, 40.76, 34.03, 33.89, 31.73, 29.51, 29.49, 29.47, 29.35, 29.33, 29.17, 29.14, 28.97, 28.95, 28.05, 24.72, 24.67, 24.41, 23.44, 22.47, 13.76.

When R=i15:0 fatty acid:

¹H NMR of 16 (500 MHz, CD₃OD/CDCl₃=1:3) δ 5.18 (m, 1H), 4.15 (dd, J=12.0, 6.8 Hz, 2H), 3.94 (m, 2H), 3.85 (m, 2H), 3.28 (m, 2H), 2.30 (dd, J=14.5, 6.9 Hz, 4H), 1.59 (m, 4H), 1.54-1.46 (m, 2H), 1.44 (s, 9H), 1.33-1.21 (m, 32H), 1.12 (m, 4H), 0.84 (d, J=6.6 Hz, 12H).

¹³C NMR of 16 (125 MHz, CD₃OD/CDCl₃=1:3) δ 173.92, 173.54, 148.03, 70.39, 64.56, 63.53, 62.54, 40.96, 38.98, 34.14, 33.99, 29.86, 29.63, 29.58, 29.44, 29.43, 29.26, 29.23, 29.06, 29.04, 28.13, 27.87, 27.33, 24.82, 24.78, 24.49, 23.53, 22.41.

When R=a15:0 fatty acid:

¹H NMR of 17 (500 MHz, CD₃OD/CDCl₃=1:3) δ 5.20 (m, 1H), 4.37 (dd, J=11.9, 2.5 Hz, 1H), 4.14 (dd, J=12.0, 6.8 Hz, 1H), 3.94 (m, 2H), 3.85 (m, 2H), 3.25 (m, 2H), 2.29 (dd, J=14.1, 6.8 Hz, 4H), 1.58 (m, 4H), 1.42 (s, 9H), 1.34-1.21 (m, 34H), 1.15-1.01 (m, 4H), 0.89-0.76 (m, 12H).

¹³C NMR of 17 (126 MHz, CD₃OD/CDCl₃=1:3) δ 173.87, 173.50, 70.38, 64.58, 63.55, 62.55, 49.53, 40.93, 36.57, 34.34, 34.14, 33.99, 29.95, 29.63, 29.59, 29.58, 29.46, 29.44, 29.40, 29.27, 29.24, 29.08, 29.06, 28.16, 27.03, 24.83, 24.78, 19.01, 11.17.

When R₁=i15:0, R₂=a15:0 fatty acid:

¹H NMR of 18 (500 MHz, CD₃OD/CDCl₃=1:3) δ 5.20 (m, 1H), 4.14 (dd, J=12.0, 6.7 Hz, 1H), 3.95 (m, 2H), 3.86 (m, 2H), 3.28 (s, 2H), 2.33-2.25 (m, 4H), 1.58 (m, 4H), 1.52-1.46 (m, 1H), 1.41 (s, 9H), 1.32-1.23 (m, 33H), 1.15-1.03 (s, 4H), 0.90-0.78 (m, 12H).

When R₁=a15:0, R₂=i15:0 fatty acid:

¹H NMR of 19 (500 MHz, CD₃OD/CDCl₃=1:3) δ 5.24-5.16 (m, 1H), 4.16-4.09 (m, 1H), 3.94 (m, 2H), 3.84 (m, 2H), 3.25 (m, 2H), 2.29 (m, 4H), 1.58 (m, 4H), 1.49 (dt, J=13.3, 6.7 Hz, 1H), 1.43 (s, 9H), 1.32-1.23 (m, 33H), 1.16-1.02 (m, 4H), 0.87-0.79 (m, 12H).

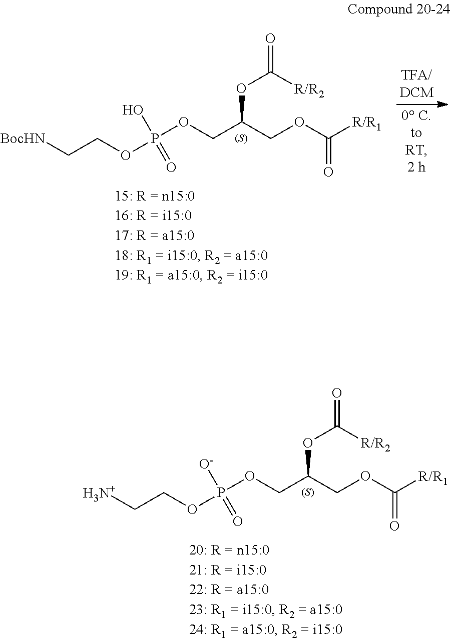

Deprotection of the Boc protective group on ethanolamine was performed in 1 mL anhydrous DCM with addition of 200 uL of trifluoroacetic acid. The mixture was stirred at room temperature for 2 h, then concentrated under reduced pressure. Then the mixture was dissolved in 10 mL of CHCl₃/H2O and 1N NaOH was added to neutralize. The mixture was extracted from CHCl₃ layer and subjected to purification by reversed phase column chromatography (92% MeOH/H₂O isocratic solvent system) to obtain final compounds.

| R | R₁ | R₂ | Mass/Mol. | Yield (%) |
|---|---|---|---|---|
| n15:0 | — | — | 16 mg | 11 mg |
| | | | (0.024 mmol) | (69%) |
| i15:0 | — | — | 20 mg | 5.6 mg |
| | | | (0.030 mmol) | (28%) |
| (S)- | — | — | 20 mg | 6.8 mg |
| a15:0 | | | (0.030 mmol) | (34%) |
| — | i15:0 | (S)- | 6.8 mg | 0.8 mg |
| | | a15:0 | (0.010 mmol) | (12%) |
| — | (S)- | i15:0 | 10 mg | 2 mg |
| | a15:0 | | (0.015 mmol) | (20%) |

When R=n15:0 fatty acid: HR-ESI-QTOF-MS (positive-ion mode) m/z 664.4926 [M+H]⁺ (calcd for C₃₅H₇₁NO₈P, 664.4917).

¹H NMR of 20 (500 MHz, CDCl₃) δ 5.20 (td, J=8.5, 5.4 Hz, 1H), 4.37 (dd, J=12.0, 3.0 Hz, 1H), 4.14 (dd, J=12.0, 7.5 Hz, 1H), 3.99 (m, 2H), 3.91 (t, J=6.5 Hz, 2H), 3.03 (m, 2H), 2.32-2.24 (m, 4H), 1.57 (m, 4H), 1.30-1.21 (m, 44H), 0.88 (t, J=6.5 Hz, 6H).

¹³C NMR of 20 (125 MHz, CDCl₃) δ 173.51, 173.28, 70.29, 63.73, 62.49, 62.08, 40.49, 34.04, 31.91, 29.78, 29.70, 29.66, 29.55, 29.35, 29.18, 24.89, 22.66, 14.08.

When R=i15:0 fatty acid: HR-ESI-QTOF-MS (positive-ion mode) m/z 664.4927 [M+H]⁺ (calcd for C₃₅H₇₁NO₈P, 664.4917).

¹H NMR of 21 (500 MHz, CDCl₃) δ 5.18 (m, 1H), 4.37 (dd, J=12.0, 2.5 Hz, 1H), 4.15 (dd, J=12.0, 6.5 Hz, 1H), 4.07 (m, 2H), 3.89 (m, 2H), 3.12 (m, 2H), 2.30 (m, 4H), 1.59 (m, 4H), 1.54-1.46 (m, 2H), 1.33-1.21 (m, 32H), 1.12 (m, 4H), 0.84 (d, J=6.6 Hz, 12H).

¹³C NMR of 21 (125 MHz, CDCl₃) δ 173.8, 173.6, 70.72, 64.03, 62.83, 62.37, 40.60, 39.30, 34.48, 34.31, 30.21 29.99, 29.95, 29.83, 29.62, 29.59, 29.45, 29.41, 28.19, 27.65, 29.16, 27.15, 25.16, 25.09, 22.86.

When R=a15:0 fatty acid: HR-ESI-QTOF-MS (positive-ion mode) m/z 664.4926 [M+H]⁺ (calcd for C₃₅H₇₁NO₈P, 664.4917).

¹H NMR of 22 (500 MHz, CDCl₃) δ 5.20 (m, 1H), 4.37 (dd, J=12.0, 2.5 Hz, 1H), 4.15 (dd, J=12.0, 6.5 Hz, 1H), 4.07 (m, 2H), 3.89 (m, 2H), 3.12 (m, 2H), 2.30 (m, 4H), 1.58 (m, 4H), c 1.34-1.21 (m, 34H), 1.15-1.05 (m, 4H), 0.87-0.84 (m, 12H).

¹³C NMR of 22 (125 MHz, CDCl₃) δ 174.0, 173.8, 70.39, 63.96, 62.66, 62.54, 40.64, 36.66, 34.39, 34.18, 34.02, 30.09, 29.79, 29.63, 29.61, 29.48, 29.41, 29.38, 29.22, 29.16, 27.15, 24.87, 24.80, 19.19, 11.38.

When R₁=i15:0, R₂=a15:0 fatty acid: HR-ESI-QTOF-MS (positive-ion mode) m/z 664.4915 [M+H]⁺ (calcd for C₃₅H₇₁NO₈P, 664.4917).

¹H NMR of 23 (500 MHz, CDCl₃) δ 5.21 (m, 1H), 4.37 (dd, J=12.0, 2.5 Hz, 1H), 4.14 (dd, J=12.0, 6.7 Hz, 1H), 4.08 (m, 2H), 3.93 (m, 2H), 3.15 (br, 2H), 2.32-2.25 (m, 4H), 1.58 (m, 4H), 1.52-1.46 (m, 1H), 1.32-1.23 (m, 33H), 1.15-1.05 (m, 4H), 0.87-0.83 (m, 12H).

¹³C NMR of 23 (125 MHz, CDCl₃) δ 173.63, 173.37, 70.55, 64.11, 62.80, 62.39, 41.23, 39.28, 36.88, 34.62, 34.47, 34.29, 30.28, 30.20, 29.99, 29.97, 29.96, 29.95, 29.93, 29.82, 29.80, 29.70, 29.63, 29.59, 29.44, 29.41, 28.18, 27.66, 27.35, 25.16, 25.09, 22.85, 19.42, 11.59.

When R₁=a15:0, R₂=i15:0 fatty acid: HR-ESI-QTOF-MS (positive-ion mode) m/z 664.4917 [M+H]⁺ (calcd for C₃₅H₇₁NO₈P, 664.4917).

¹H NMR of 24 (500 MHz, CDCl₃) δ 5.21 (m, 1H), 4.37 (dd, J=12.0, 2.5 Hz, 1H), 4.14 (dd, J=12.0, 6.7 Hz, 1H), 4.08 (m, 2H), 3.93 (m, 2H), 3.15 (br, 2H), 2.32-2.25 (m, 4H), 1.58 (m, 4H), 1.52-1.46 (m, 1H), 1.32-1.23 (m, 33H), 1.15-1.05 (m, 4H), 0.87-0.83 (m, 12H).

¹³C NMR of 24 (125 MHz, CDCl₃) δ 173.63, 173.37, 70.55, 64.11, 62.80, 62.39, 41.23, 39.28, 36.88, 34.62, 34.47, 34.29, 30.28, 30.20, 29.99, 29.97, 29.96, 29.95, 29.93, 29.82, 29.80, 29.70, 29.63, 29.59, 29.44, 29.41, 28.18, 27.66, 27.35, 25.16, 25.09, 22.85, 19.42, 11.59.

### REFERENCES

1. P. D. Cani, Human gut microbiome: hopes, threats and promises. Gut 67, 1716-1725 (2018).
2. S. Y. Geerlings, I. Kostopoulos, W. M. de Vos, C. Belzer, Akkermansia muciniphila in the Human Gastrointestinal Tract: When, Where, and How? Microorganisms 6, (2018).
3. A. Everard et al., Cross-talk between Akkermansia muciniphila and intestinal epithelium controls diet-induced obesity. Proc Natl Acad Sci USA 110, 9066-9071 (2013).
4. H. Plovier et al., A purified membrane protein from Akkermansia muciniphila or the pasteurized bacterium improves metabolism in obese and diabetic mice. Nat Med 23, 107-113 (2017).
5. B. Routy et al., Gut microbiome influences efficacy of PD-1-based immunotherapy against epithelial tumors. Science 359, 91-97 (2018).
6. E. Ansaldo et al., Akkermansia muciniphila induces intestinal adaptive immune responses during homeostasis. Science 364, 1179-1184 (2019).
7. M. T. Henke et al., Ruminococcus gnavus, a member of the human gut microbiome associated with Crohn's disease, produces an inflammatory polysaccharide. Proc Natl Acad Sci USA, (2019).
8. C. Sohlenkamp, O. Geiger, Bacterial membrane lipids: diversity in structures and pathways. FEMS Microbiol Rev 40, 133-159 (2016).
9. B. Mostofian, T. Zhuang, X. Cheng, J. D. Nickels, Branched-Chain Fatty Acid Content Modulates Structure, Fluidity, and Phase in Model Microbial Cell Membranes. J Phys Chem B 123, 5814-5821 (2019).
10. Y. M. Zhang, C. O. Rock, Membrane lipid homeostasis in bacteria. Nat Rev Microbiol 6, 222-233 (2008).
11. M. Wallace et al., Enzyme promiscuity drives branched-chain fatty acid synthesis in adipose tissues. Nat Chem Biol 14, 1021-1031 (2018).
12. N. G. Forouhi, Association between consumption of dairy products and incident type 2 diabetes—insights from the European Prospective Investigation into Cancer study. Nutr Rev 73 Suppl 1, 15-22 (2015).
13. B. Hou, B. Reizis, A. L. DeFranco, Toll-like receptors activate innate and adaptive immunity by using dendritic cell-intrinsic and -extrinsic mechanisms. Immunity 29, 272-282 (2008).
14. J. Yao, C. O. Rock, Bacterial fatty acid metabolism in modern antibiotic discovery. Biochim Biophys Acta Mol Cell Biol Lipids 1862, 1300-1309 (2017).
15. Y. Belkaid, O. J. Harrison, Homeostatic Immunity and the Microbiota. Immunity 46, 562-576 (2017).
16. J. Y. Kang et al., Recognition of lipopeptide patterns by Toll-like receptor 2-Toll-like receptor 6 heterodimer. Immunity 31, 873-884 (2009).
17. B. L. Lu, G. M. Williams, M. A. Brimble, TLR2 agonists and their structure-activity relationships. Org Biomol Chem 18, 5073-5094 (2020).
18. J. E. East, T. L. Macdonald, Cost-effective and Large-scale synthesis of 16:0 Lysophosphatidic Acid. Synthetic Communications, 42, 3614-3619 (2012).
19. K. Ishihara, H. Kurihara, H. Yamamoto, An extremely simple, convenient, and selective method for acetylating primary alcohols in the presence of secondary alcohols J. Org. Chem. 58, 3791-3793 (1993).
20. S. Doussin, N. Bbirlirakis, D. Georgin, F. Taran, P. Berthault, Novel Zwitterionic Reverse Micelles for Encapsulation of Proteins in Low-Viscosity Media Chem. Eur. J. 12, 4170-4175 (2006).
21. M. T. Henke, D. J. Kenny, C. D. Cassilly, H. Vlamakis, R. J. Xavier, J. Clardy, Ruminococcus gnavus, a member of the human gut microbiome associated with Crohn's disease, produces an inflammatory polysaccharide. Proc. Natl. Acad. Sci. U.S.A. 116, 12672-12677 (2019).
22. Fan, Y. & Pedersen, O. Gut microbiota in human metabolic health and disease. Nat Rev Microbiol 19, 55-71 (2021).
23. Routy, B., Gopalakrishnan, V., Daillere, R., Zitvogel, L., Wargo, J. A. & Kroemer, G. The gut microbiota influences anticancer immunosurveillance and general health. Nat Rev Clin Oncol 15, 382-396 (2018).
24. Schmidt, T. S. B., Raes, J. & Bork, P. The Human Gut Microbiome: From Association to Modulation. Cell 172, 1198-1215 (2018).
25. Depommier, C., Everard, A., Druart, C., Plovier, H., Hul, M. V., Vieira-Silva, S., Falony, G., Raes, J., Maiter, D., Delzenne, N. M., Barsy, M. de, Loumaye, A., Hermans, M. P., Thissen, J.-P., Vos, W. M. de & Cani, P. D. Supplementation with Akkermansia muciniphila in overweight and obese human volunteers: a proof-of-concept exploratory study. Nat Med 25, 1096-1103 (2019).
26. Derosa, L., Routy, B., Thomas, A. M., Iebba, V., Zalcman, G., Friard, S., Mazieres, J., Audigier-Valette, C., Moro-Sibilot, D., Goldwasser, F., Silva, C. A. C., Terrisse, S., Bonvalet, M., Scherpereel, A., Pegliasco, H., Richard, C., Ghiringhelli, F., Elkrief, A., Desilets, A., Blanc-Durand, F., Cumbo, F., Blanco, A., Boidot, R., Chevrier, S., Daillere, R., Kroemer, G., Alla, L., Pons, N., Chatelier, E. L., Galleron, N., Roume, H., Dubuisson, A., Bouchard, N., Messaoudene, M., Drubay, D., Deutsch, E., Barlesi, F., Planchard, D., Segata, N., Martinez, S., Zitvogel, L., Soria, J.-C. & Besse, B. Intestinal Akkermansia muciniphila predicts clinical response to PD-1 blockade in patients with advanced non-small-cell lung cancer. Nat Med 28, 315-324 (2022).
27. Takeda, K. & Akira, S. Toll-Like Receptors. Curr Protoc Immunol 109, 14.12.1-14.12.10 (2015).
28. Cani, P. D. & Vos, W. M. de. Next-Generation Beneficial Microbes: The Case of Akkermansia muciniphila. Front Microbiol 8, 1765 (2017).
29. Forouhi, N. G., Krauss, R. M., Taubes, G. & Willett, W. Dietary fat and cardiometabolic health: evidence, controversies, and consensus for guidance. Bmj 361, k2139 (2018).
30. Taormina, V. M., Unger, A. L., Schiksnis, M. R., Torres-Gonzalez, M. & Kraft, J. Branched-Chain Fatty Acids—An Underexplored Class of Dairy-Derived Fatty Acids. Nutrients 12, 2875 (2020).
31. Akira, S. & Takeda, K. Toll-like receptor signalling. Nat Rev Immunol 4, 499-511 (2004).
32. Ottman, N., Reunanen, J., Meijerink, M., Pietila, T. E., Kainulainen, V., Klievink, J., Huuskonen, L., Aalvink, S., Skurnik, M., Boeren, S., Satokari, R., Mercenier, A., Palva, A., Smidt, H., Vos, W. M. de & Belzer, C. Pili-like proteins of Akkermansia muciniphila modulate host immune responses and gut barrier function. Plos One 12, e0173004 (2017).
33. Ouyang, J., Lin, J., Isnard, S., Fombuena, B., Peng, X., Marette, A., Routy, B., Messaoudene, M., Chen, Y. & Routy, J.-P. The Bacterium Akkermansia muciniphila: A Sentinel for Gut Permeability and Its Relevance to HIV-Related Inflammation. Front Immunol 11, 645 (2020).
34. Oh, S. F., Praveena, T., Song, H., Yoo, J.-S., Jung, D.-J., Erturk-Hasdemir, D., Hwang, Y. S., Lee, C. C., Nours, J. L., Kim, H., Lee, J., Blumberg, R. S., Rossjohn, J., Park, S. B. & Kasper, D. L. Host immunomodulatory lipids created by symbionts from dietary amino acids. Nature 600, 302-307 (2021).
35. Wijayadikusumah, A. R., Zeng, W., McQuilten, H. A., Wong, C. Y., Jackson, D. C. & Chua, B. Y. Geometry of a TLR2-Agonist-Based Adjuvant Can Affect the Resulting Antigen-Specific Immune Response. Mol Pharmaceut 16, 2037-2047 (2019).
36. Jin, M. S., Kim, S. E., Heo, J. Y., Lee, M. E., Kim, H. M., Paik, S.-G., Lee, H. & Lee, J.-O. Crystal Structure of the TLR1-TLR2 Heterodimer Induced by Binding of a Tri-Acylated Lipopeptide. Cell 130, 1071-1082 (2007).
37. Jin, M. S. & Lee, J.-O. Structures of TLR-ligand complexes. Curr Opin Immunol 20, 414-419 (2008).
38. Su, L., Wang, Y., Wang, J., Mifune, Y., Morin, M. D., Jones, B. T., Moresco, E. M. Y., Boger, D. L., Beutler, B. & Zhang, H. Structural Basis of TLR2/TLR1 Activation by the Synthetic Agonist Diprovocim. J Med Chem 62, 2938-2949 (2019).
39. Wang, Y., Su, L., Morin, M. D., Jones, B. T., Mifune, Y., Shi, H., Wang, K., Zhan, X., Liu, A., Wang, J., Li, X., Tang, M., Ludwig, S., Hildebrand, S., Zhou, K., Siegwart, D. J., Moresco, E. M. Y., Zhang, H., Boger, D. L. & Beutler, B. Adjuvant effect of the novel TLR1/TLR2 agonist Diprovocim synergizes with anti-PD-L1 to eliminate melanoma in mice. Proc National Acad Sci 115, 201809232 (2018).
40. Cheng, K., Gao, M., Godfroy, J. I., Brown, P. N., Kastelowitz, N. & Yin, H. Specific activation of the TLR1-TLR2 heterodimer by small-molecule agonists. Sci Adv 1, e1400139 (2015).
41. Erturk-Hasdemir, D., Oh, S. F., Okan, N. A., Stefanetti, G., Gazzaniga, F. S., Seeberger, P. H., Plevy, S. E. & Kasper, D. L. Symbionts exploit complex signaling to educate the immune system. Proc National Acad Sci 116, 26157-26166 (2019).
42. Gaffen, S. L., Jain, R., Garg, A. V. & Cua, D. J. The IL-23-IL-17 immune axis: from mechanisms to therapeutic testing. Nat Rev Immunol 14, 585-600 (2014).
43. Zähringer, U., Lindner, B., Inamura, S., Heine, H. & Alexander, C. TLR2—promiscuous or specific?A critical re-evaluation of a receptor expressing apparent broad specificity. Immunobiology 213, 205-224 (2008).
44. Ansaldo, E. & Belkaid, Y. How microbiota improve immunotherapy. Science 373, 966-967 (2021).
45. Picelli, S. Single Cell Methods, Sequencing and Proteomics. Methods Mol Biology 1979, 25-44 (2019).
46. Wingett, S. W. & Andrews, S. FastQ Screen: A tool for multi-genome mapping and quality control. F1000research 7, 1338 (2018).
47. Ewels, P., Magnusson, M., Lundin, S. & Käller, M. MultiQC: summarize analysis results for multiple tools and samples in a single report. Bioinformatics 32, 3047-3048 (2016).
48. Robinson, M. D., McCarthy, D. J. & Smyth, G. K. edgeR: a Bioconductor package for differential expression analysis of digital gene expression data. Bioinformatics 26, 139-140 (2010).
49. Bray, N. L., Pimentel, H., Melsted, P. & Pachter, L. Near-optimal probabilistic RNA-seq quantification. Nat Biotechnol 34, 525-527 (2016).
50. Long, F., Nicholls, R. A., Emsley, P., Gražulis, S., Merkys, A., Vaitkus, A. & Murshudov, G. N. AceDRG: a stereochemical description generator for ligands. Acta Crystallogr Sect D Struct Biology 73, 112-122 (2017).
51. Emsley, P. & Cowtan, K. Coot: model-building tools for molecular graphics. Acta Crystallogr Sect D Biological Crystallogr 60, 2126-2132 (2004).
52. Goddard, T. D., Huang, C. C., Meng, E. C., Pettersen, E. F., Couch, G. S., Morris, J. H. & Ferrin, T. E. UCSF ChimeraX: Meeting modern challenges in visualization and analysis. Protein Sci 27, 14-25 (2018).
53. Morin, A., Eisenbraun, B., Key, J., Sanschagrin, P. C., Timony, M. A., Ottaviano, M. & Sliz, P. Collaboration gets the most out of software. Elife 2, e01456 (2013).

### ADDITIONAL EMBODIMENTS

Additional embodiments are provided according to the following numbered embodiments:

1. A compound of Formula (I):

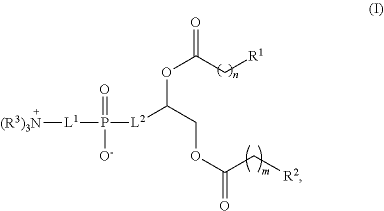

or a pharmaceutically acceptable salt, solvate, hydrate, polymorph, co-crystal, tautomer, stereoisomer, isotopically labeled derivative, or prodrug thereof, wherein:

R¹ is branched or unbranched C₁₋₄ alkyl, or C₃₋₇ carbocyclic;

R² is branched or unbranched C₁₋₄ alkyl, or C₃₋₇ carbocyclic;

at least one of R¹ and R² is branched C₁₋₄ alkyl, or C₃₋₇ carbocyclic;

R³ is —H or branched or unbranched C₁₋₆ alkyl;

L¹ is substituted or unsubstituted C₁₋₅ alkylene, substituted or unsubstituted C₁₋₈ heteroalkylene, substituted or unsubstituted carbocyclylene, substituted or unsubstituted heterocyclylene, substituted or unsubstituted arylene, substituted or unsubstituted heteroarylene, or any combination thereof;

L² is unbranched and unsubstituted C₁₋₆ heteroalkylene, or unbranched and unsubstituted C₁₋₆ alkylene;

n is an integer from 7 to 13, inclusive;

m is an integer from 7 to 13, inclusive; and

provided that the compound is not of the formula:

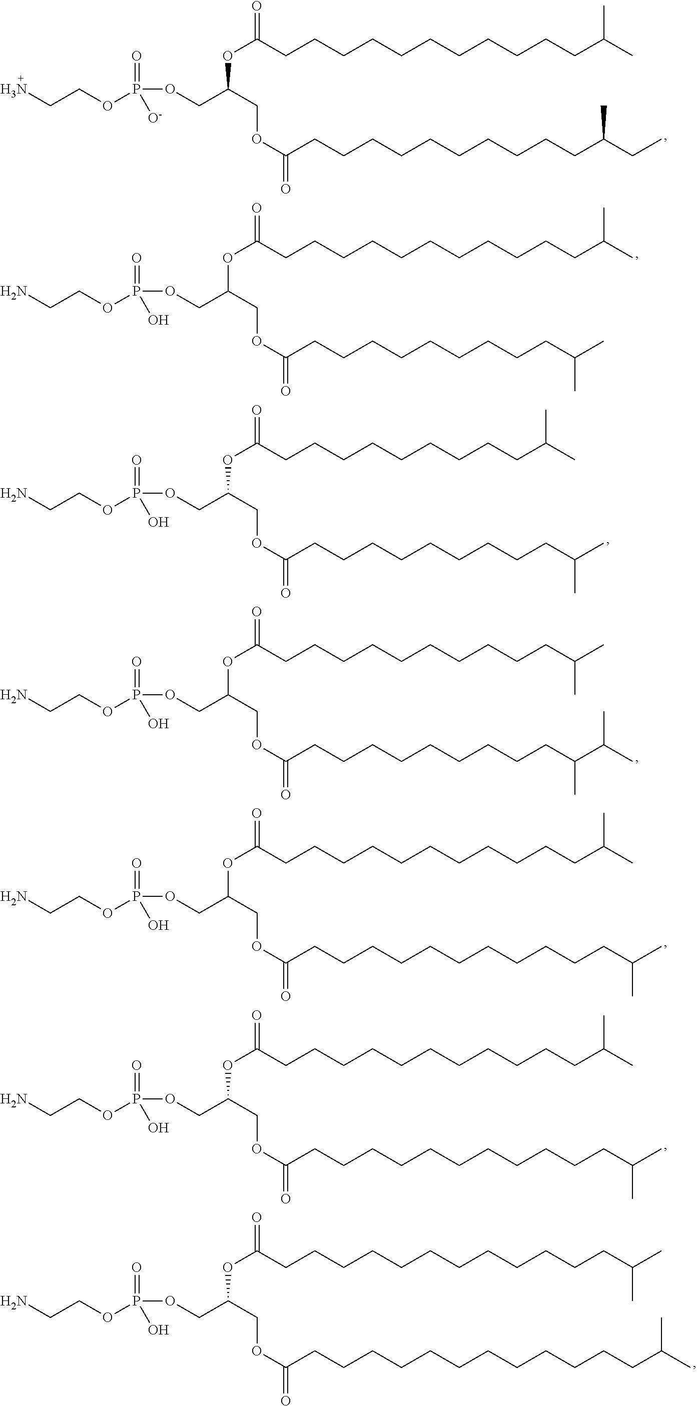

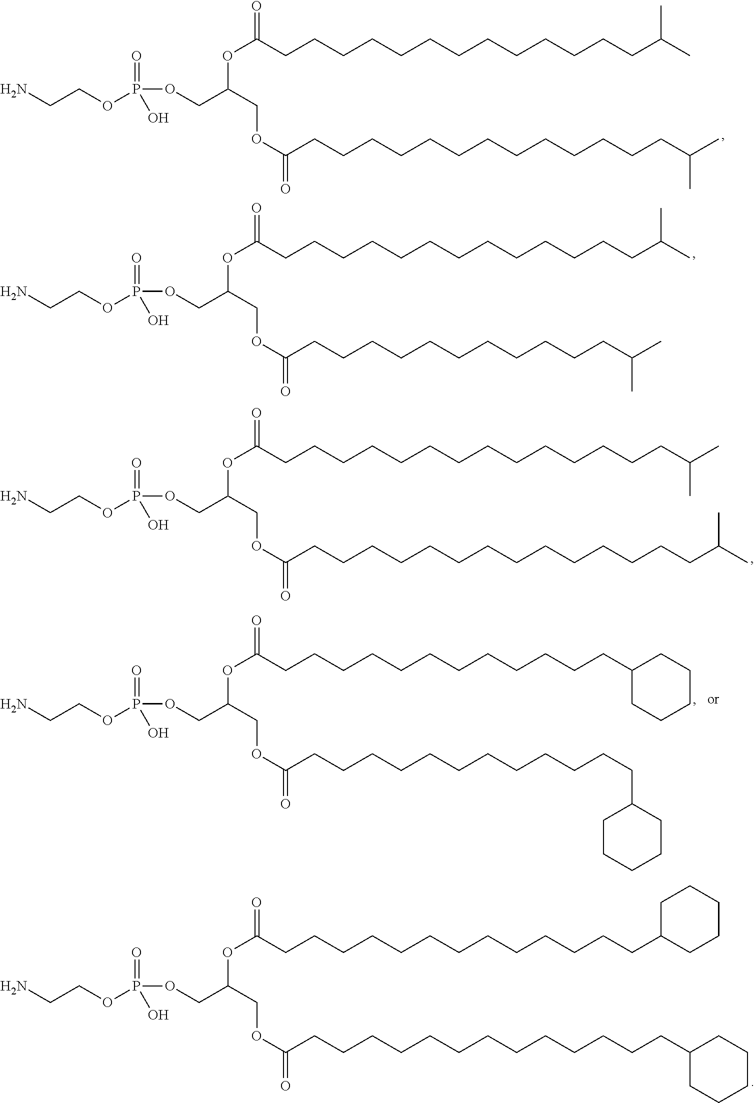

2. The compound of embodiment 1, wherein the compound is of Formula (Ia):

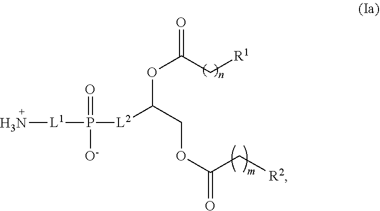

or a pharmaceutically acceptable salt, solvate, hydrate, polymorph, co-crystal, tautomer, stereoisomer, isotopically labeled derivative, or prodrug thereof.
3. The compound of embodiment 1, wherein the compound is of Formula (Ib):

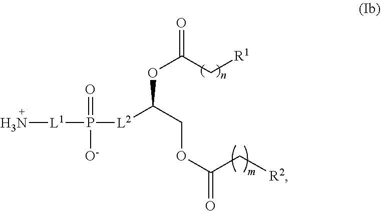

or a pharmaceutically acceptable salt, solvate, hydrate, polymorph, co-crystal, tautomer, stereoisomer, isotopically labeled derivative, or prodrug thereof.
4. The compound of embodiment 1, wherein the compound is of Formula (Ic):

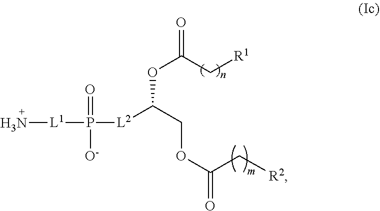

or a pharmaceutically acceptable salt, solvate, hydrate, polymorph, co-crystal, tautomer, stereoisomer, isotopically labeled derivative, or prodrug thereof.
5. The compound of embodiment 1, wherein the compound is of Formula (Id):

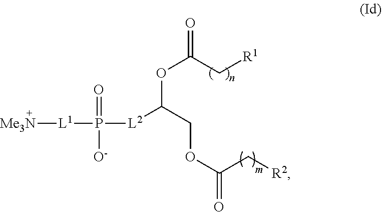

or a pharmaceutically acceptable salt, solvate, hydrate, polymorph, co-crystal, tautomer, stereoisomer, isotopically labeled derivative, or prodrug thereof.
6. The compound of embodiment 1, wherein the compound is of Formula (Ie):

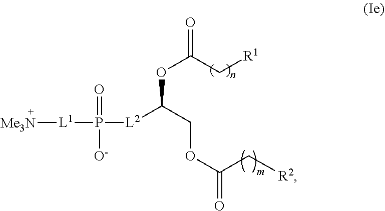

or a pharmaceutically acceptable salt, solvate, hydrate, polymorph, co-crystal, tautomer, stereoisomer, isotopically labeled derivative, or prodrug thereof.
7. The compound of embodiment 1, wherein the compound is of Formula (If):

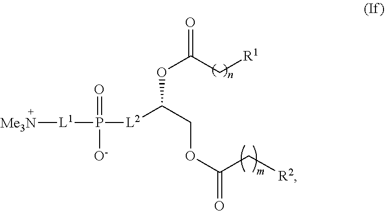

or a pharmaceutically acceptable salt, solvate, hydrate, polymorph, co-crystal, tautomer, stereoisomer, isotopically labeled derivative, or prodrug thereof.
8. The compound of any one of embodiments 1-7, wherein R¹ and R² are each independently branched C₁₋₄ alkyl, or C₃₋₄ carbocyclic.
9. The compound of any one of embodiments 1-8, wherein R¹ and R² are different.
10. The compound of any one of embodiments 1-8, wherein R¹ and R² are the same.
11. The compound of any one of embodiments 1-9, wherein exactly one of R¹ or R² is selected from the group consisting of -Me, -Et, -ⁿPr, and -ⁿBu.
12. The compound of any one of embodiments 1-11, wherein at least one of R¹ and R² is selected from the group consisting of;

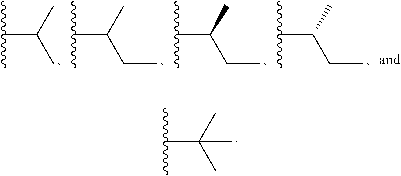

13. The compound of any one of embodiments 1-11, wherein at least one of R¹ and R² is selected from the group consisting of;

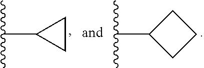

14. The compound of any one of embodiments 1-13, wherein m and n are different.
15. The compound of any one of embodiments 1-13, wherein m and n are the same.
16. The compound of any one of embodiments 1-15, wherein n is 9-13.
17. The compound of any one of embodiments 1-16, wherein m is 9-13.
18. The compound of any one of embodiments 1-13 or 15, wherein n is 11, and m is 11.
19. The compound of any one of embodiments 1-14, wherein n is 10, and m is 11.
20. The compound of any one of embodiments 1-14, wherein n is 11, and m is 10.
21. The compound of any one of embodiments 1-13 or 15, wherein n is 10, and m is 10.
22. The compound of any one of embodiments 1-18 or 20, wherein n is 11, and R¹ is

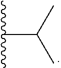 [SMILES: *C(C)C]

23. The compound of any one of embodiments 1-18 or 20, wherein n is 11, and R¹ is

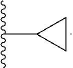 [SMILES: *C1CC1]

24. The compound of any one of embodiments 1-17, 19, or 21, wherein n is 10 and R¹ is selected from the group consisting of:

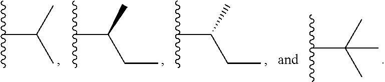

25. The compound of any one of embodiments 1-17, 19, or 21, wherein n is 10 and R¹ is

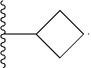 [SMILES: *C1CCC1]

26. The compound of any one of embodiments 1-19, wherein m is 11 and R² is

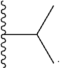 [SMILES: *C(C)C]

27. The compound of any one of embodiments 1-19, wherein m is 11 and R² is

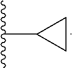 [SMILES: *C1CC1]

28. The compound of any one of embodiments 1-17 or 20-21, wherein m is 10 and R² is selected from the group consisting of:

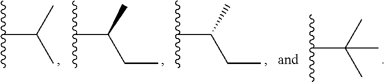

29. The compound of any one of embodiments 1-17 or 20-21, wherein m is 10 and R² is

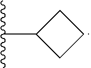 [SMILES: *C1CCC1]

30. The compound of any one of embodiments 1-17, wherein n is 10, and R¹ is

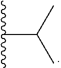 [SMILES: *C(C)C]

31. The compound of any one of embodiments 1-17, wherein n is 10, and R¹ is

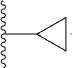 [SMILES: *C1CC1]

32. The compound of any one of embodiments 1-17, wherein n is 9 and R¹ is selected from the group consisting of:

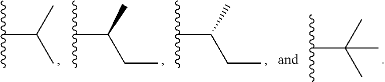

33. The compound of any one of embodiments 1-17, wherein n is 9 and R¹ is

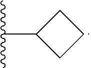 [SMILES: *C1CCC1]

34. The compound of any one of embodiments 1-17, wherein m is 10 and R² is

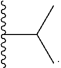 [SMILES: *C(C)C]

35. The compound of any one of embodiments 1-17, wherein m is 10 and R² is

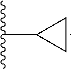 [SMILES: *C1CC1]

36. The compound of any one of embodiments 1-17, wherein m is 9 and R² is selected from the group consisting of:

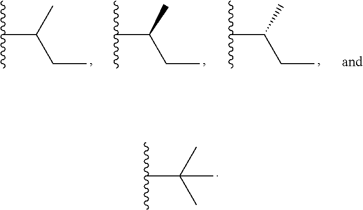

37. The compound of any one of embodiments 1-17, wherein m is 9 and R² is

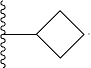 [SMILES: *C1CCC1]

38. The compound of any one of embodiments 1-17, wherein n is 12, and R¹ is

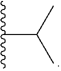 [SMILES: *C(C)C]

39. The compound of any one of embodiments 1-17, wherein n is 12, and R¹ is

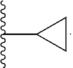 [SMILES: *C1CC1]

40. The compound of any one of embodiments 1-17, wherein n is 11 and R¹ is selected from the group consisting of:

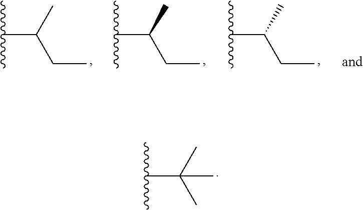

41. The compound of any one of embodiments 1-17, wherein n is 11 and R¹ is

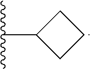 [SMILES: *C1CCC1]

42. The compound of any one of embodiments 1-17, wherein m is 12 and R² is

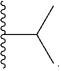 [SMILES: *C(C)C]

43. The compound of any one of embodiments 1-17, wherein m is 12 and R² is

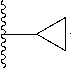 [SMILES: *C1CC1]

44. The compound of any one of embodiments 1-17, wherein m is 11 and R² is selected from the group consisting of:

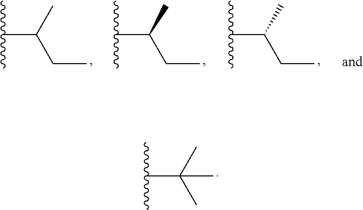

45. The compound of any one of embodiments 1-17, wherein m is 11 and R² is

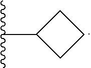 [SMILES: *C1CCC1]

46. The compound of any one of embodiments 1-45, wherein L¹ is unbranched and unsubstituted C₁₋₆ heteroalkylene comprising one or more oxygen atoms.
47. The compound of any one of embodiments 1-46, wherein L¹ is unbranched and unsubstituted C₁₋₆ alkylene.
48. The compound of any one of embodiments 1-47, wherein L¹ is selected from the group consisting of:

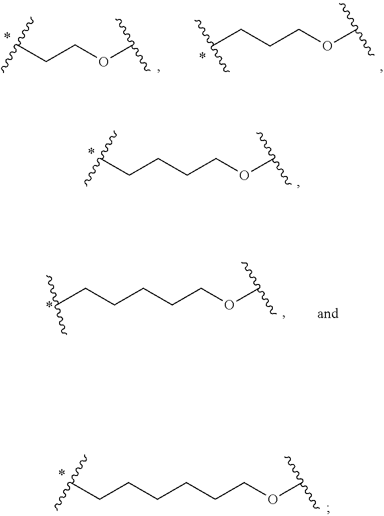 [SMILES: *CCO* ; *CCCO* ; *CCCCO* ; *CCCCCO* ; *CCCCCCO*]

wherein the attachment point labeled with “*” is attached to the nitrogen atom.
49. The compound of any one of embodiments 1-47, wherein L¹ is selected from the group consisting of:

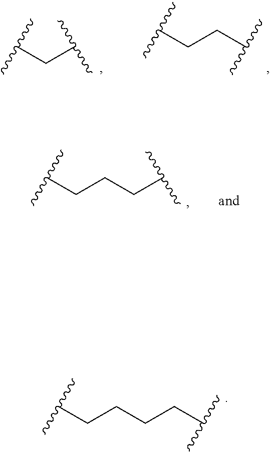

50. The compound of any one of embodiments 1-49, wherein L² is selected from the group consisting of:

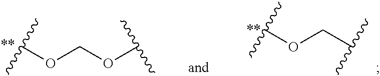

wherein the attachment point labeled with “**” is attached to the phosphorous atom.
51. The compound of any one of embodiments 1-48 or 50, wherein L¹ is unbranched and unsubstituted C₁₋₆ heteroalkylene with one or more oxygen atoms, and L² is

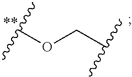 [SMILES: *CO*]

wherein the attachment point labeled with “**” is attached to the phosphorous atom.
52. The compound of any one of embodiments 1-48 or 50, wherein L¹ is

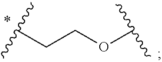 [SMILES: *CCO*]

wherein the attachment point labeled with “*” is attached to the nitrogen atom, and L² is

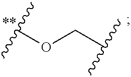 [SMILES: *CO*]

wherein the attachment point labeled with “**” is attached to the phosphorous atom.
53. The compound of any one of embodiments 1-48 or 50, wherein L¹ is

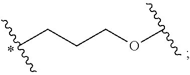 [SMILES: *CCCO*]

wherein the attachment point labeled with “*” is attached to the nitrogen atom, and L² is

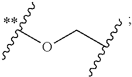 [SMILES: *CO*]

wherein the attachment point labeled with “**” is attached to the phosphorous atom.
54. The compound of any one of embodiments 1-48 or 50, wherein L¹ is

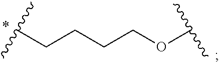 [SMILES: *CCCCO*]

wherein the attachment point labeled with “*” is attached to the nitrogen atom, and L² is

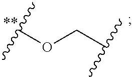 [SMILES: *CO*]

wherein the attachment point labeled with “**” is attached to the phosphorous atom.
55. The compound of embodiment 1, wherein the compound is selected from the group consisting of:

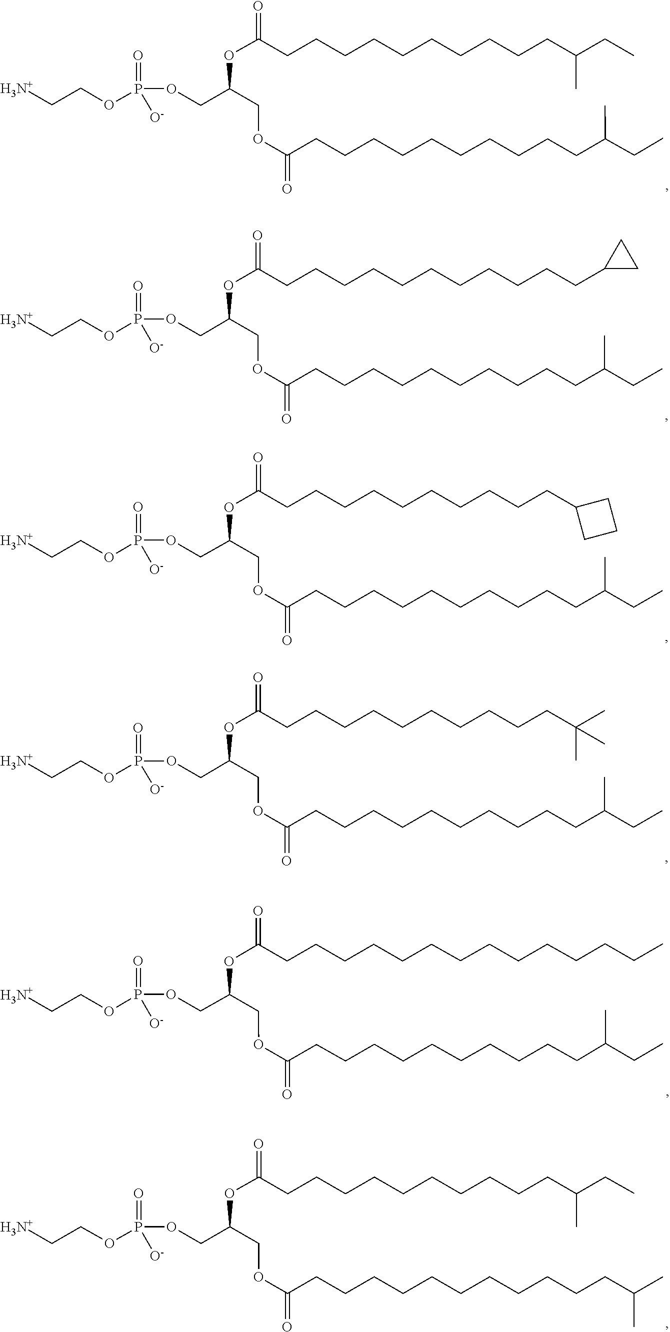 [SMILES: CCC(C)CCCCCCCCCCC(=O)OC[C@@H](COP(=O)([O-])OCC[NH3+])OC(=O)CCCCCCCCCCC(C)CC ; CCC(C)CCCCCCCCCCC(=O)OC[C@@H](COP(=O)([O-])OCC[NH3+])OC(=O)CCCCCCCCCCCC1CC1 ; CCC(C)CCCCCCCCCCC(=O)OC[C@@H](COP(=O)([O-])OCC[NH3+])OC(=O)CCCCCCCCCCC1CCC1 ; CCC(C)CCCCCCCCCCC(=O)OC[C@@H](COP(=O)([O-])OCC[NH3+])OC(=O)CCCCCCCCCCC(C)(C)C ; CCCCCCCCCCCCCCC(=O)O[C@@H](COC(=O)CCCCCCCCCCC(C)CC)COP(=O)([O-])OCC[NH3+] ; CCC(C)CCCCCCCCCCC(=O)O[C@@H](COC(=O)CCCCCCCCCCCC(C)C)COP(=O)([O-])OCC[NH3+]]

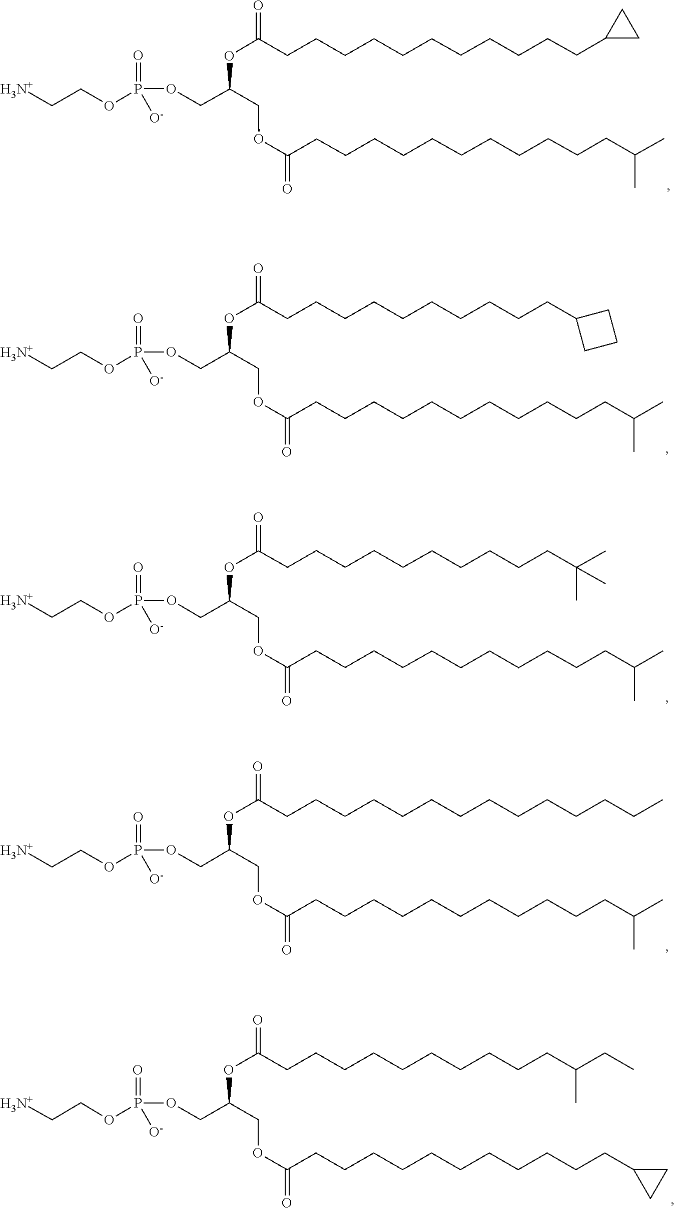 [SMILES: CC(C)CCCCCCCCCCCC(=O)OC[C@@H](COP(=O)([O-])OCC[NH3+])OC(=O)CCCCCCCCCCCC1CC1 ; CC(C)CCCCCCCCCCCC(=O)OC[C@@H](COP(=O)([O-])OCC[NH3+])OC(=O)CCCCCCCCCCC1CCC1 ; CC(C)CCCCCCCCCCCC(=O)OC[C@@H](COP(=O)([O-])OCC[NH3+])OC(=O)CCCCCCCCCCC(C)(C)C ; CCCCCCCCCCCCCCC(=O)O[C@@H](COC(=O)CCCCCCCCCCCC(C)C)COP(=O)([O-])OCC[NH3+] ; CCC(C)CCCCCCCCCCC(=O)O[C@@H](COC(=O)CCCCCCCCCCCC1CC1)COP(=O)([O-])OCC[NH3+]]

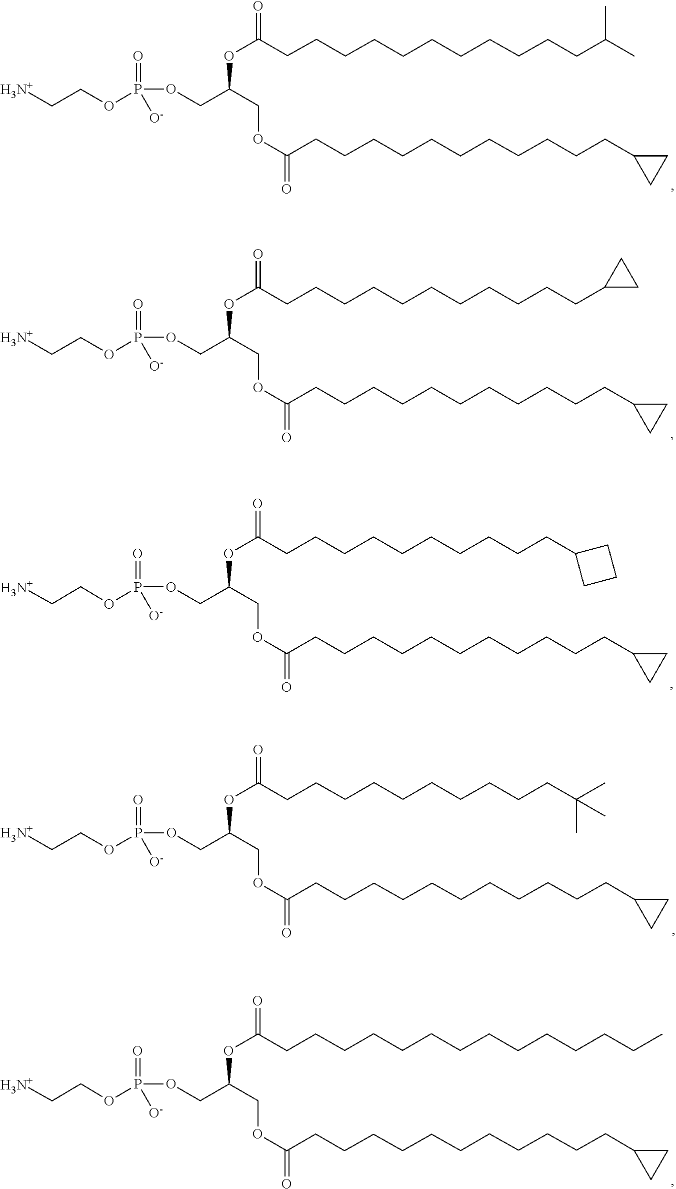 [SMILES: CC(C)CCCCCCCCCCCC(=O)O[C@@H](COC(=O)CCCCCCCCCCCC1CC1)COP(=O)([O-])OCC[NH3+] ; [NH3+]CCOP(=O)([O-])OC[C@H](COC(=O)CCCCCCCCCCCC1CC1)OC(=O)CCCCCCCCCCCC1CC1 ; [NH3+]CCOP(=O)([O-])OC[C@H](COC(=O)CCCCCCCCCCCC1CC1)OC(=O)CCCCCCCCCCC1CCC1 ; CC(C)(C)CCCCCCCCCCC(=O)O[C@@H](COC(=O)CCCCCCCCCCCC1CC1)COP(=O)([O-])OCC[NH3+] ; CCCCCCCCCCCCCCC(=O)O[C@@H](COC(=O)CCCCCCCCCCCC1CC1)COP(=O)([O-])OCC[NH3+]]

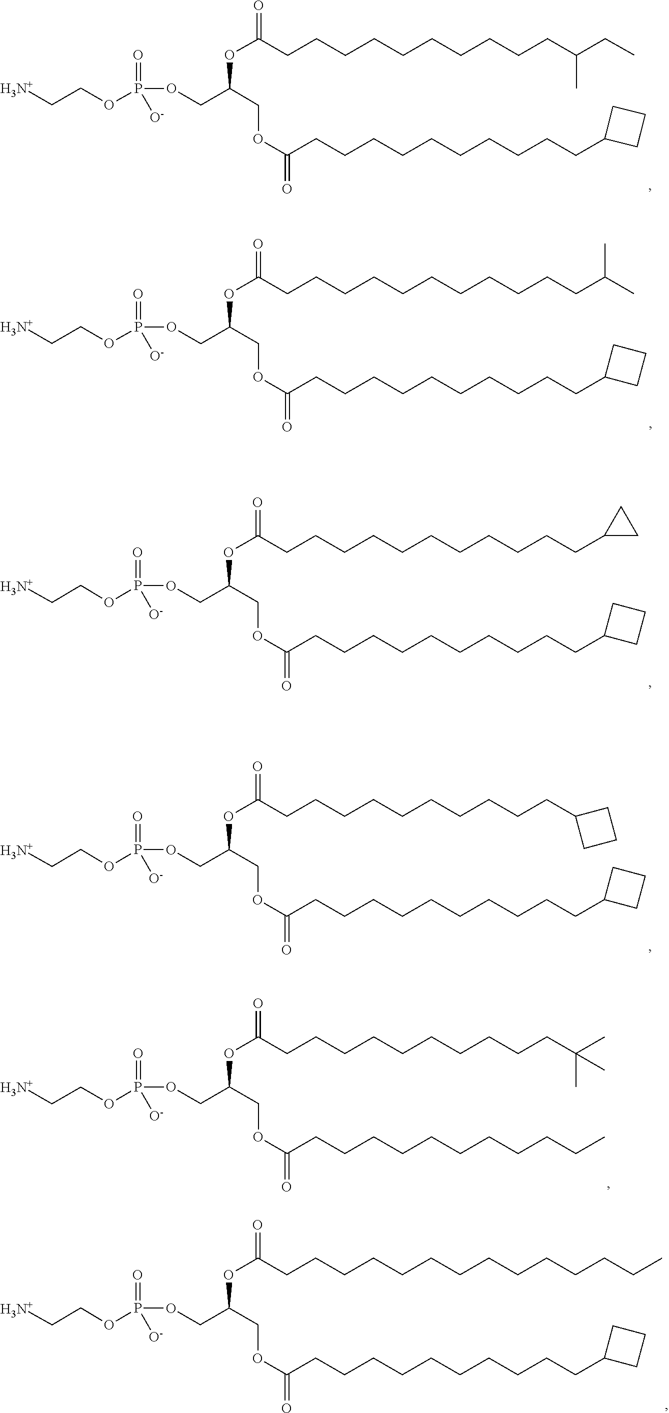 [SMILES: CCC(C)CCCCCCCCCCC(=O)O[C@@H](COC(=O)CCCCCCCCCCC1CCC1)COP(=O)([O-])OCC[NH3+] ; CC(C)CCCCCCCCCCCC(=O)O[C@@H](COC(=O)CCCCCCCCCCC1CCC1)COP(=O)([O-])OCC[NH3+] ; [NH3+]CCOP(=O)([O-])OC[C@H](COC(=O)CCCCCCCCCCC1CCC1)OC(=O)CCCCCCCCCCCC1CC1 ; [NH3+]CCOP(=O)([O-])OC[C@H](COC(=O)CCCCCCCCCCC1CCC1)OC(=O)CCCCCCCCCCC1CCC1 ; CCCCCCCCCCCC(=O)OC[C@@H](COP(=O)([O-])OCC[NH3+])OC(=O)CCCCCCCCCCC(C)(C)C ; CCCCCCCCCCCCCCC(=O)O[C@@H](COC(=O)CCCCCCCCCCC1CCC1)COP(=O)([O-])OCC[NH3+]]

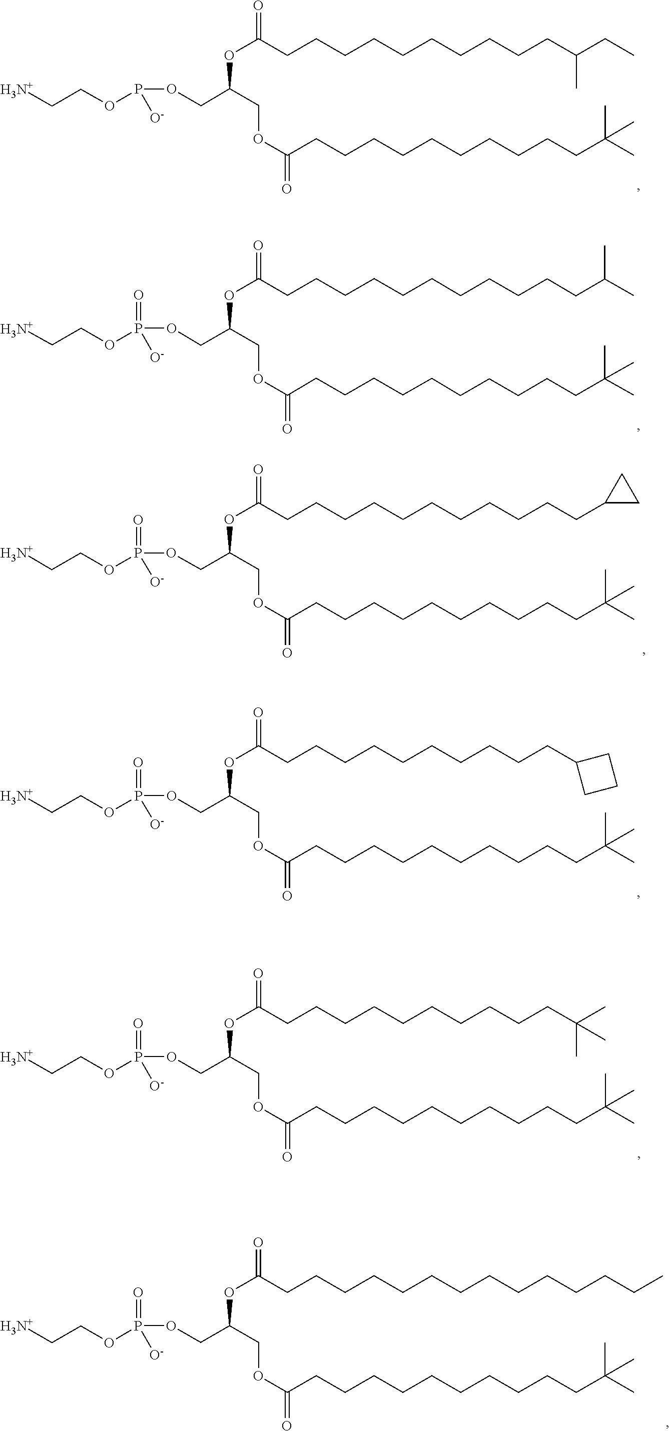 [SMILES: CCC(C)CCCCCCCCCCC(=O)O[C@@H](COC(=O)CCCCCCCCCCC(C)(C)C)COP([O-])OCC[NH3+] ; CC(C)CCCCCCCCCCCC(=O)O[C@@H](COC(=O)CCCCCCCCCCC(C)(C)C)COP(=O)([O-])OCC[NH3+] ; CC(C)(C)CCCCCCCCCCC(=O)OC[C@@H](COP(=O)([O-])OCC[NH3+])OC(=O)CCCCCCCCCCCC1CC1 ; CC(C)(C)CCCCCCCCCCC(=O)OC[C@@H](COP(=O)([O-])OCC[NH3+])OC(=O)CCCCCCCCCCC1CCC1 ; CC(C)(C)CCCCCCCCCCC(=O)OC[C@@H](COP(=O)([O-])OCC[NH3+])OC(=O)CCCCCCCCCCC(C)(C)C ; CCCCCCCCCCCCCCC(=O)O[C@@H](COC(=O)CCCCCCCCCCC(C)(C)C)COP(=O)([O-])OCC[NH3+]]

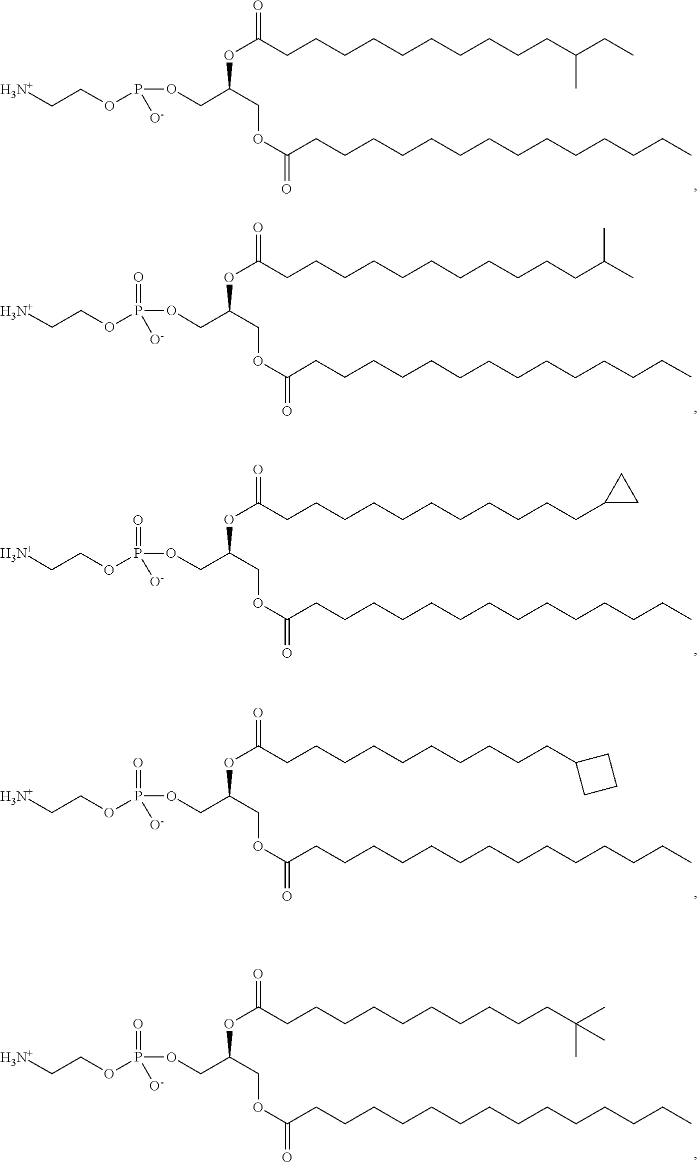 [SMILES: CCCCCCCCCCCCCCC(=O)OC[C@@H](COP([O-])OCC[NH3+])OC(=O)CCCCCCCCCCC(C)CC ; CCCCCCCCCCCCCCC(=O)OC[C@@H](COP(=O)([O-])OCC[NH3+])OC(=O)CCCCCCCCCCCC(C)C ; CCCCCCCCCCCCCCC(=O)OC[C@@H](COP(=O)([O-])OCC[NH3+])OC(=O)CCCCCCCCCCCC1CC1 ; CCCCCCCCCCCCCCC(=O)OC[C@@H](COP(=O)([O-])OCC[NH3+])OC(=O)CCCCCCCCCCC1CCC1 ; CCCCCCCCCCCCCCC(=O)OC[C@@H](COP(=O)([O-])OCC[NH3+])OC(=O)CCCCCCCCCCC(C)(C)C]

and pharmaceutically acceptable salts, hydrates, polymorphs, co-crystals, tautomers, stereoisomers, isotopically labeled derivative, or prodrug thereof.
57. The compound of embodiment 1, having the structure:

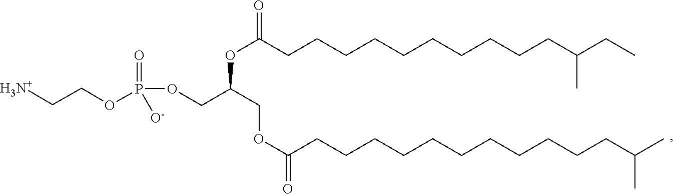 [SMILES: CCC(C)CCCCCCCCCCC(=O)O[C@@H](COC(=O)CCCCCCCCCCCC(C)C)COP(=O)([O-])OCC[NH3+]]

or a pharmaceutically acceptable salt, hydrate, polymorph, co-crystal, tautomer, stereoisomer, isotopically labeled derivative, or prodrug thereof.
57. The compound of embodiment 1, having the structure:

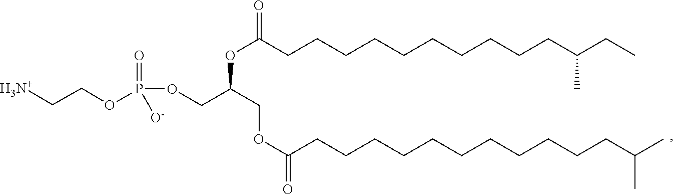 [SMILES: CC[C@H](C)CCCCCCCCCCC(=O)O[C@@H](COC(=O)CCCCCCCCCCCC(C)C)COP(=O)([O-])OCC[NH3+]]

or a pharmaceutically acceptable salt, hydrate, polymorph, co-crystal, tautomer, stereoisomer, isotopically labeled derivative, or prodrug thereof.
58. A compound of Formula (II):

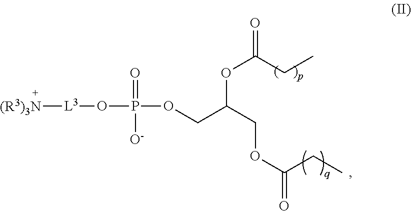

or a pharmaceutically acceptable salt, solvate, hydrate, polymorph, co-crystal, tautomer, stereoisomer, isotopically labeled derivative, or prodrug thereof, wherein:

L³ is substituted or unsubstituted C₃₋₈ alkylene, substituted or unsubstituted C₃₋₈ heteroalkylene, substituted or unsubstituted carbocyclylene, substituted or unsubstituted heterocyclylene, substituted or unsubstituted arylene, substituted or unsubstituted heteroarylene, or any combination thereof;

R³ is —H or branched or unbranched C₁₋₆ alkyl;

p is an integer from 12 to 14, inclusive; and

q is an integer from 12 to 14, inclusive;

provided that the compound is not of the formula:

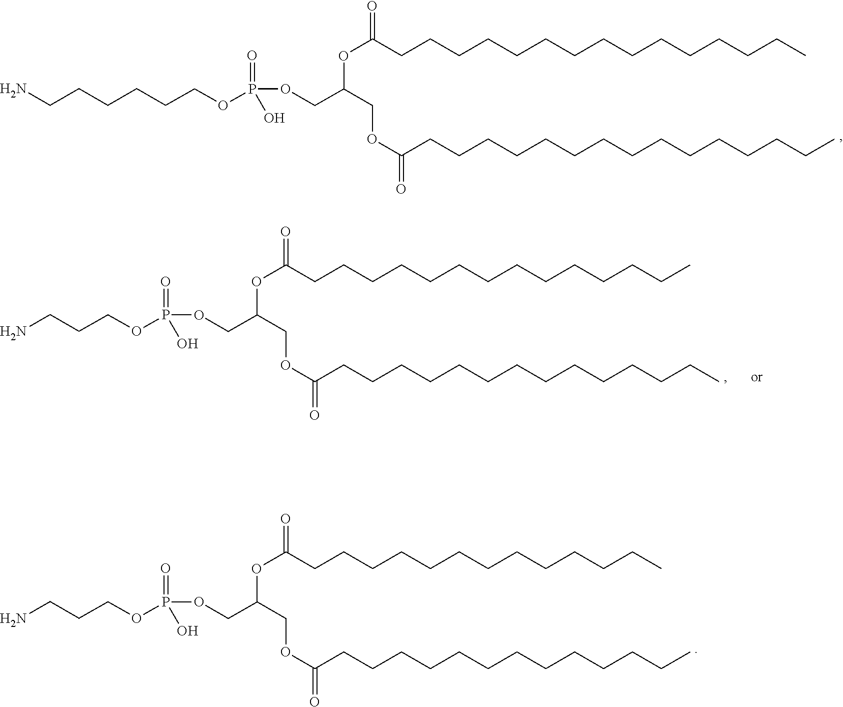 [SMILES: CCCCCCCCCCCCCCCC(=O)OCC(COP(=O)(O)OCCCCCCN)OC(=O)CCCCCCCCCCCCCCC ; CCCCCCCCCCCCCCC(=O)OCC(COP(=O)(O)OCCCN)OC(=O)CCCCCCCCCCCCCC ; CCCCCCCCCCCCCC(=O)OCC(COP(=O)(O)OCCCN)OC(=O)CCCCCCCCCCCCC]

59. The compound of embodiment 58, wherein the compound is of Formula (IIa):

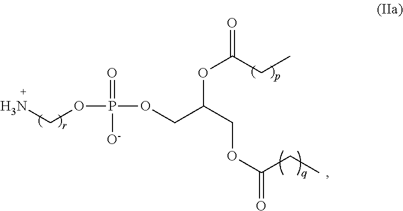

or a pharmaceutically acceptable salt, solvate, hydrate, polymorph, co-crystal, tautomer, stereoisomer, isotopically labeled derivative, or prodrug thereof, wherein r is an integer from 3 to 8, inclusive.
60. The compound of embodiment 58, wherein the compound is of Formula (IIb):

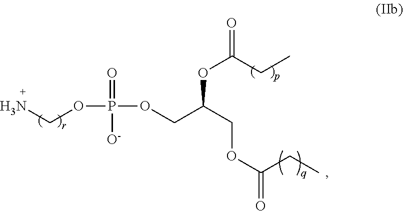

or a pharmaceutically acceptable salt, solvate, hydrate, polymorph, co-crystal, tautomer, stereoisomer, isotopically labeled derivative, or prodrug thereof, wherein r is an integer from 3 to 8, inclusive.
61. The compound of embodiment 58, wherein the compound is of Formula (IIc):

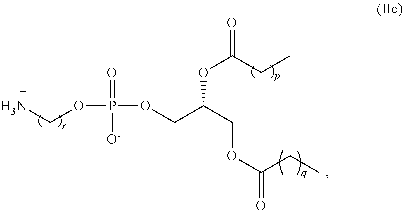

or a pharmaceutically acceptable salt, solvate, hydrate, polymorph, co-crystal, tautomer, stereoisomer, isotopically labeled derivative, or prodrug thereof, wherein r is an integer from 3 to 8, inclusive.
62. The compound of embodiment 58, wherein the compound is of Formula (IId):

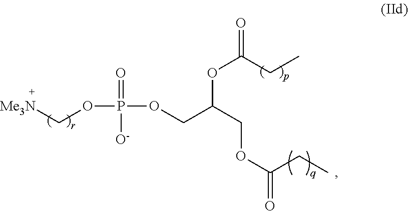

or a pharmaceutically acceptable salt, solvate, hydrate, polymorph, co-crystal, tautomer, stereoisomer, isotopically labeled derivative, or prodrug thereof, wherein r is an integer from 3 to 8, inclusive.
63. The compound of embodiment 58, wherein the compound is of Formula (IIe):

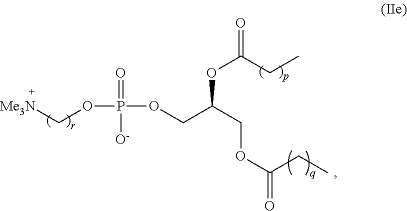

or a pharmaceutically acceptable salt, solvate, hydrate, polymorph, co-crystal, tautomer, stereoisomer, isotopically labeled derivative, or prodrug thereof, wherein r is an integer from 3 to 8, inclusive.
64. The compound of embodiment 58, wherein the compound is of Formula (IIe):

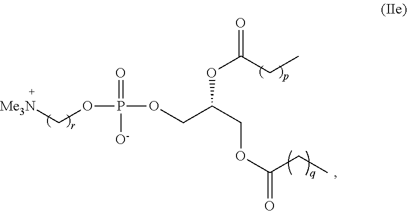

or a pharmaceutically acceptable salt, solvate, hydrate, polymorph, co-crystal, tautomer, stereoisomer, isotopically labeled derivative, or prodrug thereof, wherein r is an integer from 3 to 8, inclusive.
65. The compound of any one of embodiments 58-64, wherein L³ is selected from the group consisting of:

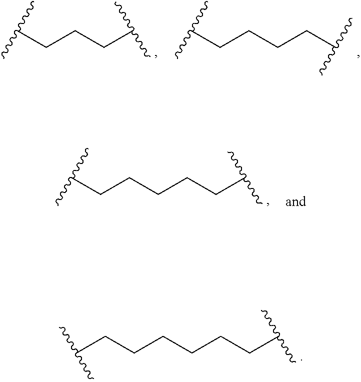 [SMILES: *CCC* ; *CCCC* ; *CCCCC* ; *CCCCCC*]

66. The compound of any one of embodiments 58-65, wherein p and q are the same.
67. The compound of any one of embodiments 58-65, wherein p and q are different.
68. The compound of any one of embodiments 58-66, wherein p is 12; and q is 12.
69. The compound of any one of embodiments 58-66, wherein p is 13; and q is 13.
70. The compound of any one of embodiments 58-66, wherein p is 14; and q is 14.
71. The compound of embodiment 58, wherein the compound is selected from the group consisting of:

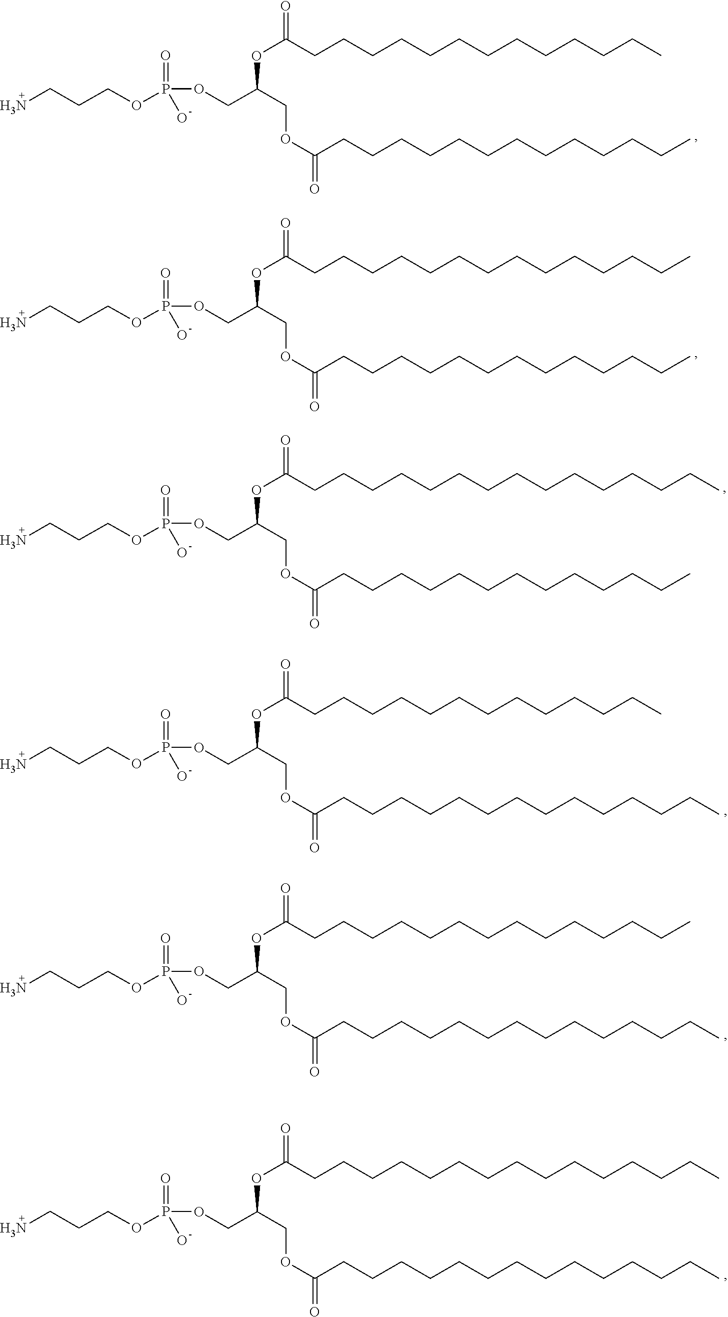

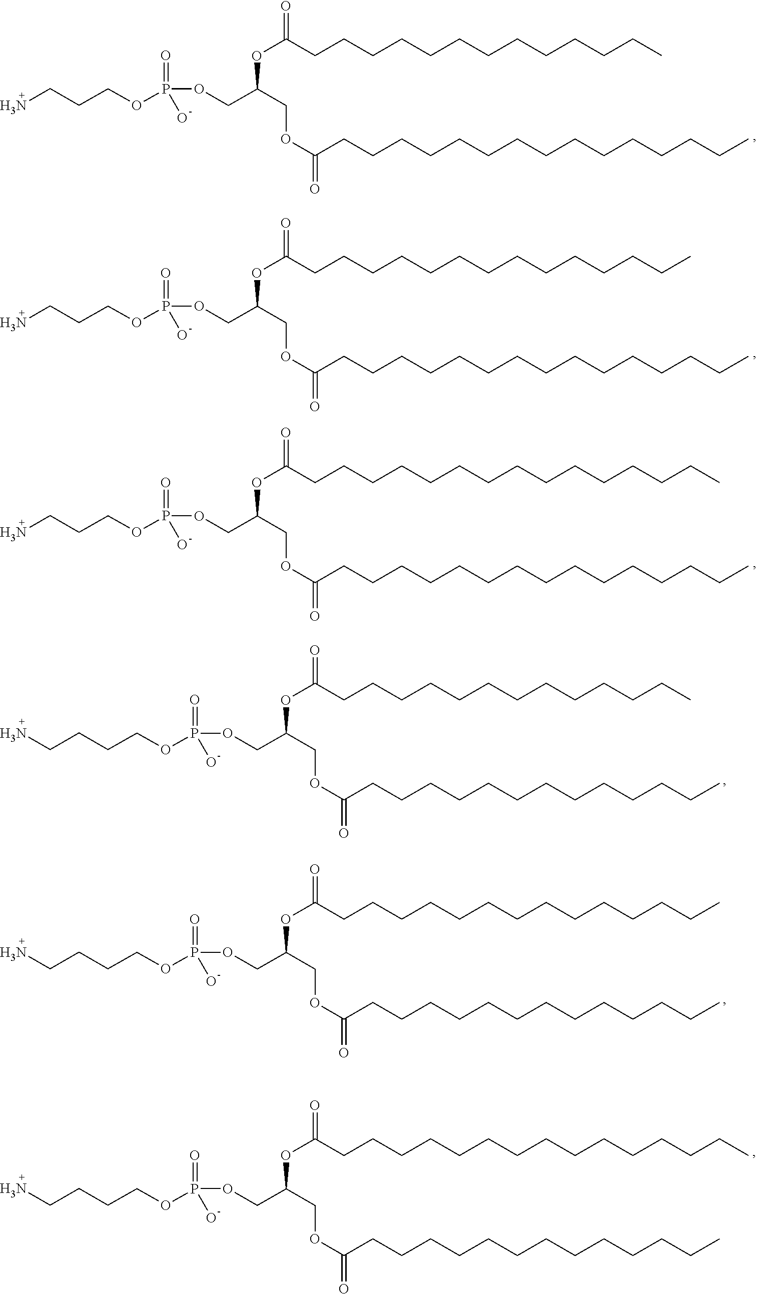

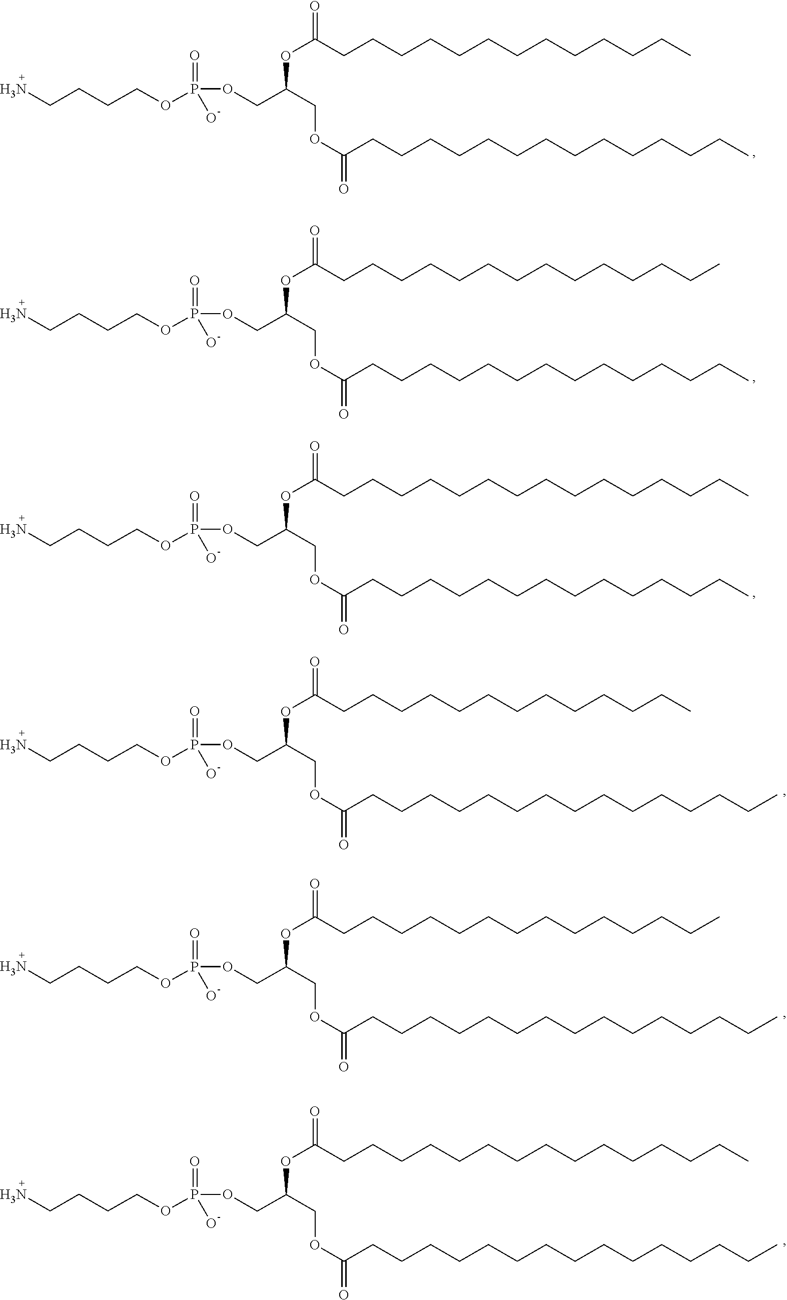

and pharmaceutically acceptable salts, hydrates, polymorphs, co-crystals, tautomers, stereoisomers, isotopically labeled derivatives, and prodrugs thereof.
72. A pharmaceutical composition comprising a compound of any one of the embodiments 1-71, or a pharmaceutically acceptable salt, solvate, hydrate, polymorph, co-crystal, tautomer, stereoisomer, isotopically labeled derivative, or prodrug thereof; and a pharmaceutically acceptable excipient.
73. The pharmaceutical composition of embodiment 72, further comprising an additional pharmaceutical agent.
74. A compound of any one of embodiments 1-71, or a pharmaceutically acceptable salt, solvate, hydrate, polymorph, co-crystal, tautomer, stereoisomer, isotopically labeled derivative, or prodrug thereof, or a pharmaceutical composition of embodiment 73 or 74, for use in treating a disease, disorder, or condition, wherein the disease, disorder, or condition is a metabolic disease, inflammatory disease, immune disorder, or proliferative disease.
75. A compound of any one of embodiments 1-71, or a pharmaceutically acceptable salt, solvate, hydrate, polymorph, co-crystal, tautomer, stereoisomer, isotopically labeled derivative, or prodrug thereof, or a pharmaceutical composition of embodiment 73 or 74, for use as a vaccine adjuvant.
76. Use of a compound of any one of embodiments 1-71, or a pharmaceutically acceptable salt, solvate, hydrate, polymorph, co-crystal, tautomer, stereoisomer, isotopically labeled derivative, or prodrug thereof, or a pharmaceutical composition of embodiment 73 or 74, for the manufacture of a medicament for treating metabolic disease, inflammatory disease, immune disorder, or proliferative disease.
77. A method of treating a metabolic disease, inflammatory disease, immune disorder, or proliferative disease in a subject in need thereof comprising administering to the subject a therapeutically effective amount of a compound of any one of embodiments 1-71, or a pharmaceutically acceptable salt, solvate, hydrate, polymorph, co-crystal, tautomer, stereoisomer, isotopically labelled derivative, or prodrug thereof, or a pharmaceutical composition of embodiment 73 or 74.
78. The method of embodiment 77, wherein the metabolic disease is type 2 diabetes or metabolic syndrome.
79. The method of embodiment 77, wherein the inflammatory disease is inflammatory bowel disease.
80. The method of embodiment 77, wherein the proliferative disease is cancer.
81. The method of any one of embodiments 77-80, wherein the compound or pharmaceutically acceptable salt, solvate, hydrate, polymorph, co-crystal, tautomer, stereoisomer, isotopically labelled derivative, or prodrug thereof is administered orally, parenterally, intramuscularly, subcutaneously, intravenously, or transdermally.
82. A method comprising contacting a cell, tissue, or biological sample with an effective amount of a compound of any one of embodiments 1-71, or a pharmaceutically acceptable salt, solvate, hydrate, polymorph, co-crystal, tautomer, stereoisomer, isotopically labeled derivative, or prodrug thereof.
83. The method of embodiment 82, wherein the cell is a dendritic cell.
84. The method of any one of embodiments 82-83, wherein the step of contacting modulates an immune response.
85. The method of any one of embodiments 82-84, wherein the step of contacting induces a cytokine release.
86. The method of any one of embodiments 82-85, wherein the step of contacting activates a TLR2 receptor.
87. The method of any one of embodiments 82-86, wherein the step of contacting induces release or production of TNFα, IL-6, IL-10, MCP-1, or combinations thereof.
88. The method of any one of embodiments 82-86, wherein the step of contacting induces release or production of TNFα, IL-6, IL-23A, IL-12B, or combinations thereof.
89. A kit comprising:

a compound of any one of embodiments 1-71, or a pharmaceutically acceptable salt, solvate, hydrate, polymorph, co-crystal, tautomer, stereoisomer, isotopically labeled derivative, or prodrug thereof; or a pharmaceutical composition of embodiments 73 or 74; and

instructions for using the compound, pharmaceutically acceptable salt, solvate, hydrate, polymorph, co-crystal, tautomer, stereoisomer, isotopically labeled derivative, prodrug, or pharmaceutical composition.

90. Use of a compound of the formula:

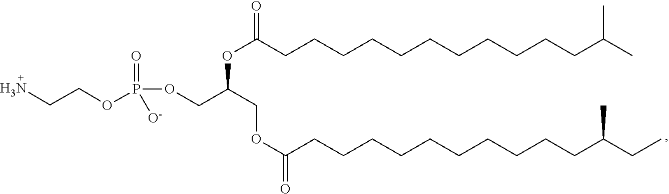 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labeled derivative, or prodrug thereof, or a pharmaceutical composition thereof, for the manufacture of a medicament for treating metabolic disease, inflammatory disease, immune disorder, or proliferative disease.
91. A method of treating a metabolic disease, inflammatory disease, immune disorder, or proliferative disease in a subject in need thereof comprising administering to the subject a therapeutically effective amount of a compound of the formula:

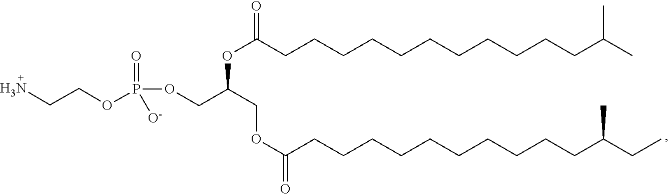 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labelled derivative, or prodrug thereof, or a pharmaceutical composition thereof.
92. The method of embodiment 91, wherein the metabolic disease is type 2 diabetes or metabolic syndrome.
93. The method of embodiment 91, wherein the inflammatory disease is inflammatory bowel disease.
94. The method of embodiment 91, wherein the proliferative disease is cancer.
95. The method of any one of embodiments 91-94, wherein the compound or pharmaceutically acceptable salt, solvate, hydrate, polymorph, co-crystal, tautomer, stereoisomer, isotopically labelled derivative, or prodrug thereof is administered orally, parenterally, intramuscularly, subcutaneously, intravenously, or transdermally.
96. A method comprising contacting a cell, tissue, or biological sample with an effective amount of a compound of the formula:

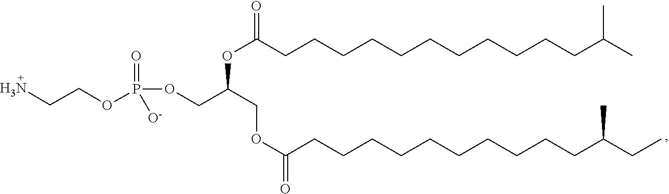 [SMILES: CC[C@H](C)CCCCCCCCCCC(=O)OC[C@@H](COP(=O)([O-])OCCN)OC(=O)CCCCCCCCCCCC(C)C]

or a pharmaceutically acceptable salt, solvate, hydrate, polymorph, co-crystal, tautomer, stereoisomer, isotopically labeled derivative, or prodrug thereof.
97. The method of embodiment 96, wherein the cell is a dendritic cell.
98. The method of any one of embodiments 96 or 97, wherein the step of contacting modulates an immune response.
99. The method of any one of embodiments 96-98, wherein the step of contacting induces a cytokine release.
100. The method of any one of embodiments 96-99, wherein the step of contacting activates a TLR2 receptor.
101. The method of any one of embodiments 95-99, wherein the step of contacting induces release or production of TNFα, IL-6, IL-10, MCP-1, or combinations thereof.
102. The method of any one of embodiments 95-99, wherein the step of contacting induces release or production of TNFα, IL-6, IL-23A, IL-12B, or combinations thereof.
103. A method of treating a metabolic disease, inflammatory disease, immune disorder, or proliferative disease in a subject in need thereof comprising administering to the subject a TLR2 agonist, wherein the amount of the TLR2 agonist administered is approximately 1% of the TLR2 agonist's EC₅₀.
104. The method of embodiment 103, wherein the TLR2 agonist is a compound of any one of embodiments 1-71, or a pharmaceutically acceptable salt, solvate, hydrate, polymorph, co-crystal, tautomer, stereoisomer, isotopically labelled derivative, or prodrug thereof.
105. The method of embodiment 103 or 104, wherein the metabolic disease is type 2 diabetes or metabolic syndrome.
106. The method of embodiment 103 or 104, wherein the inflammatory disease is inflammatory bowel disease.
107. The method of embodiment 103 or 104, wherein the proliferative disease is cancer.
108. The method of any one of embodiments 103-107, wherein the compound or pharmaceutically acceptable salt, solvate, hydrate, polymorph, co-crystal, tautomer, stereoisomer, isotopically labelled derivative, or prodrug thereof is administered orally, parenterally, intramuscularly, subcutaneously, intravenously, or transdermally.
109. The method of any one of embodiments 103-108, wherein the TLR2 agonist modulates an immune response.
110. The method of any one of embodiments 103-109, wherein the TLR2 agonist induces a cytokine release.
111. The method of any one of embodiments 103-110, wherein the TLR2 agonist activates a TLR2 receptor.
112. The method of any one of embodiments 103-111, wherein the TLR2 agonist induces release or production of TNFα, IL-6, IL-10, MCP-1, or combinations thereof.
113. The method of any one of embodiments 103-111, wherein the TLR2 agonist induces release or production of TNFα, IL-6, IL-23A, IL-12B, or combinations thereof.
114. A method comprising administering to a subject an effective amount of a TLR2 agonist, wherein the effective amount of the TLR2 agonist is approximately 1% of the TLR2 agonist's EC₅₀.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 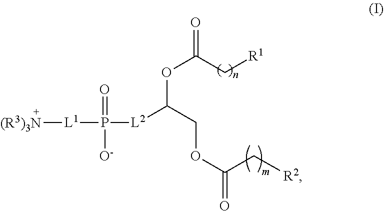
or a pharmaceutically acceptable salt, solvate, hydrate, polymorph, co-crystal, tautomer, stereoisomer, isotopically labeled derivative, or prodrug thereof, wherein:
R¹ is branched or unbranched C₁₋₄ alkyl, or C₃₋₇ carbocyclic;
R² is branched or unbranched C₁₋₄ alkyl, or C₃₋₇ carbocyclic;
at least one of R¹ and R² is branched C₁₋₄ alkyl, or C₃₋₇ carbocyclic;
R³ is —H or branched or unbranched C₁₋₆ alkyl;
L¹ is substituted or unsubstituted C₁₋₅ alkylene, substituted or unsubstituted C₁₋₈ heteroalkylene, substituted or unsubstituted carbocyclylene, substituted or unsubstituted heterocyclylene, substituted or unsubstituted arylene, substituted or unsubstituted heteroarylene, or any combination thereof;
L² is unbranched and unsubstituted C₁₋₆ heteroalkylene, or unbranched and unsubstituted C₁₋₆ alkylene;
n is an integer from 7 to 13, inclusive;
m is an integer from 7 to 13, inclusive; and
provided that the compound is not of the formula:

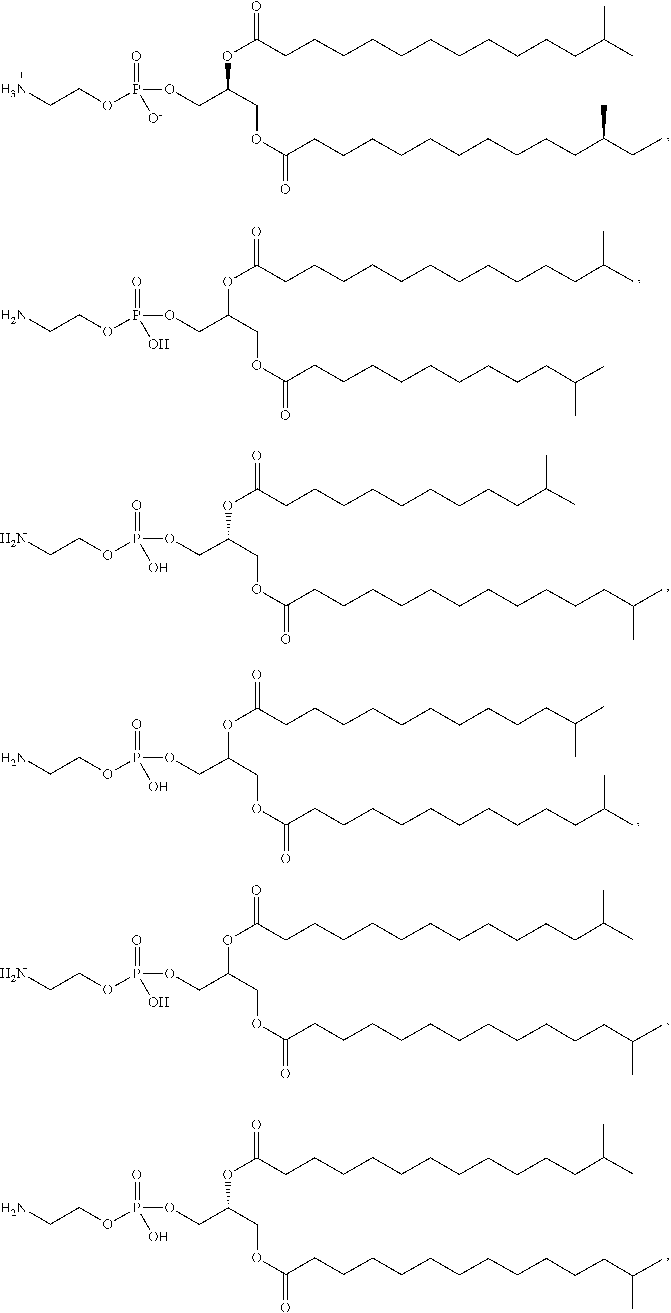

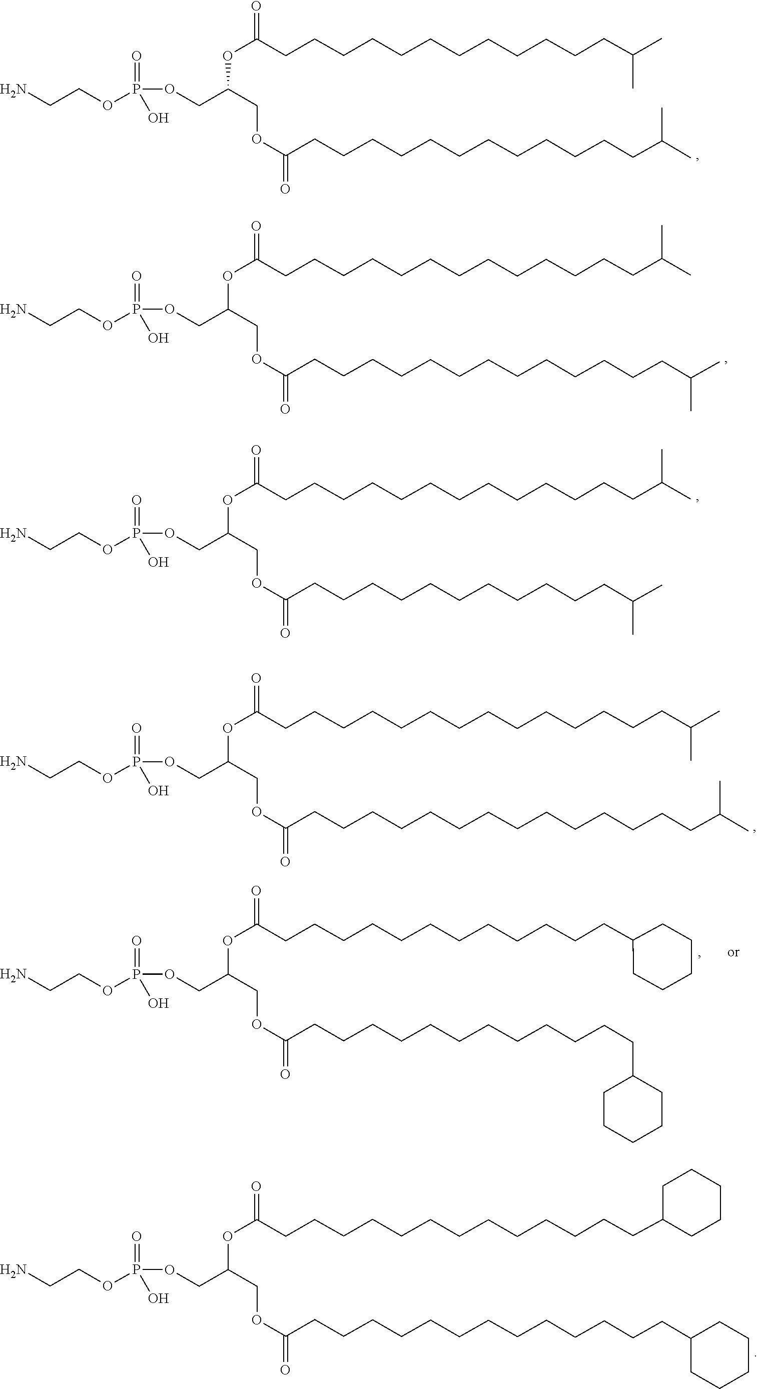

2. The compound of claim 1, wherein the compound is of Formula (Ia): 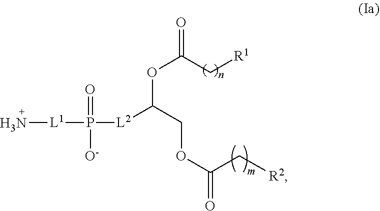
or a pharmaceutically acceptable salt, solvate, hydrate, polymorph, co-crystal, tautomer, stereoisomer, isotopically labeled derivative, or prodrug thereof.

3. The compound of claim 1 or 2, wherein the compound is of Formula (Ib): 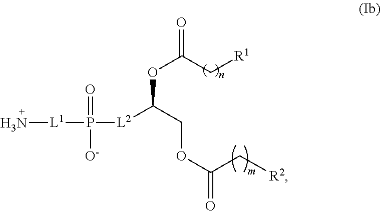
or a pharmaceutically acceptable salt, solvate, hydrate, polymorph, co-crystal, tautomer, stereoisomer, isotopically labeled derivative, or prodrug thereof.

4. The compound of claim 1 or 2, wherein the compound is of Formula (Ic): 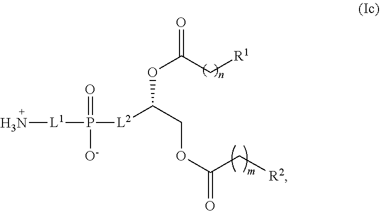
or a pharmaceutically acceptable salt, solvate, hydrate, polymorph, co-crystal, tautomer, stereoisomer, isotopically labeled derivative, or prodrug thereof.

5. The compound of claim 1, wherein the compound is of Formula (Id): 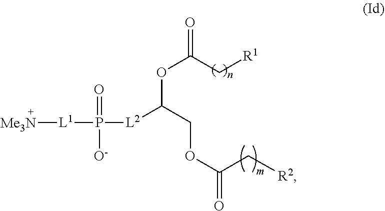
or a pharmaceutically acceptable salt, solvate, hydrate, polymorph, co-crystal, tautomer, stereoisomer, isotopically labeled derivative, or prodrug thereof.

6. The compound of claim 1 or 5, wherein the compound is of Formula (Ie): 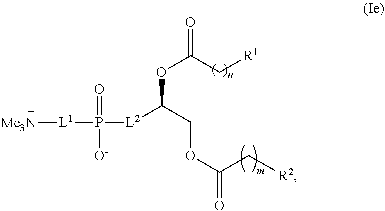
or a pharmaceutically acceptable salt, solvate, hydrate, polymorph, co-crystal, tautomer, stereoisomer, isotopically labeled derivative, or prodrug thereof.

7. The compound of claim 1 or 5, wherein the compound is of Formula (If): 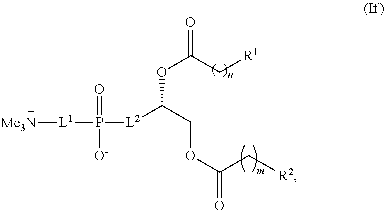
or a pharmaceutically acceptable salt, solvate, hydrate, polymorph, co-crystal, tautomer, stereoisomer, isotopically labeled derivative, or prodrug thereof.

8. The compound of any one of claims 1-7, wherein R¹ and R² are each independently branched C₁₋₄ alkyl, or C₃ 4 carbocyclic.

9. The compound of any one of claims 1-8, wherein R¹ and R² are different.

10. The compound of any one of claims 1-8, wherein R¹ and R² are the same.

11. The compound of any one of claims 1-8, wherein exactly one of R¹ or R² is selected from the group consisting of -Me, -Et, -ⁿPr, and -ⁿBu.

12. The compound of any one of claims 1-11, wherein at least one of R¹ and R² is selected from the group consisting of:

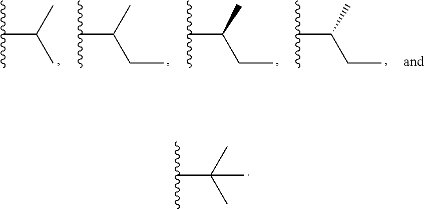

13. The compound of any one of claims 1-11, wherein at least one of R¹ and R² is selected from the group consisting of:

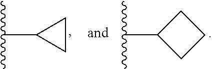

14. The compound of any one of claims 1-13, wherein m and n are different.

15. The compound of any one of claims 1-13, wherein m and n are the same.

16. The compound of any one of claims 1-15, wherein n is 9-13.

17. The compound of any one of claims 1-16, wherein m is 9-13.

18. The compound of any one of claims 1-13 or 15-17, wherein n is 11, and m is 11.

19. The compound of any one of claims 1-14, 16, or 17, wherein n is 10, and m is 11.

20. The compound of any one of claims 1-14, 16, or 17, wherein n is 11, and m is 10.

21. The compound of any one of claims 1-13 or 15-17, wherein n is 10, and m is 10.

22. The compound of any one of claims 1-18 or 20, wherein n is 11, and R¹ is

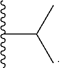 [SMILES: *C(C)C]

23. The compound of any one of claims 1-18 or 20, wherein n is 11, and R¹ is

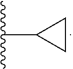 [SMILES: *C1CC1]

24. The compound of any one of claims 1-17, 19, or 21, wherein n is 10, and R¹ is selected from the group consisting of:

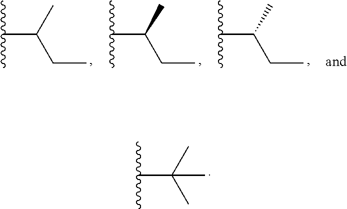

25. The compound of any one of claims 1-17, 19, or 21, wherein n is 10, and R¹ is

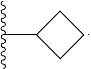 [SMILES: *C1CCC1]

26. The compound of any one of claims 1-19, wherein m is 11, and R² is

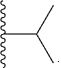 [SMILES: *C(C)C]

27. The compound of any one of claims 1-19, wherein m is 11, and R² is

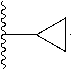 [SMILES: *C1CC1]

28. The compound of any one of claims 1-17 or 20-25, wherein m is 10 and R² is selected from the group consisting of:

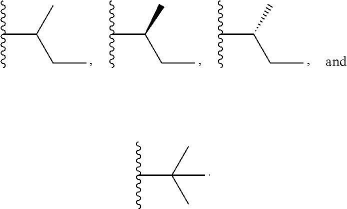

29. The compound of any one of claims 1-17 or 20-25, wherein m is 10 and R² is

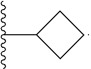 [SMILES: *C1CCC1]

30. The compound of any one of claims 1-17, 19, 21, or 26-29, wherein n is 10, and R¹ is

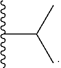 [SMILES: *C(C)C]

31. The compound of any one of claims 1-17, 19, 21, or 26-29, wherein n is 10, and R¹ is

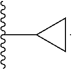 [SMILES: *C1CC1]

32. The compound of any one of claims 1-17 or 26-29, wherein n is 9, and R¹ is selected from the group consisting of:

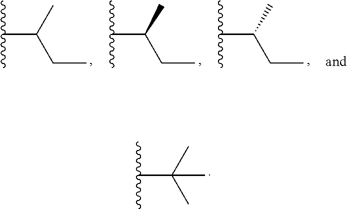

33. The compound of any one of claims 1-17 or 26-29, wherein n is 9, and R¹ is

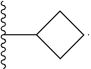 [SMILES: *C1CCC1]

34. The compound of any one of claims 1-17, 20-25, or 30-33, wherein m is 10, and R² is

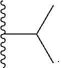 [SMILES: *C(C)C]

35. The compound of any one of claims 1-17, 20-25, or 30-33, wherein m is 10, and R² is

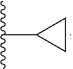 [SMILES: *C1CC1]

36. The compound of any one of claims 1-17, 22-25, or 30-33, wherein m is 9, and R² is selected from the group consisting of:

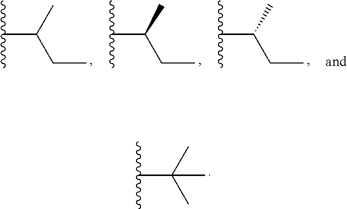

37. The compound of any one of claims 1-17, 22-25, or 30-33, wherein m is 9, and R² is

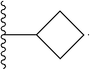 [SMILES: *C1CCC1]

38. The compound of any one of claims 1-17, 26-29, or 34-37, wherein n is 12, and R¹ is

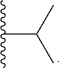 [SMILES: *C(C)C]

39. The compound of any one of claims 1-17, 26-29, or 34-37, wherein n is 12, and R¹ is

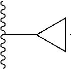 [SMILES: *C1CC1]

40. The compound of any one of claims 1-18, 20, 26-29, or 34-37, wherein n is 11, and R¹ is selected from the group consisting of:

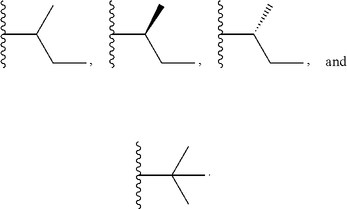

41. The compound of any one of claims 1-18, 20, 26-29, or 34-37, wherein n is 11, and R¹ is

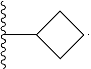 [SMILES: *C1CCC1]

42. The compound of any one of claims 1-17, 22-25, 30-33, or 38-41, wherein m is 12, and R² is

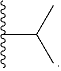 [SMILES: *C(C)C]

43. The compound of any one of claims 1-17, 22-25, 30-33, or 38-41, wherein m is 12, and R² is

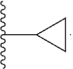 [SMILES: *C1CC1]

44. The compound of any one of claims 1-19, 22-25, 30-33, or 38-41, wherein m is 11, and R² is selected from the group consisting of:

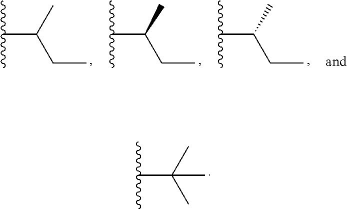

45. The compound of any one of claims 1-19, 22-25, 30-33, or 38-41, wherein m is 11, and R² is

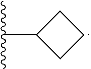 [SMILES: *C1CCC1]

46. The compound of any one of claims 1-45, wherein L¹ is unbranched and unsubstituted C₁₋₆ heteroalkylene comprising one or more oxygen atoms.

47. The compound of any one of claims 1-45, wherein L¹ is unbranched and unsubstituted C₁₋₆ alkylene.

48. The compound of any one of claims 1-45, wherein L¹ is selected from the group consisting of: 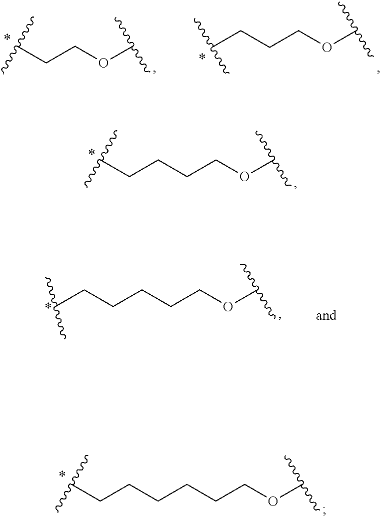 [SMILES: *CCO* ; *CCCO* ; *CCCCO* ; *CCCCCO* ; *CCCCCCO*]
wherein the attachment point labeled with “*” is attached to the nitrogen atom.

49. The compound of any one of claims 1-45 or 47, wherein L¹ is selected from the group consisting of:

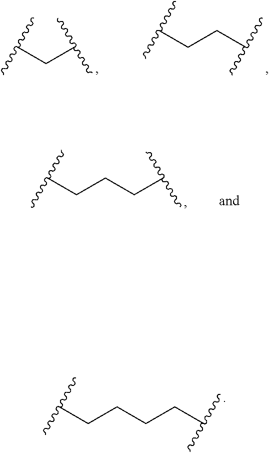

50. The compound of any one of claims 1-49, wherein L² is selected from the group consisting of: 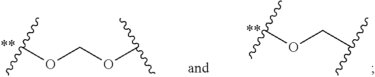
wherein the attachment point labeled with “**” is attached to the phosphorous atom.

51. The compound of any one of claims 1-46, 48, or 50, wherein L¹ is unbranched and unsubstituted C₁₋₆ heteroalkylene comprising one or more oxygen atoms, and L² is 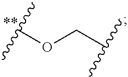 [SMILES: *CO*]
wherein the attachment point labeled with “**” is attached to the phosphorous atom.

52. The compound of any one of claims 1-46, 48, or 50, wherein L¹ is 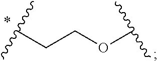 [SMILES: *CCO*]
wherein the attachment point labeled with “*” is attached to the nitrogen atom, and L² is 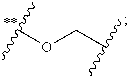 [SMILES: *CO*]
wherein the attachment point labeled with “**” is attached to the phosphorous atom.

53. The compound of any one of claims 1-46, 48, or 50, wherein L¹ is 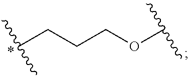 [SMILES: *CCCO*]
wherein the attachment point labeled with “*” is attached to the nitrogen atom, and L² is 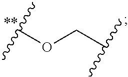 [SMILES: *CO*]
wherein the attachment point labeled with “**” is attached to the phosphorous atom.

54. The compound of any one of claims 1-46, 48, or 50, wherein L¹ is 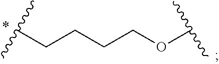 [SMILES: *CCCCO*]
wherein the attachment point labeled with “*” is attached to the nitrogen atom, and L² is 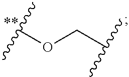 [SMILES: *CO*]
wherein the attachment point labeled with “**” is attached to the phosphorous atom.

55. The compound of claim 1, wherein the compound is selected from the group consisting of: 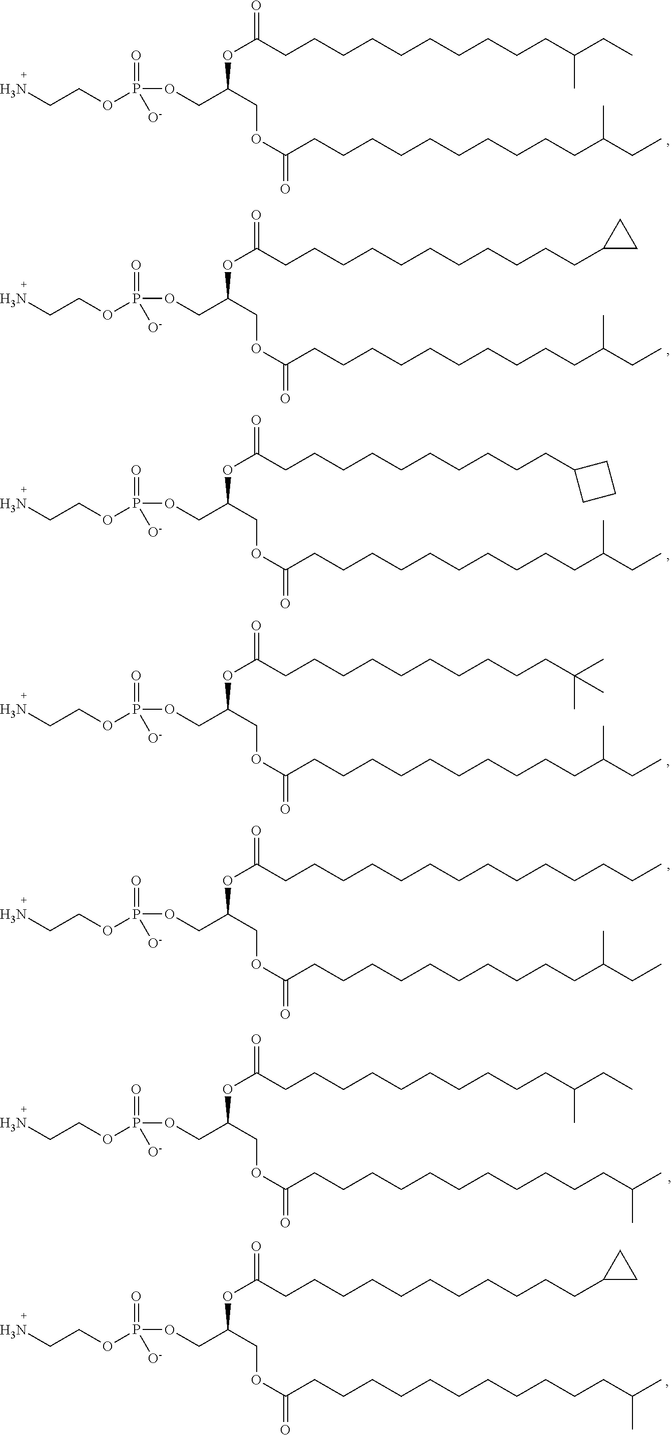 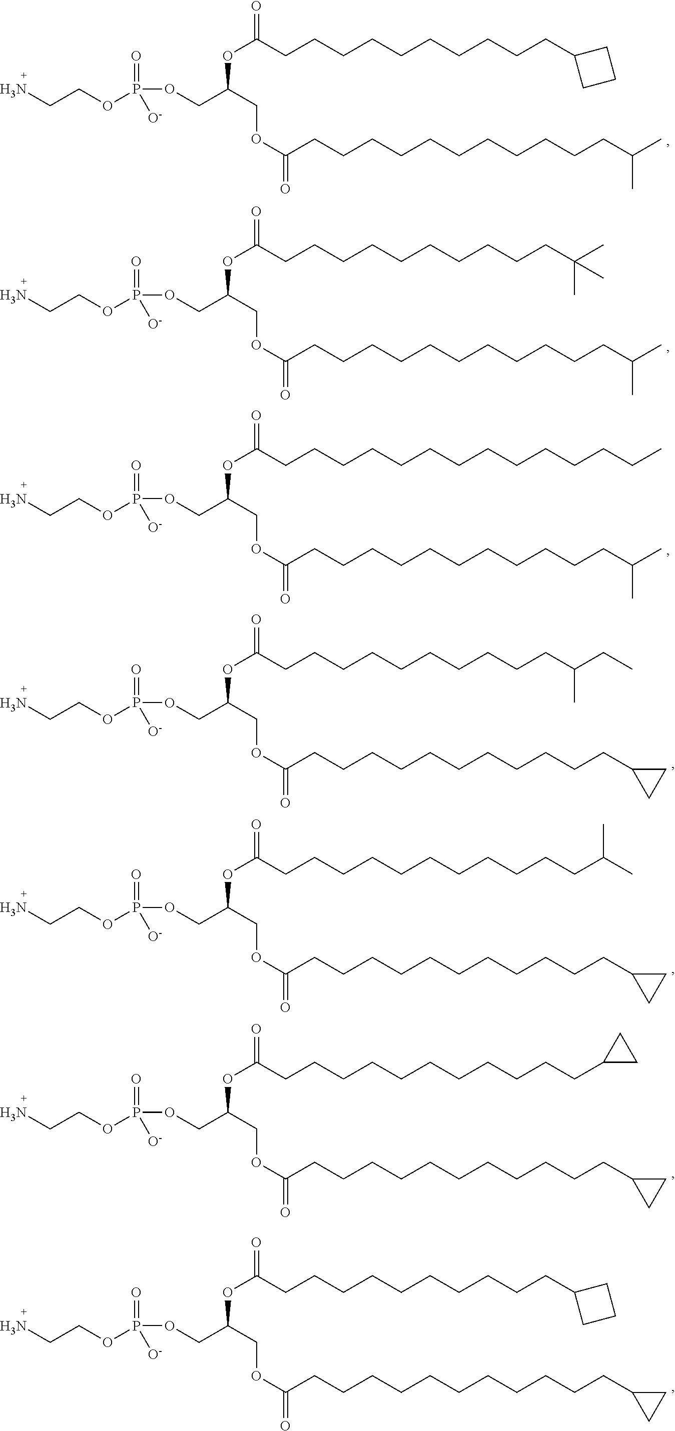 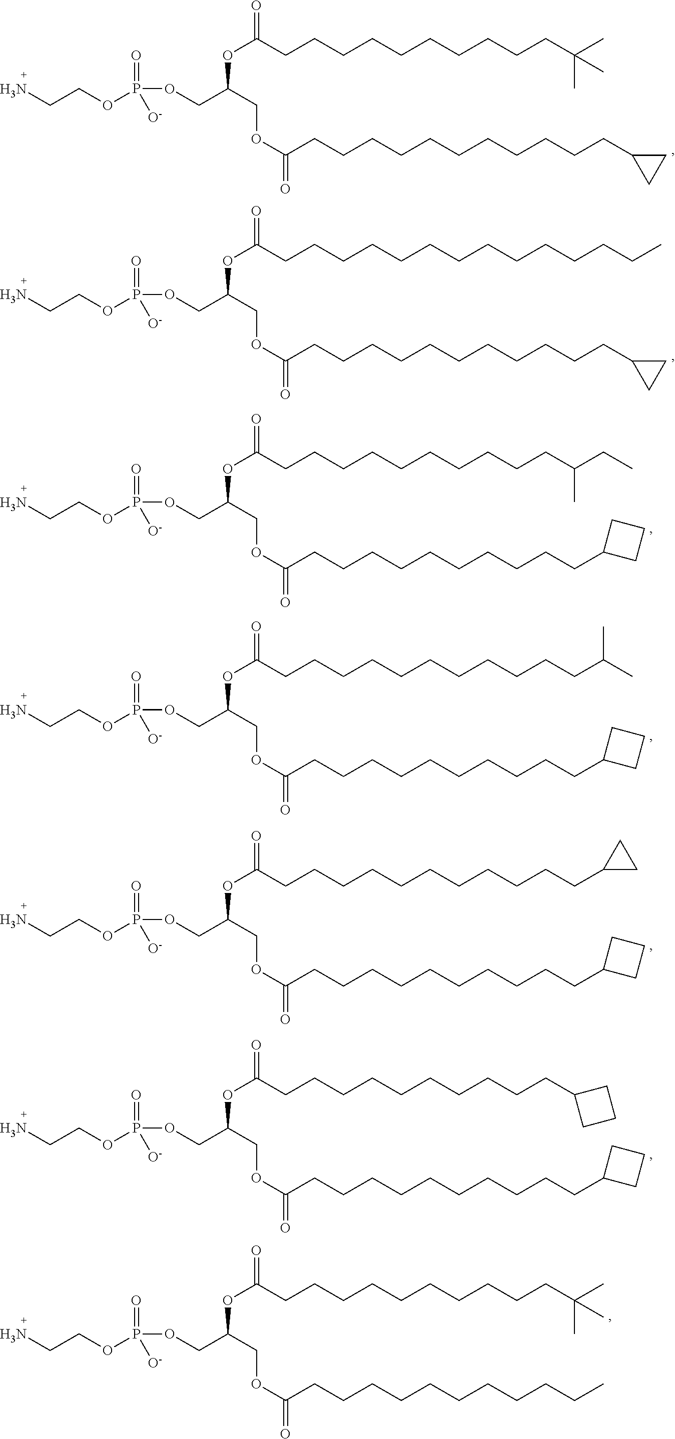 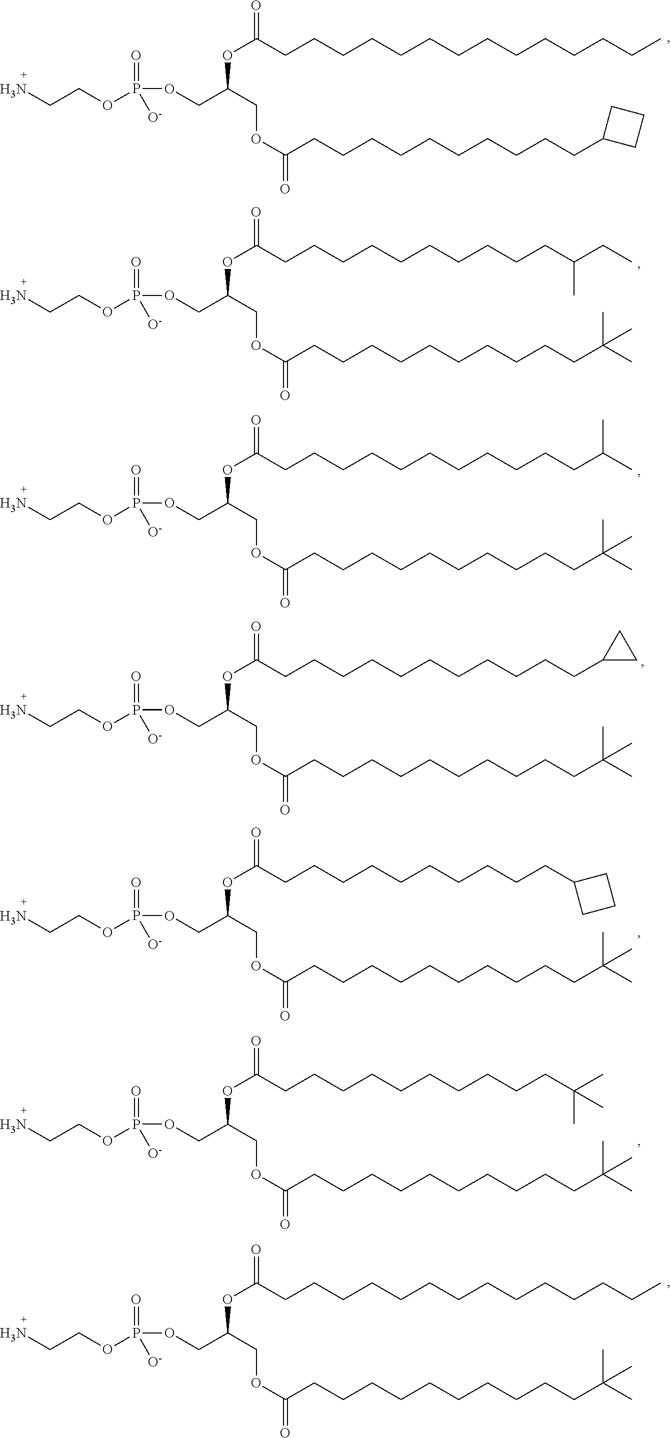 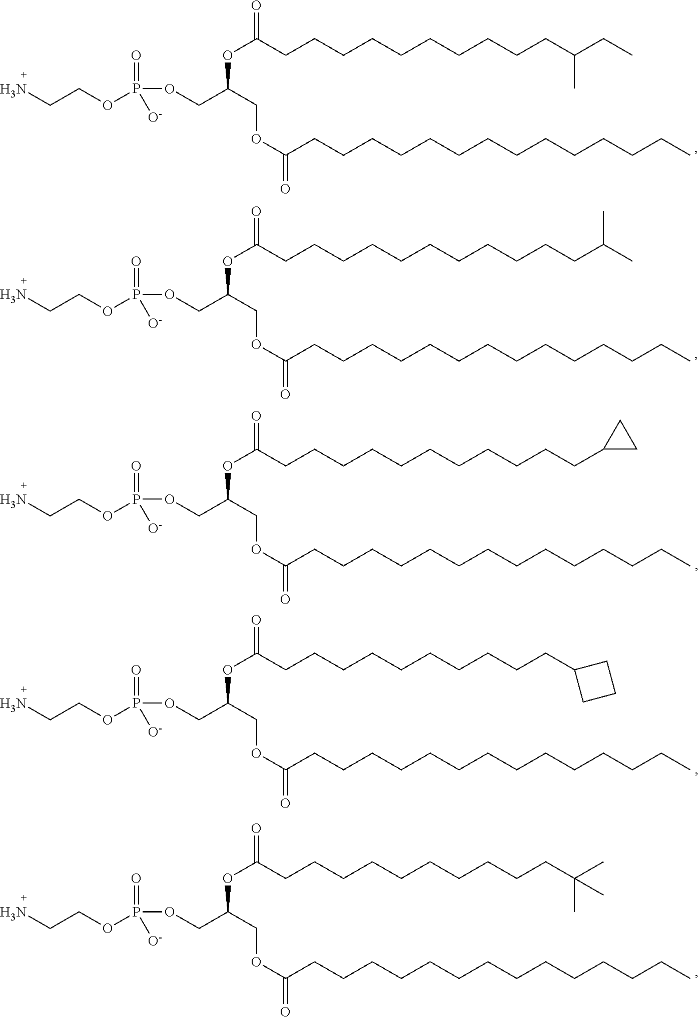 [SMILES: CCCCCCCCCCCCCCC(=O)OC[C@@H](COP(=O)([O-])OCCN)OC(=O)CCCCCCCCCCC(C)CC ; CCCCCCCCCCCCCCC(=O)OC[C@@H](COP(=O)([O-])OCCN)OC(=O)CCCCCCCCCCCC(C)C ; CCCCCCCCCCCCCCC(=O)OC[C@@H](COP(=O)([O-])OCCN)OC(=O)CCCCCCCCCCCC1CC1 ; CCCCCCCCCCCCCCC(=O)OC[C@@H](COP(=O)([O-])OCCN)OC(=O)CCCCCCCCCCC1CCC1 ; CCCCCCCCCCCCCCC(=O)OC[C@@H](COP(=O)([O-])OCCN)OC(=O)CCCCCCCCCCC(C)(C)C]
and pharmaceutically acceptable salts, hydrates, polymorphs, co-crystals, tautomers, stereoisomers, isotopically labeled derivatives, and prodrugs thereof.

56. The compound of claim 1, having the structure: 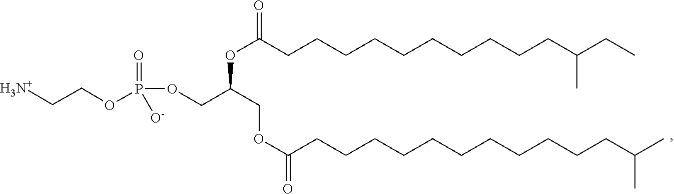 [SMILES: CCC(C)CCCCCCCCCCC(=O)O[C@@H](COC(=O)CCCCCCCCCCCC(C)C)COP(=O)([O-])OCC[NH3+]]
or a pharmaceutically acceptable salt, hydrate, polymorph, co-crystal, tautomer, stereoisomer, isotopically labeled derivative, or prodrug thereof.

57. The compound of claim 1, having the structure: 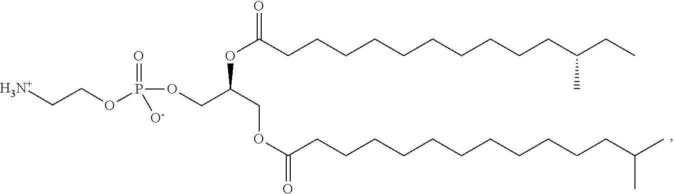 [SMILES: CC[C@H](C)CCCCCCCCCCC(=O)O[C@@H](COC(=O)CCCCCCCCCCCC(C)C)COP(=O)([O-])OCC[NH3+]]
or a pharmaceutically acceptable salt, hydrate, polymorph, co-crystal, tautomer, stereoisomer, isotopically labeled derivative, or prodrug thereof.

58. A compound of Formula (II): 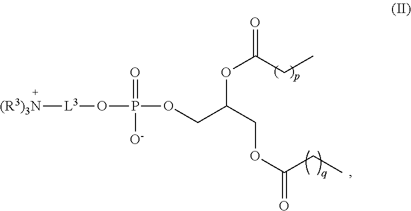
or a pharmaceutically acceptable salt, solvate, hydrate, polymorph, co-crystal, tautomer, stereoisomer, isotopically labeled derivative, or prodrug thereof, wherein:
L³ is substituted or unsubstituted C₃₋₈ alkylene, substituted or unsubstituted C₃₋₈ heteroalkylene, substituted or unsubstituted carbocyclylene, substituted or unsubstituted heterocyclylene, substituted or unsubstituted arylene, substituted or unsubstituted heteroarylene, or any combination thereof;
R³ is —H or branched or unbranched C₁₋₆ alkyl;
p is an integer from 12 to 14, inclusive; and
q is an integer from 12 to 14, inclusive;
provided that the compound is not of the formula:

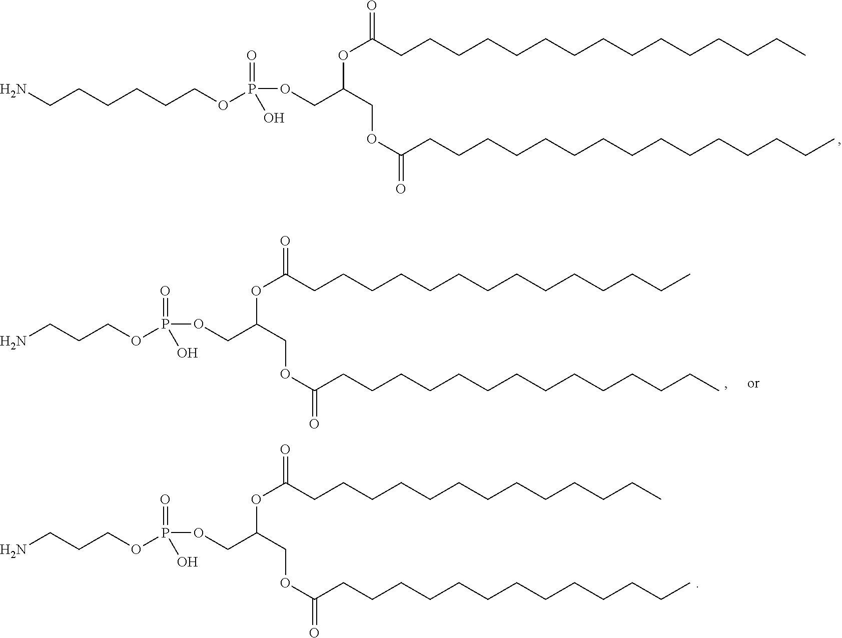

59. The compound of claim 58, wherein the compound is of Formula (IIa): 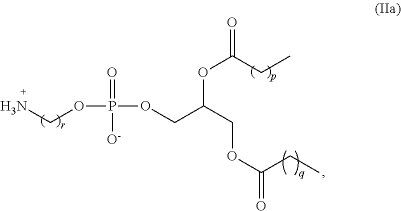
or a pharmaceutically acceptable salt, solvate, hydrate, polymorph, co-crystal, tautomer, stereoisomer, isotopically labeled derivative, or prodrug thereof, wherein r is an integer from 3 to 8, inclusive.

60. The compound of claim 58 or 59, wherein the compound is of Formula (IIb): 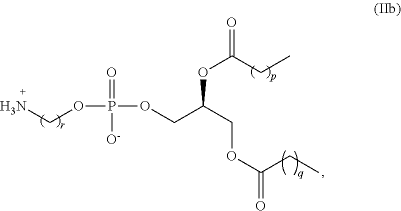
or a pharmaceutically acceptable salt, solvate, hydrate, polymorph, co-crystal, tautomer, stereoisomer, isotopically labeled derivative, or prodrug thereof, wherein r is an integer from 3 to 8, inclusive.

61. The compound of claim 58 or 59, wherein the compound is of Formula (Iic): 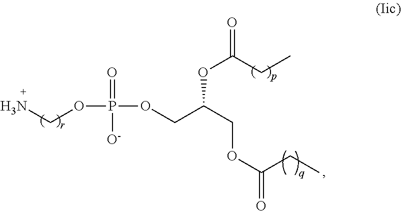
or a pharmaceutically acceptable salt, solvate, hydrate, polymorph, co-crystal, tautomer, stereoisomer, isotopically labeled derivative, or prodrug thereof, wherein r is an integer from 3 to 8, inclusive.

62. The compound of claim 58, wherein the compound is of Formula (Iid): 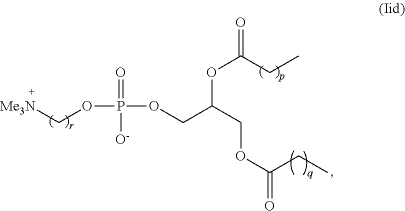
or a pharmaceutically acceptable salt, solvate, hydrate, polymorph, co-crystal, tautomer, stereoisomer, isotopically labeled derivative, or prodrug thereof, wherein r is an integer from 3 to 8, inclusive.

63. The compound of claim 58 or 62, wherein the compound is of Formula (IIe):

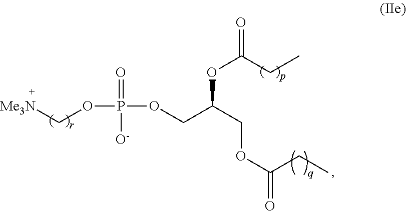

64. The compound of claim 58 or 62, wherein the compound is of Formula (IIe): 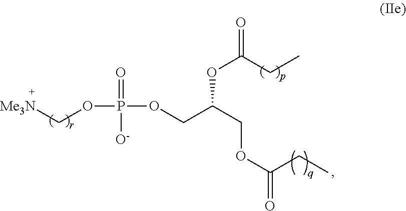
or a pharmaceutically acceptable salt, solvate, hydrate, polymorph, co-crystal, tautomer, stereoisomer, isotopically labeled derivative, or prodrug thereof, wherein r is an integer from 3 to 8, inclusive.

65. The compound of any one of claims 58-64, wherein L³ is selected from the group consisting of:

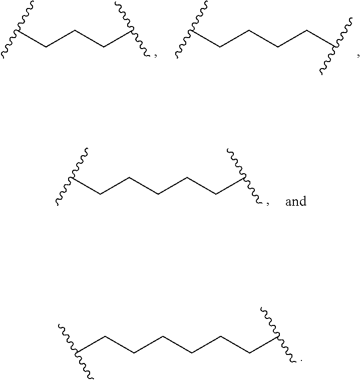 [SMILES: *CCC* ; *CCCC* ; *CCCCC* ; *CCCCCC*]

66. The compound of any one of claims 58-65, wherein p and q are the same.

67. The compound of any one of claims 58-65, wherein p and q are different.

68. The compound of any one of claims 58-66, wherein p is 12; and q is 12.

69. The compound of any one of claims 58-66, wherein p is 13; and q is 13.

70. The compound of any one of claims 58-66, wherein p is 14; and q is 14.

71. The compound of claim 58, wherein the compound is selected from the group consisting of: 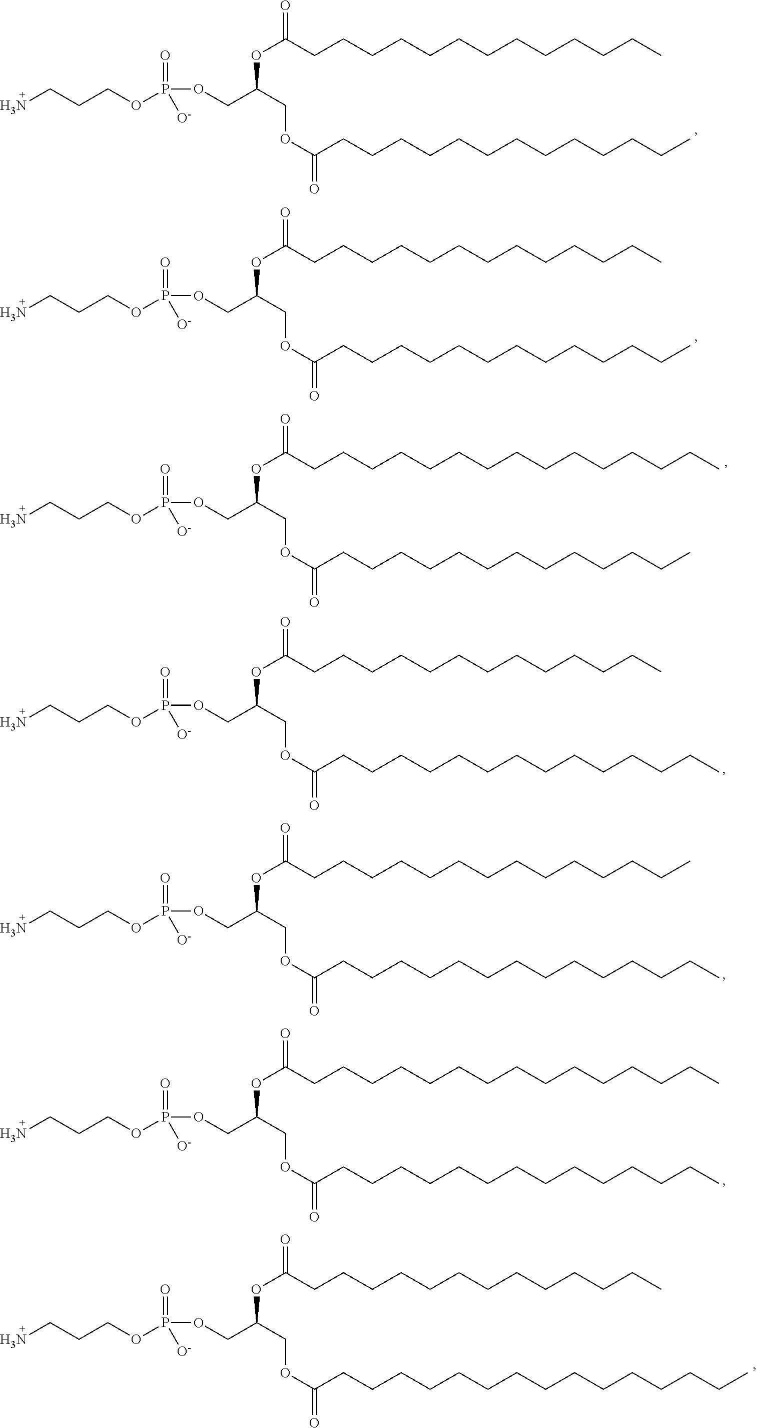 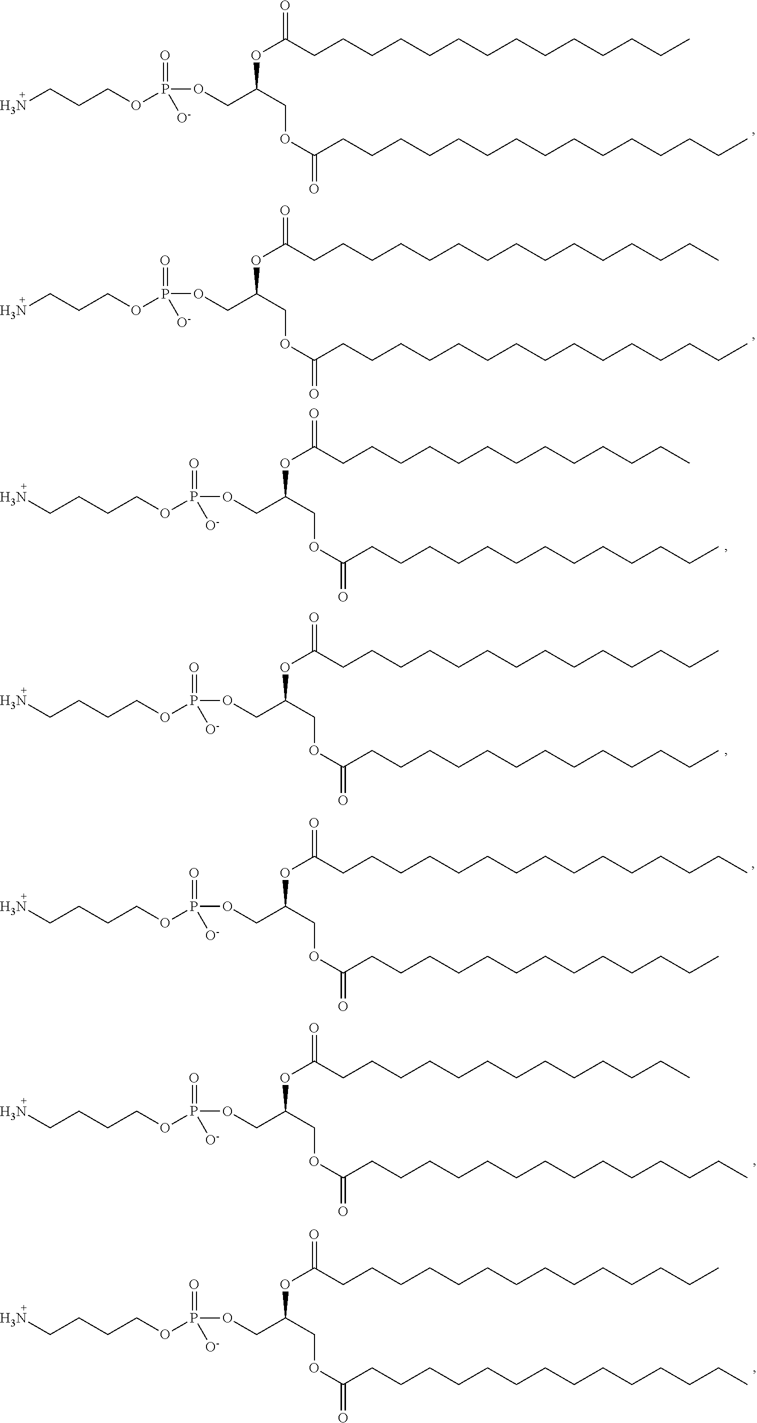 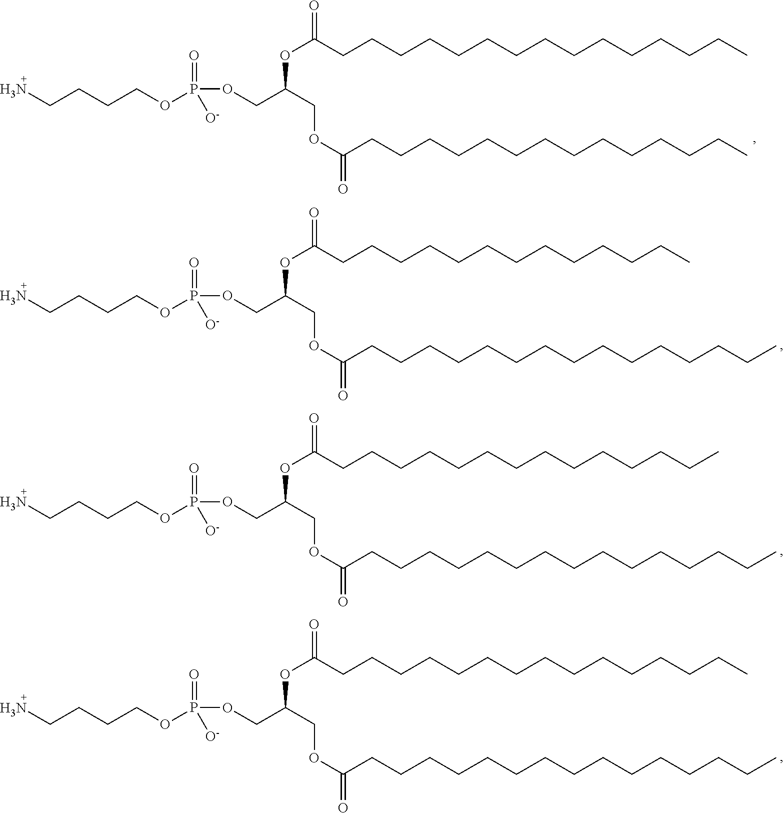 [SMILES: CCCCCCCCCCCCCCCC(=O)O[C@@H](COC(=O)CCCCCCCCCCCCCC)COP(=O)([O-])OCCCCN ; CCCCCCCCCCCCCCCC(=O)OC[C@@H](COP(=O)([O-])OCCCCN)OC(=O)CCCCCCCCCCCCC ; CCCCCCCCCCCCCCCC(=O)OC[C@@H](COP(=O)([O-])OCCCCN)OC(=O)CCCCCCCCCCCCCC ; CCCCCCCCCCCCCCCC(=O)OC[C@@H](COP(=O)([O-])OCCCCN)OC(=O)CCCCCCCCCCCCCCC]
and pharmaceutically acceptable salts, hydrates, polymorphs, co-crystals, tautomers, stereoisomers, isotopically labeled derivatives, and prodrugs thereof.

72. A pharmaceutical composition comprising a compound of any one of the claims 1-71, or a pharmaceutically acceptable salt, solvate, hydrate, polymorph, co-crystal, tautomer, stereoisomer, isotopically labeled derivative, or prodrug thereof; and a pharmaceutically acceptable excipient.

73. The pharmaceutical composition of claim 72, further comprising an additional pharmaceutical agent.

74. A compound of any one of claims 1-71, or a pharmaceutically acceptable salt, solvate, hydrate, polymorph, co-crystal, tautomer, stereoisomer, isotopically labeled derivative, or prodrug thereof, or a pharmaceutical composition of claim 73 or 74, for use in treating a disease, disorder, or condition, wherein the disease, disorder, or condition is a metabolic disease, inflammatory disease, immune disorder, or proliferative disease.

75. A compound of any one of claims 1-71, or a pharmaceutically acceptable salt, solvate, hydrate, polymorph, co-crystal, tautomer, stereoisomer, isotopically labeled derivative, or prodrug thereof, or a pharmaceutical composition of claim 73 or 74, for use as a vaccine adjuvant.

76. Use of a compound of any one of claims 1-71, or a pharmaceutically acceptable salt, solvate, hydrate, polymorph, co-crystal, tautomer, stereoisomer, isotopically labeled derivative, or prodrug thereof, or a pharmaceutical composition of claim 73 or 74, for the manufacture of a medicament for treating metabolic disease, inflammatory disease, immune disorder, or proliferative disease.

77. A method of treating a metabolic disease, inflammatory disease, immune disorder, or proliferative disease in a subject in need thereof comprising administering to the subject a therapeutically effective amount of a compound of any one of claims 1-71, or a pharmaceutically acceptable salt, solvate, hydrate, polymorph, co-crystal, tautomer, stereoisomer, isotopically labelled derivative, or prodrug thereof, or a pharmaceutical composition of claim 73 or 74.

78. The method of claim 77, wherein the metabolic disease is type 2 diabetes or metabolic syndrome.

79. The method of claim 77, wherein the inflammatory disease is inflammatory bowel disease.

80. The method of claim 77, wherein the proliferative disease is cancer.

81. The method of any one of claims 77-80, wherein the compound or pharmaceutically acceptable salt, solvate, hydrate, polymorph, co-crystal, tautomer, stereoisomer, isotopically labelled derivative, or prodrug thereof is administered orally, parenterally, intramuscularly, subcutaneously, intravenously, or transdermally.

82. A method comprising contacting a cell, tissue, or biological sample with an effective amount of a compound of any one of claims 1-71, or a pharmaceutically acceptable salt, solvate, hydrate, polymorph, co-crystal, tautomer, stereoisomer, isotopically labeled derivative, or prodrug thereof.

83. The method of claim 82, wherein the cell is a dendritic cell.

84. The method of any one of claims 82-83, wherein the step of contacting modulates an immune response.

85. The method of any one of claims 82-84, wherein the step of contacting induces a cytokine release.

86. The method of any one of claims 82-85, wherein the step of contacting activates a TLR2 receptor.

87. The method of any one of claims 82-86, wherein the step of contacting induces release or production of TNFα, IL-6, IL-10, MCP-1, or combinations thereof.

88. The method of any one of claims 82-86, wherein the step of contacting induces release or production of TNFα, IL-6, IL-23A, IL-12B, or combinations thereof.

89. A kit comprising:
a compound of any one of claims 1-71, or a pharmaceutically acceptable salt, solvate, hydrate, polymorph, co-crystal, tautomer, stereoisomer, isotopically labeled derivative, or prodrug thereof; or a pharmaceutical composition of claims 73 or 74; and
instructions for using the compound, pharmaceutically acceptable salt, solvate, hydrate, polymorph, co-crystal, tautomer, stereoisomer, isotopically labeled derivative, prodrug, or pharmaceutical composition.

90. Use of a compound of the formula: 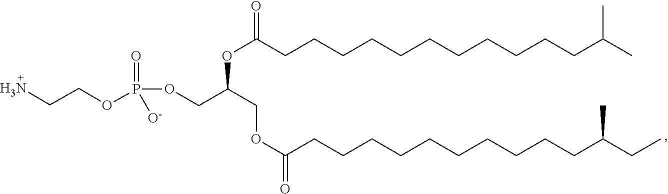 [SMILES: CC[C@H](C)CCCCCCCCCCC(=O)OC[C@@H](COP(=O)([O-])OCCN)OC(=O)CCCCCCCCCCCC(C)C]
or a pharmaceutically acceptable salt, solvate, hydrate, polymorph, co-crystal, tautomer, stereoisomer, isotopically labeled derivative, or prodrug thereof, or a pharmaceutical composition thereof, for the manufacture of a medicament for treating metabolic disease, inflammatory disease, immune disorder, or proliferative disease.

91. A method of treating a metabolic disease, inflammatory disease, immune disorder, or proliferative disease in a subject in need thereof comprising administering to the subject a therapeutically effective amount of a compound of the formula: 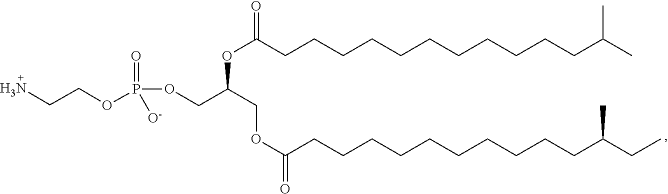 [SMILES: CC[C@H](C)CCCCCCCCCCC(=O)OC[C@@H](COP(=O)([O-])OCCN)OC(=O)CCCCCCCCCCCC(C)C]
or a pharmaceutically acceptable salt, solvate, hydrate, polymorph, co-crystal, tautomer, stereoisomer, isotopically labelled derivative, or prodrug thereof, or a pharmaceutical composition thereof.

92. The method of claim 91, wherein the metabolic disease is type 2 diabetes or metabolic syndrome.

93. The method of claim 91, wherein the inflammatory disease is inflammatory bowel disease.

94. The method of claim 91, wherein the proliferative disease is cancer.

95. The method of any one of claims 91-94, wherein the compound or pharmaceutically acceptable salt, solvate, hydrate, polymorph, co-crystal, tautomer, stereoisomer, isotopically labelled derivative, or prodrug thereof is administered orally, parenterally, intramuscularly, subcutaneously, intravenously, or transdermally.

96. A method comprising contacting a cell, tissue, or biological sample with an effective amount of a compound of the formula: 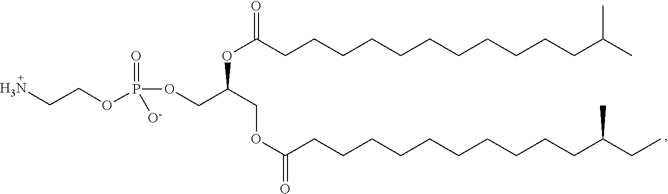 [SMILES: CC[C@H](C)CCCCCCCCCCC(=O)OC[C@@H](COP(=O)([O-])OCCN)OC(=O)CCCCCCCCCCCC(C)C]
or a pharmaceutically acceptable salt, solvate, hydrate, polymorph, co-crystal, tautomer, stereoisomer, isotopically labeled derivative, or prodrug thereof.

97. The method of claim 96, wherein the cell is a dendritic cell.

98. The method of any one of claims 96 or 97, wherein the step of contacting modulates an immune response.

99. The method of any one of claims 96-98, wherein the step of contacting induces a cytokine release.

100. The method of any one of claims 96-99, wherein the step of contacting activates a TLR2 receptor.

101. The method of any one of claims 95-99, wherein the step of contacting induces release or production of TNFα, IL-6, IL-10, MCP-1, or combinations thereof.

102. The method of any one of claims 95-99, wherein the step of contacting induces release or production of TNFα, IL-6, IL-23A, IL-12B, or combinations thereof.

103. A method of treating a metabolic disease, inflammatory disease, immune disorder, or proliferative disease in a subject in need thereof comprising administering to the subject a TLR2 agonist, wherein the amount of the TLR2 agonist administered is approximately 1% of the TLR2 agonist's EC₅₀.

104. The method of claim 103, wherein the TLR2 agonist is a compound of any one of claims 1-71, or a pharmaceutically acceptable salt, solvate, hydrate, polymorph, co-crystal, tautomer, stereoisomer, isotopically labelled derivative, or prodrug thereof.

105. The method of claim 103 or 104, wherein the metabolic disease is type 2 diabetes or metabolic syndrome.

106. The method of claim 103 or 104, wherein the inflammatory disease is inflammatory bowel disease.

107. The method of claim 103 or 104, wherein the proliferative disease is cancer.

108. The method of any one of claims 103-107, wherein the compound or pharmaceutically acceptable salt, solvate, hydrate, polymorph, co-crystal, tautomer, stereoisomer, isotopically labelled derivative, or prodrug thereof is administered orally, parenterally, intramuscularly, subcutaneously, intravenously, or transdermally.

109. The method of any one of claims 103-108, wherein the TLR2 agonist modulates an immune response.

110. The method of any one of claims 103-109, wherein the TLR2 agonist induces a cytokine release.

111. The method of any one of claims 103-110, wherein the TLR2 agonist activates a TLR2 receptor.

112. The method of any one of claims 103-111, wherein the TLR2 agonist induces release or production of TNFα, IL-6, IL-10, MCP-1, or combinations thereof.

113. The method of any one of claims 103-111, wherein the TLR2 agonist induces release or production of TNFα, IL-6, IL-23A, IL-12B, or combinations thereof.

114. A method comprising administering to a subject an effective amount of a TLR2 agonist, wherein the effective amount of the TLR2 agonist is approximately 1% of the TLR2 agonist's EC₅₀.
